SEQUENCE LISTING (128 sequences)

SEQ ID NO: 1 - DNA, 1061 bp, Homo sapiens
tgccctgcgcccgcaacccgagccgcacccgccgcggacggagcccatgcgcggggcgaaccgcgcgcccccgcccccgccccgccccggcctcggccccggccctggccccgggggcagtcgcgcctgtgaacggtggggcaggagaccctgtaggaggaccccgggccgcaggcccctgaggagcgatgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctgacgcagcacaagctcaggacatggaggtgccggatgcaggaaggaggtgcagacggaaggaggaggaaggaaggacggaagcaaggaaggaaggaagggctgctggagcccagtcaccccgggaccgtgggccgaggtgactgcagaccctcccagggaggctgtgcacagactgtcttgaacatcccaaatgccaccggaaccccagcccttagctcccctcccaggcctctgtgggcccttgtcgggcacagatgggatcacagtaaattattggatggtcttgaaaaaaaaaaaaaaaaaa

SEQ ID NO: 2 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 3 - DNA, 1251 bp, Homo sapiens
tgccctgcgcccgcaacccgagccgcacccgccgcggacggagcccatgcgcggggcgaaccgcgcgcccccgcccccgccccgccccggcctcggccccggccctggccccgggggcagtcgcgcctgtgaacggtggggcaggagaccctgtaggaggaccccgggccgcaggcccctgaggagcgatgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggcagccgctctggctctagctccagctccgggaccctctgggaccccccgggacccatgtgacccagcggcccctcgcgctggagtggaggatgccttctacacgttggtgcgtgagatccggcagcacaagctgcggaagctgaaccctcctgatgagagtggccccggctgcatgagctgcaagtgtgtgctctcctgacgcaggtgagggggactcccagggcggccgccacgcccaccggatgaccccggctccccgcccctgccggtctcctggcctgcggtcagcagcctcccttgtgccccgcccagcacaagctcaggacatggaggtgccggatgcaggaaggaggtgcagacggaaggaggaggaaggaaggacggaagcaaggaaggaaggaagggctgctggagcccagtcaccccgggaccgtgggccgaggtgactgcagaccctcccagggaggctgtgcacagactgtcttgaacatcccaaatgccaccggaaccccagcccttagctcccctcccaggcctctgtgggcccttgtcgggcacagatgggatcacagtaaattattggatggtcttgaaaaaaaaaaaaaaaaaa

SEQ ID NO: 4 - protein, 170 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGSRSGSSSSSGTLWDPPGPM

SEQ ID NO: 5 - DNA, 1169 bp, Homo sapiens
tgccctgcgcccgcaacccgagccgcacccgccgcggacggagcccatgcgcggggcgaaccgcgcgcccccgcccccgccccgccccggcctcggccccggccctggccccgggggcagtcgcgcctgtgaacggtggggcaggagaccctgtaggaggaccccgggccgcaggcccctgaggagcgatgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctgacgcaggtgagggggactcccagggcggccgccacgcccaccggatgaccccggctccccgcccctgccggtctcctggcctgcggtcagcagcctcccttgtgccccgcccagcacaagctcaggacatggaggtgccggatgcaggaaggaggtgcagacggaaggaggaggaaggaaggacggaagcaaggaaggaaggaagggctgctggagcccagtcaccccgggaccgtgggccgaggtgactgcagaccctcccagggaggctgtgcacagactgtcttgaacatcccaaatgccaccggaaccccagcccttagctcccctcccaggcctctgtgggcccttgtcgggcacagatgggatcacagtaaattattggatggtcttgaaaaaaaaaaaaaaaaaa

SEQ ID NO: 6 - protein, 189 aa, Homo sapiens; identical to SEQ ID NO: 2

SEQ ID NO: 7 - DNA, 1246 bp, Rattus norvegicus
gcccggcgcccgcagcccgagccgcacccgccgcggacggagcccatgcgcgggcccagtcggcgcccgtccgcgcccccgccctgccccggccccggccccgggggcagtcgcgccagcaagcggtggggcaggagctcctggtttggcagcccctgtagaagcgatgacagaatacaagcttgtggtggtgggcgctggaggcgtgggaaagagtgccctgaccatccagctgatccagaaccattttgtggacgagtatgatcccactatagaggactcctaccggaaacaggtagtcattgatggggagacgtgtttactggacatcttagacacagcaggtcaagaagagtatagtgccatgcgggaccagtacatgcgcacaggggagggcttcctctgtgtatttgccatcaacaacaccaagtcctttgaagacatccatcagtacagggagcagatcaagcgggtgaaagattcagatgatgtgccaatggtgctggtgggcaacaagtgtgacctggccgctcgcactgttgagtctcggcaggcccaggaccttgctcgcagctatggcatcccctacattgaaacatcagccaagacccggcagggtgtggaggatgccttctacacactagtacgtgagattcggcagcataaactgcggaaactgaacccgcctgatgagagtggccctggctgcatgagctgcaagtgtgtgctgtcctgacaccaggctcaggacgaggaggtgccggatgcagggaggaggtgctgtcggaaggaaggaaagaggagggaaggaaggaaacgatgctggagccagtccagtccagggatggtggacagatgtgaccaagaccttcgcatggacaatttgaacagactgtcatgaactatccctgttgccactggcacccaagtcctccgcccctctcagctcccttgggcgcctatgagggcacatgttgaatcacagtaaattatttgatggtcttgacttgtctctggctggaagtaggaggtgtagtgcctgtggcctcacagaagatactggggacctgggatctatcatctgccttgttcctgtttctagaagaggctggggaggctggcctgggacctgggactgtgcagcccttctcttctgcctgcctcaactgtcttgtgtttcctgcagaaagttataaataacacaagaatagtcaggtgatatgaaaaaaaaaaaaaaaaaaaaaaa

SEQ ID NO: 8 - protein, 189 aa, Rattus norvegicus; identical to SEQ ID NO: 2

SEQ ID NO: 9 - DNA, 1392 bp, Rattus norvegicus
gcccggcgcccgcagcccgagccgcacccgccgcggacggagcccatgcgcgggcccagtcggcgcccgtccgcgcccccgccctgccccggccccggccccgggggcagtcgcgccagcaagcggtggggcaggagctcctggtttggcagcccctgtagaagcgatgacagaatacaagcttgtggtggtgggcgctggaggcgtgggaaagagtgccctgaccatccagctgatccagaaccattttgtggacgagtatgatcccactatagaggactcctaccggaaacaggtagtcattgatggggagacgtgtttactggacatcttagacacagcaggtcaagaagagtatagtgccatgcgggaccagtacatgcgcacaggggagggcttcctctgtgtatttgccatcaacaacaccaagtcctttgaagacatccatcagtacagggagcagatcaagcgggtgaaagattcagatgatgtgccaatggtgctggtgggcaacaagtgtgacctggccgctcgcactgttgagtctcggcaggcccaggaccttgctcgcagctatggcatcccctacattgaaacatcagccaagacccggcagggtgtggaggatgccttctacacactagtacgtgagattcggcagcataaactgcggaaactgaacccgcctgatgagagtggccctggctgcatgagctgcaagtgtgtgctgtcctgacaccaggtgaggcagggaccagcaagacatctggggcagtgacctcagctagccagatgaacttcatatccactttgatgtccctgctcccccaattctgccaatccccctgcctgcagtcagtcatgtcctttgtgcccgtcccggcacaggctcaggacgaggaggtgccggatgcagggaggaggtgctgtcggaaggaaggaaagaggagggaaggaaggaaacgatgctggagccagtccagtccagggatggtggacagatgtgaccaagaccttcgcatggacaatttgaacagactgtcatgaactatccctgttgccactggcacccaagtcctccgcccctctcagctcccttgggcgcctatgagggcacatgttgaatcacagtaaattatttgatggtcttgacttgtctctggctggaagtaggaggtgtagtgcctgtggcctcacagaagatactggggacctgggatctatcatctgccttgttcctgtttctagaagaggctggggaggctggcctgggacctgggactgtgcagcccttctcttctgcctgcctcaactgtcttgtgtttcctgcagaaagttataaataacacaagaatagtcaggtgatatgaaaaaaaaaaaaaaaaaaaaaaa

SEQ ID NO: 10 - protein, 189 aa, Rattus norvegicus; identical to SEQ ID NO: 2

SEQ ID NO: 11 - DNA, 504 bp, Mus musculus
atggcggtaaatgattgcttcagtctgacctatcctcacaacccacacccaggagacttgattgaagtgttccgtccttgctatcagcactgggcactgtacttgggtgatggctacgtgatcaacattgcacctatagatggcattcgctcatcatttacaagtgctaagtccgtgttcagcacaaaggccttggtgaaaatgcagcttttgaaggatgttgtgggaaatgacacatacagaataaataacaagtacgacacaacataccctcctcttcctgtggaggaggtgatacaacggtcagagttccctattgggcaggaagtagcctatgacttgctggtcaacaactgtgagcattttgtaaccttgctgcgctatggagaaggagtgtcagagcaggccaaccgagcaatcggcaccatcggattggtggcagctggtattgatatcttcacattcctcggcttgtttcccaaaagacaaagaacgaaatattag

SEQ ID NO: 12 - protein, 167 aa, Mus musculus
MAVNDCFSLTYPHNPHPGDLIEVFRPCYQHWALYLGDGYVINIAPIDGIRSSFTSAKSVFSTKALVKMQLLKDVVGNDTYRINNKYDTTYPPLPVEEVIQRSEFPIGQEVAYDLLVNNCEHFVTLLRYGEGVSEQANRAIGTIGLVAAGIDIFTFLGLFPKRQRTKY

SEQ ID NO: 13 - DNA, 1455 bp, Cavia porcellus
cgggctctgcggcggggcagctgcgccctgcgcgcgtgccgcgcttcccggggccccagctgtggccgggcgctcagcgaggcgccgcccgggctcggggacgcgggcccgcggccatgtcgctcggtgcgggacgccgcgcgtctctgtccagctccgcttccctgtcgcccgcgctccccggccgcccgcgctcccccgctgtcccctgcgccctggacccctagccgctgctccgtggaccgcttgggacgtgtcggccggggccgcgtctccccgccgcccttcccccgcgccgcgtgcagccggcgcctgtggacgcacacgggcctgcgccgctgtaggcgcggctgtcgggacgctgcctgcctcgacacgcccgccggcgggcaaggacctgttcgggcggccccgccagcaggtggggcaagagcccccgagtgccctgagcgtcagtccctggaggagccatgacggagtacaagcttgtggtggtgggtgcaggaggtgtggggaagagcgccctgactatccagctgatccagaaccattttgtggacgaatatgaccccaccatagaggattcataccggaagcaggtggtcattgatggggagacctgtctgctggatatcttggacacagctggccaagaggagtatagtgccatgcgggaccaatacatgcgcactggggagggcttcctttgtgtgttcgccatcaacaataccaaatctttcgaggacatccatcagtacagggagcagatcaagcgggtgaaggactcagatgatgtgcccatggtgctggtgggaaacaaatgtgacctggctacacgcaccgtggagtctcggcaggcccaagaccttgcccgcagttacggcattccctacattgagacatcagccaagacccgccagggagtagaagacgccttctatacactagttcgagagatccggcagcacaaactgcggaagctgaatccaccagatgagagtgggccagggtgcatgagctgcaaatgtgtgctatcctgatccaggtgagcagggcccagctggctccccgagccacagccctagccggccagccagctaagctgcacatctactctgatgttcctgggcttcctgaacctgctggtccccttgtctacgatcagtggcgtccttcatgctccatcctggcccaggctcaggacacagaggtaccagatgtggggaagaggtacagaaggaaggaaggaaggaaggaaggaagtgccactggagtccagccagcccagggatggtggatagaggcgactgcagaccctcctgtggatgctttgcaagctgtcatgaactgtcccaaacaccactggcctcccagccctaactcccctcccagtttttccagcctctgacaggtgcaggttgatttgcagtaaattatttgatggtcttgactcca

SEQ ID NO: 14 - protein, 189 aa, Cavia porcellus
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLATRTVESRQAQDLARSYGIPYIETSAKTRQGVEDAFYTLVREIRQHKLRKLNPPDESGPGCMSCKCVLS

SEQ ID NO: 15 - DNA, 1694 bp, Cavia porcellus
cgggctctgcggcggggcagctgcgccctgcgcgcgtgccgcgcttcccggggccccagctgtggccgggcgctcagcgaggcgccgcccgggctcggggacgcgggcccgcggccatgtcgctcggtgcgggacgccgcgcgtctctgtccagctccgcttccctgtcgcccgcgctccccggccgcccgcgctcccccgctgtcccctgcgccctggacccctagccgctgctccgtggaccgcttgggacgtgtcggccggggccgcgtctccccgccgcccttcccccgcgccgcgtgcagccggcgcctgtggacgcacacgggcctgcgccgctgtaggcgcggctgtcgggacgctgcctgcctcgacacgcccgccggcgggcaaggacctgttcgggcggccccgccagcaggtgcctatgacctaggctttttgcctacccagcaacttctaatttgggtgcctggttgggagcggctcagctgtcaccctgcctcggcgccggcccctgcttccttacaggccacgaggtgcagcggggtgcggtgagaccctcagcccgctggaggtgccacaggtgccgccatcctggctggcgcccttccagccactccgctgggtggcgcttggctgagctggtcctcctcacaggtggggcaagagcccccgagtgccctgagcgtcagtccctggaggagccatgacggagtacaagcttgtggtggtgggtgcaggaggtgtggggaagagcgccctgactatccagctgatccagaaccattttgtggacgaatatgaccccaccatagaggattcataccggaagcaggtggtcattgatggggagacctgtctgctggatatcttggacacagctggccaagaggagtatagtgccatgcgggaccaatacatgcgcactggggagggcttcctttgtgtgttcgccatcaacaataccaaatctttcgaggacatccatcagtacagggagcagatcaagcgggtgaaggactcagatgatgtgcccatggtgctggtgggaaacaaatgtgacctggctacacgcaccgtggagtctcggcaggcccaagaccttgcccgcagttacggcattccctacattgagacatcagccaagacccgccagggagtagaagacgccttctatacactagttcgagagatccggcagcacaaactgcggaagctgaatccaccagatgagagtgggccagggtgcatgagctgcaaatgtgtgctatcctgatccaggtgagcagggcccagctggctccccgagccacagccctagccggccagccagctaagctgcacatctactctgatgttcctgggcttcctgaacctgctggtccccttgtctacgatcagtggcgtccttcatgctccatcctggcccaggctcaggacacagaggtaccagatgtggggaagaggtacagaaggaaggaaggaaggaaggaaggaagtgccactggagtccagccagcccagggatggtggatagaggcgactgcagaccctcctgtggatgctttgcaagctgtcatgaactgtcccaaacaccactggcctcccagccctaactcccctcccagtttttccagcctctgacaggtgcaggttgatttgcagtaaattatttgatggtcttgactcca

SEQ ID NO: 16 - protein, 189 aa, Cavia porcellus; identical to SEQ ID NO: 14

SEQ ID NO: 17 - DNA, 1180 bp, Cavia porcellus
actgggcctacagtccccaccgcccgcgccgcgagcccgcagcccgagccgcacccgctgcggacggagcccatgcgcgggcccagccagcgcccgcccgcgcccccgccccgccccggccccgggggcagttgcaccagcgagtggtggggcaagagcccccgagtgccctgagcgtcagtccctggaggagccatgacggagtacaagcttgtggtggtgggtgcaggaggtgtggggaagagcgccctgactatccagctgatccagaaccattttgtggacgaatatgaccccaccatagaggattcataccggaagcaggtggtcattgatggggagacctgtctgctggatatcttggacacagctggccaagaggagtatagtgccatgcgggaccaatacatgcgcactggggagggcttcctttgtgtgttcgccatcaacaataccaaatctttcgaggacatccatcagtacagggagcagatcaagcgggtgaaggactcagatgatgtgcccatggtgctggtgggaaacaaatgtgacctggctacacgcaccgtggagtctcggcaggcccaagaccttgcccgcagttacggcattccctacattgagacatcagccaagacccgccagggagtagaagacgccttctatacactagttcgagagatccggcagcacaaactgcggaagctgaatccaccagatgagagtgggccagggtgcatgagctgcaaatgtgtgctatcctgatccaggtgagcagggcccagctggctccccgagccacagccctagccggccagccagctaagctgcacatctactctgatgttcctgggcttcctgaacctgctggtccccttgtctacgatcagtggcgtccttcatgctccatcctggcccaggctcaggacacagaggtaccagatgtggggaagaggtacagaaggaaggaaggaaggaaggaaggaagtgccactggagtccagccagcccagggatggtggatagaggcgactgcagaccctcctgtggatgctttgcaagctgtcatgaactgtcccaaacaccactggcctcccagccctaactcccctcccagtttttccagcctctgacaggtgcaggttgatttgcagtaaattatttgatggtcttgactcca

SEQ ID NO: 18 - protein, 189 aa, Cavia porcellus; identical to SEQ ID NO: 14

SEQ ID NO: 19 - DNA, 1270 bp, Cavia porcellus
cgggctctgcggcggggcagctgcgccctgcgcgcgtgccgcgcttcccggggccccagctgtggccgggcgctcagcgaggcgccgcccgggctcggggacgcgggcccgcggccatgtcgctcggtgcgggacgccgcgcgtctctgtccagctccgcttccctgtcgcccgcgctccccggccgcccgcgctcccccgctgtcccctgcgccctggacccctagccgctgctccgtggaccgcttgggacgtgtcggccggggccgcgtctccccgccgcccttcccccgcgccgcgtgcagccggcgcctgtggacgcacacgggcctgcgccgctgtaggcgcggctgtcgggacgctgcctgcctcgacacgcccgccggcgggcaaggacctgttcgggcggccccgccagcaggtgcctatgacctaggctttttgcctacccagcaacttctaatttgggtgcctggttgggagcggctcagctgtcaccctgcctcggcgccggcccctgcttccttacaggccacgaggtgcagcggggtgcggtgagaccctcagcccgctggaggtgccacaggtgccgccatcctggctggcgcccttccagccactccgctgggtggcgcttggctgagctggtcctcctcacaggtggggcaagagcccccgagtgccctgagcgtcagtccctggaggagccatgacggagtacaagcttgtggtggtgggtgcaggaggtgtggggaagagcgccctgactatccagctgatccagaaccattttgtggacgaatatgaccccaccatagaggattcataccggaagcaggtggtcattgatggggagacctgtctgctggatatcttggacacagctggccaagaggagtatagtgccatgcgggaccaatacatgcgcactggggagggcttcctttgtgtgttcgccatcaacaataccaaatctttcgaggacatccatcagtacagggagcagatcaagcgggtgaaggactcagatgatgtgcccatggtgctggtgggaaacaaatgtgacctggctacacgcaccgtggagtctcggcaggcccaagaccttgcccgcagttacggcattccctacattgagacatcagccaagacccgccagggcagccgctctggctctggcttcagctccgggacccactgggaacccctgggatccatgtgacccatcggcccccatgctggagtagaagacgccttctatacactagtt

SEQ ID NO: 20 - protein, 170 aa, Cavia porcellus
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLATRTVESRQAQDLARSYGIPYIETSAKTRQGSRSGSGFSSGTHWEPLGSM

SEQ ID NO: 21 - DNA, 696 bp, Canis lupus
ggctgagagcagcttcctcagcaggtgggacaggagacctcgttggcgtcctgtgctgcgtcccgtgaggagccatgacggagtataagctggtggtggtgggcgctggaggcgtgggcaagagcgccctgaccatccagctcatccagaaccacttcgtggatgagtacgaccccaccatcgaggactcctatcggaagcaagtggtcatcgacggggagacgtgcctgctggacatcctggacacagcgggccaggaggagtacagcgccatgcgggaccagtacatgcgcacgggggagggctttctctgtgtatttgccatcaacaacaccaagtcctttgaggacatccaccagtacagggagcagatcaagcgagtgaaggactctgacgacgtgcccatggtgctggtggggaacaagtgtgacctggctgctcgcaccgtggagtcccggcaggcgcaggacctcgcccgcagctacggcatcccctacatcgagacgtcagccaagacgcgccagggcagccggtctggctctggctccagctccgggaccctctgggaccctccgggacccccgtgacccagcggcccctagcgctggcgtggaggatgccttctacacgctggtgcgagagattcgacagcacaaggtgcgcaagctgagcccgcccgacgagggaggcccaggc

SEQ ID NO: 22 - protein, 170 aa, Canis lupus
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGSRSGSGSSSGTLWDPPGPP

SEQ ID NO: 23 - DNA, 956 bp, Canis lupus
gctgagagcagcttcctcagcaggtgggacaggagacctcgttggcgtcctgtgctgcgtcccgtgaggagccatgacggagtataagctggtggtggtgggcgctggaggcgtgggcaagagcgccctgaccatccagctcatccagaaccacttcgtggatgagtacgaccccaccatcgaggactcctatcggaagcaagtggtcatcgacggggagacgtgcctgctggacatcctggacacagcgggccaggaggagtacagcgccatgcgggaccagtacatgcgcacgggggagggctttctctgtgtatttgccatcaacaacaccaagtcctttgaggacatccaccagtacagggagcagatcaagcgagtgaaggactctgacgacgtgcccatggtgctggtggggaacaagtgtgacctggctgctcgcaccgtggagtcccggcaggcgcaggacctcgcccgcagctacggcatcccctacatcgagacgtcagccaagacgcgccagggcgtggaggatgccttctacacgctggtgcgagagattcgacagcacaaggtgcgcaagctgagcccgcccgacgagggaggcccaggctgcatgagctgcaagtgcctgctgtcctgacgtcccctccagggccacgttggcagccccgctggtcctctgtgccccaggcgcacaggctcggggcgaggaggtgccggaagctgggaggaggcgcggaaggaggaaggaggagggcgaggaaggaaggaagcgcccccggggcccggccagcccaggccccctggacagggggagcacggacctcccagggcgctttgcacagactgtcgtgaactgaggccacgggcccccccggctgtcactctcccccagtcccgtccttgcccgcggggcaaaggctgagtcgcagtaaattatttgatggtcttga

SEQ ID NO: 24 - protein, 189 aa, Canis lupus
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKVRKLSPPDEGGPGCMSCKCLLS

SEQ ID NO: 25 - DNA, 593 bp, Felis catus
gccgcgatcccctgaggaacgatgacggaatataagctcgtggtggtgggcgctggaggtgtggggaagagtgccctgaccatccagctcatccagaaccacttcgtggatgagtatgaccccaccatcgaggactcctatcggaagcaagtggttattgatggcgagacgtgcctactggacattttggacacggcgggccaggaggagtatagcgccatgcgggaccagtacatgcgcactggagaaggcttcctctgtgtgtttgccatcaacaataccaagtcctttgaggacatccaccagtacagggagcaaatcaagcgagtgaaggactccgatgacgtgcccatggtgttggtggggaacaagtgcgacctggccgcgcgcaccgtggagtcccggcaggcgcaggacctcgcccgcagctacggcatcccgtacatcgagacgtcggccaagactcgccagggtgtggaggacgctttctacacgctggtccgagagatccggcaacacaaggtgcggaagctgagcccgcccgacgagggtggccccggctgcatgagctgcaagtgcctgctctcctgacg

SEQ ID NO: 26 - protein, 189 aa, Felis catus; identical to SEQ ID NO: 24

SEQ ID NO: 27 - DNA, 675 bp, Felis catus
gccgcgatcccctgaggaacgatgacggaatataagctcgtggtggtgggcgctggaggtgtggggaagagtgccctgaccatccagctcatccagaaccacttcgtggatgagtatgaccccaccatcgaggactcctatcggaagcaagtggttattgatggcgagacgtgcctactggacattttggacacggcgggccaggaggagtatagcgccatgcgggaccagtacatgcgcactggagaaggcttcctctgtgtgtttgccatcaacaataccaagtcctttgaggacatccaccagtacagggagcaaatcaagcgagtgaaggactccgatgacgtgcccatggtgttggtggggaacaagtgcgacctggccgcgcgcaccgtggagtcccggcaggcgcaggacctcgcccgcagctacggcatcccgtacatcgagacgtcggccaagactcgccagggcagccgctctggctctggctccagctccgggaccctctgggaccctccgggacccccgtgacccagcggcccctagcgctggtgtggaggacgctttctacacgctggtccgagagatccggcaacacaaggtgcggaagctgagcccgcccgacgagggtggccccggctgcatgagctgcaagtgcctgctctcctgacg

SEQ ID NO: 28 - protein, 170 aa, Felis catus; identical to SEQ ID NO: 22

SEQ ID NO: 29 - DNA, 978 bp, Bos taurus
ccgccgcggacgggacccatgcgtcgggcgagccctgcgccccggccccggcccctgccgccgcccccgctccggccccggcccggggggcagtcgagccagtgagcggtgggaccggcgaccactgcagcgcctcgtgctgcggtctcttgaggagcaatgacggagtataagctcgtggtggtgggcgccggtggcgtggggaagagcgccctgactatccagctcattcagaatcacttcgtggacgagtacgaccccaccatcgaggactcctaccggaagcaagtggtcatcgatggggagacgtgcctgctggacatcctggacacagcgggccaggaggaatacagcgccatgcgagaccagtacatgcgcaccggggagggctttctctgcgtgtttgctatcaaccacgtcaagtccttcgaggacatccaccagtaccgggagcagatcaagcgggtgaaggactcggatgacgtgcccatggtgttggttgggaacaagtgcgacctggccgcgcgcaccgtggagtctcggcaggcccaggacctcgcccgcagctacggcatcccgtacatcgagacctccgccaagacccgccagggcgtggaggatgctttctacaccctggtgcgcgagatccggcagcacaaggtgcgcaagctgagcccgccggacgagggcggccccggctgcctgagctgcaggtgcctgctctcctgacggcagcgtgggcgcggagcgctgggtgccatgcaggaggcggcgcagcaggggtggagggaggtgccgccagagcccagccccccaggccagtgggcagtgcccgcgggcctcccgggacgcttcgcacagactctggtgaactgatgctgctggcccccagcctcgctctcctccagccctgtcctggcccagcgggcgcaggccgagtcgcagtaaattatttcatggtcttgaaaaaaaaaaaaa

SEQ ID NO: 30 - protein, 189 aa, Bos taurus
MTEYKLVVVGAGGVGKSALTIQLIQNHFVDEYDPTIEDSYRKQVVIDGETCLLDILDTAGQEEYSAMRDQYMRTGEGFLCVFAINHVKSFEDIHQYREQIKRVKDSDDVPMVLVGNKCDLAARTVESRQAQDLARSYGIPYIETSAKTRQGVEDAFYTLVREIRQHKVRKLSPPDEGGPGCLSCRCLLS

SEQ ID NO: 31 - DNA, 1060 bp, Bos taurus
ccgccgcggacgggacccatgcgtcgggcgagccctgcgccccggccccggcccctgccgccgcccccgctccggccccggcccggggggcagtcgagccagtgagcggtgggaccggcgaccactgcagcgcctcgtgctgcggtctcttgaggagcaatgacggagtataagctcgtggtggtgggcgccggtggcgtggggaagagcgccctgactatccagctcattcagaatcacttcgtggacgagtacgaccccaccatcgaggactcctaccggaagcaagtggtcatcgatggggagacgtgcctgctggacatcctggacacagcgggccaggaggaatacagcgccatgcgagaccagtacatgcgcaccggggagggctttctctgcgtgtttgctatcaaccacgtcaagtccttcgaggacatccaccagtaccgggagcagatcaagcgggtgaaggactcggatgacgtgcccatggtgttggttgggaacaagtgcgacctggccgcgcgcaccgtggagtctcggcaggcccaggacctcgcccgcagctacggcatcccgtacatcgagacctccgccaagacccgccagggcagccgctctggctctggctccagctccgggaccctctgggaccctccgggacccccgtgacccagccgcccctctcgctggcgtggaggatgctttctacaccctggtgcgcgagatccggcagcacaaggtgcgcaagctgagcccgccggacgagggcggccccggctgcctgagctgcaggtgcctgctctcctgacggcagcgtgggcgcggagcgctgggtgccatgcaggaggcggcgcagcaggggtggagggaggtgccgccagagcccagccccccaggccagtgggcagtgcccgcgggcctcccgggacgcttcgcacagactctggtgaactgatgctgctggcccccagcctcgctctcctccagccctgtcctggcccagcgggcgcaggccgagtcgcagtaaattatttcatggtcttgaaaaaaaaaaaaa

SEQ ID NO: 32 - protein, 170 aa, Bos taurus
MTEYKLVVVGAGGVGKSALTIQLIQNHFVDEYDPTIEDSYRKQVVIDGETCLLDILDTAGQEEYSAMRDQYMRTGEGFLCVFAINHVKSFEDIHQYREQIKRVKDSDDVPMVLVGNKCDLAARTVESRQAQDLARSYGIPYIETSAKTRQGSRSGSGSSSGTLWDPPGPP

SEQ ID NO: 33 - DNA, 1531 bp, Bos taurus
cccatttaaaacatcgggagaactttctagatttgcagctggcctcctgctgcctcaggacagctgtctagctgcataaaacactccgtttgctcaaaatcgcaaataaactctgatattacttcagcatcgtcctttccagcccgaccaatttccagtggaaacctggctggcagaagacacacagagtgaacttctggtggtgggtagagaggtgcccagcaagatgtggggagaatgaaccggccacggctcactgcgtttgatgaggccctgcaggaggcaaacttcagctggaatggggagagcccaggcgttctgagacctgtggctcgaaaaatacaacggcttctcaactcaaatcagtgacaagaaaaatgaagaaaggcgtggagttagaaagaccccatgagattcggcctcggggagaaaccggttgccaggcacttgctgcccttgggtggtgctaggaggaccaaggcctgagacccccggttccagtgattgagtctgcagagacagaaatgctgcagcagctgccagtggagtggctttgcatggttggcactggatacaagtgtgctaggaagcacgcccgccgagggccacggcctggaccgaggttgaccgtggtcatcagagaccaggagtccgggcttctctcacacaggcagtgggaccggcgaccactgcagcgcctcgtgctgcggtctcttgaggagcaatgacggagtataagctcgtggtggtgggcgccggtggcgtggggaagagcgccctgactatccagctcattcagaatcacttcgtggacgagtacgaccccaccatcgaggactcctaccggaagcaagtggtcatcgatggggagacgtgcctgctggacatcctggacacagcgggccaggaggaatacagcgccatgcgagaccagtacatgcgcaccggggagggctttctctgcgtgtttgctatcaaccacgtcaagtccttcgaggacatccaccagtaccgggagcagatcaagcgggtgaaggactcggatgacgtgcccatggtgttggttgggaacaagtgcgacctggccgcgcgcaccgtggagtctcggcaggcccaggacctcgcccgcagctacggcatcccgtacatcgagacctccgccaagacccgccagggcgtggaggatgctttctacaccctggtgcgcgagatccggcagcacaaggtgcgcaagctgagcccgccggacgagggcggccccggctgcctgagctgcaggtgcctgctctcctgacggcagcgtgggcgcggagcgctgggtgccatgcaggaggcggcgcagcaggggtggagggaggtgccgccagagcccagccccccaggccagtgggcagtgcccgcgggcctcccgggacgcttcgcacagactctggtgaactgatgctgctggcccccagcctcgctctcctccagccctgtcctggcccagcgggcgcaggccgagtcgcagtaaattatttcatggtcttga

SEQ ID NO: 34 - protein, 189 aa, Bos taurus; identical to SEQ ID NO: 30

SEQ ID NO: 35 - DNA, 962 bp, Gallus gallus
gggttgatggccggaccgtcgattccctgacgactacgagcacctacacctgaggtggtggtgctcctggaggccctgagctggggtcagctggaagatgaccgagtacaagctggtggtagtgggagctggaggtgtcgggaagagcgctttgacgatacagctcattcagaaccattttgttgatgagtacgaccccacaatagaggattcctacagaaagcaagtcgtcatcgatggagagacctgtttgctggacatcctggatacggcggggcaggaggagtacagtgccatgcgagaccagtacatgagaacgggggaaggattcctgtgcgtctttgccattaacaacaccaagtcctttgaggacatccaccagtacagggagcagatcaagagggtgaaagactcagatgatgtccccatggtgctggtgggaaataaatgtgatctgccagcacggacagtggagacccggcaagcgcaggacctggcccggagttacgggatcccctacatagaaacgtcggccaaaaccagacagggcgtcgaagatgccttctataccttagtgcgggagatccgtcagcataaactgcgcaagctgaacccaccagatgagagtggccctggctgcatgaactgtaaatgcgtgatatcgtgactgtgctgactggaccctgacttggagaggtgtccgctgtgcagagcacaaggaaagaggtgatgcgaaggaagaaacaaatggattcaggggaggagtggagggggagggagagaggaagaagaggacgggaggagtgccagcccctccaaggactatctcgcacttcacccaggccggcagcagatgacttttggttctttccccatcccctcctcctttggcctcctccaccccggcaactgtacaaagccacagattgaatcacagtaaattattatttgatggtctcgac

SEQ ID NO: 36 - protein, 189 aa, Gallus gallus
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLPARTVETRQAQDLARSYGIPYIETSAKTRQGVEDAFYTLVREIRQHKLRKLNPPDESGPGCMNCKCVIS

SEQ ID NO: 37 - DNA, 5436 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagggagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 38 - protein, 189 aa, Homo sapiens [sequence omitted]

SEQ ID NO: 39 - DNA, 5312 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 40 - protein, 188 aa, Homo sapiens [sequence omitted]

SEQ ID NO: 41 - DNA, 661 bp, Rattus norvegicus
atgactgagtataaacttgtggtagttggagctggtggcgtaggcaagagtgccttgacgatacagctaattcagaatcactttgtggatgaatatgatcctacgatagaggactcctacaggaaacaagtagtaattgatggagaaacctgtctcttggatattctcgacacagcaggtcaagaggagtacagtgcaatgagggaccagtacatgagaactggggagggctttctttgtgtatttgccataaataatactaaatcatttgaagatattcaccattatagagaacaaattaaaagagtaaaggactctgaagatgtgcctatggtcctagtagggaataagtgtgacttgccttctagaacagtagacacgaaacaggctcaggagttagcaaggagttatgggattccattcattgagacctcagcgaagacaagacagggtgttgacgatgccttctatacattagtccgagaaattcgaaaacataaagaaaagatgagcaaagatgggaaaaagaagaagaagaagtcaaggacaaggtgtatagtcatgtgaatagtttgtactctttcttaaggcacacttaagtaaagtgtgatttttgtacattacactaaattattagcatttgttttagcattacctaatc

SEQ ID NO: 42 - protein, 188 aa, Rattus norvegicus
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQELARSYGIPFIETSAKTRQGVDDAFYTLVREIRKHKEKMSKDGKKKKKKSRTRCIVM

SEQ ID NO: 43 - DNA, 4670 bp, Mus musculus
aggcggcggccgcggcggctgaggcggcagcgctgtggcggcggctgagacggcaggggaaggcggcggcggctcggcccggagtcccgctcccgcgccatttcggacccggagcgagcgcggcgcgggcctgaaggcggcggcgggagcctgaggcgcggcggctccgcggcgcggagagaggcctgctgaaaatgactgagtataaacttgtggtggttggagctggtggcgtaggcaagagcgccttgacgatacagctaattcagaatcactttgtggatgagtatgaccctacgatagaggactcctacaggaaacaagtagtaattgatggagaaacctgtctcttggatattctcgacacagcaggtcaagaggagtacagtgcaatgagggaccagtacatgagaactggggagggctttctttgtgtatttgccataaataatactaaatcatttgaagatattcaccattatagagaacaaattaaaagagtaaaggactctgaagatgtgcctatggtcctggtagggaataagtgtgatttgccttctagaacagtagacacgaaacaggctcaggagttagcaaggagttacgggattccgttcattgagacctcagcaaagacaagacagggtgttgacgatgccttctatacattagtccgagaaattcgaaaacataaagaaaagatgagcaaagatgggaagaagaagaagaagaagtcaaggacaaggtgtacagttatgtgaatactttgtactctttcttaaggcacacttaagtaaaagtgtgatttttgtacattacactaaattattagcatttgttttagcattacctaatctttttttttcttctgttcgtgcaaactgtcagcttttatctcaaatgcttattttaaaagaacagtggaaaccttcttttttctaagtgccagtattccctgggttttggacttaaactagcaatgcctgtggaagagactaaagacctgagactctgtcttgggatttggtgcatgcagttgattccttgctagttctcttaccaactgtgaacactgatgggaagcaggataatgaagcttccggaccatccctgctctgtgtccatctactcatccaatggagtcattagcagtcaatcgccgcttcactggacactgaggggtcacagacttaggctccctttgagtcgcgtccagcgtgtcctagactttatcatctttcagaggcgtaggcagactgttcacaaaggctttctgtagctttccactgcaattaatcttggtcactccctcaaatagtatattttttctagaaaaggggaaaaatggaaaaaaaaaggcaatggaaaatgttgaaatccattcagtttccatgttagctaaattactgtaagattcctataatagcttttcctggtaaggcagacccagtatgaaatagtaataaccatttgggctatatttacatgctactaaatttttgtaataattcaaacaactttagcatatataaaaagttctcataagaattaagtacaattcccctttgtcagattgttcttatcctaactttcaagtcttttttgaatttctgttgttgaaagtagttttaatggttgtgaagctgaagatgatctgagacagttatagcttggcaggtgttgaggagaccagagttgcagggttgggccttacgtgaacctgtgacgaacgctactgggttttgcagcactgctgcattcaatgttggcgacgcattgtttggtcaacataggggataaggagactttgatggcttagtataatgcattctcaccatgtaacagtcctactgacaaatcaagaaatttgtttataataataaaaaatttttaaaaatttcgatgttcgcttcaaggttgagattttggggtaggaggctacaacaagagtaaatcttaaagcaaggttttaagaaggtttgaaaatgcaggtttgactagtctctcaactctagctaaacaaacattcccaagtacttcccaaatctgataggtatttaaaattatctaatgctttaagaatagttaacaggaaaaaaatctcctcagtgcacttaaagcaacccttcacatcatttgaaatgagatggaaatatcactggactatgaggactggatgtctgtctgattttaagcaaatcactgtctgcttggttttgaatcatctcaaagacattaacctcccagccgtgtaacatagtttacatgttgacacacctagttatcaagctcagcacaatctgtaactgttttacatggattaacatcttcactgccagtcttgggcaaattgtgcaagaggtaaaatttatatttcagtatccattctcccatttcaggactcccctccaacattatgctggctttcagcctgtctctcacctgcccatcacttagtgtagttttaataatttcccccacttcaaactttgtttccactatggacaacttcatgaactttgcccactaaggtaggtacatcaaagctgccctatggctttcttccccgggactgaaaataacagacaccatagtgggatttaaactaatagatggttttcagggccactacaacaattcaatctcaatcctttggacttcattcctgctgcccaggccactggtgcctcagtaggaattttcaaaattagtgtgaacagacagagcacagtccagtggaaggtgagcttaatcttcatctagccatcatcatggtaagtgatagattctattgttttaataaatacagtctaacaatgaaaaacacttcgaagtttcaatcataaagctgtctttttaaaaattttatttactcaacatttattcagtgcttgtcatattctgggaattacactaggcactcagggtgcggtgtcctcaatccttggccagtggtatgtagcatgatctgtaataccactaaataaggcatatagcatatgacttagacataatgaaatacatgatttgagttttgcagagaggagtttgggtttgtacattcccttcccccccagtttagcaagaattgtttgctgtgaatccaatgcaacttttaaatcaaactactttatataattatttcatttttctaaaggaacagaagtaccctaaactatttttttgaaatgttctaaactgtacatattcatagaacattctttgggtgaattttaagtcttaaaatgcaattagtaatacttctcatttctattcagaggaacaggtgtacttcaaaagctgcagtgtataatcagatatttttaatggacaatgtgttaaagaagtggtaattaccactatgtaaatttgaattgtgttacactttggttaacaaaaggggaaagaatcctagaaacaaatatgttatctagttactgcagccttaaagtccttgttgaagttaaaaagcaatgctaagttacagtcataggcattaacatgtttatgggaaggatatagtaggcaaatacaatttgagtaaatattttcagtagggaattttaggctctactgactgagtcacactgcataggaatttagatcttaacttttataggttatcgacctttgccaccattgcacaattttgtcctaacataaatacaagttctgtgaggcatgtcaaaagttacagtttgcataaattcatctcattttgtattccactgattttacattttcctcaaacatacatacatacatacatacaacacacacacactcacacatgaagggttttttttttgtaggcaataaaaatttaactaatttccatttgttaaaaagtagtgatttattgagaattatgcagtcattttttaaacccaaaagttatttaaaggtgaatttatactcaataacttctgtgtaatactgggtagcatgaattctgcattgaaaaattgaacagataataccaatagctgtaaattctgtcaaaacatgaaaattatttctaaagaagtacattagttttcaaagaacagttattagaatcagatctgtggtttagttcaataatttgaagtgcctgtttgggatggtggtaggcattttagatgaatttgggaaaaataaagttctgcagaaatgccagtttcagaccccgctaacccgctgagtgggctgtgtgctgtgttagctccagtgccccaatcccgtttcatgtcttcatgttgaaacacttctgcatttttatttgagtgccaatttcttactagtgctatttcttagtgtaacatgtttacctgggatgtattttaactatttttgtatagtgtaaactgaaacatgcacattttgtacattgtgctttccttctttccattccttttctttctgttttgtttgtttgtttgtttgtttgtttgttatgggacatatgcagtgtgatccagttgttttccatcctttggttgcgctgacctagggaatgttggtcatatcaaacattaaatttaaaagtgaccactcttaattaaaattaacttttaaatgtttataggagtacgtgctgtgaagtgatctgaaatttgtaatatttttgtcatgaaccgtactgctcctaatcattgtaatgtaataaaaatagttatggtgactatgaa

SEQ ID NO: 44 - protein, 188 aa, Mus musculus
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQELARSYGIPFIETSAKTRQGVDDAFYTLVREIRKHKEKMSKDGKKKKKKSRTRCTVM

SEQ ID NO: 45 - DNA, 2068 bp, Oryctolagus cuniculus
acacacgcgcccacccagccacccggccaccagccgccatgggcaaggacaggcagccccgcgggcagcagaggcagggggacgccgccgggcccgacgacccggggccgaagaaaggcgccgggacccgcgagcagcgcggggaggaggaggcccagacgtgctgcggctgccgcttcccgctgctgctcgcgctgctgcagctggccctgggcgtcgccgtgaccgtggtgggcttcctcatggcgagcgtcagctcctccctgctagtcagagccactccatattgggctgggatcattgtctgcgtggtggcctatcttggcttgtttatgctttgtgtctcgtaccaagttgatgaacggacgtgtatccagttttctatgaaactgctgtacttcgtgctgagcgccctggggctggtggtgtgcgtgctggccgtggccttcgccgcccaccactactcgctgctcacgcacctgacctgtgagaacgcccccgactcctgccagtgcaagctgccgtcctcggagccgctgagccggaccttcgtgtaccgggatgtgactgactgtaccagcatcacgggcaccttccaggtgttcctgctcgtccagatggttctgaacttggtctgtggccttgtgtgcttggtggcctgctttgtgatgtggaaacacaggtatcaggtcttttatgtgggggtcaggatgtgccccctgtcggcttccgaaggccagcagcagaaggtgtaggaatcttgctcagaattgggagagaaaatggcacactggctagctgaggttaaaaagaaaaattatttttaaggagaaagagagaaaacttttgccaatatttactgctctaaatgaatattttttatatttttcaagaaacaaaagagcatttcttcaggtttctattgtatttttaaacattcgtataggttcaacaagatacacattgatttgcgggatattcaaagtcaaaagcacaccaaactgggaagcaatggcacagaactgtcctcacagtctggggtttactcttcatgctcactttcgccaccactgacgtacggctttctggttaatacggtctaaggtttgtagtggctgccccaggtccttcccgcctcctaccacaatcccccactgcgtcttagagaaaggcaaggtgtggagaagtcactgggcaaccaggtggaatctcttcatttcccctactgcggatgttgtcaaggcccaaaacatgagcgaacttcaaaaacctcatgggaagtggagttcgaagtttattttgctgccaaaaaattaagatccacacatatatagggatcttcagaaagttcacagaaaatgcataatatgggaaaaaaaaagattcacggatttcagaattttgtttggaccaaactaaagttatcttttaatgccatttctgaagtgccctcatagcttggaaagccaagcagaaaagaggctttgcaaaaatacaagtaattataaacacctgggccagggcggctgtctcagctgccttcgcttggctctgtacgtagatcactcgcgcggggcttggcagggctctctgcttctcaataattgaaatatggtggtagttgtattcttaatgatgtagaaggtttaaaaataattacattacgcttccattctatcatctacaacaaatcattcaacctaatttctagctaattgttaattataattatgctcagaagtctatttaatgagctctggctgtacttaggcagctctgccagtgtaaagagaaattattctcgtaagagaagaggcctaaagattctttcttctgaaagtcaagcgttataagggaaaacttttttttaattaatagctcaggataaaaacaccaatttaaacaaaaacaagagcatttataataggaagtacttgtacaaatagcacgtttgtggcacattgcagagtgtctctctttgcagctaaatagctttgaagaaggctggcgagtgcagatgtattctgtgcacaaaactgtatttggctcataaccctattattgattc

SEQ ID NO: 46 - protein, 238 aa, Oryctolagus cuniculus
MGKDRQPRGQQRQGDAAGPDDPGPKKGAGTREQRGEEEAQTCCGCRFPLLLALLQLALGVAVTVVGFLMASVSSSLLVRATPYWAGIIVCVVAYLGLFMLCVSYQVDERTCIQFSMKLLYFVLSALGLVVCVLAVAFAAHHYSLLTHLTCENAPDSCQCKLPSSEPLSRTFVYRDVTDCTSITGTFQVFLLVQMVLNLVCGLVCLVACFVMWKHRYQVFYVGVRMCPLSASEGQQQKV

SEQ ID NO: 47 - DNA, 1039 bp, Cavia porcellus
ggtggcttctcggccagacctcccggcccccgccatttcggaccgggagccagcgcgacgcgggcactgagggcggcggcgggggccacaggctcggcggctcccaggtgcgggagagaggcctgctgaaaatgactgaatataaacttgtggtagttggagctggtggcgtaggcaagagtgccttgacgatacagctaattcagaatcactttgtggatgaatatgatcctacaatagaggattcctacaggaaacaagtagtaattgatggagaaacctgtctcttggatattctcgacacagcaggtcaagaggagtacagtgcaatgagggaccagtacatgaggactggggagggctttctttgtgtatttgccataaataatactaaatcttttgaagatattcaccattatagagaacaaattaaaagagttaaagactctgaagatgtacctatggtcctagtaggaaataaatgtgatttgccttctagaacagtagacacaaaacaagctcaggacttagcaagaagttatggaattccttttattgaaacatcagcaaagacaagacagagagtggaggatgctttttatacattggtgagagagattcgacaatacagattgaaaaaaatcagcaaagaagaaaagactcctggctgtgtgaaaattaaaaaatgcattataatgggtgttgatgatgccttctatacgttagttcgagaaattcgaaaacataaagaaaagatgagcaaagatggtaaaaagaagaaaaagaagtcgaagacaaagtgtataattatgtaaatacaatttgtacttttttcttaaggcatacttaagtaaaagtggtaatttttgtacattacactaaattattagcttttgttttagcattacttaattcttttcctatttcatgcaaactgttagcttttatcttaaatgctcattttaaaatgacagtggaaaccttttatttcctcttaagtgccagtattccctgcattttggtttttgaactagcaatg

SEQ ID NO: 48 - protein, 227 aa, Cavia porcellus
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIMGVDDAFYTLVREIRKHKEKMSKDGKKKKKKSKTKCIIM

SEQ ID NO: 49 - DNA, 835 bp, Cavia porcellus
acaggctcggcggctcccaggtgcgggagagaggcctgctgaaaatgactgaatataaacttgtggtagttggagctggtggcgtaggcaagagtgccttgacgatacagctaattcagaatcactttgtggatgaatatgatcctacaatagaggattcctacaggaaacaagtagtaattgatggagaaacctgtctcttggatattctcgacacagcaggtcaagaggagtacagtgcaatgagggaccagtacatgaggactggggagggctttctttgtgtatttgccataaataatactaaatcttttgaagatattcaccattatagagaacaaattaaaagagttaaagactctgaagatgtacctatggtcctagtaggaaataaatgtgatttgccttctagaacagtagacacaaaacaagctcaggacttagcaagaagttatggaattccttttattgaaacatcagcaaagacaagacagggtgttgatgatgccttctatacgttagttcgagaaattcgaaaacataaagaaaagatgagcaaagatggtaaaaagaagaaaaagaagtcgaagacaaagtgtataattatgtaaatacaatttgtacttttttcttaaggcatacttaagtaaaagtggtaatttttgtacattacactaaattattagcttttgttttagcattacttaattcttttcctatttcatgcaaactgttagcttttatcttaaatgctcattttaaaatgacagtggaaaccttttatttcctcttaagtgccagtattccctgcattttggtttttgaactagcaatg

SEQ ID NO: 50 - protein, 188 aa, Cavia porcellus
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCIIM

SEQ ID NO: 51 - DNA, 4993 bp, Canis lupus
gctgaaaatgactgaatataaacttgtggtagttggagctggtggcgtaggcaagagtgccttgacgatacagctaattcagaatcactttgtggatgaatatgatcctacaatagaggattcctacaggaaacaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaagactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcaacaaagaagaaaagactcctggctgtgtgaaaattaaaaaatgcattgtaatgggtgttgacgatgccttctatacattagttcgagaaattcgaaaacataaagaaaagatgagcaaagatggtaaaaagaagaaaaagaagtcaaagacaaagtgtataattatgtaaatacaatttgtacttttttcttaaggcatacttaagtaaaagtggtaatttttgtacattacactaaattattagcatttgttttagcattacctaattttctgctccatccaaactgttagcttttatcttgaatgcttattttaaaatgacagtggaaactttttcctctaagtgccagtattccctgagttttggttttgaactagcaatgcctgtgaaaaagaaactgaatacctgagatttctgtcttggggtttttggtgcatgcagttgattacttcctatttttcttaccaattgtgaactttggtgtgaaacaaattaatgaagctttcgaatcatccctattctgtgttttacctagtcacatacatggattaattactaattataacttcagttgatatttcatgattggttttactgaaacattgagggaacatgaatttatgggctgcttcttataggtataatgtcctatagtttcagtcacccttaatgaatgtaaagctacactgttcacaaaggttttctccatcttttcactgctatttgtcatagccacgctcccaaaaatattatattttttctataaaaaagggaaaaaatagaaaaaaatacaaggcaatggaaaatattaaaaggcatttactttccatattagataaattcctataatactctgaatagcttttcctgttaaggcagacccagtatgtaatgaggattatagcaaccattttggggctatatttacatgctactaaatttttgtattaattgaaaaagttttaacatgtataaaaaattcccataggaattaaatatagtctccctgtgtcagattgctctttcttagcataactttaaatcttttcttgatcttcaatcttagaaaatagttttaattcttgtagtgatgttaaagattatttgggccagttagtttttaatagatgttaaagagaccacagttccaaggccaggccttgtgtgaacctttaagcttcattaagagtttcatagtacagactgcatccctgtggtctcccagggtcatcatgcattgattgggtggtcaaaagtggggacaaagagtgtttagataagatgcatcctcactgtatggtggtcctgctgacagatcaggaccatcacttttgttttttaaaaaaccaacagagctttttaaaaacattatttaaaatgagatttttgggggcagggggtggcaagacttgaattttttttaaacaatgaagtaaaaaggtttcaaaatctctagtgttggctagttctcaacattggctaaagtaacatttcataaacactttacaagtattggtccatatttaagaatatctaatgcttaaataatagattaataacaattctttcagtgcatttaaaatgtatttttaaatatctgaagtgagatggtgtgttgaggtgaaaatatcactggactaggaggaaggtgacttagattctagttacgtgtcttttacaacttcagttttgggcaaatcactcactatccatttcttcatgttaagtcatctcaaaggctatatctagcatcaactatgtgatttacattcagtttacataaggatatacctatttgtcaatctcagcacaatctgtaactttttacctatgttctcttcagcgccagtcttaggcaaagttgtgcaagaggtgaggtttatttttgagaatctgatctccggtagcaggtactcctctcccatgttagtgtcatcttgcctgcctaccttctacatgccccatgacttgatgctttctaattccccgaacctcaagatgtagtgctgctttggatatctccatgaggtaataagtcacattagtcaggctcaacataatctgacagatactgtagtgggatttgatctaatagctaattttcaggtggtaactgtatcaatttaattttgatcttttgaacatcatctctgctacctggtccattagtgactaagtaggaaaagtaggaattttcatatctgtgatgtgtagacagaccctatccagtagaagaatttaataaatttaattaataaatactgaaagatttccttagataatccaaaactaggactagccctggtaacggtgatacattccattattttaataagtaaaatcttcttacaatgaaaaatactttaaaatttaattcataaagcttactttttagcagaattcatttattcaacaaatacttgagtgcctgctagatgccaggttctacacaaggcaccggggatattatggtattcccaacaagggacataatccctatccttaagtagtactgttattttagagtggtctgtagtatattagtgaggcatttggcacatgacccagagataatataatgcatattttagttttgcacagaagggatatggtctctaaggttttttccagctctaaaataattgttcgctctgattccaataaactgtttaatcaagctactttatataaatcactttacttcattattttaaagaagtaaacttgactatattgttttttatttgggataattatgtgattctgttgggatacttatatagtacacattaaattgtatgtcagatgataacattaaaattcccaagtgtaatattctacttggtctctgtgtatcataattaaaatagatttaaatattgagttcaaaaatagttttatttatctgggtgtgaataaacagatgcctgaactaattcacagaaaaggaaacttctgtgtaaaaagtcagtccaatttctgaaatgctatgctaaactacaggtttatggaacattagatagggtgttaagactttatatagtacttcctcttgtttctatacaagagaaagaaatggccatacttcaggaattgcagtgcataactgagggatttttaggactcttgaatttttgatgtagccgggcaactttttttaggcagtggtaattatcctttattatgtgaattttgaatggtttgacaaaacgtttgtttttgtagagattttaaaaggggagcgctaatcctagaaataaatattatgtaattattacggccttaaagataaaaatccttgttgaaagttgaaaaaaattgctaaattacatagtcttagacattaacatgtttgtggaagaatgtagcagaggtatgtagtataatttgagtgaatattcccaattaggaattctaggctctagtttaactgagtcacactgcataggaatttagaacctaacttctaggttatcaaaatctttgccaccattgcacaattttgtcctaatatatagagaaactttgtgaggcatgttcagttgcggtttgcacaagttcatctcatttgtattccagtgattttttttcttctaaccatttttttaaacaacatgtacacattgttttttttggtaggcaatgaaaactgtcatttccattgtcaaacagtaattcctcgataactgtattaatggtttttaaaaaaccatcagttactttaaaactgaatttatatttaataacttctgtattagtattgggtagcatgaaatctctattgagaaattgaacagcatacaactagtagctgtaaattccttcagaaagtgaaaattatttcttcctaaagatatcttgacatcagtgcttgaagaatagtcataactagattaataattgttttagttaaacagttttaagtgcctgtttcagatgatgataggcaatttagatgaatttaggaaaaatcaaagtttttacttgcagaaatgtccattatagggggcccccctcctcatagagctgaatgggttatgtaatgttttatccaaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgcttttttttgtgggacatatgcagtgtgatccagttgttttccatcatttggttgcgctgacctaggaatgttggtcatatcaaacattaaatttaaaaatgaccactcttttaattaaaattaacttttaaatgtttataggagtatgtgctgtgaagtgatctgaaatttgtaatatttttgtcatgaactgtactgctcctaattattgtaatgtaataaaaatagttatggtgactatga

SEQ ID NO: 52 - protein, 4993 aa, Canis lupus
GCTGAAAATGACTGAATATAAACTTGTGGTAGTTGGAGCTGGTGGCGTAGGCAAGAGTGCCTTGACGATACAGCTAATTCAGAATCACTTTGTGGATGAATATGATCCTACAATAGAGGATTCCTACAGGAAACAAGTAGTAATTGATGGAGAAACCTGTCTCTTGGATATTCTCGACACAGCAGGTCAAGAGGAGTACAGTGCAATGAGGGACCAGTACATGAGGACTGGGGAGGGCTTTCTTTGTGTATTTGCCATAAATAATACTAAATCATTTGAAGATATTCACCATTATAGAGAACAAATTAAAAGAGTTAAAGACTCTGAAGATGTACCTATGGTCCTAGTAGGAAATAAATGTGATTTGCCTTCTAGAACAGTAGACACAAAACAGGCTCAGGACTTAGCAAGAAGTTATGGAATTCCTTTTATTGAAACATCAGCAAAGACAAGACAGAGAGTGGAGGATGCTTTTTATACATTGGTGAGAGAGATCCGACAATACAGATTGAAAAAAATCAACAAAGAAGAAAAGACTCCTGGCTGTGTGAAAATTAAAAAATGCATTGTAATGGGTGTTGACGATGCCTTCTATACATTAGTTCGAGAAATTCGAAAACATAAAGAAAAGATGAGCAAAGATGGTAAAAAGAAGAAAAAGAAGTCAAAGACAAAGTGTATAATTATGTAAATACAATTTGTACTTTTTTCTTAAGGCATACTTAAGTAAAAGTGGTAATTTTTGTACATTACACTAAATTATTAGCATTTGTTTTAGCATTACCTAATTTTCTGCTCCATCCAAACTGTTAGCTTTTATCTTGAATGCTTATTTTAAAATGACAGTGGAAACTTTTTCCTCTAAGTGCCAGTATTCCCTGAGTTTTGGTTTTGAACTAGCAATGCCTGTGAAAAAGAAACTGAATACCTGAGATTTCTGTCTTGGGGTTTTTGGTGCATGCAGTTGATTACTTCCTATTTTTCTTACCAATTGTGAACTTTGGTGTGAAACAAATTAATGAAGCTTTCGAATCATCCCTATTCTGTGTTTTACCTAGTCACATACATGGATTAATTACTAATTATAACTTCAGTTGATATTTCATGATTGGTTTTACTGAAACATTGAGGGAACATGAATTTATGGGCTGCTTCTTATAGGTATAATGTCCTATAGTTTCAGTCACCCTTAATGAATGTAAAGCTACACTGTTCACAAAGGTTTTCTCCATCTTTTCACTGCTATTTGTCATAGCCACGCTCCCAAAAATATTATATTTTTTCTATAAAAAAGGGAAAAAATAGAAAAAAATACAAGGCAATGGAAAATATTAAAAGGCATTTACTTTCCATATTAGATAAATTCCTATAATACTCTGAATAGCTTTTCCTGTTAAGGCAGACCCAGTATGTAATGAGGATTATAGCAACCATTTTGGGGCTATATTTACATGCTACTAAATTTTTGTATTAATTGAAAAAGTTTTAACATGTATAAAAAATTCCCATAGGAATTAAATATAGTCTCCCTGTGTCAGATTGCTCTTTCTTAGCATAACTTTAAATCTTTTCTTGATCTTCAATCTTAGAAAATAGTTTTAATTCTTGTAGTGATGTTAAAGATTATTTGGGCCAGTTAGTTTTTAATAGATGTTAAAGAGACCACAGTTCCAAGGCCAGGCCTTGTGTGAACCTTTAAGCTTCATTAAGAGTTTCATAGTACAGACTGCATCCCTGTGGTCTCCCAGGGTCATCATGCATTGATTGGGTGGTCAAAAGTGGGGACAAAGAGTGTTTAGATAAGATGCATCCTCACTGTATGGTGGTCCTGCTGACAGATCAGGACCATCACTTTTGTTTTTTAAAAAACCAACAGAGCTTTTTAAAAACATTATTTAAAATGAGATTTTTGGGGGCAGGGGGTGGCAAGACTTGAATTTTTTTTAAACAATGAAGTAAAAAGGTTTCAAAATCTCTAGTGTTGGCTAGTTCTCAACATTGGCTAAAGTAACATTTCATAAACACTTTACAAGTATTGGTCCATATTTAAGAATATCTAATGCTTAAATAATAGATTAATAACAATTCTTTCAGTGCATTTAAAATGTATTTTTAAATATCTGAAGTGAGATGGTGTGTTGAGGTGAAAATATCACTGGACTAGGAGGAAGGTGACTTAGATTCTAGTTACGTGTCTTTTACAACTTCAGTTTTGGGCAAATCACTCACTATCCATTTCTTCATGTTAAGTCATCTCAAAGGCTATATCTAGCATCAACTATGTGATTTACATTCAGTTTACATAAGGATATACCTATTTGTCAATCTCAGCACAATCTGTAACTTTTTACCTATGTTCTCTTCAGCGCCAGTCTTAGGCAAAGTTGTGCAAGAGGTGAGGTTTATTTTTGAGAATCTGATCTCCGGTAGCAGGTACTCCTCTCCCATGTTAGTGTCATCTTGCCTGCCTACCTTCTACATGCCCCATGACTTGATGCTTTCTAATTCCCCGAACCTCAAGATGTAGTGCTGCTTTGGATATCTCCATGAGGTAATAAGTCACATTAGTCAGGCTCAACATAATCTGACAGATACTGTAGTGGGATTTGATCTAATAGCTAATTTTCAGGTGGTAACTGTATCAATTTAATTTTGATCTTTTGAACATCATCTCTGCTACCTGGTCCATTAGTGACTAAGTAGGAAAAGTAGGAATTTTCATATCTGTGATGTGTAGACAGACCCTATCCAGTAGAAGAATTTAATAAATTTAATTAATAAATACTGAAAGATTTCCTTAGATAATCCAAAACTAGGACTAGCCCTGGTAACGGTGATACATTCCATTATTTTAATAAGTAAAATCTTCTTACAATGAAAAATACTTTAAAATTTAATTCATAAAGCTTACTTTTTAGCAGAATTCATTTATTCAACAAATACTTGAGTGCCTGCTAGATGCCAGGTTCTACACAAGGCACCGGGGATATTATGGTATTCCCAACAAGGGACATAATCCCTATCCTTAAGTAGTACTGTTATTTTAGAGTGGTCTGTAGTATATTAGTGAGGCATTTGGCACATGACCCAGAGATAATATAATGCATATTTTAGTTTTGCACAGAAGGGATATGGTCTCTAAGGTTTTTTCCAGCTCTAAAATAATTGTTCGCTCTGATTCCAATAAACTGTTTAATCAAGCTACTTTATATAAATCACTTTACTTCATTATTTTAAAGAAGTAAACTTGACTATATTGTTTTTTATTTGGGATAATTATGTGATTCTGTTGGGATACTTATATAGTACACATTAAATTGTATGTCAGATGATAACATTAAAATTCCCAAGTGTAATATTCTACTTGGTCTCTGTGTATCATAATTAAAATAGATTTAAATATTGAGTTCAAAAATAGTTTTATTTATCTGGGTGTGAATAAACAGATGCCTGAACTAATTCACAGAAAAGGAAACTTCTGTGTAAAAAGTCAGTCCAATTTCTGAAATGCTATGCTAAACTACAGGTTTATGGAACATTAGATAGGGTGTTAAGACTTTATATAGTACTTCCTCTTGTTTCTATACAAGAGAAAGAAATGGCCATACTTCAGGAATTGCAGTGCATAACTGAGGGATTTTTAGGACTCTTGAATTTTTGATGTAGCCGGGCAACTTTTTTTAGGCAGTGGTAATTATCCTTTATTATGTGAATTTTGAATGGTTTGACAAAACGTTTGTTTTTGTAGAGATTTTAAAAGGGGAGCGCTAATCCTAGAAATAAATATTATGTAATTATTACGGCCTTAAAGATAAAAATCCTTGTTGAAAGTTGAAAAAAATTGCTAAATTACATAGTCTTAGACATTAACATGTTTGTGGAAGAATGTAGCAGAGGTATGTAGTATAATTTGAGTGAATATTCCCAATTAGGAATTCTAGGCTCTAGTTTAACTGAGTCACACTGCATAGGAATTTAGAACCTAACTTCTAGGTTATCAAAATCTTTGCCACCATTGCACAATTTTGTCCTAATATATAGAGAAACTTTGTGAGGCATGTTCAGTTGCGGTTTGCACAAGTTCATCTCATTTGTATTCCAGTGATTTTTTTTCTTCTAACCATTTTTTTAAACAACATGTACACATTGTTTTTTTTGGTAGGCAATGAAAACTGTCATTTCCATTGTCAAACAGTAATTCCTCGATAACTGTATTAATGGTTTTTAAAAAACCATCAGTTACTTTAAAACTGAATTTATATTTAATAACTTCTGTATTAGTATTGGGTAGCATGAAATCTCTATTGAGAAATTGAACAGCATACAACTAGTAGCTGTAAATTCCTTCAGAAAGTGAAAATTATTTCTTCCTAAAGATATCTTGACATCAGTGCTTGAAGAATAGTCATAACTAGATTAATAATTGTTTTAGTTAAACAGTTTTAAGTGCCTGTTTCAGATGATGATAGGCAATTTAGATGAATTTAGGAAAAATCAAAGTTTTTACTTGCAGAAATGTCCATTATAGGGGGCCCCCCTCCTCATAGAGCTGAATGGGTTATGTAATGTTTTATCCAAAAGTTTCCAATTCCACTGTCTTGTGTTTTCATGTTGAAAATACTTTTGCATTTTTCCTTTGAGTGCCAATTTCTTACTAGTACTATTTCTTAATGTAACATGTTTACCTGGAATGTATTTTAACTATTTTTGTATAGTGTAAACTGAAACATGCACATTTTGTACATTGTGCTTTTTTTTGTGGGACATATGCAGTGTGATCCAGTTGTTTTCCATCATTTGGTTGCGCTGACCTAGGAATGTTGGTCATATCAAACATTAAATTTAAAAATGACCACTCTTTTAATTAAAATTAACTTTTAAATGTTTATAGGAGTATGTGCTGTGAAGTGATCTGAAATTTGTAATATTTTTGTCATGAACTGTACTGCTCCTAATTATTGTAATGTAATAAAAATAGTTATGGTGACTATGA

SEQ ID NO: 53 - DNA, 4876 bp, Canis lupus
gctgaaaatgactgaatataaacttgtggtagttggagctggtggcgtaggcaagagtgccttgacgatacagctaattcagaatcactttgtggatgaatatgatcctacaatagaggattcctacaggaaacaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaagactctgaagatgtacctatggtcctagtaggaaataaatgtgatttgccttctagaacagtagacacaaaacaggctcaggacttagcaagaagttatggaattccttttattgaaacatcagcaaagacaagacagggtgttgacgatgccttctatacattagttcgagaaattcgaaaacataaagaaaagatgagcaaagatggtaaaaagaagaaaaagaagtcaaagacaaagtgtataattatgtaaatacaatttgtacttttttcttaaggcatacttaagtaaaagtggtaatttttgtacattacactaaattattagcatttgttttagcattacctaattttctgctccatccaaactgttagcttttatcttgaatgcttattttaaaatgacagtggaaactttttcctctaagtgccagtattccctgagttttggttttgaactagcaatgcctgtgaaaaagaaactgaatacctgagatttctgtcttggggtttttggtgcatgcagttgattacttcctatttttcttaccaattgtgaactttggtgtgaaacaaattaatgaagctttcgaatcatccctattctgtgttttacctagtcacatacatggattaattactaattataacttcagttgatatttcatgattggttttactgaaacattgagggaacatgaatttatgggctgcttcttataggtataatgtcctatagtttcagtcacccttaatgaatgtaaagctacactgttcacaaaggttttctccatcttttcactgctatttgtcatagccacgctcccaaaaatattatattttttctataaaaaagggaaaaaatagaaaaaaatacaaggcaatggaaaatattaaaaggcatttactttccatattagataaattcctataatactctgaatagcttttcctgttaaggcagacccagtatgtaatgaggattatagcaaccattttggggctatatttacatgctactaaatttttgtattaattgaaaaagttttaacatgtataaaaaattcccataggaattaaatatagtctccctgtgtcagattgctctttcttagcataactttaaatcttttcttgatcttcaatcttagaaaatagttttaattcttgtagtgatgttaaagattatttgggccagttagtttttaatagatgttaaagagaccacagttccaaggccaggccttgtgtgaacctttaagcttcattaagagtttcatagtacagactgcatccctgtggtctcccagggtcatcatgcattgattgggtggtcaaaagtggggacaaagagtgtttagataagatgcatcctcactgtatggtggtcctgctgacagatcaggaccatcacttttgttttttaaaaaaccaacagagctttttaaaaacattatttaaaatgagatttttgggggcagggggtggcaagacttgaattttttttaaacaatgaagtaaaaaggtttcaaaatctctagtgttggctagttctcaacattggctaaagtaacatttcataaacactttacaagtattggtccatatttaagaatatctaatgcttaaataatagattaataacaattctttcagtgcatttaaaatgtatttttaaatatctgaagtgagatggtgtgttgaggtgaaaatatcactggactaggaggaaggtgacttagattctagttacgtgtcttttacaacttcagttttgggcaaatcactcactatccatttcttcatgttaagtcatctcaaaggctatatctagcatcaactatgtgatttacattcagtttacataaggatatacctatttgtcaatctcagcacaatctgtaactttttacctatgttctcttcagcgccagtcttaggcaaagttgtgcaagaggtgaggtttatttttgagaatctgatctccggtagcaggtactcctctcccatgttagtgtcatcttgcctgcctaccttctacatgccccatgacttgatgctttctaattccccgaacctcaagatgtagtgctgctttggatatctccatgaggtaataagtcacattagtcaggctcaacataatctgacagatactgtagtgggatttgatctaatagctaattttcaggtggtaactgtatcaatttaattttgatcttttgaacatcatctctgctacctggtccattagtgactaagtaggaaaagtaggaattttcatatctgtgatgtgtagacagaccctatccagtagaagaatttaataaatttaattaataaatactgaaagatttccttagataatccaaaactaggactagccctggtaacggtgatacattccattattttaataagtaaaatcttcttacaatgaaaaatactttaaaatttaattcataaagcttactttttagcagaattcatttattcaacaaatacttgagtgcctgctagatgccaggttctacacaaggcaccggggatattatggtattcccaacaagggacataatccctatccttaagtagtactgttattttagagtggtctgtagtatattagtgaggcatttggcacatgacccagagataatataatgcatattttagttttgcacagaagggatatggtctctaaggttttttccagctctaaaataattgttcgctctgattccaataaactgtttaatcaagctactttatataaatcactttacttcattattttaaagaagtaaacttgactatattgttttttatttgggataattatgtgattctgttgggatacttatatagtacacattaaattgtatgtcagatgataacattaaaattcccaagtgtaatattctacttggtctctgtgtatcataattaaaatagatttaaatattgagttcaaaaatagttttatttatctgggtgtgaataaacagatgcctgaactaattcacagaaaaggaaacttctgtgtaaaaagtcagtccaatttctgaaatgctatgctaaactacaggtttatggaacattagatagggtgttaagactttatatagtacttcctcttgtttctatacaagagaaagaaatggccatacttcaggaattgcagtgcataactgagggatttttaggactcttgaatttttgatgtagccgggcaactttttttaggcagtggtaattatcctttattatgtgaattttgaatggtttgacaaaacgtttgtttttgtagagattttaaaaggggagcgctaatcctagaaataaatattatgtaattattacggccttaaagataaaaatccttgttgaaagttgaaaaaaattgctaaattacatagtcttagacattaacatgtttgtggaagaatgtagcagaggtatgtagtataatttgagtgaatattcccaattaggaattctaggctctagtttaactgagtcacactgcataggaatttagaacctaacttctaggttatcaaaatctttgccaccattgcacaattttgtcctaatatatagagaaactttgtgaggcatgttcagttgcggtttgcacaagttcatctcatttgtattccagtgattttttttcttctaaccatttttttaaacaacatgtacacattgttttttttggtaggcaatgaaaactgtcatttccattgtcaaacagtaattcctcgataactgtattaatggtttttaaaaaaccatcagttactttaaaactgaatttatatttaataacttctgtattagtattgggtagcatgaaatctctattgagaaattgaacagcatacaactagtagctgtaaattccttcagaaagtgaaaattatttcttcctaaagatatcttgacatcagtgcttgaagaatagtcataactagattaataattgttttagttaaacagttttaagtgcctgtttcagatgatgataggcaatttagatgaatttaggaaaaatcaaagtttttacttgcagaaatgtccattatagggggcccccctcctcatagagctgaatgggttatgtaatgttttatccaaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgcttttttttgtgggacatatgcagtgtgatccagttgttttccatcatttggttgcgctgacctaggaatgttggtcatatcaaacattaaatttaaaaatgaccactcttttaattaaaattaacttttaaatgtttataggagtatgtgctgtgaagtgatctgaaatttgtaatatttttgtcatgaactgtactgctcctaattattgtaatgtaataaaaatagttatggtgactatga

SEQ ID NO: 54 - protein, 188 aa, Canis lupus; identical to SEQ ID NO: 50

SEQ ID NO: 55 - DNA, 1394 bp, Felis catus
gctgaaaatgactgaatataaacttgtggtagttggagctggtggcgtaggcaagagtgccttgacgatacagctaattcagaatcactttgtggatgaatatgatcctacaatagaggattcctacaggaaacaagtagtaattgatggagaaacctgtctcttggatattctcgacacagcaggtcaagaggagtacagtgcaatgagggaccagtacatgaggactggggagggctttctttgcgtatttgccataaataatactaaatcatttgaagatattcaccactatagagaacaaataaaaagagttaaagactctgaagatgtacctatggtcctagtaggaaataaatgtgatttgccttctagaacagtagatacaaaacaggctcaggacttagcaagaagttatggaattccttttattgaaacatcagcaaagacaagacagggtgttgacgatgccttctatacattagttcgagaaattcgaaaacataaagaaaagatgagcaaagatggtaaaaagaagaaaaagaagtcaaagacaaagtgtataattatgtaaatacaatttgtacttttttcttaaggcatacttaagtaaaagtggtaatttttgtacattacactaaattattagcatttgttttagcattacctaattttctgctccatccaaactgttagcttttatcttgaatgcttattttaaatgacagtggaaactttttttcctctaagtgccagtattccctgagttttggtttttgaactagcaatgcctgtgaaaaagaaactgaatacctgagatttctgtcttggggtttttggtgcatgcagttgattacttcctatttttcttaccaattgtgaactttggtgtgaaacaaattaatgaaactttcgaatcatccctattctgtgtttcatgtagtcacatacatggattaattactaattataacttcagttgagatttcatgattcgttttactgaaacattgagggaacatgaatttatgggcttcttgtagattcatcttgtaggtattaatgtcctatagtttcagtcacccttaatgaatgtaaagttacactgttcataaaagtttctccatcttttcactgctgtttgtcatcgtcacgctcccccaaaatattatattttttctataaaaagggaaaaaaggaaaaaaatacaaggcaatggaaaatattaaaaggcatttactttctgtattagataaattcctataatactctgaatagcttttcctgttaaggcagacccagtatgtgatgaggattatagcaaccattttggggctatatttacatgctactaaatttttgtaataattgaaaaaattttaacatgtataaaa

SEQ ID NO: 56 - protein, 188 aa, Felis catus; identical to SEQ ID NO: 50

SEQ ID NO: 57 - DNA, 1512 bp, Felis catus
tgctgaaaatgactgaatataaacttgtggtagttggagctggtggcgtaggcaagagtgccttgacgatacagctaattcagaatcactttgtggatgaatatgatcctacaatagaggattcctacaggaaacaagtagtaattgatggagaaacctgtctcttggatattctcgacacagcaggtcaagaggagtacagtgcaatgagggaccagtacatgaggactggggagggctttctttgcgtatttgccataaataatactaaatcatttgaagatattcaccactatagagaacaaataaaaagagttaaagactctgaagatgtacctatggtcctagtaggaaataaatgtgatttgccttctagaacagtagatacaaaacaggctcaggacttagcaagaagttatggaattccttttattgaaacatcagcaaagacaagacagagagtggaggatgctttttatacattggtgagagagatccgacagtacagattgaaaaaaatcaacaaagaagaaaagactcctggctgtgtgaaaattaaaaaatgcattgtaatgggtgttgacgatgccttctatacattagttcgagaaattcgaaaacataaagaaaagatgagcaaagatggtaaaaagaagaaaaagaagtcaaagacaaagtgtataattatgtaaatacaatttgtacttttttcttaaggcatacttaagtaaaagtggtaatttttgtacattacactaaattattagcatttgttttagcattacctaattttctgctccatccaaactgttagcttttatcttgaatgcttattttaaatgacagtggaaactttttttcctctaagtgccagtattccctgagttttggtttttgaactagcaatgcctgtgaaaaagaaactgaatacctgagatttctgtcttggggtttttggtgcatgcagttgattacttcctatttttcttaccaattgtgaactttggtgtgaaacaaattaatgaaactttcgaatcatccctattctgtgtttcatgtagtcacatacatggattaattactaattataacttcagttgagatttcatgattcgttttactgaaacattgagggaacatgaatttatgggcttcttgtagattcatcttgtaggtattaatgtcctatagtttcagtcacccttaatgaatgtaaagttacactgttcataaaagtttctccatcttttcactgctgtttgtcatcgtcacgctcccccaaaatattatattttttctataaaaagggaaaaaaggaaaaaaatacaaggcaatggaaaatattaaaaggcatttactttctgtattagataaattcctataatactctgaatagcttttcctgttaaggcagacccagtatgtgatgaggattatagcaaccattttggggctatatttacatgctactaaatttttgtaataattgaaaaaattttaacatgtataaaa

SEQ ID NO: 58 - protein, 227 aa, Felis catus
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKINKEEKTPGCVKIKKCIVMGVDDAFYTLVREIRKHKEKMSKDGKKKKKKSKTKCIIM

SEQ ID NO: 59 - DNA, 2144 bp, Bos taurus
ggcggtggcggcggcggcggcggtggcggtggcggttcggccagtactcccggcccccgccatttctgactgggagcgagcgcggcgcaggcactgaaggcagcggcgggggccagaggctcggcggctcccaggtgagggagagaggcctgctgaaaatgactgaatataaacttgtggtagttggagctggtggcgtaggcaagagtgccttgacgatacagctaattcagaatcactttgtggatgaatatgatcctacgatagaggattcctacaggaaacaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaataaaaagagttaaagactctgaagatgtacctatggttctagtaggaaataaatgtgatttgccttctagaacagtagacacaaaacaggctcaggacttagcaagaagttatggaattccttttattgaaacatcagcaaagacaagacagggtgttgacgatgccttctatacattagttcgagaaattcgaaaacataaagaaaagatgagcaaagatggtaaaaagaagaaaaagaagtcaaagacaaagtgtataattatgtaaatacaatttgtacttttttcttaaggcatacttaagtaaaagtggtaatttttgtatattacactaaattattagcatttgttttagcattatctaattttctttctgctccatccatactgttagcttttatcttgaatgcttattttaaaatgacagtggaaacttttttcctctaagtgccagtattccctgcgttttggtttttgaactagcaatgcctgtgaaaaagaaactgaacacccaagatttttgtcttggggtttttggtgcatgcagttgattacttcctatttttcttatcaattgtgaactttagtgtgaaacaaattaatgaggctttcaaatcatccctattgtattgttttatctagtcacacacatggattaattactaattataacttcagttgagatttcatgattggttttactgaaacatcgagggaacatgaatttatgggcttcctatagtttcatcttgtaggtatcattgtcctatagtttcagttacccttaatgaatgtcaggttacactgttcacaaaggttttcttctttccactgctatttgtcaaatggtcacgttccctaaaatactatattttttctataaaaaaaagaaaaaaatggaaaaaaatacaaggcaatggaaaatattaaaaggccacttactttccacattaggtaaattcctataatgctctgaatagctttttatgttaaggcagacccagtaggtaatgaggattagaacaagcattttgggactatatttacatgctttaaatttttgtaataacaaaaaaattttaacatgtataaagaattctcataggaattaaatacagtctccctgtgtcagattgctctttcttagcataaatctttttcttgaacttcaatctttaaaagtagttttaattctactgatagtgatgtaaaagattatttgggccagttagcttggtaggtgttacagagaccagggtggcatagccgggccttgtgtgaacctttaagctacatggagagtttcacagtgtggactgcatccctgtggtcttccattgttgccatgccttggttggtcaaaaacaaggacttgcagagagattgaatagctcagcaaggtacattctcattatgtcgtagtcctactcaggaacatcacttttttaaaataaaaaaccccaaaaaacagaacttaaaaaaaaaaacaacattattttaaatgagattttcggtggggtggaaagattttaattttttttaaacgatgaaatgaaaaaatgtcaaaatcttgagtattggctagttctctttaacactggctaaagtaacatttttgtaaacacttcagtacagtctggtccattattaagaatatctaatgcttatacaataaagtaatgctaac

SEQ ID NO: 60 - protein, 188 aa, Bos taurus; identical to SEQ ID NO: 50

SEQ ID NO: 61 - DNA, 1010 bp, Bos taurus
ggcggtggcggcggcggcggcggtggcggtggcggttcggccagtactcccggcccccgccatttctgactgggagcgagcgcggcgcaggcactgaaggcagcggcgggggccagaggctcggcggctcccaggtgagggagagaggcctgctgaaaatgactgaatataaacttgtggtagttggagctggtggcgtaggcaagagtgccttgacgatacagctaattcagaatcactttgtggatgaatatgatcctacgatagaggattcctacaggaaacaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaataaaaagagttaaagactctgaagatgtacctatggttctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattgtaatgggtgttgacgatgccttctatacattagttcgagaaattcgaaaacataaagaaaagatgagcaaagatggtaaaaagaagaaaaagaagtcaaagacaaagtgtataattatgtaaatacaatttgtacttttttcttaaggcatacttaagtaaaagtggtaatttttgtatattacactaaattattagcatttgttttagcattatctaattttctttctgctccatccatactgttagcttttatcttgaatgcttattttaaaatgacagtggaaactt

SEQ ID NO: 62 - protein, 227 aa, Bos taurus
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIVMGVDDAFYTLVREIRKHKEKMSKDGKKKKKKSKTKCIIM

SEQ ID NO: 63 - DNA, 797 bp, Bos taurus
gcggtggcggcggcggcggcggtggcggtggcggttcggccagtactcccggcccccgccatttctgactgggagcgagcgcggcgcaggcactgaaggcagcggcgggggccagaggctcggcggctcccaggtgagggagagaggcctgctgaaaatgactgaatataaacttgtggtagttggagctggtggcgtaggcaagagtgccttgacgatacagctaattcagaatcactttgtggatgaatatgatcctacgatagaggattcctacaggaaacaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaataaaaagagttaaagactctgaagatgtacctatggttctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattgtaatgtaatctgggtgttgacgatgccttctatacattagttcgagaaattcgaaaacataaagaaaagatgagcaaa

SEQ ID NO: 64 - protein, 189 aa, Bos taurus
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIVM

SEQ ID NO: 65 - DNA, 1112 bp, Gallus gallus
caggtctgctaaaaaatgacagagtataagcttgttgtcgttggagctggtggtgtgggcaagagcgccttgacaatacagctcattcagaaccactttgtggatgagtatgaccctaccatagaggattcctacagaaagcaagtagtaattgatggggaaacctgtctcttggatattcttgatacagcaggtcaagaagaatatagtgcaatgagggaccaatatatgagaacaggagaaggctttctgtgtgtttttgctataaacaatacaaaatcttttgaagatattcaccattatagggaacaaataaagagagttaaagactctgaagatgtcccaatggtgctagtaggaaacaaatgtgatttgccttccagaacagtagatacaaaacaagctcaggatttagcaagaagttatggaattccttttattgaaacatcagcaaagacaagacagggtgttgatgatgccttctatacattagttcgagaaatcagaaaacacaaagagaagatgagcaaagatggtaaaaagaagaaaaagaagacaaagacaaagtgtataattatgtaaatacaatgtatccttattcttaagacgtactgaagtaatttttgtacattacactaaattattagcatttgtttttagcattactttactttctgcttcatgatcctgttagctttacctgaatgcttgttttaaatgacagtggaaacttcattcctcttaaagtgccagtattctttgagtgttggttcttgaactagcaatgcctgtgaagaaaaataaaaacaaatgaaaaaaaaaaaaacacacaaaaacctgagaactgtcttaggactctttggtgcatgcacagttgctaacttcctatttttcttactgattgtgaacttctgttccgtgcgtaaacaaaacaatgaaacgatctacacgttctaacatcccccttcatttgtactctcttattttttacatctggttgggaaaacggaccagttagtgacaaagactttattttcagacttccttctaatttcgactgactgcaatatagagagaccagaagcctttatagtcttcctgtagattttgct

SEQ ID NO: 66 - protein, 188 aa, Gallus gallus
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKTKTKCIIM

SEQ ID NO: 67 - DNA, 4454 bp, Homo sapiens
gaaacgtcccgtgtgggaggggcgggtctgggtgcggcctgccgcatgactcgtggttcggaggcccacgtggccggggcggggactcaggcgcctggggcgccgactgattacgtagcgggcggggccggaagtgccgctccttggtgggggctgttcatggcggttccggggtctccaacatttttcccggctgtggtcctaaatctgtccaaagcagaggcagtggagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaagttttgtcagaaaagagccactttcaagctgcactgacaccctggtcctgacttccctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctctgccaccccaggtttttcatctgaaaagcagttcatgtctgaaacagagaaccaaaccgcaaacgtgaaattctattgaaaacagtgtcttgagctctaaagtagcaactgctggtgattttttttttctttttactgttgaacttagaactatgctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaactttcacagtgaagtgcctttttcctagaagtggtttgtagacttcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcacatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaattacattttcactgtatatcatggtatcttaatgatgtatataattgccttcaatccccttctcaccccaccctctacagcttcccccacagcaataggggcttgattatttcagttgagtaaagcatggtgctaatggaccagggtcacagtttcaaaacttgaacaatccagttagcatcacagagaaagaaattcttctgcatttgctcattgcaccagtaactccagctagtaattttgctaggtagctgcagttagccctgcaaggaaagaagaggtcagttagcacaaaccctttaccatgactggaaaactcagtatcacgtatttaaacatttttttttcttttagccatgtagaaactctaaattaagccaatattctcatttgagaatgaggatgtctcagctgagaaacgttttaaattctctttattcataatgttctttgaagggtttaaaacaagatgttgataaatctaagctgatgagtttgctcaaaacaggaagttgaaattgttgagacaggaatggaaaatataattaattgatacctatgaggatttggaggcttggcattttaatttgcagataataccctggtaattctcatgaaaaatagacttggataacttttgataaaagactaattccaaaatggccactttgttcctgtctttaatatctaaatacttactgaggtcctccatcttctatattatgaattttcatttattaagcaaatgtcatattaccttgaaattcagaagagaagaaacatatactgtgtccagagtataatgaacctgcagagttgtgcttcttactgctaattctgggagctttcacagtactgtcatcatttgtaaatggaaattctgcttttctgtttctgctccttctggagcagtgctactctgtaattttcctgaggcttatcacctcagtcatttcttttttaaatgtctgtgactggcagtgattctttttcttaaaaatctattaaatttgatgtcaaattagggagaaagatagttactcatcttgggctcttgtgccaatagcccttgtatgtatgtacttagagttttccaagtatgttctaagcacagaagtttctaaatggggccaaaattcagacttgagtatgttctttgaataccttaagaagttacaattagccgggcatggtggcccgtgcctgtagtcccagctacttgagaggctgaggcaggagaatcacttcaacccaggaggtggaggttacagtgagcagagatcgtgccactgcactccagcctgggtgacaagagagacttgtctccaaaaaaaaagttacacctaggtgtgaattttggcacaaaggagtgacaaacttatagttaaaagctgaataacttcagtgtggtataaaacgtggtttttaggctatgtttgtgattgctgaaaagaattctagtttacctcaaaatccttctctttccccaaattaagtgcctggccagctgtcataaattacatattccttttggtttttttaaaggttacatgttcaagagtgaaaataagatgttctgtctgaaggctaccatgccggatctgtaaatgaacctgttaaatgctgtatttgctccaacggcttactatagaatgttacttaatacaatatcatacttattacaatttttactataggagtgtaataggtaaaattaatctctattttagtgggcccatgtttagtctttcaccatcctttaaactgctgtgaatttttttgtcatgacttgaaagcaaggatagagaaacactttagagatatgtggggtttttttaccattccagagcttgtgagcataatcatatttgctttatatttatagtcatgaactcctaagttggcagctacaaccaagaaccaaaaaatggtgcgttctgcttcttgtaattcatctctgctaataaattataagaagcaaggaaaattagggaaaatattttatttggatggtttctataaacaagggactataattcttgtacattatttttcatctttgctgtttctttgagcagtctaatgtgccacacaattatctaaggtatttgttttctataagaattgttttaaaagtattcttgttaccagagtagttgtattatatttcaaaacgtaagatgatttttaaaagcctgagtactgacctaagatggaattgtatgaactctgctctggagggaggggaggatgtccgtggaagttgtaagacttttatttttttgtgccatcaaatataggtaaaaataattgtgcaattctgctgtttaaacaggaactattggcctccttggccctaaatggaagggccgatattttaagttgattattttattgtaaattaatccaacctagttctttttaatttggttgaatgttttttcttgttaaatgatgtttaaaaaataaaaactggaagttcttggcttagtcataattctt

SEQ ID NO: 68 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 69 - DNA, 1326 bp, Rattus norvegicus
gccgttcatggcggtttcggggtctccaacagcttctcaggttgaaatccaaaagcctcccgaggcggggtctgcggagtttgagatttttgcaggtgtgaaatgactgagtacaaactggtggtggttggagcaggtggcgttgggaaaagtgctttgacaatccagctaatccagaaccactttgtggatgaatatgatcccaccatagaggattcttaccgaaaacaagtggtgattgacggtgagacctgtctactggacatactggacacagctggacaagaggagtacagtgccatgagagaccaatacatgaggacaggcgaagggttcctctgtgtgtttgccatcaataatagcaaatcctttgcagatattaacctctacagggagcaaattaagcgcgtgaaagactctgatgatgtacccatggtgctggtagggaacaagtgtgacttgccaacaaggacagttgacacaaagcaagcccacgagctggccaagagttatggaattccattcattgaaacctcagccaagacccgacagggtgtggaggatgccttttacacgcttgtaagggagatacgccagtaccggatgaagaagctcaacagcagtgaggatggcactcaaggctgtatggggctgccctgtgtggtgatgtagtaagaccctttaaaagttctgtcatcagaaacgagccactttcaagcctcactgatgccctggttctgacatccctggaggagacgtgtttctgctgctctctgcatctcagagaagctcctgcttcctgcttccccaacttagttactgagcacagccatctaacctgagacctcttcagaataactacctcctcactcggctgtccgaccagagaaatgaacctgtttctccccagtagttctctgccctgggtttcccctagaaacaaacacacctgccagctggctttgtcctccgaaaagcagtttacattgatgcagagaaccaaactatagacaagcaattctgttgtcaacagtttcttaagctctaaggtaacaattgctggtgatttccccctttgcccccaactgttgaacttggccttgttagttttgggggaaatgtcaaaaattaatctcttcccgagaatagaattagtgttgctgattgcctgatttgcaatgtgatcagctatattctataagctggcgtctgctctgtattcataaatgcaaacatgagtactgacgtaagtgcatccctagtcttctcagctgcatgcaattaaatccaacgttcacaacaaaaaaaaaaaaaaaaaaaaaaaaa

SEQ ID NO: 70 - protein, 189 aa, Rattus norvegicus
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSEDGTQGCMGLPCVVM

SEQ ID NO: 71 - DNA, 4470 bp, Mus musculus
gggactggggcgccttgggcgcctagtgattacgtagcgggtggggccggaagtgccgctccctggcgggggctgttcatggcggtttcggggtctccaacagcttctcaggttgaagtccaaaagcctcccgaggcggggtctgcggagtttgaggtttttgctggtgtgaaatgactgagtacaaactggtggtggttggagcaggtggtgttgggaaaagcgccttgacgatccagctaatccagaaccactttgtggatgaatatgatcccaccatagaggattcttaccgaaagcaagtggtgattgatggtgagacctgcctgctggacatactggacacagctggacaagaggagtacagtgccatgagagaccagtacatgaggacaggcgaagggttcctctgtgtatttgccatcaataatagcaaatcatttgcagatattaacctctacagggagcaaattaagcgtgtgaaagattctgatgatgtccccatggtgctggtaggcaacaagtgtgacttgccaacaaggacagttgacacaaagcaagcccacgaactggccaagagttacggaattccattcattgagacctcagccaagacccgacagggtgtggaggatgccttttacacactggtaagggagatacgccagtaccgaatgaaaaagctcaacagcagtgacgatggcactcaaggttgtatggggctgccctgtgtgctgatgtagtaagacactttgaaagttctgtcatcagaaaagagccactttgaagctgcactgatgccctggttctgacatccctggaggagacctgttcctgctgctctctgcatctcagagaagctcctgcttcctgcttccccgactcagttactgagcacagccatctaacctgagacctcttcagaataactacctcctcactcggctgtctgaccagagaaatagacctgtctctcccggtcgttctctgccctgggttcccctagaaacagacacagcctccagctggctttgtcctctgaaaagcagtttacattgatgcagagaaccaaactagacatgccattctgttgacaacagtttcttatactctaaggtaacaactgctggtgattttcccctgcccccaactgttgaacttggccttgttggtttggggggaaaatgtcataaattactttcttcccaaaatataattagtgttgctgattgatttgtaatgtgatcagctatattccataaactggcatctgctctgtattcataaatgcaaacacgaatactctcaactgcatgcaattaaatccaacattcacaacaaagtgcctttttcctaaaagtgctctgtaggctccattacagtttgtaattggaatagatgtgtcaagaaccattgtataggaaagtgactctgagccatctacctttgagggaaaggtgtatgtacctgatggcagatgctttgtgtatgcacatgaagatagtttccctgtctgggattctcccaggagaaagatggaactgaaacaattacaagtaatttcatttaattctagctaatcttttttttttttttttttttttttggtagactatcacctataaatatttggaatatcttctagcttactgataatctaataattaatgagcttccattataatgaattggttcataccaggaagccctccatttatagtatagatactgtaaaaattggcatgttgttactttatagctgtgattaatgattcctcagaccttgctgagatatagttattagcagacaggttatatctttgctgcatagtttcttcatggaatatatatctatctgtatgtggagagaacgtggccctcagttcccttctcagcatccctcatctctcagcctagagaagttcgagcatcctagaggggcttgaacagttatctcggttaaaccatggtgctaatggaccgggtcatggtttcaaaacttgaacaagccagttagcatcacagagaaacagtccatccatatttgctccctgcctattattcctgcttacagacttttgcctgatgcctgctgttagtgctacaaggataaagcttgtgtggttctcaccaggactggaagtacctggtgagctctggggtaagcctagatatctttacattttcagacccttattcttagccacgtggaaactgaagccagagtccatacctccatctccttccccccccaaaaaaattagattaatgttctttatatagcttttttaaagtatttaaaacatgtctataagttaggctgccaactaacaaaagctgatgtgtttgttcaaataaagaggtatccttcgctactcgagagaagaatgtaaaatgccattgattgttgtcacttggaggcttgatgtttgccctgataattcattagtgggttttgtttgtcacatgatacctaagatgtaactcagctcagtaattctaatgaaaacataaattggataccttaattgaaaaaagcaaacctaattccaaaatggccattttctcttctgatcttgtaatacctaaaattctgaggtccttgggattcttttgtttataacaggatcttgctgtgtagtcctagctggcctcaaactcacaatactcttcctggatcaatctcccaagtgctgggattacaggcacattccaccacacacacctgactgagctcgttcctaatgagttttcattaagcaaattccccatcaccttgaaactaatcagaagggggaagaaacatttgctatgctcctgagtgctaacactgggatcattcacatggggtttgcattcctaggcaaactaaactgctgccttttacaacaaggctcagtcatcttcctgaagctgctgagaccagcacttggtcttgttttgttttaatatgtctatatgactggtggtggatccctaaatagtttattaattaaactccagttaaggagaaagttactcaccttgacccgtttgaccatatcccgtgtgtgtgtgtgtgtgtgtgtgtgtgtgtgtgtgtgtgtgtgtgtgcacgcgtatgtacgtacgtatgtatgtaggtatgtaggtggtttccagtataaacacagaaacaaatggagccaattcaggtttcagatgcccttactaacatatattcccacggggtgtgggttttggcacaacagtgacaaacttaaaagccaagtaagagccgggcgtggtggcgcacgcctttaatcccagcacttgggaggcagaggcaggcggatttctgagttctaggccatcctggtctacagtgagttccaggacagccagtgctacacagagaaaccctgtctcgaaaagccaaaaaaaaaaaaaaaaaaaaaaaaaaaaagccaagtaggtccagttggtatagtatcaaagtgtttttagagtaattagtgaaggtctgctttacctcaaagttgcagagcctctcttcctgagtttaagtgcctggccggcagtcacaaattaacatgttgctgtaaggcagttagttgaagctttgttcacacattggagagtatgaaaataaagtgttctaagagcgctgatactggatctgtgtaaacctggtaaatgccgtttgtccaggacttagcgtgtgtgagttggtagctcagtacgagtttactagttccgcagtgtgtacaatggaggcgggtttgttttagctggccacctgtagaatcagcctttaaactgctgtgaactttgtcatgacttgaatatgaagatagacaaaaactctgtaaagacaaatgtttgttttcccccttacagaacgtgtgagcttggttttatcttcctttgtatttagtcataacctctcaagctggcagctccgaccaaggatcagaagctgtgtgcgttccacctggtggaattagctcagctctatatgagaagtggagttaatggaaaacgtgttgactgggtggtttctatttaaaagagtgatgataattcttgaacagtagtttttattttgctatttctttaagctgactgatgtgccacaaaattattttaaggtatttgtgttttaagagtgttctcatgagattagttgtagatattttttaaaatacaactggtttttaaaatctgagtattgctctaagcaagtgtttagactcttacgggaaggtgggtggaagttgtttggcttccgtatttccatgcgtgccgtcagacataggtcagaacgccaactgtgcatcctgctgtttaaagacctcttggcctctgtgaccctcatgaaggggctgatattttaagttgactgtttgattgtaaattaatcctttctaatttttaaagacttgcttgactgttttccttgttaaataattttaaaaaaataaaaaactggaagttctttgcttaactgta

SEQ ID NO: 72 - protein, 189 aa, Mus musculus
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVLM

SEQ ID NO: 73 - DNA, 570 bp, Cavia porcellus
atgactgagtataaactggtggtggttggagcaggtggtgtcgggaaaagtgcactgaccatccagctaattcagaaccactttgtcgatgaatatgatcccaccatagaggattcttaccgaaaacaggtggttatagatggtgaaacttgtctgttggatattctggatacagctggacaagaggagtacagtgccatgagagaccaatacatgaggacaggcgaaggcttcctctgtgtgtttgccatcaataatagcaaatcatttgcagatattaacctctacagggagcagattaaacgagtaaaagactcagatgatgtacctatggtgctggtagggaacaagtgtgatttgccaacaaggactgttgacacaaaacaagcccatgaactggccaagagttacgggattccattcattgaaacctcagccaagaccagacagggtgttgaagatgcattttacacactcgtaagagaaatacgccagtacagaatgaaaaaactcaacagcaatgatgatgggactcaaggttgtatggggttgccatgtgtggtgatgtaa

SEQ ID NO: 74 - protein, 189 aa, Cavia porcellus
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSNDDGTQGCMGLPCVVM

SEQ ID NO: 75 - DNA, 1220 bp, Cavia porcellus
gttccggggtcctcaacgtttctcagggttgagattctatatccttttgaagctggggcggcagagcttgaggttcttgctggtgtgaaatgactgagtataaactggtggtggttggagcaggtggtgtcgggaaaagtgcactgaccatccagctaattcagaaccactttgtcgatgaatatgatcccaccatagaggattcttaccgaaaacaggtggttatagatggtgaaacttgtctgttggatattctggatacagctggacaagaggagtacagtgccatgagagaccaatacatgaggacaggcgaaggcttcctctgtgtgtttgccatcaataatagcaaatcatttgcagatattaacctctacagggagcagattaaacgagtaaaagactcagatgatgtacctatggtgctggtagggaacaagtgtgatttgccaacaaggactgttgacacaaaacaagcccatgaactggccaagagttacgggattccattcattgaaacctcagccaagaccagacagggtgttgaagatgcattttacacactcgtaagagaaatacgccagtacagaatgaaaaaactcaacagcaatgatgatgggactcaaggttgtatggggttgccatgtgtggtgatgtaacaagatatttaacaaagttctatcagaaaagagccactttcaagctgcactgataccctggtcctgacttccctggaggagaagtatccctgttgctctcttcatctcagagaagctcctgctgtttgtccacctctcagtgtatgagcacagtctctgcttgagaacttctcagaataactacctcctcacttggttgtctgaccagagaaatgcacctcttgttaattccccaataattttctgccctgggctctccccaacaaaaaacaaacacttctgccatccaaaaagcaacttggtctgaaacagaaccaaactgtagattgaaattctcttaaaaagtcttgagctctaaagttagcaaccgctggtgatttttattttcctttttatttttgaacttggaactgacctatgttagattttggagaaatgtcataaagtactgttgtgccaagaagataattatgttgctgaatggttgatttatagtgttatcagctatattttacaaactggcatctgctctgtattcataaatacaaaaatgaagccagg

SEQ ID NO: 76 - protein, 189 aa, Cavia porcellus; identical to SEQ ID NO: 74

SEQ ID NO: 77 - DNA, 1307 bp, Canis lupus
tgattacgtagcgggcggggccggaagtgccgctccctagtgggggctgttcatggcggttccggggtctccaacctttctcctagttgtggtcctaaatacgtcggaagcggaggcggcgaagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttaccgaaaacaggtggttatagacggtgaaacctgtctgttggatatactggatacagctggtcaagaagagtacagtgccatgagagaccaatacatgaggacaggcgaaggcttcctctgtgtatttgccatcaataatagcaaatcatttgcagacattaacctctacagggaacagattaagcgagtaaaagattcagatgatgtacctatggtgctagtaggaaacaagtgtgatttgccaacaaggacagttgacacaaaacaagcccatgaactggccaagagttatgggattccattcattgaaacctcagccaagaccagacagggtgtcgaggatgccttttacacactggtaagagaaatacgtcagtaccgaatgaagaaactcaacagcagtgatgatgggactcaaggttgtatggggttaccatgtgtggtgatgtaacaagacacttttaaagttctagcatcagaaaagagccactgtcaagctgcactgacaccctggtcctgacttccctggaggagaagtattcctgttgctatcttcagtctcacaaagaagctcctgctacttccccaactctcagtagatcagtacaatgttctctatttgagaagttctccgaacaactacctcctcacttggttgtctgaccagagaaatgaacctcttgttccttcccgctgtttttccaccctgaattctcccccaacacacataaacaaacctctgccatcccaggtttttcatctgaaaaataattcatgctctgaaacagagaacaaaactgtagacatgaaattctgtaggaaacaaggtcttgagctcaaaagtagcaactgctggtgacctttttttcccccctttttactgttgaacttggaactatgttggtttttggagaaatgtcataagttactgttttgctgagaatatagttaagttgacatttggtttgtttgtaatatcattagctattttctataaattggcatctgctctgcattcataaatacacgagtgaattctga

SEQ ID NO: 78 - protein, 189 aa, Canis lupus; identical to SEQ ID NO: 68

SEQ ID NO: 79 - DNA, 3104 bp, Felis catus
aaaaaataaataaatttaagaaaccattttaaaattatgcacagttgcagcctggaaaacttaaggtggcgccttatagtatcaatcttaggagctttatttggtgcatttaacgcaactggtaattgcaaaatccacttcgcctgtgtaagtgaaaaatatagactgttatcttgttggccctatgaaattctgcacttggtatttagcatatactctaccttcattactatctggcaagatgttctgccttagcactcagttgcattcttttccttttctttcctgttcattatgctttaattctgaggaccatatgagggtagaatatattaaaaattacaaaaattataaaaatttgtataggcaaaccatttccttaagttgatggccaaatgttaaaatgttatttttcatatcatttataatcttgtcacagtccacttaacgaagtttggttagatttcagtgaaaattatcttccagagtagtttttttttttttttcctgggattagggaggggggtaactttactgcaattagtatgtatggtgcagaatttcatgcaaatgaggtgtgccagcagtgtggtaatttaatcgtatttaaacaaaaacaaacaaaaaaaaaacgaatgcacaaacttgctgctgcttagatcactgcagcttctaggacccagtttcttttactgatttcaaaacaaaacaaaacaaaaaaataaaaaaagttgtgcctgaaatgaatcttgttttttttataagtagccgcctggttcctgtgtcctgtgaaatacaggcacttgacccttggtgtagcttctgttcgactttatatcacgggaatggattggtctgatttcttggccctcatcttgaattggccacatccagggtccctggccagtggactgaaggctttgtctaagaggacaagggcagctcaggggatgtgggggagggcgcttttatcttccccgttgtcgtttgaggttttgatcttctctgggtaaagaggccgtttatctttgtaaacacaaaacatttttgctttctccagttttctgttaatggcgaaagaatggaagcgaataaagttttactgatttttgagactctagcacctagcgctttcatttttgaaacgtcctgtgtgggaggggcgggtctgggtgcggcccgccgcgtgactcctgagtcgggggcccacgtggctggggcggggactcggacgccccgggcgccgactgattacgtagcgggcggggccggaagtgccgctccctagtgggggctgttcatggcggttccggggtctccatcctttttcccagttgttctaaatcagtcggaagcggaggcagcgaagtttgaggttctcgctggtgtgaaatgactgagtacaaactggtggtggttggagcaggtggtgttgggaaaagcgcactgacaatccagctaatccagaaccactttgtagatgaatatgatcccaccatagaggattcttaccgaaaacaggtggttatagacggtgaaacctgtctgttggacatactggatacagctggtcaagaagagtacagtgccatgagagaccaatacatgaggacaggcgaaggcttcctctgtgtatttgccatcaacaatagcaaatcatttgcagatattaacctttacagggaacagattaagcgagtaaaagactccgatgatgtacctatggtgctagtaggaaacaagtgtgatttgccaacaaggaccgtcgacacaaaacaagcccacgaactggccaagagttatgggattccattcattgaaacctcagccaagaccagacagggtgttgaagatgccttttacacactggtaagagaaatacgtcagtaccgaatgaagaaactcaacagcagtgatgacgggactcaaggttgtatggggttaccgtgtgtggtgatgtaacaagatacttttaaagttctagcatcagaaaagagccactgtcaagctgcactgacaccctggtcctgacttccctggaggagaagcgttcctgttgctattttcagtttcacaaagaagctcctgctatttccccaactctccgtagatcagtacattattctctgtttgagaagttctccgaataactacctcctcacttggttgtctgaccagagaaatgaacctcttgttactccccactgtttttccaccctggttctcccccagcacatataaacaaacctcccaggtttttcatctgaaaagtaattcatgctctgaaacagagaaccaaactgtagacatgaaattctgtaggaaacaatgtcttgagctctaaagtagcaactgctggtgactttttttttttttttttcctttttactgttgaacttggaactatgttggtttttggagaaatgtcgtaagttactgttttgctgagtatatagttaagtttaccattcggtttgtttgtaatgtcattggctatactctgtacctggcatctgctctgcattcataaatacaaaagtgaattctgacttttgagtctatcctagtgttctcaacttccacataattaaatctaacttttgcagcaaagtgcctttttcctagaagtggtttgtagatttgctttataatactttggtggaatagatgtctcaaaaaccattatacatgaaaatgaatgtctgagatacgtctatgatctgtctacctttgagggaaaaatataccgacataatagcagatgccatgtcttacgtgtatgaagttggatttccagagacctgatttgggtctcttccaagagaaagatgaaactggaaacaattatgaataacttcacttaatttttacctaatctctacttcggggtgggagggcagggagtaggttaccacttacaaaatatatgcaatttgtttcttctagcttactgataatgaacttccattcttatttaaatttaggtcatatcctaaagctttacatttgcaggtgttcgaaattgtaagtttaatgcagttttatttaatagctatgatcaatgattttcaagcctcagatgtattaacggacacattttcact

SEQ ID NO: 80 - protein, 189 aa, Felis catus; identical to SEQ ID NO: 68

SEQ ID NO: 81 - DNA, 4283 bp, Bos taurus
ggccgctccctagtgggggctgttcatggcggttccggggtctcccaacaattttcccggttgtggtcgtaatctatccgaagtggaggcagtggagctagaggttcttgctggtgtgaaatgactgagtacaaactggtggtggttggagcaggtggtgttgggaaaagtgcactgacaatccagctaatccagaaccactttgtagatgaatatgatcccaccatagaggattcctaccgaaaacaggtggttatagatggtgaaacctgtctgttggacatactggatacagctggacaagaggagtacagtgccatgagagaccaatacatgaggacaggcgaaggcttcctttgtgtgtttgccatcaataatagcaaatcatttgcagatattaacctctacagggaacagataaagcgtgtaaaggactcggatgatgtacctatggtgctagtaggaaacaagtgtgatttgccaacaaggacagttgacacaaaacaagcccatgaactggccaaaagttatgggattccattcattgaaacctcagccaagaccagacagggtgttgaagatgccttttacacactggtaagagaaatacgtcagtaccgaatgaaaaagctcaacagcagtgatgatggcactcaaggctgtatggggttgccgtgtgtggtgatgtaacaagatacttttaaagttctcacatcagaaaagagccactgtcaagctgcactgacaccctggtcctgacttccctggaggagaagtattcctgttgctatcttcagtttcaaaaagaagctcctgctatttccccaactctcagtagatcaatataatattctctatttgagaagttctcaagaataactacctcctcacttggttgtctgaccagagaattgaacctcttgttactcccagtatttttccaccctgggttctcccccagcacacacaaacgcacctctgccacccaggtttttcatctgaaaagcaattaatactctgaaacagagaaccaaactgtagaaacatgaaattctgtagaaaacaatgtcttgagctctaaagtagcaactgctggtgattttttttttttttttttcctttttattgttgaacttggaactatgttggtttgtggagaaatgtcataaattactgttttgctgagaatatagttaatgttgctctctggtttgtttgtaatgttatcagctatattctataaactggcatctactctgtatttagaaatacaaaaatgaatactgaccttttgagtctaccctcatcttctcgactttcttgtaattaaatgtaactttcacgatgaagtgccttttgcctgggagtgactcgtagacttcctttaaaatacttcagtggaatagatgtctcagaaactgttatacataagaataaatgtctgagatatgtctatgacccatctagctttgagggaaagatataccaatatgatagcagatgccatttcttacatctataacgttgattttctggagacctattttggggctctccgagagaaagatgagactataaatgattaggaataatttcacttaatttttacataacctccactttttgttttgtagtttactacctgcaaaacatataatttgattccttttagcttacagataatctaatgttaaatgaacttccattcatattttaatttggatcatatcaggaagtctacatttgcaggtgttcaaaaattgtaaaagtgtgatgcagttttatttaatagctgtgatcaatgattttcaagcctcaaatatgttaatagacacattttcactgtatatcatggtattaataattattgatgtatataattgtccttggtccccttctctgttcatcacctcatggcaatggcttgattaattatttcagctgagtaaagcatggtgctaatagaccagggtcacagtgtcaaaacttcagtgagccagtaagcatcacagagaaagaaattctttcacatttgctcaccattaactccagctaatagttttgccagatgtgtgtggttagtcctgcaaggaaaggagaagtcagttaatacaaattcttaaccaggactggaaaaacttgttttcctgagaagggtcagcttagaagtctttatctggactctatttttagccacatggaaatcaaattaagctgatcttttttctcaagtttttgagagtgaggatgcctcagatcaacatttttaaaatattctttattcttacgttcttttaagggtttaaaacaacgttgagtaattagtctgggcataccaggtaacaagctgataagtttgtgctgaacaagaagtagcctttggattgaaattgctgttttgagaagggatagaaaatataattaataattatgagacttgacttttctatttgcagataatatcctgataattctgatgaaaatagacttggataatttttgataaaagaatcgttccaaaatggccacttgctgttcttgtcttctaatgtgtaaatacttactgaggtcctcttctaatatgagttgtcatttattaagcaaattccacattgccttgaaatgaattcggaagagaagaaaaagtcatagtatacccagagaatgaaaaatccagagaattgtgctccttagtgttaattctgaagccttcgtagtccacacccatagacagaaactctctgccactttgcttctgctcctcttggagcattgcgctgtcatttccttgaggatagattgaggcttgtcaactcagttgtattgtcttcctcctcttcctcttgtctgtgtgactgacagtgtgactcttactaatgtcagatgcggggatgcggggaggtgggggggagtagctcattttaggctcttgcaccctttaccgttgtatgtgtgtgtcttttagttttctcaagaatgttctaagcacagaagtatctaaatggggccaaaattcagacttgaaaatgttcttttaatagcttcttaaaaagttacactttggtgtgaattttggcaggatagagtgacaaactcttaaacgctgaataacttcagttagtgtgttatagtttttagaatatgtttgtgattgctgaaaacaattatagtttacctcaaaatctgaaagtctctttccccaagttaagtgcctggccagctgtcaaagattacatattactttatgtttgtttgttttttaaaggttgcacattcaagattgtgaaaataaggtgttctgtctgaaagctaccatgcctgtctgtaaatgaatccactgagtgctgtacttgttccaacagcttactacagaatgctacttggtaatatcatactcgttacagttttcacttcaggagtgtactaggtagaatgatcctgtgtgtattgtagtgggctccatgtttagtcttttcagcatcctttaaactgctgtgaatttttgtcttgacttgaaagcaaggatagagaaacactttaaagagatactttgggtttttttccattccagaattggtgagcatagttagattttgctttacatttacagtcatgaactcttaagctggcagctacaaccaagaaccaaaagagggtgcattctgcttcttgtaattcatctttgctaataaattatgagaagcaaagataattaattagagaaactattttatttgggtggtttctataaacaagggactataattcttaaacattatttttcatttttgctgtttctttaagaaacctaatgtgccacaacattattttaaggtgtttcttaaaagaattgtttttaaaagtgttctcattttcagagtaattgtagatatatttcaaaatataactgataatttttaaaggcctgagtactgacctaagaagcagttgtatgaattctctggggggaagggaggagctcagtgaaagttgtatgacttttatatttctgtgccatcaaataaaggtaaaaatgtcttttgtgcagttttgctgttcaaacagaaactattggcctccttggccctaaatgaaagggctggtattttaagttgactattttattgtaaattaatccatcttaatttttttaaatttggttgaatgttctcttgttaaatgtttaaaaaataaaaactggaagttctttgcttagtcataaaaaaaaaaaaaaaaaaaaaaaaaaaaaaaaaaaaaaaaaaaaaaataaaaaaaaaaaaaaa

SEQ ID NO: 82 - protein, 189 aa, Bos taurus; identical to SEQ ID NO: 68

SEQ ID NO: 83 - DNA, 4825 bp, Gallus gallus
gcgccgggaccggaagccggaagctttgcagaagggtgttccgcgttcgcggtgcgggagcggtcagccggggtggcggggctggggccggccggggcaggcggctccgcgctccgcactgggccgctgggagggcgatgactgaatacaagctggtggtggtgggagctggcggcgtcgggaagagcgcgttgaccatccagctcatccagaaccacttcgtggacgagtacgaccccaccatcgaggattcgtacagaaagcaggttgtcatcgatggagagacgtgcttgttggacattctggacactgcaggacaggaagaatacagtgctatgcgtgatcagtacatgagaactggggaaggattcctttgtgtgtttgccattaacaacagtaaatcattcgctgatattaacctttacagagagcaaatcaagagagtgaaagattcagacgatgtgccaatggtgctggtggggaataagtgcgatttgccaacaaggacagtagacaccaaacaggctcaagagttagcaaaaagctacggcattcccttcatagagacatcagccaaaacgagacagggtgtggaagatgcgttttacacactggtgagggagattcggcagtaccggatgaaaaagctcaacagcaacgaagatgggaatcagggctgtatggggttgtcctgcattgtgatgtgataagatgccaggttcagatgtagctgctggacaagtctcgatgctactgtattgtgtctcatgctgatgccctgcagtattttggtgccagcgaccagactcttggtaccagttaattagctcaggatcctttcctgtgctccatctgaagaaaacatctctggtatctacctccttgctcagctcacagagcagtcatatctcttggtgtactgggattcttttctagctgtgttgtctgggtttgttcaagaagaaaaccagtcacaagaaaagtgaattacagagactaaatgctgtgaaaaagatcacactttacctccagagtaaaagctagaagtggcgtttgacccctttgcattggattcagatttgcggtgttgtcagaggagtggcagaagtaattttgccattacaaaggtttctgtcaccagtcggattggtatctgctgtctgtgcacccacacagtgtatctgcaacatctgcattgtgccagaagtatcacttaactgatgaactgatcctttatttttctgtaataaaaaggagatatctttgctaacttaagtgcctgtttgctcagaaggttggaggttgtatgctgttcccttgggctgaggagaaccccaaggatgaatttcttgggtgctcattgtcttgagcaggcaagttttgtgtgggtgatctcttttcatggcaggatattaaaatgggaatttgtagtctggaagatggagcagctgtttgtgagactcttgagttagggagagaaatgtataccacgtctgttctcgatccatcagaatggatccatccacctctttgtgtgtggaactgtgtatagtctgtattggttttctacagcacttggatctctttggaccaaattagcgagctgttcattttaacataactgccagtatttatagacaatttcttacggacagataatgaatttagaaactggaggttactttgggcagctgttcctcagctctgtctgtaacttgcaaattattctgagttattttctgcagaacctccttccttatcacgggaggagcctgggagttgaggttgactgtaattgggtcaatggttgtcacagacttaaggtgtccaggctgattggaggaggcactgagccctaacagagcactgagctgacttctaattgcagcatccttgcaaaatgaggaagggagttcagtgatgtctgcactgaagatgtatgatacactgatagcagttctgggtatgttgtaacagcttcaaagtagaaccgcagtactgcgtgagctgtgtgacttcttcctagaacacagcactgtcaccccatatggttgggacgtgcaggtgagaccaacacctaccaggttccctggcgtaccgtggccttctcagttcttgtgccagtgatactgggttctgttctgtggtgtcagacagcgtcctgtagcaaagctgaattcccacttagtctggtgagagaataaagagccatcagccaacagagggagcgttcattctgctggagcagtgcgagctgtaagcattacgagaggcgtagtttcagtttgttgcagtcaggttcctatattttcaaagctgaaatcagaaataagtaaatacggagaaaataagctgttgcttttaatgctctttcctccactaattgtactcttaattttcttcttgggaggccgaggatccatctgcataactttagctgtgatgctccagataagtgtttagaattcattttatctttgactgatgggactgataagaagttaacgcacaatatttttacatacaacatcgttttccagtgacctcctgagcggtgggaagcattatgggatagcaccggctgtgactcgagttcatttgaaggcgatctcttgcctgcaggttaaatgggacggagtcagaatcactgtgagccgtctgtaatcagcaaacagtctgtgggcttttcttactgtgttctctctgtttgccttagtttggtgcaggaagagttccttgtgacagcgtcctttgaggtgtgttgcaggagctgaccatttgctccttgagctgtgtgatgaactgttgtccacttaatggagttacagaagcagcttctgggagtcgcatctggtcgcatacattcagtgttttgggaagctgtcagtgtggtgtttgcactgtgtttgaatggtgttcatggtgggtctgttatgctcctggatgatttggggagatgtggggctgcttccgtggcagacaggatcagctcagggcgctgctgcctatggctgtgggaaacctcacagttggtgtttgaatagtggccaagtatgtcaattaaaaatacattttgaagggaggtttgtcatagctctgtactttggcatgctctgcttactgaaaacatactagctgtagctcaaaaaaagttgtgaatcctcagaataatacaggagctggcaattgtggctgctttctctttgtgttccttttctcttgggttggatgaagctttaaaaaggaaggagccctggtgagggttggtcagtgtgcatttcattcttggaaccagagaggaagttgcatcaactttcaggacgctgcagagctcacttgcacaggtggtgctccagtctatgtgatttttggggtcaaatcttgagatgatcttacaaaatcagattttgtacccatcatgagcatgaggtgagtggttgtgctcggtttctagctgcatgtatgtatacagacacgtgtatgcagacatgtctatgtgtgagtagttcgagtcagtcaaggttactggcagcacctaaagcgtatgcaccacataatgcatgcaggcaaaagtcctatcttaggagccatctcttcatgggtttgggtttatataggcagtatttttaaacagaatatccgaagcactttctggagttctgtggtaatgcagtgacacctatttggatgaaggaagatgtgtctgaggagcacgtaagcagatttgctgccctaacagagaggttttggtaaccgtggaaaaggttttctcctggatctgtgtgtgctcttggtgagctgcaatccatgacagggcacaaccagatgagaaggaaacccggccatcccatgcttgagcacagctctgactcagtagttccaccagatgtgccctttcagtcaaagtgttctgatctcttagagctttctgtagttcaagttaccactcactctccagcttgctcggttaatgtctgttggcggcgttgagttggacttgggaaaggtgtgtgtggtaggaacaagcagagtgtgatgtgcttctgttatcaggacttaagctagagtggttggcagataggaaatgcagctattccttgaaagcaagcagatcatggatggtcagccaaactgccctggctttggtgggagctgcactgcagaaggaccaaaccccaacaagatttggcacatttgtttagaagataagcacagatggttttgcacaaggcagctcctcataatggtggctttgtagatttagtccaaatgttcttatttagatctagcagcacatcactgtgtccgtgcccatctaacctcgctatcctaagtagagcagaccccaaacaaccttgttcaaaaactaccagtgcaaataactgaactaaatatttgttactgctgactgagaacagctgttcgagtgtagcattgtggcttgttaatgtgagtgccccaactctatggtcttattaaagaaacccaaacattgctcagattttgttcttattgtcatcataagacttgaatagtgatggtaatgcttacgtagacgtgtcttgtgagtgcacttcagtgatttagaaagaactggatttcaagcaactttggacctgtggggggagggagattaatgaaggtttgaatcacattctaattctatgtacagtccttcattactccacaagcctaaatcctatacagcctccaggatagctggaaactgttgagatctggactttttttttttaatccaagggctaacttgttgtaacttggtataattatctgctttcggaaatgcatctctgttggtttgaaa

SEQ ID NO: 84 - protein, 189 aa, Gallus gallus
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAQELAKSYGIPFIETSAKTRQGVEDAFYTLVREIRQYRMKKLNSNEDGNQGCMGLSCIVM

SEQ ID NO: 85 - DNA, 2949 bp, Homo sapiens
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttctttttataaaaa

SEQ ID NO: 86 - protein, 765 aa, Homo sapiens
AALSGGGGGGAEPGQALFNGDMEPEAGAGAGAAASSAADPAIPEEVWNIKQMIKLTQEHIEALLDKFGGEHNPPSIYLEAYEEYTSKLDALQQREQQLLESLGNGTDFSVSSSASMDTVTSSSSSSLSVLPSSLSVFQNPTDVARSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETALTSGSSPSAPASDSIGPQILTSPSPSKSIPIPQPFRPADEDHRNQFGQRDRSSSAPNVHINTIEPVNIDDLIRDQGFRGDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 87 - DNA, 3906 bp, Rattus norvegicus
atggcggcgctgagtggcggcggtggcagcagcagcggtggcggtggcggcggcggcggcggcggtggtggcggcggcggcggcggcgccgaacagggacaggctctgttcaatggcgacatggagccggaggccggcgctggcgccgcggcctcttcggccgcggacccggccattcctgaagaggtgtggaatatcaagcaaatgattaagttgacacaggaacatatagaggccctattggacaagtttggtggggagcataacccaccgtcaatatacctggaggcctatgaagagtacaccagcaagctagatgcccttcagcagagagagcagcagctgttggaatccctggtttttcaaactcccacagatgtatcacggaacaaccccaagtcaccacagaaacctatcgttcgtgtcttcctgcccaacaaacagaggacagtggtgcccgcaagatgtggtgtaacggtccgagacagtctaaagaaagcactaatgatgaggggtctcatcccagagtgctgtgctgtttacagaattcaggacggagagaagaaaccaattggctgggacactgacatttcctggcttactggagaggagctacatgttgaagtactagagaatgttcctctgacaacccacaacttcgtacggaaaacttttttcaccttagcattttgtgacttttgccgaaagctgcttttccagggtttccgctgtcaaacatgtggttataagtttcaccagcgttgtagtacagaggttccactgatgtgtgttaattatgaccaacttgatttgctgtttgtctccaagttctttgagcatcacccagtaccacaggaggaggccttctcagcagagactacccttccatctggatgctcttccgcacccccctcagactctattgggccccaaatcctcaccagtccatctccttcaaaatccattccaattccacagcccttccggccagcagatgaagatcatcgcaatcagtttgggcaacgagaccgctcctcctccgctcccaatgttcatataaacacaatcgaacctgtcaatattgatgaaaaattcccagaagtggaattacaggatcaaagggatttgattagagaccaggggtttcgtggggatggagcccctttgaaccagctgatgcgctgtcttcggaaataccaatcccggactcccagccccctcctccattctgtccccagtgaaatagtgtttgattttgagcctggcccagtgttcagagggtcaaccacaggcttgtcggccaccccacctgcctcattacctggctcactcactaacgtgaaagccttacagaaatctccaggacctcagcgggaaaggaagtcctcctcctcctcctcctccacggaagacagaagtcggatgaaaacacttggtagaagagattcaagtgatgattgggagattcctgatggacagattacagtgggacagagaattggatctgggtcctttggaactgtctacaagggaaagtggcatggcgacgtggcagtgaaaatgctgaatgtgacagcacccacacctcagcagttacaggccttcaaaaacgaagtcggagtactcaggaaaactcgacatgtgaacatcctccttttcatgggctattctacaaagccacagctggctattgttacacagtggtgtgaaggctccagcttatatcaccatctccacatcattgagaccaaatttgagatgatcaaacttatagatattgcacggcagactgcacagggcatggattacttacacgccaagtcaatcatccacagagacctcaagagtaataatatatttcttcatgaagacctcacggtaaaaataggtgactttggtttagccacagtgaagtcccgatggagtgggtcccatcagtttgaacagttgtctggatctattttgtggatggcacccgaagtaatcagaatgcaagataaaaacccatatagctttcagtcagacgtgtatgcatttgggattgttctgtatgaactgatgactggtcagctaccttattcaaacatcaacaacagggatcagataatttttatggtgggacgaggatacctatctccagatctcagtaaggtacggagtaactgtccaaaagccatgaagagattaatggcagagtgcctcaaaaagaaaagagacgagagaccactctttccccaaattctcgcctctattgagctgctggcccgctcattgccaaaaattcaccgcagtgcatcagaaccctccttgaatcgggctggtttccaaacagaagattttagtctgtatgcttgtgcttctccaaaaacacccatccaagcagggggatatggagaatttgcagccttcaagtagccactccatcatggcagcatctactctttatttcttaagtcttgtgttcatacaatttgttaacatcaaaacacagttctgttcctcaaattttttttaaagatacaaaattttcaatgcataagctcgtgtggaacagaatggaatttcctattcaacaaaagagggaagaatgttttaggaaccagaattctctgctgcccgtgtttcttcttcaacacaaatatcatgtgcatacaactctgcccattcccaagaagaaagaggagagaccccgaattctgcccttttggtggtcaggcatgatggaaagaatttgctgctgcagcttgggaaaaattgctatggaaagtctgccagtcaactttgcccttctaaccaccagatccatttgtggctggtcatctgatggggcgatttcaatcaccaagcatcgttcttgcctgttgtgggattatgtcgtggagcactttccctatccaccaccgttaatttccgagggatggagtaaatgcagcataccctttgtgtagcacctgtccagtcctcaaccaatgctatcacagtgaagctctttaaatttaagtggtgggtgagtgttgaggagagactgccttgggggcagagaaaaggggatgctgcatcttcttcctcacctccagctctctcacctcgggttgccttgcacactgggctccgcctaaccactcgggctgggcagtgctggcacacattgccgccttttctcattgggtccagcaattgagcagagggttgggggattgtttcctccacaatgtagcaaattctcaggaaaatacagtccatatcttcctctcagctcttccagtcaccaaatacttacgtggctcctttgtccaggacataaaacaccgtggacaacacctaattaaaagcctacaaaactgcttactgacagttttgaatgtgagacatttgtgtaatttaaatgtaaggtacaggtcttaatttcttctattaagtttcttctatttttatttaaacgaagaaaataattttcaggtttaattggaataaacgaatacttcccaaaagactatataccctgaaaattatatttttgttaattgtaaacaacttttaaaaaatggttattatccttttctctacctaaaattatgggaaatcttagcataatgacaattatttatactttttaaataaatggtacttgctggatccacactaacatctttgctaacattcccattgtttcttccaacttcactcctacactacatcctccatcctctttctagtcttttatctataatatgcaacctaaaataaaagtggtggtgtctccattcattcttcttcttccttttttccccaagcctggtcttcaaaaggttgggtaatttagtagctgagttccctaggtagaaatagaactattagggacattggggttgtaggaaagcgtgaggcctgtcaccagttgttctt

SEQ ID NO: 88 - protein, 804 aa, Rattus norvegicus
MAALSGGGGSSSGGGGGGGGGGGGGGGGGAEQGQALFNGDMEPEAGAGAAASSAADPAIPEEVWNIKQMIKLTQEHIEALLDKFGGEHNPPSIYLEAYEEYTSKLDALQQREQQLLESLVFQTPTDVSRNNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPVPQEEAFSAETTLPSGCSSAPPSDSIGPQILTSPSPSKSIPIPQPFRPADEDHRNQFGQRDRSSSAPNVHINTIEPVNIDEKFPEVELQDQRDLIRDQGFRGDGAPLNQLMRCLRKYQSRTPSPLLHSVPSEIVFDFEPGPVFRGSTTGLSATPPASLPGSLTNVKALQKSPGPQRERKSSSSSSSTEDRSRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGEFAAFK

SEQ ID NO: 89 - DNA, 9728 bp, Mus musculus
ccctcaggctcggctgcgccggggccgccggcgggttccagaggtggcctccgccccggccgctccgcccacgccccccgcgcctccgcgcccgcctccgcccgccctgcgcctcccttccccctccccgccccgcggcggccgctcggcccggctcgcgcttcgaagatggcggcgctgagtggcggcggtggcagcagcagcggtggcggcggcggcggtggcggcggcggtggcggtggcgacggcggcggcggcgccgagcagggccaggctctgttcaatggcgacatggagccggaggccggcgctggcgccgcggcctcttcggctgcggacccggccattcctgaagaggtatggaatatcaagcaaatgattaagttgacacaggaacatatagaggccctattggacaaatttggtggagagcataacccaccatcaatatacctggaggcctatgaagagtacaccagcaagctagatgcccttcagcaaagagaacagcagcttttggaatccctggtttttcaaactcccacagatgcatcacggaacaaccccaagtcaccacagaaacctatcgttagagtcttcctgcccaacaaacagaggacagtggtacccgcaagatgtggtgttacagttcgagacagtctaaagaaagcactgatgatgagaggtctcatcccagaatgctgtgctgtttacagaattcaggatggagagaagaaaccaattggctgggacacggacatttcctggcttactggagaggagttacatgttgaagtactggagaatgtcccacttacaacacacaactttgtacggaaaacttttttcaccttagcattttgtgacttttgccgaaagctgcttttccagggtttccgttgtcaaacatgtggttataaatttcaccagcgttgtagtacagaggttccactgatgtgtgtaaattatgaccaacttgatttgctgtttgtctccaagttctttgagcatcacccagtaccacaggaggaggcctccttcccagagactgcccttccatctggatcctcttccgcacccccctcagactctactgggccccaaatcctcaccagtccatctccttcaaaatccattccaattccacagcccttccgaccagcagatgaagatcatcgcaatcagtttgggcaacgagaccggtcctcctcagctcccaatgttcatataaacacaattgagcctgtgaatatcgatgaaaaattcccagaagtggaattacaggatcaaagggatttgattagagaccaggggtttcgtggtgatggagcccccttgaaccaactgatgcgctgtcttcggaaataccaatcccggactcccagccccctcctccattctgtccccagtgaaatagtgtttgattttgagcctggcccagtgttcagagggtcaaccacaggcttgtccgccaccccgcctgcctcattacctggctcactcactaacgtgaaagccttacagaaatctccaggtcctcagcgggaaaggaagtcatcttcttcctcatcctcggaggacagaagtcggatgaaaacacttggtagaagagattcaagtgatgactgggagattcctgatggacagattacagtgggacagagaattggatctgggtcatttggaactgtctacaagggaaagtggcatggtgatgtggcagtgaaaatgttgaatgtgacagcacccacacctcaacagctacaggccttcaaaaatgaagtaggagtgctcaggaaaactcgacatgtgaatatcctccttttcatgggctattctacaaagccacaactggcaattgttacacagtggtgtgagggctccagcttatatcaccatctccacatcattgagaccaaatttgagatgatcaaacttatagatattgctcggcagactgcacagggcatggattacttacacgccaagtcaatcatccacagagacctcaagagtaataatatatttcttcatgaagacctcacggtaaaaataggtgactttggtctagccacagtgaaatctcggtggagtgggtcccatcagtttgaacagttgtctggatctattttgtggatggcaccagaagtaatcagaatgcaagataaaaacccgtatagctttcagtcagacgtgtatgcgtttgggattgttctgtacgaactgatgaccggccagctaccttattcaaacatcaacaacagggatcagataatttttatggtgggacgaggatacctatctccagatctcagtaaggtacggagtaactgtccaaaagccatgaagagattaatggcagagtgcctcaaaaagaaaagagacgagagaccactctttccccaaattctcgcctccattgagctgctggcccgctcattgccaaaaattcaccgcagtgcatcagaaccttccttgaatcgggctggtttccaaacagaagattttagtctgtatgcttgtgcttctccgaaaacacccatccaagcagggggatatggagaatttgcagccttcaagtagccagtccatcatggcagcatctactctttatttcttaagtcttgtgttcatacagtttgttaacatcaaaacacagttctgttcctcaaaaaattttttaaagatacaaaattttcaatgcataagttcatgtggaacagaatggaatttcctattcaacaaaagagggaagaatgttttaggaaccagaattctctgctgcccgtgtttcttcttcaacataactatcacgtgcatacaagtctgcccattcccaagaagaaagaggagagaccctgaattctgcccttttggtggtcaggcatgatggaaagaatttgctgctgcagcttgggaaaattgctatggaaagtctgccagtcgactttgcccttctaaccaccagatcagcctgtggctggtcatctgatggggcgatttccatcaccaagcatcgttcttgcctattctgggattatgttgtggagcactttccctgtccagcaccgttcatttctgagggatggagtaaatgcagcattcccttgtgtagcgcctgttcagtcctcagcagctgctgtcacagcgaagctttttacagttaagtggtgggggagagttgaggagagcctgcctcggggcagagaaaagggggtgctgcatcttcttcctcacctccagctctctcacctcgggttgccttgctcactgggctccgcctaaccactcaggctgctcagtgctggcacacattgccttcttttctcattgggtccagcaattgaggagagggttgggggattgtttcctcctcaatgtagcaaattctcaggaaaatacagtccatatcttcctctcagctcttccagtcaccaaatacttacgtggctcctttgtccaggacataaaacaccgtggacaacacctaattaaaagcctacaaaactgcttactgacagttttgaatgtgagacacttgtgtaatttaaatgtaaggtacaggttttaatttctgagtttcttctatttttatttaaaagaagaaaataattttcagttttaattggaataaatgagtacttcccacaagactatataccctgaaaattatatttttgttaattgtaaacaacttttaaagaataattattatccttttctctacctaaaaattatggggaatcttagcataatgacaattatttatactttttaaataaatggtacttgctggatccacactaacatctttgctaacaatcccattgtttcttccaacttaactcctacactacatcctacatcctctttctagtcttttatctataatatgcaacctaaaataaacgtggtggcgtctccattcattctccctcttcctgttttccccaagcctggtcttcaaaaggttgggtaatcggtccctgagctccctagctggcaatgcaactattagggacattggagttgcaggagagcaggaagcctgtccccagctgttcttctagaaccctaaatcttatctttgcacagatcaaaagtatcacctcgtcacagttctccttagcctttacttacaggtaatataaataaaaatcaccatagtagtaaagaaaacaactggatggattgatgaccagtacctctcagagccaggaatcttgaatctccaggatttatacgtgcaaatttaaggagatgtacttagcaacttcaagccaagaacttccaaaatactagcgaatctaaaataaaatggaattttgagttatttttaaagttcaaattataattgataccactatgtatttaagcctactcacagcaagttagatggattttgctaaactcattgccagactgtggtggtggtggtggtagtgtgcacctttaatccaagcaactcagcaatcagaatgaggtaaatctctgtgaatacaaggcctgcctagtctgcagcgctagttccaggatagccagggctacacacacaaaaaccctctctcaaaaaaaacaaaattaattagttgataataaaaaataactaaagtatcatcaaaggaaggcctactggaagttttatatattcccagtaaattgaaaaatattctgaagttattaaccagttagcaacaatgtgtttttaagtcttacataaacagagcaaagtcttcaaatgtttcagagctgagaagataattgtgcttgatatgaaaaatagcctctccatatgatgtgccacattgaaaggcgtcattacccttttaaatacttcttaatgtggctttgttccctttacccaggattagctagaaagagctaggtaggcttcggccacagttgcacatttcgggcctgctgaagaatgggagctttgaaggctggccttggtggaggagcccctcagtgctggagggtggggcgtgtacgcagcatggaagtggtctagacagagtgcaaagggacagacttctttctcattttagtatagggtgatgtctcacttgaaatgagaaagtagagttgatattaaacgaagctgtgcccagaaaccaggctcagggtattgtgagattttctttttaaatagagaatataaaagatagaaataaatatttaaaccttccttcttattttctatcaaatagattttttttatcatttgcaaacaacataaaaaaaggtttcttttgtggggttttctttccttcttttttttttttttttttttttaagactgcagataatcttgttgagctcctcggaaaatacaaggaagtccgtgtttgtgcagagcgctttatgagtaactgtatagacagtgtggctgcttcactcatcccagagggctgcagctgtcggcccatgaagtggctgcagtgcctcgtgagatctgctttgttttgtttggagtgaagtctttgaaaggtttgagtgcaactatataggactgtttttaaataagtagtattcctcatgaactttctcattgttaagctacaggacccaaactctaccactaagatattattaacctcaaaatgtagtttatagaaggaatttgcaaatagaatatccagttcgtacttatatgcatcttcaacaaagattctctgtgacttgttggatttggttcctgaacagcccatttctgtatttgaggttaggagggcataatgaggcatcctaaaagacaatctgatataaactgtatgctagatgtatgctggtaggggagaaagcattctgtaaagacatgatttaagacttcagctctgtcaaccagaaaccttgtaaatacttcctgtcttggtgcagccccgcccctttgatcacacgatgttgtcttgtgcttgtcagacactgtcagagctgctgttcgtccctctgcagatctcacctgtccccactgcacacccacctcctgcctcttgcagacctcagcatctagctttagttggaaacagttcagggttcaggtgacttcttaaaaaaaaaaaaaaaccctacctcctcagaatgaggtaatgaatagttatttatttaaagtatgaagagtcaggagcgctcgaacatgaaggtgatttaagatggttcctttcgtgtgtattgtagctgagcacttgtttttgtcctaaagggcattatacatttaagcagtgattctgtttaaagatgtttttctttaaaggtgtagctcagagtatctgttgttggaattggtgccagagtctgcttaatagatttcagaatcctaagcttaagtcagtcgcatgaagttaagtagttatggtaacactttgctagccatgatataattctactttttaggagtaggtttggcaaaactgtatgccttcaaagtgagttggccacagctttgtcacatgcacagatactcatctgaagagactgcccagctaagagggcggaaggatacccttttttcctacgattcgcttctttgtccacgttggcattgttagtactagtttatcagcaccttgaccagcagatgtcaaccaataagctatttttaaaaccatagccagagatggagaggtcactgtgagtagaaacagcaggacgcttacaggagtgaaatggtgtagggaggctctagaaaaatatcttgacaatttgccaaatgatcttactgtgccttcatgatgcaataaaaaagctaacattttagcagaaatcagtgatttacgaagagagtggccagtctggtttaactcagctgggataatatttttagagtgcaatttagactgcgaagataaatgcactaaagagtttatagccaattcacatttgaaaaataagaaaatggtaaattttcagtgaaatatttttttaaagcacataatccctagtgtagccagaaatatttaccacatagagcagctaggctgagatacagtccagtgacatttctagagaaaccttttctactcccacgggctcctcaaagcatggaaattttatacaaaatgtttgacattttaagatactgctgtagtttagttttgaaatagtatgtgctgagcagcaatcatgtactaactcagagagagaaaacaacaacaaattgtgcatctgatttgttttcagagaaatgctgccaacttagatactgagttctcagagcttcaagtgtaaacttgcctcccaagtcctgtttgcaaatgaagttggctagtgctactgactgctccagcacatgatggaaggcagggggctgtctctgaagtgtcttctataaagggacaatagaatagtgagagacctggtcagtgtgtgtcagctggacactccatgctatgggacttgcatcttctgtcctcaccatccccaagacattgtgctttcctcagttgtcctctagctgtttcactcagacaccaagatgaattactgatgccagaaggggccaaaatggccagtgtgttttgggggttgtatcagttgactggacaataactttaatagtttcagatcatttatttttacttccattttgacagacatttaaatggaaatttagtcctaacttttgtcatttgaaaggaaaaattaacagttcctataagatacttttgaggtggaatctgacatcctaattttttttcttttcagtgggtttgcagcgagggtcttgtatgcactaggcaagggttctaccactaagccacatttcccaggaaataaaatgttaacagttaaaacatacacacaaatacacaaacaccttattaccactttagtaaagtgagagatgtgcgtcctttgtctcagtctccacgatttcagctgccccttgtatgaataactcagtctcgctaaactgtttacttttatttacctggtttgactagttgcagctatataaccagttgtgcatgaggacaacagccagtgtgtttgttttgtttttggttttttgtggtacattttttgtaaagaattctgtagattgaagtgctctttgaaaacagaactgagatatatttattcttgttagcatcaaaaaacattttgtgcaaatgatttgcttttcctggcaggctgagtaccatatccagcgcccacaattgcgggttcccatctaccatgtccacaggggagacagacgggaagcacatgaggggtgtgtttacagagttgtaggagttatgtagttctcttgttgccttggaaatcactgttgttttaagactgttgaacccgtgtgtttggctgggctgtgagttacatgaagaaactgcaaactagcatatgcagacaaagctcacagactaggcgtaaatggaggaaaatggaccaaaataaggcagggtgacacataaaccttgggcttcggagaaaactaagggtggagatgaactataatcacctgaatacaatgtaagagtgcaataagtgtgcttattctaagctgtgaacttcttttaaatcattcctttctaatacatttatgtatgttccattgctgactaaaaccagctatgagaacatatgcctttttattcatgttaactaccagtttaagtggctaaccttaatgtcttatttatcttcattttgtattagtttacataccaggtatgtgtgtgtgctgtactcttcttccctttatttgaaaacacttttcactgggtcatctccttggccattccacaacacaactttggtttggctttcaatgtcaccttatttgatggcctgtgtcccagtagcagaatttatggtattcccattgctggctgctcttccgaccctttgcttctacagcacttgtctctcctaagatagtcagaaactaactgatcaggggatggacttcaccattcatcgtgtctcttcaattctattaaatagaccactcttgggctttagaccaggaaaaaggagacagctctagccatctaccaagcctcaccctaaaaggtcacccgtacttcttggtctgaggacaagtctccactccagtaagggagaggggaggaaatgcttcctgtttgaaatgcagtgaattcctatggctcctgtttcaccacccgcacctatggcaacccatatacattcctcttgtctgtaactgccaaaggttgggtttatgtcacttcagttccactcaagcattgaaaaggttctcatggagtctggggtgtgcccagtgaaaagatggggactttttcattatccacagacctctctatacctgctttgcaaaaattataatggagtaactatttttaaagcttatttttcaattcataagaaaaagacatttattttcaatcaaatggatgatgtctcttatcccttatccctcaatgtttgcttgaattttgtttgttccctatacctactccctaattctttagttccttcctgctcaggtcccttcatttgtactttggagtttttctcatgtaaatttgtataatggaaaatattgttcagtttggatagaaagcatggagaaataaataaaaaaagatagctgaaaatcaaattgaagaaatttatttctgtgtaaagttatttaaaaactctgtattatatttaaagaaaaaagcccaaccccccaaaaagtgctatgtaattgatgtgaatatgcgaatactgctataataaagattgactgcatggagaaa

SEQ ID NO: 90 - protein, 804 aa, Mus musculus
MAALSGGGGSSSGGGGGGGGGGGGGDGGGGAEQGQALFNGDMEPEAGAGAAASSAADPAIPEEVWNIKQMIKLTQEHIEALLDKFGGEHNPPSIYLEAYEEYTSKLDALQQREQQLLESLVFQTPTDASRNNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPVPQEEASFPETALPSGSSSAPPSDSTGPQILTSPSPSKSIPIPQPFRPADEDHRNQFGQRDRSSSAPNVHINTIEPVNIDEKFPEVELQDQRDLIRDQGFRGDGAPLNQLMRCLRKYQSRTPSPLLHSVPSEIVFDFEPGPVFRGSTTGLSATPPASLPGSLTNVKALQKSPGPQRERKSSSSSSSEDRSRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGEFAAFK

SEQ ID NO: 91 - DNA, 2232 bp, Oryctolagus cuniculus
atggggaatgtgtggaatatcaaacaaatgattaagttgacacaggagcatatagaggccctattggacaaatttggtggggagcataatccaccatcaatatatctggaggcctacgaagaatacaccagcaagctagatgccctccaacaaagagaacagcagttattggaatccctagtttttcaaaatcccacagatgtgtcacggagcaaccccaagtcaccacaaaaacctattgttagagtcttcctgcccaacaaacagaggacagtggtacctgcaagatgtggagttacggttcgagacagtctaaagaaagcgctgatgatgagaggtctgatcccagaatgctgtgctgtttacagaattcaggatggagagaagaagccaattggctgggacactgatatttcctggctcactggagaagagctgcatgtggaagtgttagagaatgtcccactcaccacacataactttgtacggaaaacttttttcaccttagcattttgtgacttctgtagaaagctgcttttccagggtttccgctgtcaaacatgtggctacaaatttcaccagcgttgtagtacggaagttccactgatgtgtgttaattatgaccaacttgatttgctgtttgtctccaagttctttgaacaccacccagtaccacaggaggaggcctccttagcagagactgccctcacatctgggtcatcgccttccgcacctccctcagactctattgggcaccaaattctcaccagtccgtccccttcaaaatccattccgattccacagtccttccgaccagcagatgaagatcatcgaaatcagtttgggcaacgagaccggtcttcatcagcgcctaatgttcacattaacacaatagaacctgtcaatattgatgaaaaattcccagaagtggaattacaggatcaaagggacttgattagagaccaagggtttcgtggtgatggagcccctttgaaccagctgatgcgctgtcttcggaaataccaatcccggactcccagtcccctcctaccttctgtccccagtgacatagtgtttgattttgagcctggcccagtgttcagaggatcgaccacgggtttgtctgccactccccctgcctcattacctggctcactcactagtgtgaaagctgtacagagatccccaggacctcagcgagagaggaagtcgtcttcctcctcagaagacaggaatcgaatgaaaactcttggtagacgggattcaagtgatgattgggagattcctgatgggcagatcaccgtgggacagagaattggatctggatcatttggaaccgtctacaagggaaaatggcacggtgatgtggcagtaaaaatgttgaatgtgacagcacctacacctcagcagttacaggccttcaaaaatgaagtaggagtactcaggaaaacacgacatgtgaatatcctacttttcatgggctattccacaaagccacagctggctattgttacccagtggtgtgagggctccagtttatatcaccatctccacatcattgagaccaaattcgagatgatcaaacttatagatattgcacggcagactgcacagggcatggattacttacacgccaagtcaatcatccacagagacctcaagagtaataatatatttcttcatgaagacctcacagtaaaaataggtgattttggtctagccacagtgaaatctcgatggagtgggtcccatcagtttgaacaattgtctggatccattttgtggatggcaccagaagtaatcagaatgcaagacaaaaacccatatagctttcagtcagatgtatatgcatttgggattgttctgtatgaattgatgactgggcagttaccttactcaaacatcaacaacagggaccagatcatttttatggtgggacgtggctacctgtctccagacctcagtaaggtacggagtaactgtccgaaagccatgaagagattaatggcagagtgcctcaaaaagaaaagagatgagagaccactctttccccaaattctcgcctccattgagctgctggcccgctcattgccaaaaatccaccgcagtgcatcagaaccctccttgaatcgggctggtttccagacagaggattttagtctatatgcttgtgcttctccaaaaacacccatccaggcagggggatatggagaatttgcagccttcaagtag

SEQ ID NO: 92 - protein, 743 aa, Oryctolagus cuniculus
MGNVWNIKQMIKLTQEHIEALLDKFGGEHNPPSIYLEAYEEYTSKLDALQQREQQLLESLVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPVPQEEASLAETALTSGSSPSAPPSDSIGHQILTSPSPSKSIPIPQSFRPADEDHRNQFGQRDRSSSAPNVHINTIEPVNIDEKFPEVELQDQRDLIRDQGFRGDGAPLNQLMRCLRKYQSRTPSPLLPSVPSDIVFDFEPGPVFRGSTTGLSATPPASLPGSLTSVKAVQRSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGEFAAFK

SEQ ID NO: 93 - DNA, 2186 bp, Cavia porcellus
atggcggcgctcagcggcggcggtggcgcggagcagggccaggctctgttcaacggggacatggagctcgaggccggcgccggcgccgcagcctcttcggctgcagaccctgccattcccgaggaggtatggaatatcaaacaaatgattaagttgacgcaggaacacatagaggccctattggacaaatttggtggagagcataatccaccatcaatatacctggaggcctatgaagaatacaccagcaaactagatgccctccaacaaagagaacagcagttactggaatccctcgggaatggaactgatttttctgtttctagctctgcatcactggacaccgttacatcttcttcttcttctagcctttcagtactaccttcatctctttcagtttttcaaaatcctacagatgtgtcacggagcaaccccaaatcaccacaaaaacctattgttagagtcttcctgcccaacaaacagaggacagtggtacctgcaaggtgtggagttacagtccgagacagtctgaagaaagcactcatgatgagaggtcttatcccagagtgctgtgctgtgtacagaattcaggatggagaaaagaaaccaattggctgggacactgacatttcctggcttactggggaagaattacatgtagaagtattggagaatgttccacttacaacacacaattttgtatgtatctttatattttttttgctgtttgtctccaagttctttgaacaccacccaataccacaggaggaggcttccttagcagagaccacccttacatctggatcatccccttctgcacccccctcagagtccattgggcccccaattctcaccagcccatctccttcaaaatccattccaattccacagcctttccggccaggagaggaagatcatcgaaatcaatttgggcagcgagaccggtcctcatctgctcccaatgtgcatataaacacaatagaacctgtcaatattgatgatttgattagagaccaagggtttcgtagtgatggaggatcaactacaggtttgtctgccaccccacctgcctcattacctggctcactcactaatgtgaaagccttacagaaatctccaggacctcagcgagaaaggaagtcatcttcatcctcagaagacagaaatcgaatgaaaacgcttggtagacgggactcaagtgatgattgggagattcctgatgggcagattacagtgggacaaagaattggatctgggtcatttggaacagtctacaaggggaagtggcatggtgacgtggcagtgaaaatgttgaatgtgacagcacccacacctcaacagttacaggccttcaaaaatgaagtaggagtactcaggaaaacacgacatgtgaatatcctactcttcatgggctattccacaaagccacagctagctattgttacccagtggtgtgagggctccagcttataccaccatctccacatcatcgagaccaaatttgagatgatcaaacttatagatattgcacgacagactgcccagggcatggattacttacacgccaagtcaatcatccacagagacctcaagagtaataatatatttcttcacgaagacctcacggttaaaataggtgattttggtctagccacagtgaaatctcgatggagtgggtcccatcagtttgaacagttgtctggatccattttgtggatggcaccagaagtaatcagaatgcgagataaaaacccatacagttttcagtccgatgtatatgcatttgggattgttctatatgaattgatgactgggcagttaccctattcaaatatcaacaacagggaccagataatttttatggtgggacgaggatatctatctccagatctcagcaaggtacggagtaactgtccaaaagccatgaagaggttaatggcggagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctctatgcttgtgcttctccaaaaacacccatccaggcagggggatatggtgcgtttcctgtccactgatgcaaattaaatgagtgagaaataaa

SEQ ID NO: 94 - protein, 719 aa, Cavia porcellus
MAALSGGGGAEQGQALFNGDMELEAGAGAAASSAADPAIPEEVWNIKQMIKLTQEHIEALLDKFGGEHNPPSIYLEAYEEYTSKLDALQQREQQLLESLGNGTDFSVSSSASLDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVCIFIFFLLFVSKFFEHHPIPQEEASLAETTLTSGSSPSAPPSESIGPPILTSPSPSKSIPIPQPFRPGEEDHRNQFGQRDRSSSAPNVHINTIEPVNIDDLIRDQGFRSDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMRDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 95 - DNA, 3229 bp, Canis lupus
gtaatgctggattttcatggaataagtttgacctgtgctgcagtggcctccagcaaggtacccgcaagatgtggagttacagtccgggacagtctaaagaaagctctgatgatgagaggtctaatcccagagtgctgtgctgtttacagaattcaggatggagagaagaaaccgattggctgggacactgatatttcctggctcactggagaggaattgcatgtagaagtgttggaaaatgttccgcttaccacacacaactttgtacggaaaacttttttcaccttagcattttgtgacttttgtcgaaagctgcttttccagggttttcgctgtcaaacatgtggttataaatttcaccagcgttgtagtacagaggttccactgatgtgtgttaattatgaccaacttgatttgctgtttgtctccaagttctttgaacaccacccaataccacaggaggaggcctccatagcagagactgcccttacgtctggatcatccccttctgctcccccctccgattctcctgggcccccaattctgaccagtccgtctccttcaaaatccattccaattccacagcctttccgaccagcagatgaagatcatcgaaatcagtttggacaacgagaccggtcctcatcagctccaaatgtgcatataaacacaatagaacccgtcaacattgatgacttgattagagaccaagggtttcgtagtgatggaggatcaaccacaggtttgtctgccaccccccctgcctcattgcctggctcactcactaatgtaaaagcattacagaaatctccaggacctcagcgggaaagaaaatcatcttcatcctcagaagataggaatcgaatgaaaacacttggtagacgggattcaagtgatgattgggagatacctgatgggcagatcacagtgggacagagaattggatccgggtcatttgggacagtctacaagggaaagtggcatggtgacgtggcagtgaaaatgttgaatgtgacagcacccacacctcagcagttacaggccttcaaaaatgaagtaggagtactcaggaaaactcgacatgtgaatatcctactctttatgggctattcaacaaagccccaactggctattgttacccagtggtgtgagggctccagcttatatcaccatctccacatcattgagaccaaatttgagatgataaagcttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtgatccgaatgcaagacaaaaacccatatagcttccagtcagatgtatacgcatttgggattgttctatatgaattgatgacagggcagttaccttattcaaacatcaacaacagggaccagataatttttatggtgggacgaggatatctttctccagatctcagtaaggtacggagtaactgtccaaaagccatgaagagattgatggcagagtgcctaaaaaagaaaagagatgagaggccactctttccccaaattctcgcctctattgagctgctggcccgctcattgccaaaaattcaccgcagtgcatcagaaccctccttgaatcgggctggcttccaaacagaggattttagtctctatgcttgcgcttctccaaaaacacccatccaggcagggggatacggagaatttgcagccttcaagtagccacaccatcatggcaacaactactcttatttcttaagtcttgtgttcgtacaatttgttaacatcaaaacacagttctgttcctcaaatctttttttaaagatacagaattttcaatgcataagctggtgtggaacagaatggaatttcccatccaacaaaagagggaagaatgttttaggaaccagaattctctgctgccagtgtttcttcttcaacacaaataccacgtgcatacaagtctgcccactcccaggaaggaagaggagagcctgagttctgaccttttgatggtcaggcatgatggaaagaaactgctgctacagcttgggagattggctgtggagagcctgcccgtcagctctgcccttctaaccgccagatgagtgtgtggctggtcacctgacagggcagctgcaatcgccaagcatcgttctctttcctgtcctgggattttgtcgtggagctctttccccctagtcaccaccggttcatttctgagggatggaacaaaaatgcagcatggcctttctgtgtggtgcatgtccggtctttgacaaatttttatcaagtgaagctcttgtatttaaatggagaatgagaggcgagggggggggatcacgttttggtgtaggggcaaagggaatgctgcatctttttcctgacccactgggtttctggcctttgtttccttgctcactgagggtgtctgcctataaccacgcaggctggaaagtgctggcacacattgccttctcttctcactgggtccagcaatgaagacaagtgttggggatttttttttttgccctccacaatgtagcaagttctcaggaaaatacagttaatatcttcctcctaagctcttccagtcatcaagtacttatgtggctactttgtccagggcacaaaatgccatggcggtatccaattaaaagcctacaaaactgcttgataacagttttgaatgtgtgagacatttatgtaatttaaatgtaaggtacaagttttaatttctgagtttctctattatatttttattaaaaagaaaataattttcagatttaattgaattggaataaaataatacttcccaccagaattatatatcctggaaaattgtatttttgttatataaacaacttttaaagaaagatcattatccttttctctacctaaatatggggagtcttagcataatgacagatatttataatttttaaattaatggtacttgctggatccacactaacatctttgctaatatctcatgttttcctccaacttactcctacactacatcctccatcctctttccagtcttttatctagaatatgcaacctaaaataaaaatggtggtgtctccattca

SEQ ID NO: 96 - protein, 617 aa, Canis lupus
MLDFHGISLTCAAVASSKVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASIAETALTSGSSPSAPPSDSPGPPILTSPSPSKSIPIPQPFRPADEDHRNQFGQRDRSSSAPNVHINTIEPVNIDDLIRDQGFRSDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGEFAAFK

SEQ ID NO: 97 - DNA, 1889 bp, Canis lupus
ggaatatcaaacaaatgattaagttgacacaggaacatatagaagccctattggacaagtttggtggggagcataatccaccatcaatatatctggaggcctatgaagaatacaccagcaaactagatgccctccaacagcgagaacaacagttattggaatccctggggaatggaactgatttttctgtttctagctctgcatcaacggacaccgttacatcttcttcctcttctagcctttcagtgctaccttcatctctttcagtttttcaaaatcccacagatatatcacggagcaatcccaagtcaccacaaaaacctatcgttagagtcttcctgcccaataaacagaggacggtggtacccgcaagatgtggagttacagtccgggacagtctaaagaaagctctgatgatgagaggtctaatcccagagtgctgtgctgtttacagaattcaggatggagagaagaaaccgattggctgggacactgatatttcctggctcactggagaggaattgcatgtagaagtgttggaaaatgttccgcttaccacacacaactttgtacggaaaacttttttcaccttagcattttgtgacttttgtcgaaagctgcttttccagggttttcgctgtcaaacatgtggttataaatttcaccagcgttgtagtacagaggttccactgatgtgtgttaattatgaccaacttgatttgctgtttgtctccaagttctttgaacaccacccaataccacaggaggaggcctccatagcagagactgcccttacgtctggatcatccccttctgctcccccctccgattctcctgggcccccaattctgaccagtccgtctccttcaaaatccattccaattccacagcctttccgaccagcagatgaagatcatcgaaatcagtttggacaacgagaccggtcctcatcagctccaaatgtgcatataaacacaatagaacccgtcaacattgatgacttgattagagaccaagggtttcgtagtgatggaggatcaaccacaggtttgtctgccaccccccctgcctcattgcctggctcactcactaatgtaaaagcattacagaaatctccaggacctcagcgggaaagaaaatcatcttcatcctcagaagataggaatcgaatgaaaacacttggtagacgggattcaagtgatgattgggagatacctgatgggcagatcacagtgggacagagaattggatccgggtcatttgggacagtctacaagggaaagtggcatggtgacgtggcagtgaaaatgttgaatgtgacagcacccacacctcagcagttacaggccttcaaaaatgaagtaggagtactcaggaaaactcgacatgtgaatatcctactctttatgggctattcaacaaagccccaactggctattgttacccagtggtgtgagggctccagcttatatcaccatctccacatcattgagaccaaatttgagatgataaagcttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtgatccgaatgcaagacaaaaacccatatagcttccagtcagatgtatacgcatttgggattgttctatatgaattgatgacagggcagttaccttattcaaacatcaacaacagggaccagctcagatcatgatcacggtgtcatgagatcaagccccac

SEQ ID NO: 98 - protein, 615 aa, Canis lupus
MIKLTQEHIEALLDKFGGEHNPPSIYLEAYEEYTSKLDALQQREQQLLESLGNGTDFSVSSSASTDTVTSSSSSSLSVLPSSLSVFQNPTDISRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASIAETALTSGSSPSAPPSDSPGPPILTSPSPSKSIPIPQPFRPADEDHRNQFGQRDRSSSAPNVHINTIEPVNIDDLIRDQGFRSDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQLRS

SEQ ID NO: 99 - DNA, 3521 bp, Felis catus
  misc_feature (630)..(649): n is a, c, g, or t
atgcctaacctcagtctctgccaccacggccaatttgctcatgtgcccactgtgtcggcactgggatattttgtgatttgccttggccattgtccactgtccttgacattgcctgaaggagaaccactgatgctaatgttgaaggtgacctttgcaggctctccactactcataccaaagatgcggccccctgataatcccagagccactgtctgcacatgggcaaaacaggctacattctgtgcagactggggagaaaggttccaagaacacagtgccatagttttgggcagagagtttcaacacagcatagtgtctatggcagtatctggatttggccgggggaagtgcccaagaggagacagtcaggctgtgtcctacggccaaggacctgcacttatttttgcatgcagtggtttagcacagggaagagaacgaagtaggaaatcggagccatggaaacggcagagcggaggaaacgtgcacgcgcgagggtgggcacgaaaggaaagaaccctccccagaagactgcgcgagggcgctcctaggattacgtcacgcaccccgcgaaaactgaaatgtactgtgtgtggtcttttaattgaactatcttccttatgtgcacttaannnnnnnnnnnnnnnnnnnngcggcggcggcggtggcgcggagcagggccaggctctgttcaacggggacatggagcccgaagccggcgccgcggcctcttcggctgcggaccctgccattcccgaggaggtgtggaatatcaaacaaatgattaagttgacacaggaacatatagaggccctattggacaaatttggtggggagcataatccaccatcaatatatctagaggcctatgaagaatacaccagcaagctagatgccctccaacagagagaacaacagttattggaatccctggggaatggaactgatttttctgtttctagctctgcatcaacagacaccgttacatcttcctcctcttctagcctttcagtgctaccttcatctctttcagtttttcaaaaccccacagatgtgtcacggagcaatcccaagtcaccacagaaacctatcgttagagtcttcctgcctaataaacagaggacagtggtacctgcaagatgtggagttacagtccgggacagtctaaagaaagctctgatgatgagaggtctaatccctgagtgctgtgctgtttacagaattcaggatggagagaagaaaccaattggctgggacactgatatctcctggctcaccggagaggaattgcatgtagaagtgttggaaaatgttccacttacaactcacaactttgtatgtacggaaaacgttttcaccttagcattttgtgacttttgtcgaaagctgcttttccaaggttttcgctgtcaaacgtgtggttataaatttcaccagcgttgtagtacagaggttccactgatgtgtgttaattatgaccaacttgatttgctgtttgtctccaagttctttgaacaccacccaataccacaggaggaggcctccatagcagagactgccctaacgtctggatcgtccccttctgcccccccctccgattctactgggccccaaattctcaccagtccgtctccttcaaaatccattccaattccacagcctttccgaccagcagatgaagatcatcgaaatcaatttggacagcgagaccggtcctcatcagctccaaatgtgcatataaatacaatagaacctgtcaatattgatgacttgattagagaccaggggtttcgtagtgatggaggatcaaccacaggcttgtctgccaccccccctgcctcattgccgggctctctcactaatgtaaaagcattacagaaatctccagggcctcagcgggaaaggaaatcttcttcatcctcagaagataggaatcgaatgaaaacacttggtagaagggattcaagtgatgattgggagattcctgatgggcagatcacagtgggacagagaattggatccgggtcatttgggacagtctacaagggaaagtggcatggtgatgtggcagtgaaaatgttgaatgtgacagcacccacacctcagcagttacaggccttcaaaaatgaagtaggagtactcaggaaaactcggcatgtgaacatcctgctcttcatgggctattcaacaaagccccagctggctattgtcacccagtggtgtgagggctccagcttataccaccatctccacatcatcgagaccaaattcgagatgatcaagctgatagatattgctcggcagactgcgcagggcatggattacttacacgccaagtcaatcatccacagagacctcaagagtaataatatttttcttcacgaagacctcacagtaaaaataggtgattttggtctagccacagtgaaatctcgatggagtgggtcccatcagtttgaacagttgtctggatccattttgtggatggcaccagaagtaattcgaatgcaagataaaaacccatatagctttcagtcagatgtatatgcatttgggattgttctatatgaattgatgactggacagttaccttattcaaacatcaacaacagggaccagataatttttatggtgggacgaggatatctttctccagatctcagtaaggtacgaagtaactgtccaaaagccatgaagagattgatggcagagtgcctaaaaaagaaaagagatgagaggccactgtttccccaaattcttgcctctattgagctgctggcccgctcattgccaaaaattcaccgcagtgcatcagaaccctccttgaatcgggctggcttccagacagaggattttagtctctatgcttgtgcttctccaaaaacacccatccaggcagggggatatggtgcgtttcccgtccactgagataagttagatgagtgcgcgagtgcagggggccggggccaaggaggtggaaatgtgcgtgcttctgtactaagttggatagcatcttcttttttaaaaaaagatgaaccaaagaatgtgtatgtttttaaagactagatataattatttcctgatctaaaatgtatacttagctttggattttcaatatccaagggttttcaaaatgcacagacattgctgaacatttgcagtacctcttctggaggctttacttcctgttacaaattggttttgtttactggcttatcctaattattaaacttcaattaaacttttctcctgcaccttttgttatgagctatcacatgtcccttagggactcgcaagagcagtactgcccccgtgtacgggcttgcaggtagaaaggggatgacgggttttaacacctgtgtgaggcaaggcagtccgaacagatctcatttaggaagccacgagagttgaataagttatttttattcttagtattttttctgtaactactttttattataacttggaaaatatggatgtcctttatacaccttagcaatagactgaatttctttttataaatt

SEQ ID NO: 100 - protein, 974 aa, Felis catus
  misc_feature (210)..(217): Xaa can be any naturally occurring amino acid
MPNLSLCHHGQFAHVPTVSALGYFVICLGHCPLSLTLPEGEPLMLMLKVTFAGSPLLIPKMRPPDNPRATVCTWAKQATFCADWGERFQEHSAIVLGREFQHSIVSMAVSGFGRGKCPRGDSQAVSYGQGPALIFACSGLAQGRERSRKSEPWKRQSGGNVHARGWARKERTLPRRLREGAPRITSRTPRKLKCTVCGLLIELSSLCALXXXXXXXXGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVCTENVFTLAFCDFCRKLLFQGFRCQTCGYKFHQRCSTEVPLMCVNYDQLDLLFVSKFFEHHPIPQEEASIAETALTSGSSPSAPPSDSTGPQILTSPSPSKSIPIPQPFRPADEDHRNQFGQRDRSSSAPNVHINTIEPVNIDDLIRDQGFRSDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 101 - DNA, 3853 bp, Bos taurus
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaaattctcgcctctattgagctgctggcccgctcattgccaaaaattcaccgcagtgcatcagaaccctccttgaatcgggctggcttccaaacagaggattttagtctatatgcttgtgcttctccaaaaacacccattcaggcagggggatatggtacgtttcctgttcactgaaacaaaccgagtgagtgacagcatgtaggagggtagggacaaaagaaagtgaacaaatgtttgcttatatatttgttaaattgaataggattttctttttctttaaaggtgaacaagagaacatgtgtgtttttaaagtttggatatagttttcttcccagtctaaaacccatagttagcattacattttcaacatcgaatttttttttaattcatagacattgctgaaaatttataataccttttccagaggctttacttcccattccaagtttgttttgtttacttggttagtctaatcattaaactttaaactttccccacctaccttttgctgttagctatcccgcatccattaggggctccaagaacagcactgtctgcgtgtgtgtgttggcaggtgggaagctgatggtaagttaggctgtgttagtgaaggtaaactgaccaggtctaattaggagtcactagaattgaataagcttatttttattaatattttttcttataactatttctttttgtaataatttagaaaatataattgttctttattcccttacagcagtataaattattggtgcaggtaaccaaagatattactgaggagtggcatgtttgacatgagtgacatggtttaactttggatttttagttaatatttctttatatattaaggatgtcttacacattatagaagtcaaatttactgacaaaggtattgcctcctcttcctccccaaaaacacagcaaaattctctgggaactcgtagcattgttggttttcttttggatgactatggttgccaaacaaccaagtaattgattttttttaaattattattgctttagattatactcacctctcatgatgcctgttagcaatcacctttatccatgtgtcttgtaaaatatctttcctccttatattctttgcccaacaagagtctacttgttatgaatgagtactattttctttttttgattccccagtataattagtatgtttagtgctttctaggacttccactttcttatgttaaaaaaaaaaacaaactaatgtggcagtcagtatattcttactgtgaatcagagtctttactgggaatcaaagtgaaagaagcagctgttctgacttcagagtcagcctagggaccaaaaccagcctcttaaatacaccttcatttattcagtttggatttgtgatgattttcattatagctgacagttcaaggttattcagtggcacacagatagcatctgcataaatgcctttcttcttgaaaataaaggagaaaattgggaagactttacaccaatagtttagtctttaagtaccacagataacacacaccataaat

SEQ ID NO: 102 - protein, 764 aa, Bos taurus
AALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGTFPVH

SEQ ID NO: 103 - DNA, 4936 bp, Bos taurus
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaagtaggaaagactctcctaagcaagagacaaaattcagaagttatcagggaaaaagataagcagattctcgcctctattgagctgctggcccgctcattgccaaaaattcaccgcagtgcatcagaaccctccttgaatcgggctggcttccaaacagaggattttagtctatatgcttgtgcttctccaaaaacacccattcaggcagggggatatgaagcagatttggctcttacatcaaataaaaatagagtagaagttgggatttagagatttcctgacatgcaagaaggaataagcaagaaaaaaaggtttgttttccccaaatcatatctattgtcttttacttctattttttcttaaattttttgtgatttcagagacatgtagagttttattgatacctaaactatgagttcttttttttttttttttttcattattttgatttttttggccaagaggcatatgggatcttagcttgagaaagcaacaattttcttgatgtcattttgggtgagggcacatattgctgtgaacagtgtggtgatagccaccagggaccaaactcacacccgctgcattgaaaggtgaagtcttaaacactggaccagcagagaaattcctactctatgagttctttttgtcatcccctccccgcaccctccacccccaacctaaagtctgatgatgaaatcaacaactattccattagaagcagtagattctggtagcatgatctttagtttgttagtaagattttgtgctttgtggggttgtgtcgttttaaggctaatatttaagtttgtcaaatagaatgctgttcagattgtaaaaatgagtaataaacatctgaagttttttttaagttatttttaacatggtatatacagttgagcttagagtttatcattttctgatattctcttacttagtagatgaattctagccattttttataaagatttctgttaagcaaatcctgttttcacatgggcttcctttaagggattttagattctgctggatatggtgactgctcataagactgttgaaaattacttttaagatgtattagaatacttctgaaaaaaaatagcaaccttaaaaccataagcaaaagtagtaagggtgtttatacatttctagagtccctgtttaggtaatagcctcctatgattgtactttaaatgttttgctctccaaggttttagtaacttggctttttttctaatcagtgccaaactcccccagtttttttaactttaaatatgaggtaataaatcttttacccttccttgatcttttgacttataataccttggtcagttgtttcttaaaaggaatccttaaatggaaagagacaatatcactgtctgcagttctgattagtagttttattcagaatggaaaaacagattattcatttttgaaaattgttcaggggtatgttcattgttaggaccttggactttggagtcagtgcctagctatgcattccaggtctgccattttctggctgtgaaattttggacaagttacttaaccactttaaaccccagctttaagaagtaaattaaccccagtaaattaagaagtaatagcagccacttcgtagagttgttatgaggctcagatgcagtgcaaatgtgtataaagtattcagggagtcacctggtatactataatagacactagaatagttgccaatattatcagcatacaatctgaggattctgtcagccaatcattagcaatctgttgtttgttgggacatgccagtgttctccagttgaaatcagtagcaatctaaaaatggatagattattcctcatttaaatagtgtgttcatataagtgattgcttggatccttatcagaagttgctgttactgaaaaatgataaggctgactaaattgtgatagttgtcagttactaaccaactcccagaaatgaataagaggaacctatctctagttcctagtagaaggtatggacaaaatagtaggtgaaaaataatgtcttgaacccccaaattaagtaagctttaaagagtacaatacctcaaagggtctttgcggtttaaaatttgtatgctgagaatgatgttcattgacatgtgcctatatgtaattttttgatagtttaaaaggtgaaatgaactacagatgggagaggtctgaattttcttgccttcagtcaaatgtgtaatgtggacatattatttgacctgtgaattttatcttttaaaaaagattaattcctgcttcttccttcctaatagttgcattataataatgaaaatgagttgataatttggggggaaagtattctacaaatcaaccttattattttaccattggtttctgagaaattttgttcatttgaaccgtttatagcttgattagaatcatagcatgtaaaacccaactgagggattatctgcagacttaatgtagtattatgtaagttgtcttctttcatttcgaccttttttgcttttgttgttgctagatctgtagtatgtagctagtcacctttcagcgaggtttcagcgaggcttttctgtgtctctaggttatttgagataacttttttaaaattagctcttgtcctcc

SEQ ID NO: 104 - protein, 797 aa, Bos taurus
MAALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQVGKTLLSKRQNSEVIREKDKQILASIELLARSLPKIHRSASEPSLNRAGFQTEDFSLYACASPKTPIQAGGYEADLALTSNKNRVEVGI

SEQ ID NO: 105 - DNA, 4154 bp, Bos taurus
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaagtaggaaagactctcctaagcaagagacaaaattcagaagttatcagggaaaaagataagcagattctcgcctctattgagctgctggcccgctcattgccaaaaattcaccgcagtgcatcagaaccctccttgaatcgggctggcttccaaacagaggattttagtctatatgcttgtgcttctccaaaaacacccattcaggcagggggatatggctgagcacattgtccatcacccacaagtggctggttctcatcgcagaatctacgtagggaatcgggcgtgaaattcacttaagagatagagcagaggaagtgttctgtttacaggaatggagatgagagttatgagtaagttgcttagtcagttggctttgttttgaaaattattgtgttatatttgtgttaacctacttgtgttttgacagtatatgtcacataggaagaaacctcagactagcataataacaaagctcagactaggcacagatgtacacagaatggaccaaaatgggatgggggaaggtatgggaataagtctaggggtagggaaaaattgatgtgagggtgggaaataaactgtaattacctgaaataaaatgtaagagtgcaataagtgtgctttttattctaagctgtgaatgggttttttaaaaaaagcattccttcccaatgcatttgcctatgttccatagctgattaaaaccagctatataaacatatgcctttttattcatgttaattaccaatataaatggctaacctttacgtcttatttatcttcatgttatgttagtttacatacagggatgtgtgtgtgtgtgtatgctataaattttccctccttcgtttaaaaacgcgtttgttggatcctctctgtttccttaggccatgccacagctcatagtctcagcttggccttcctgtcacctgatctgaaggactatcacagtgacgtagctcgttcattggttgtacacactctaacccttttccttgctcagcaattactgtgtcttctaaaacaggagtgtacaaccatgagattgcaattaattgtttgacatatgtccctttgaattctatttattagttatgattgattgctctttggtttggaccaagaaaaacgaaatcccacctccccaccttttcacttatttcttactttgaggacaattctgtaagagagaggaaagggaactccttcatgttttaactgcagcaagttaatggccctggtttacaccaaacattatggtgattcacattcacattcctctcctctcttgctgccagaggtttgggttttgttcagttctgctcaagcactgaaaaagttttcatggagtctggagagtgcccagtgaaaagatggtttttaattgtccacagacctttctgttcctgctttgcaaaaattacaaaggagtaactatttttaaagcttatttttcaattcataaaaaagacatttattttcagtcagatgatgtctccttgtcccttaatcctcaatgtttgcttgaatcttttttttttttctgattttctcccatccccacttcttgatacttcttgagttctctttcctgctcaggtcctttcatttgtactttggagttttttctcatgtaaatttgtacaatggaaaatattgttcagtttggatagaacgcatggagaattaaataaaaaagatagctgaaattcagattgaaatttatttgtgtaaagttatttaaaaactctgtactatataaaaggcaaaaaaagttctatgtacttgatgtgaatatgcgaatactgctataataaagattgactgcatgga

SEQ ID NO: 106 - protein, 781 aa, Bos taurus
MAALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQVGKTLLSKRQNSEVIREKDKQILASIELLARSLPKIHRSASEPSLNRAGFQTEDFSLYACASPKTPIQAGGYG

SEQ ID NO: 107 - DNA, 7914 bp, Bos taurus
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaaattctcgcctctattgagctgctggcccgctcattgccaaaaattcaccgcagtgcatcagaaccctccttgaatcgggctggcttccaaacagaggattttagtctatatgcttgtgcttctccaaaaacacccattcaggcagggggatatggagaatttgcagccttcaagtagccacaccatcatgacagcatctactcttatttcttaagtcttgtgttcgtacaatttgttaacatcaaaacacagttctgttcctcaactctttttaaagttaaaatttttcagtgcataagctggtgtggaacagaaggaaatttcccatccaacaaaagagggaagaatgttttaggaaccagaattctctgctgccagtgtttcttcttcaacacaaatatcacaagtctgcccactcccaggaagaaagaggagagaccctgagttctgaccttttgatggtcaggcatgatggaaagaaactgctgctacagcttgggagatttgctctgggaagtctgccagtcaactttgcccttctaaccaccagatcaatatgtggctgatcatctgatggggcagttgcaatcaccaagccttgttctctttcctgttctgggattgtgttgtggaacccttttccctagccaccaccagttcatttctgagggatggaacaaaaatgcagcatgcccttcctgtgtggtgcatgttcagtccttgacaaatttttaccaaaatgaagctactttatttaaaaggagggtgagaggtgaggaggtcactttgggtgtggcggaaagggaatgctgcatctttttcctgggctgctggggctctggccttggcttgccagccggaagcgctggcacgcatcgccttcttttcccattgggtccagcaatgaagacgagtgtttggggttttttttttctccaccatgtagcaagttctcaggaaaatacaattgatatcttcctcctaagctcttccaatcagtcaccaagtacttatgtggttactttgtccagggcacaaaatgcctgtatctaattaaaagcctacaaaactgcttgataacagttttgaatgtgagacatttatgtaatttaaatgtaaggtacaagttttaatttctgagtttcttctattatatttttattaaaaaaagaaaataattttcagattgaattggagtaaaataatattacttcccactagaattatatatcctggaaaattgtatttttgttacataagcagcttttaaagaaagatcattacccttttctctacataaatatatggggagtcttagcctaatgacaaatatttataatttttaaattaatggtacttgctggatccatactaacatctttactaatacctcattgtttcttccaacttactcctacactacatcctacatcttcttcctagtcttttatctagaatatgcaacctcaaataaaaatggtggtgtcctcattcattctcctccttccttttttcccaagcctgatcttcaaaaggttggttaatttggcagctgagttcctccccaggcagagaatagaccaattttaggtgtattgggactgagggaggatgtgtaaagattaacatcagtaaagaaccgctgtggagtaattaagaactttgttctttataactggagaatataacctaaccctaacatccctcagcctttactaaagtgtggcgtaaatcacagtagtagcaaagaaagtgactctggatgtgttcctggccagtacctcccttatcatgaatgtagactctctcatcaagatttaggaatataaatcaaatcaaatgtgcccagccaagctatgtagtaagggacttgaacaatattaggcagaacctataaaataaatcagggaattagaaattatttaaagttttcaaattgtaaattgccccggtgtctttcagcctactgccattatttttgctacaatacctacatttcagaggagggcctactgaaaattccatgcaagtggaaaataatcctcaagttattaatgagtttgaaaagcaatgagttcttaagtctttgtgagtagagcaagatcctacaaaattcagaaatagtaaaaatggattcatgctgatttgaagagcatctgtgtgcataatataatgctgcatctcttttaaaagcagtctatttttctttttaaatttgtccccatagatgcttttgaacatgaacatgcttatgttaccttttccgaggttgggaagagccaggagctctcaggcagggccccctccctcagctgggcaggagctgctcaggaggagctagttatagaggaagcttagcgttggcattttcaaaattcaaggtgataacgctttcttcttcctttctgttttagaatagattgctgtctgatttgaaaaagggaaatagatttgatctcaaatgaatctgtgcccagaagccaggctcagggtattcagagatttgtatagtgccctcaaaaaataacaaaattttagctttccttttttcttcttttctccatcaaattcttttttctctagtttacaaatgacatggaaaaggaatttcccctgagttttgtatgcctttttttttttggcttagactatagataggcgtgttgagctcctaagaaaatacaaggaggaactctttgttgtgcagagcactttatgagtagtttgtgtggataatatgtgactgcttccctgacgagcttgtgaggctgtacttatgtctttcctgtaaggcagcttcagtgccttctgtagtgtatataaggaaagattacgccttctgaaaaatctcagagcaaccataagattattttaaaatatgtagtatgactgatggactttttcatcattaaattagtctagcatctaaacttttaccactgaaataatattgaccaaaaagcaatttataaaaggtatttgtgaatagaaaatacaatgtgatcatttgtacttatgtgcaccttaaaagaggaattctgtctagctgtcaaattctggttccttaacatccagtccttgattgtgattgagatctggtaggacgtgctggggcacgctagcagataaaatcccgtatactttaggatagatgttacatttatgtcagtgttggcaaagagcattgtgtagtaataaagaattcaagacttcagcaatgtcaacctgaaactttgtaaatatttcctagattgttatttgatgcagtcacagctctttatcacacaatgttgtctttccctcatcaggcaattttagaactgctgcacacccctcctcagatctcacctgcccctcctgtacattcacctctccagccttgtgcacacctcatttagctttagtttgaaacacattgcagggttcaggtgacctcttcaaaaactacctcctcagaatgaggtaatgaatagttatttattttaaaatatgaaaagtcaggagctctagaatatgaagatgatctaagattttaacttttatgtatacttgttgagcactctccttttgtcctaaagggcattatacatttaagcagtaatactgaaaaatgtagctcagagtaactgaatgttgttgaaagtggtgccagaatctgttttaggggtacgtatcagaatcttaatcttaaatcggttacatgaaattaaatagttaatggtaacacttgactaacagatataattttaattttcggtaggcttttagcaagacagtaagtacatcttcataatgagttagccacagcttcatcacatgcacagattttcctgttgagagactgcccagttaagagggtagaatgatgaaccatttttcaggattctcttctttgtccaaactggcattgtgagtgctagaatatcagcactttcaaactagtgattccaactattaggctattaaaaagcaaaacaaaccaaacaaaccatagccagacatgggaagtttactatgagtataaacagcaaatagcttacaggtcatacattgaaatggtgtaggtaaggcgttagaaaaataccttgacaatttgccaaatgatcttactgtgccttcatgatgcaataaaaaaaaaaaaaatttagcataaatcagtgatttgtgaagagagcagccaccctggtctaactcagctgtgttaatattttttagcgtgcaatttagactgcaaagataaatgcactaaagagtttatagccaaaatcacatttaaaaaatgagagaaaacacaggtaaattttcagtgaacaaaattatttttttaaagtacataatccctagtatagtcagatatatttatcacatagagcaaataggttgaaatcacaattcagtgacatttctagagaaactttttctactcccataggttcttcaaagcatggaacttttatataacagaaatgtgtgacggtcattttaaattgctgtagtttggggctgaagtactgtgtgctgggcagcaatcacatgtattaactagtgagaaaggagaaattaagatataggacagaatttgattttcttgttcccagattactgctgccaacctagacactgagtttccagaggctgaaacgtaaacttgcagctcagcaactgttttgcaaagttagtgggactgtcctgcttatgctgttcaaaaatgctctgagggccaggtggggcctccaggggctcctctctgaggggacatcagactagctaacgacctggcgggcggatgtgaaccggacacactccatggtgtgcttcttgtatcggtccctcgccaccctcaagaaaggcttcagcgggttctctagacgtctccactaaggtgtgttactaacagccatgggttgttgagcacccgaggagtgcaatagcatctctgcatgattgtatattggcccgaagagaatgaagtggccagtgtactcatgttccatgttgctagctctggtaaactgaaaatactggtaagatttttgttttatcagtacactagagagtaagctttgttttgttgtttttagataatgttttcacttccatttggaaagacatttaaattgagtttcagtcctaaattttgccagtcatggtaattagcagtttctatcaggtatttttaaggtagaagaggatagaaacataagttctaaaagcttaaggtaaccgtggtttattttaaaatgtttaggggtggttagtctctacctcaaaaaaagtgagtgaatcttttatttcagcattcacaagttcggctgttgtttttgtaatacattttttttttaaccttttgacccccctttacctaagtgtcaatgtagttttattaattactaagtcagtttcattaaaatgtttatttagcagttttgactaattgcaatgattaatatagccagttgtgcatgaggacacagccagtgagtatatctgggttttttttgtgatgctttttttcttaagacttctgtagatttatgaagtactcattgaaaacaactaaaatacgtttattcgtgttaatatggaaaaaaaaaaa

SEQ ID NO: 108 - protein, 766 aa, Bos taurus
MAALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGEFAAFK

SEQ ID NO: 109 - DNA, 4670 bp, Bos taurus
ggtgtgtcatagtgcagcagattgaatgcagaagatatgaaaattcagatgtcttctgttaaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaagtaggaaagactctcctaagcaagagacaaaattcagaagttatcagggaaaaagataagcagattctcgcctctattgagctgctggcccgctcattgccaaaaattcaccgcagtgcatcagaaccctccttgaatcgggctggcttccaaacagaggattttagtctatatgcttgtgcttctccaaaaacacccattcaggcagggggatatgaagcagatttggctcttacatcaaataaaaatagagtagaagttgggatttagagatttcctgacatgcaagaaggaataagcaagaaaaaaaggtttgttttccccaaatcatatctattgtcttttacttctattttttcttaaattttttgtgatttcagagacatgtagagttttattgatacctaaactatgagttcttttttttttttttttttcattattttgatttttttggccaagaggcatatgggatcttagcttgagaaagcaacaattttcttgatgtcattttgggtgagggcacatattgctgtgaacagtgtggtgatagccaccagggaccaaactcacacccgctgcattgaaaggtgaagtcttaaacactggaccagcagagaaattcctactctatgagttctttttgtcatcccctccccgcaccctccacccccaacctaaagtctgatgatgaaatcaacaactattccattagaagcagtagattctggtagcatgatctttagtttgttagtaagattttgtgctttgtggggttgtgtcgttttaaggctaatatttaagtttgtcaaatagaatgctgttcagattgtaaaaatgagtaataaacatctgaagttttttttaagttatttttaacatggtatatacagttgagcttagagtttatcattttctgatattctcttacttagtagatgaattctagccattttttataaagatttctgttaagcaaatcctgttttcacatgggcttcctttaagggattttagattctgctggatatggtgactgctcataagactgttgaaaattacttttaagatgtattagaatacttctgaaaaaaaatagcaaccttaaaaccataagcaaaagtagtaagggtgtttatacatttctagagtccctgtttaggtaatagcctcctatgattgtactttaaatgttttgctctccaaggttttagtaacttggctttttttctaatcagtgccaaactcccccagtttttttaactttaaatatgaggtaataaatcttttacccttccttgatcttttgacttataataccttggtcagttgtttcttaaaaggaatccttaaatggaaagagacaatatcactgtctgcagttctgattagtagttttattcagaatggaaaaacagattattcatttttgaaaattgttcaggggtatgttcattgttaggaccttggactttggagtcagtgcctagctatgcattccaggtctgccattttctggctgtgaaattttggacaagttacttaaccactttaaaccccagctttaagaagtaaattaaccccagtaaattaagaagtaatagcagccacttcgtagagttgttatgaggctcagatgcagtgcaaatgtgtataaagtattcagggagtcacctggtatactataatagacactagaatagttgccaatattatcagcatacaatctgaggattctgtcagccaatcattagcaatctgttgtttgttgggacatgccagtgttctccagttgaaatcagtagcaatctaaaaatggatagattattcctcatttaaatagtgtgttcatataagtgattgcttggatccttatcagaagttgctgttactgaaaaatgataaggctgactaaattgtgatagttgtcagttactaaccaactcccagaaatgaataagaggaacctatctctagttcctagtagaaggtatggacaaaatagtaggtgaaaaataatgtcttgaacccccaaattaagtaagctttaaagagtacaatacctcaaagggtctttgcggtttaaaatttgtatgctgagaatgatgttcattgacatgtgcctatatgtaattttttgatagtttaaaaggtgaaatgaactacagatgggagaggtctgaattttcttgccttcagtcaaatgtgtaatgtggacatattatttgacctgtgaattttatcttttaaaaaagattaattcctgcttcttccttcctaatagttgcattataataatgaaaatgagttgataatttggggggaaagtattctacaaatcaaccttattattttaccattggtttctgagaaattttgttcatttgaaccgtttatagcttgattagaatcatagcatgtaaaacccaactgagggattatctgcagacttaatgtagtattatgtaagttgtcttctttcatttcgaccttttttgcttttgttgttgctagatctgtagtatgtagctagtcacctttcagcgaggtttcagcgaggcttttctgtgtctctaggttatttgagataacttttttaaaattagctcttgtcctcc

SEQ ID NO: 110 - protein, 761 aa, Bos taurus
MKIQMSSVKVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQVGKTLLSKRQNSEVIREKDKQILASIELLARSLPKIHRSASEPSLNRAGFQTEDFSLYACASPKTPIQAGGYEADLALTSNKNRVEVGI

SEQ ID NO: 111 - DNA, 4816 bp, Bos taurus
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagagcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaagtaggaaagactctcctaagcaagagacaaaattcagaagttatcagggaaaaagataagcagattctcgcctctattgagctgctggcccgctcattgccaaaaattcaccgcagtgcatcagaaccctccttgaatcgggctggcttccaaacagaggattttagtctatatgcttgtgcttctccaaaaacacccattcaggcagggggatatgaagcagatttggctcttacatcaaataaaaatagagtagaagttgggatttagagatttcctgacatgcaagaaggaataagcaagaaaaaaaggtttgttttccccaaatcatatctattgtcttttacttctattttttcttaaattttttgtgatttcagagacatgtagagttttattgatacctaaactatgagttcttttttttttttttttttcattattttgatttttttggccaagaggcatatgggatcttagcttgagaaagcaacaattttcttgatgtcattttgggtgagggcacatattgctgtgaacagtgtggtgatagccaccagggaccaaactcacacccgctgcattgaaaggtgaagtcttaaacactggaccagcagagaaattcctactctatgagttctttttgtcatcccctccccgcaccctccacccccaacctaaagtctgatgatgaaatcaacaactattccattagaagcagtagattctggtagcatgatctttagtttgttagtaagattttgtgctttgtggggttgtgtcgttttaaggctaatatttaagtttgtcaaatagaatgctgttcagattgtaaaaatgagtaataaacatctgaagttttttttaagttatttttaacatggtatatacagttgagcttagagtttatcattttctgatattctcttacttagtagatgaattctagccattttttataaagatttctgttaagcaaatcctgttttcacatgggcttcctttaagggattttagattctgctggatatggtgactgctcataagactgttgaaaattacttttaagatgtattagaatacttctgaaaaaaaatagcaaccttaaaaccataagcaaaagtagtaagggtgtttatacatttctagagtccctgtttaggtaatagcctcctatgattgtactttaaatgttttgctctccaaggttttagtaacttggctttttttctaatcagtgccaaactcccccagtttttttaactttaaatatgaggtaataaatcttttacccttccttgatcttttgacttataataccttggtcagttgtttcttaaaaggaatccttaaatggaaagagacaatatcactgtctgcagttctgattagtagttttattcagaatggaaaaacagattattcatttttgaaaattgttcaggggtatgttcattgttaggaccttggactttggagtcagtgcctagctatgcattccaggtctgccattttctggctgtgaaattttggacaagttacttaaccactttaaaccccagctttaagaagtaaattaaccccagtaaattaagaagtaatagcagccacttcgtagagttgttatgaggctcagatgcagtgcaaatgtgtataaagtattcagggagtcacctggtatactataatagacactagaatagttgccaatattatcagcatacaatctgaggattctgtcagccaatcattagcaatctgttgtttgttgggacatgccagtgttctccagttgaaatcagtagcaatctaaaaatggatagattattcctcatttaaatagtgtgttcatataagtgattgcttggatccttatcagaagttgctgttactgaaaaatgataaggctgactaaattgtgatagttgtcagttactaaccaactcccagaaatgaataagaggaacctatctctagttcctagtagaaggtatggacaaaatagtaggtgaaaaataatgtcttgaacccccaaattaagtaagctttaaagagtacaatacctcaaagggtctttgcggtttaaaatttgtatgctgagaatgatgttcattgacatgtgcctatatgtaattttttgatagtttaaaaggtgaaatgaactacagatgggagaggtctgaattttcttgccttcagtcaaatgtgtaatgtggacatattatttgacctgtgaattttatcttttaaaaaagattaattcctgcttcttccttcctaatagttgcattataataatgaaaatgagttgataatttggggggaaagtattctacaaatcaaccttattattttaccattggtttctgagaaattttgttcatttgaaccgtttatagcttgattagaatcatagcatgtaaaacccaactgagggattatctgcagacttaatgtagtattatgtaagttgtcttctttcatttcgaccttttttgcttttgttgttgctagatctgtagtatgtagctagtcacctttcagcgaggtttcagcgaggcttttctgtgtctctaggttatttgagataacttttttaaaattagctcttgtcctcc

SEQ ID NO: 112 - protein, 757 aa, Bos taurus
MAALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLEPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQVGKTLLSKRQNSEVIREKDKQILASIELLARSLPKIHRSASEPSLNRAGFQTEDFSLYACASPKTPIQAGGYEADLALTSNKNRVEVGI

SEQ ID NO: 113 - DNA, 2499 bp, Bos taurus
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaagtaggaaagactctcctaagcaagagacaaaattcagaagttatcagggaaaaagataagcaggaaaagtatgtttctttagtacattccaggcatttgggattacagtaaaaacaatattctcgcctctattgagctgctggcccgctcattgccaaaaattcaccgcagtgcatcagaa

SEQ ID NO: 114 - protein, 744 aa, Bos taurus
MAALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQVGKTLLSKRQNSEVIREKDKQEKYVSLVHSRHLGLQ

SEQ ID NO: 115 - DNA, 2404 bp, Bos taurus
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaagatctctcttcccaccatagacacaaaaatttcagatggctacaggtttacatgtaaaaaacagaattataacaaatgatttttat

SEQ ID NO: 116 - protein, 726 aa, Bos taurus
MAALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQDLSSHHRHKNFRWLQVYM

SEQ ID NO: 117 - DNA, 2331 bp, Bos taurus
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccaggtgctttgtcctccatgggagtgtaataaatgctgtgcaagggcttacttcccatgagagaagtgagtgaccaacagaaggataatttttatggtgggacgaggatatctgtctccagatctcagtaaggtacggagtaactgtccaa

SEQ ID NO: 118 - protein, 681 aa, Bos taurus
MAALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQVLCPPWECNKCCARAYFP

SEQ ID NO: 119 - DNA, 2319 bp, Bos taurus
ctcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccaggtgctttgtcctccatgggagtgtaataaatgctgtgcaagggcttacttcccatgagagaagtgagtgaccaacagaaggtctgtgcaaggaaaagagacaaagccacggatcagaagcacatggccataactga

SEQ ID NO: 120 - protein, 681 aa, Bos taurus; identical to SEQ ID NO: 118

SEQ ID NO: 121 - DNA, 2661 bp, Bos taurus
tcagctgcgccgggtctcacaagacggttcccgaggtggcccaggcgccgtcccaccgccgacgccgcccgggccgcccgggccgtccctccccgctgccccccgtcctccgcctccgcctccccccgccctcagcctcccttccccctccccgcccagcagcggtcgctcgggcccggctctcggttataagatggcggcgctgagtggcggcggcggcggcggcggcggtggcgcggagcagggccaggctctgttcaacggggacatggagcccgaggccggcgccgcggcctcttcggctgcggaccccgccattcccgaggaggtgtggaatatcaaacaaatgattaagttgacacaggagcatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagctctgcatcaacggacaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagtctgtgcaaggaaaagagacaaagccacggatcagaagcacatggccataactgaagattttgtgaactctcacaaggaaaaaatttgctctttgaacaataagaaggaactcactaaaatgtaactgagaactgttcaacaggttgaaagctgaaagatgccattggaactgacaaaatgtttcttaaacataaatgatgaaacagtgaaactacataatatctcctctggctgaaacattcaagaagtttaaaatgcttaagttaaaaataaaatcctagtaaacaatggacttactgtgcaacatagagaatatcttacgataacctgtaatggaaaagaatctgaaaaagaatgtatataactgaatcactttgctgtaaactagaatctgacacaacactgtaaatcactacacttttctgttgcatgccaaagattatttaataacgtcattaaaaaattattttaataatta

SEQ ID NO: 122 - protein, 679 aa, Bos taurus
MAALSGGGGGGGGGAEQGQALFNGDMEPEAGAAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQSVQGKETKPRIRSTWP

SEQ ID NO: 123 - DNA, 7434 bp, Bos taurus
acaccgttacatcttcttcctcttctagcctttcagtgctgccttcatctctttcagtttttcaaaatcccacagatgtgtcacggagcaaccccaagtcaccacaaaaacctatcgttagagtcttcctgcccaataaacagaggacagtggtacctgcacggtgtggagtcacagtccgggacagcctgaagaaggcactgatgatgagaggtctaatcccagagtgctgtgctgtttacagaattcaggatggggagaagaaaccaattggctgggacactgatatttcctggcttactggagaggagttgcatgtagaagtgttggagaatgttccacttacaacacacaactttgtacggaaaacttttttcaccttagcattttgtgacttctgtagaaagctgcttttccagggattccgctgtcaaacatgtggttataaatttcaccagcgttgtagtacagaggttccactgatgtgtgttaattatgaccaactagatttgctgtttgtctccaagttctttgaacaccacccaataccacaggaggaggcctccttagcagagactacccttccatgtggctcatccccttctgcacccccctccgattctattgggcccccaattctcaccagtccatctccttcaaaatccattccaattccacagcctttccgaccagcagatgaagatcatcgaaatcagtttggacaacgagaccggtcctcatcagctccaaatgtgcatataaacacaatagaacccgtcaatattgatgacttgattagagaccaagggtttcgtagtgatggaggatcaaccacaggtttatccgccacaccccctgcctcattacctggctcactctctaatgtgaaagcattgcagaaatctccaggacctcagcgagaaagaaagtcctcttcatcctcagaagacaggaatcgaatgaaaacgcttggtagacgggattcaagtgacgattgggagattcctgatggacagatcacagtgggacaaagaattggatcagggtcatttgggacagtctacaagggaaagtggcatggtgatgtggcagtgaaaatgttgaatgtgacagcacccacacctcagcagttacaggccttcaaaaatgaagtaggagtactcaggaaaacgcgacatgtgaatatcctcctcttcatgggttattcaacaaagccacaactggctattgttacccagtggtgtgagggctccagtttatatcatcatctccacatcattgagaccaaattcgagatgatcaaacttatagatattgcacggcagactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccatatagctttcagtcagatgtatatgcatttgggattgttctgtatgaattgatgaccggacagttaccttattcaaatatcaacaacagggaccagataatttttatggtgggacgaggatatctgtctccagatctcagtaaggtacggagtaactgtccaaaagccatgaagagattaatggcagagtgcctaaaaaagaaaagagatgaaagaccactctttccccaagtaggaaagactctcctaagcaagagacaaaattcagaagttatcagggaaaaagataagcagattctcgcctctattgagctgctggcccgctcattgccaaaaattcaccgcagtgcatcagaaccctccttgaatcgggctggcttccaaacagaggattttagtctatatgcttgtgcttctccaaaaacacccattcaggcagggggatatggagaatttgcagccttcaagtagccacaccatcatgacagcatctactcttatttcttaagtcttgtgttcgtacaatttgttaacatcaaaacacagttctgttcctcaactctttttaaagttaaaatttttcagtgcataagctggtgtggaacagaaggaaatttcccatccaacaaaagagggaagaatgttttaggaaccagaattctctgctgccagtgtttcttcttcaacacaaatatcacaagtctgcccactcccaggaagaaagaggagagaccctgagttctgaccttttgatggtcaggcatgatggaaagaaactgctgctacagcttgggagatttgctctgggaagtctgccagtcaactttgcccttctaaccaccagatcaatatgtggctgatcatctgatggggcagttgcaatcaccaagccttgttctctttcctgttctgggattgtgttgtggaacccttttccctagccaccaccagttcatttctgagggatggaacaaaaatgcagcatgcccttcctgtgtggtgcatgttcagtccttgacaaatttttaccaaaatgaagctactttatttaaaaggagggtgagaggtgaggaggtcactttgggtgtggcggaaagggaatgctgcatctttttcctgggctgctggggctctggccttggcttgccagccggaagcgctggcacgcatcgccttcttttcccattgggtccagcaatgaagacgagtgtttggggttttttttttctccaccatgtagcaagttctcaggaaaatacaattgatatcttcctcctaagctcttccaatcagtcaccaagtacttatgtggttactttgtccagggcacaaaatgcctgtatctaattaaaagcctacaaaactgcttgataacagttttgaatgtgagacatttatgtaatttaaatgtaaggtacaagttttaatttctgagtttcttctattatatttttattaaaaaaagaaaataattttcagattgaattggagtaaaataatattacttcccactagaattatatatcctggaaaattgtatttttgttacataagcagcttttaaagaaagatcattacccttttctctacataaatatatggggagtcttagcctaatgacaaatatttataatttttaaattaatggtacttgctggatccatactaacatctttactaatacctcattgtttcttccaacttactcctacactacatcctacatcttcttcctagtcttttatctagaatatgcaacctcaaataaaaatggtggtgtcctcattcattctcctccttccttttttcccaagcctgatcttcaaaaggttggttaatttggcagctgagttcctccccaggcagagaatagaccaattttaggtgtattgggactgagggaggatgtgtaaagattaacatcagtaaagaaccgctgtggagtaattaagaactttgttctttataactggagaatataacctaaccctaacatccctcagcctttactaaagtgtggcgtaaatcacagtagtagcaaagaaagtgactctggatgtgttcctggccagtacctcccttatcatgaatgtagactctctcatcaagatttaggaatataaatcaaatcaaatgtgcccagccaagctatgtagtaagggacttgaacaatattaggcagaacctataaaataaatcagggaattagaaattatttaaagttttcaaattgtaaattgccccggtgtctttcagcctactgccattatttttgctacaatacctacatttcagaggagggcctactgaaaattccatgcaagtggaaaataatcctcaagttattaatgagtttgaaaagcaatgagttcttaagtctttgtgagtagagcaagatcctacaaaattcagaaatagtaaaaatggattcatgctgatttgaagagcatctgtgtgcataatataatgctgcatctcttttaaaagcagtctatttttctttttaaatttgtccccatagatgcttttgaacatgaacatgcttatgttaccttttccgaggttgggaagagccaggagctctcaggcagggccccctccctcagctgggcaggagctgctcaggaggagctagttatagaggaagcttagcgttggcattttcaaaattcaaggtgataacgctttcttcttcctttctgttttagaatagattgctgtctgatttgaaaaagggaaatagatttgatctcaaatgaatctgtgcccagaagccaggctcagggtattcagagatttgtatagtgccctcaaaaaataacaaaattttagctttccttttttcttcttttctccatcaaattcttttttctctagtttacaaatgacatggaaaaggaatttcccctgagttttgtatgcctttttttttttggcttagactatagataggcgtgttgagctcctaagaaaatacaaggaggaactctttgttgtgcagagcactttatgagtagtttgtgtggataatatgtgactgcttccctgacgagcttgtgaggctgtacttatgtctttcctgtaaggcagcttcagtgccttctgtagtgtatataaggaaagattacgccttctgaaaaatctcagagcaaccataagattattttaaaatatgtagtatgactgatggactttttcatcattaaattagtctagcatctaaacttttaccactgaaataatattgaccaaaaagcaatttataaaaggtatttgtgaatagaaaatacaatgtgatcatttgtacttatgtgcaccttaaaagaggaattctgtctagctgtcaaattctggttccttaacatccagtccttgattgtgattgagatctggtaggacgtgctggggcacgctagcagataaaatcccgtatactttaggatagatgttacatttatgtcagtgttggcaaagagcattgtgtagtaataaagaattcaagacttcagcaatgtcaacctgaaactttgtaaatatttcctagattgttatttgatgcagtcacagctctttatcacacaatgttgtctttccctcatcaggcaattttagaactgctgcacacccctcctcagatctcacctgcccctcctgtacattcacctctccagccttgtgcacacctcatttagctttagtttgaaacacattgcagggttcaggtgacctcttcaaaaactacctcctcagaatgaggtaatgaatagttatttattttaaaatatgaaaagtcaggagctctagaatatgaagatgatctaagattttaacttttatgtatacttgttgagcactctccttttgtcctaaagggcattatacatttaagcagtaatactgaaaaatgtagctcagagtaactgaatgttgttgaaagtggtgccagaatctgttttaggggtacgtatcagaatcttaatcttaaatcggttacatgaaattaaatagttaatggtaacacttgactaacagatataattttaattttcggtaggcttttagcaagacagtaagtacatcttcataatgagttagccacagcttcatcacatgcacagattttcctgttgagagactgcccagttaagagggtagaatgatgaaccatttttcaggattctcttctttgtccaaactggcattgtgagtgctagaatatcagcactttcaaactagtgattccaactattaggctattaaaaagcaaaacaaaccaaacaaaccatagccagacatgggaagtttactatgagtataaacagcaaatagcttacaggtcatacattgaaatggtgtaggtaaggcgttagaaaaataccttgacaatttgccaaatgatcttactgtgccttcatgatgcaataaaaaaaaaaaaaatttagcataaatcagtgatttgtgaagagagcagccaccctggtctaactcagctgtgttaatattttttagcgtgcaatttagactgcaaagataaatgcactaaagagtttatagccaaaatcacatttaaaaaatgagagaaaacacaggtaaattttcagtgaacaaaattatttttttaaagtacataatccctagtatagtcagatatatttatcacatagagcaaataggttgaaatcacaattcagtgacatttctagagaaactttttctactcccataggttcttcaaagcatggaacttttatataacagaaatgtgtgacggtcattttaaattgctgtagtttggggctgaagtactgtgtgctgggcagcaatcacatgtattaactagtgagaaaggagaaattaagatataggacagaatttgattttcttgttcccagattactgctgccaacctagacactgagtttccagaggctgaaacgtaaacttgcagctcagcaactgttttgcaaagttagtgggactgtcctgcttatgctgttcaaaaatgctctgagggccaggtggggcctccaggggctcctctctgaggggacatcagactagctaacgacctggcgggcggatgtgaaccggacacactccatggtgtgcttcttgtatcggtccctcgccaccctcaagaaaggcttcagcgggttctctagacgtctccactaaggtgtgttactaacagccatgggttgttgagcacccgaggagtgcaatagcatctctgcatgattgtatattggcccgaagagaatgaagtggccagtgtactcatgttccatgttgctagctctggtaaactgaaaatactggtaagatttttgttttatcagtacactagagagtaagctttgttttgttgtttttagataatgttttcacttccatttggaaagacatttaaattgagtttcagtcctaaattttgccagtcatggtaattagcagtttctatcaggtatttttaaggtagaagaggatagaaacataagttctaaaagcttaaggtaaccgtggtttattttaaaatgtttaggggtggttagtctctacctcaaaaaaagtgagtgaatcttttatttcagcattcacaagttcggctgttgtttttgtaatacattttttttttaaccttttgacccccctttacctaagtgtcaatgtagttttattaattactaagtcagtttcattaaaatgtttatttagcagttttgactaattgcaatgattaatatagccagttgtgcatgaggacacagccagtgagtatatctgggttttttttgtgatgctttttttcttaagacttctgtagatttatgaagtactcattgaaaacaactaaaatacgtttattcgtgttaatatggaaaaaaaaaaa

SEQ ID NO: 124 - protein, 603 aa, Bos taurus
MMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETTLPCGSSPSAPPSDSIGPPILTSPSPSKSIPIPQPFRPADEDHRNQFGQRDRSSSAPNVHINTIEPVNIDDLIRDQGFRSDGGSTTGLSATPPASLPGSLSNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQVGKTLLSKRQNSEVIREKDKQILASIELLARSLPKIHRSASEPSLNRAGFQTEDFSLYACASPKTPIQAGGYGEFAAFK

SEQ ID NO: 125 - DNA, 2295 bp, Equus caballus
  misc_feature (79)..(79): n is a, c, g, or t
atgaagacgctgagcggcggcggcggcggcgcggagcagggccaggctctgttcaacggggacatggaacccggaggcnccgcgccggcgcccgcggcctcgtcggccgcggaccctgccattcccgaggaggtatggaatatcaaacaaatgattaaattgacacaggaacatatagaggccctattggacaaatttggtggggagcataatccaccatcaatatatctggaggcctatgaagaatacaccagcaagctagatgccctccaacaaagagaacaacagttattggaatccctggggaatggaactgatttttctgtttctagttctgcatcaacggacaccgttacatcttcttcctcttctagcctttcagtgctaccttcatctctttcagtttttcaaaatcccacagatgtgtcacggagcaaccctaagtcaccacaaaaacctatcgttagagtcttcctgcccaacaaacagaggacagtggtacctgcaaggtgtggcgttacagtccgggacagtctaaagaaagcactgatgatgagaggtctaatcccagagtgctgtgctgtttacagaattcaggatggagagaagaaaccaattggctgggacactgatatttcctggctcactggagaggaattgcatgtagaagtgttggagaatgttccacttacaacacacaactttgtacggaaaacttttttcaccttagcattttgtgacttttgtcgaaagctgcttttccagggtttccgctgtcaaacatgtggttataaatttcaccagcgttgtagtacagaggttccactgatgtgtgttaattatgaccaacttgatttgctgtttgtctccaagttctttgaacaccacccagtatcacaggaggaggcctccttagcagagactgcccttacatctggatcatccccttctgcacccccctccgattccattgggccccaaattctcaccagtccatctccttcaaaatccattccaattccacagcctttccgaccagcagatgaagatcatcgaaatcagtttggacaacgagaccggtcctcatcagctccaaatgtacatataaacacaatagaacctgtcaatattgatgacttgattagagaccaagggtttcgtagtgatggaggatcaaccacaggtttatctgccaccccccctgcctcattacctggctcactcactaatgtgaaggcattacagaaatctccaggacctcaacgggaaaggaaatcatcttcatcctcagaagacaggaatcgaatgaaaactcttggtagacgggattcaagtgacgattgggagattcctgatgggcagatcacagtgggacaaagaattggatctgggtcatttgggacagtctacaagggaaagtggcatggtgatgtggcagtgaaaatgttgaatgtgacagcacccacacctcagcagttacaggccttcaaaaatgaagtaggagtactcaggaaaactcgacatgtgaatatcctactcttcatgggctattcaacaaagccacaactggctattgttacccagtggtgtgagggctccagcttatatcaccatctccacatcattgagaccaaatttgagatgatcaaacttatagatattgctcggcaaactgcacagggcatggattacttacacgccaagtcaatcatccacagagacctcaagagtaataatatttttcttcatgaagacctcacagtaaaaataggtgattttggtctagccacagtgaaatctcgatggagtgggtcccatcagtttgaacagttgtctggatccattttgtggatggcaccagaagtaatcagaatgcaagataaaaacccgtatagctttcaatcagatgtatatgcctttgggattgttctgtatgaattgatgactggacagttaccttattcaaacatcaacaacagggaccagataatttttatggtgggaagaggatatctatctccagatctcagtaaggtacggagtaactgtccaaaagccatgaagagattaatggcagagtgcctaaaaaagaaaagagacgagagaccactcttcccccaaattctcgcctctattgagctgctggcccgctcattgccaaaaattcaccgcagtgcatcagagccctccttgaatcgggctggcttccagacagaggattttagtctatatgcttgtgcttctccgaaaacacccatccaggcagggggatatggtgcgtttcctgtccactga

SEQ ID NO: 126 - protein, 764 aa, Equus caballus
  misc_feature (27)..(27): Xaa can be any naturally occurring amino acid
MKTLSGGGGGAEQGQALFNGDMEPGGXAPAPAASSAADPAIPEEVWNIKQMIKLTQEHIEALLDKFGGEHNPPSIYLEAYEEYTSKLDALQQREQQLLESLGNGTDFSVSSSASTDTVTSSSSSSLSVLPSSLSVFQNPTDVSRSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPVSQEEASLAETALTSGSSPSAPPSDSIGPQILTSPSPSKSIPIPQPFRPADEDHRNQFGQRDRSSSAPNVHINTIEPVNIDDLIRDQGFRSDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 127 - DNA, 2678 bp, Gallus gallus
tccccctccctcgccccagcgcttcgatccaagatggcggcgctgagcagcggcagcagcgccgagggggcctcgctcttcaacggggacatggagcccgagccgccgccgcccgtgctgggcgcctgctacgccgggagcggcggcggcgacccggccatcccggaggaggtgtggaatatcaaacagatgattaaattaacacaagaacatatagaagcgctgttagacaagtttggaggagagcataacccaccatcaatatatttagaggcctatgaggagtacaccagcaaactagatgctctacagcagagagaacagcagttattggaatccatgggaaatggaactgatttctctgtttccagttcagcttcaacggacacagttgcatcatcttcctcctctagcctctctgtagcaccttcatccctttcagtttatcaaaatcctactgatatgtcgcggaataaccctaagtctccacagaagcctattgttagagtcttcctgcccaacaagcaaaggactgtggttccggcaagatgtggggtgacagtccgagacagcctgaagaaagctctgatgatgagaggtcttattccagaatgctgtgctgtttacagaatacaggatggagagaagaagccaattggctgggacactgacatttcctggctaaccggagaggagttacacgtggaggtcttggagaatgtgccactcacaacacacaattttgtacgaaaaacattcttcacgttagcgttctgcgacttctgtcgaaagctgcttttccagggattccgatgccagacatgtggctacaaatttcaccagcgctgtagcacagaagtgccactgatgtgtgttaactacgaccaactcgatttgctgtttgtctccaagttctttgaacatcaccccatatcgcaggaggagaccaccttaggagagaccaccccggcatcgggatcgtacccctcagtgcccccatcagattctgttggaccaccaattctccctagtccttctccttcaaaatccattccaatcccacagcccttccgaccagcagatgaagaccatcggaatcagtttgggcaacgcgaccgatcctcttcagctcccaatgttcacatcaatacaattgagccagtcaatattgatgacttgattagagaccagggtgtacgaggagagggagcccctttgaaccagctgatgcgctgtcttcggaaataccaatcccggactcccagtcccctccttcattctgtccccagtgaaatagtgtttgattttgagcctggcccagtgttcagaggttcaactgcaggtttgtctgcaacacctcctgcatctttgcctgggtcacttaccaatgtgaaagcattacagaaatcaccaggcccccaacgggaaaggaaatcatcctcatcctcagaagacagaaataggatgaaaacccttggtcgacgagattcaagtgatgattgggaaataccagatgggcagatcacagttggacaaaggataggatctggatcatttggaacagtctacaaaggaaagtggcatggtgacgtggcagtgaaaatgttgaatgttacagcacccacacctcaacagttacaggctttcaaaaatgaagtaggagtgctcaggaaaacacggcatgtgaatatcctactttttatgggttattcaacaaaacctcagttggctattgttacacagtggtgtgaggggtccagcttatatcaccatctgcacataattgagaccaagtttgaaatgatcaaactaattgatattgcacgacagactgcacaaggcatggattatttgcatgccaagtcaatcatccacagagacctcaagagtaataatatttttcttcatgaagacctcacagtaaaaataggtgacttcggtctggctacagtgaaatcacgatggagtggatctcatcaatttgaacagttatctggatcaattctatggatggcaccggaagtgatcaggatgcaagacaaaaacccatatagctttcagtcagatgtgtatgcattcgggattgtgctttatgaactgatgactggacagttaccatactcaaacatcaacaacagggaccagataatttttatggtgggacgaggatatctatctccagacctcagtaaagtaagaagtaactgtccaaaagctatgaagagactaatggcagaatgcttgaaaaagaaaagagatgagagacctctttttccacagattcttgcctccattgagcttctggcccggtcgttgccaaaaattcaccgcagtgcatctgagccgtcactaaaccgggctggcttccagaccgaggatttcagtctgtatgcttgtgcttctccaaaaacgcccatccaagcagggggatacggtgggtttccagtacactgaaaagaaatgtgaaagcgtgtgcctgtttgctcatgtgctggtgtgttcctgtgtgtgcaacgcatacgtacgttctcagttcctaccagcgactttttaaggtttactgagggaatgaagactcatttcctaacatggggcattgaacgtcctgagcacaagtcagtgctggtaaggaatgtcttgggaacagctggcaagaagaattagaaggtacttaaagg

SEQ ID NO: 128 - protein, 806 aa, Gallus gallus
MAALSSGSSAEGASLFNGDMEPEPPPPVLGACYAGSGGGDPAIPEEVWNIKQMIKLTQEHIEALLDKFGGEHNPPSIYLEAYEEYTSKLDALQQREQQLLESMGNGTDFSVSSSASTDTVASSSSSSLSVAPSSLSVYQNPTDMSRNNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPISQEETTLGETTPASGSYPSVPPSDSVGPPILPSPSPSKSIPIPQPFRPADEDHRNQFGQRDRSSSAPNVHINTIEPVNIDDLIRDQGVRGEGAPLNQLMRCLRKYQSRTPSPLLHSVPSEIVFDFEPGPVFRGSTAGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGGFPVH

(19) United States
(12) Patent Application Publication
(10) Pub. No.: US 2016/0310477 A1
(43) Pub. Date: Oct. 27, 2016

(54) CANCER TREATMENTS USING COMBINATIONS OF MEK TYPE 1 AND ERK INHIBITORS

(71) Applicant: BIOMED VALLEY DISCOVERIES, INC., Kansas City, MO (US)

(72) Inventors: Saurabh Saha, Wellesley Hills, MA (US); Gary DeCrescenzo, Parkville, MO (US); Jeffrey James Roix, Boston, MA (US)

(21) Appl. No.: 15/105,945

(22) PCT Filed: Dec. 19, 2014
(86) PCT No.: PCT/US14/71744

§ 371 Date: Jun. 17, 2016

Related U.S. Application Data
(60) Provisional application No. 61/919,606, filed on Dec. 20, 2013.

(51) Int. Cl. A61K 31/4439; A61K 31/365; A61K 31/5377; A61K 45/06

(52) U.S. Cl. CPC A61K 31/4439; A61K 45/06; A61K 31/365; A61K 31/5377

(57) ABSTRACT

The present invention provides, inter alia, methods, kits, and pharmaceutical compositions for treating or ameliorating the effects of a cancer in a subject in need thereof. The method comprises administering to the subject an effective amount of (i) a first anti-cancer agent, which is BVD-523 or a pharmaceutically acceptable salt thereof and (ii) a second anti-cancer agent, which is a type 1 MEK inhibitor or a pharmaceutically acceptable salt thereof, to treat or ameliorate the effects of the cancer. Additional methods for effecting cancer cell death are also provided.

### CROSS REFERENCE TO RELATED APPLICATIONS

This application claims benefit of U.S. Patent Application Ser. No. 61/919,606, filed on Dec. 20, 2013 which application is incorporated by reference herein in its entirety.

### FIELD OF INVENTION

The present invention provides, inter alia, methods, pharmaceutical compositions, and kits for treating or ameliorating the effects of a cancer in a subject using a first anti-cancer agent, which is BVD-523 or a pharmaceutically acceptable salt thereof and a second anti-cancer agent, which is a type 1 MEK inhibitor or a pharmaceutically acceptable salt thereof.

### INCORPORATION BY REFERENCE OF SEQUENCE LISTING

This application contains references to amino acids and/or nucleic acid sequences that have been filed concurrently herewith as sequence listing text file “0375611.txt”, file size of 468 KB, created on Dec. 19, 2014. The aforementioned sequence listing is hereby incorporated by reference in its entirety pursuant to 37 C.F.R. §1.52(e)(5).

### BACKGROUND OF THE INVENTION

Within cellular signaling networks, RAS and RAF play significant roles in the regulation of various biological processes including cell growth, proliferation, differentiation, inflammatory responses, and programmed cell death. Notably, mutations in RAS genes were the first genetic alterations identified in human cancer. Activating mutations of HRAS, NRAS, and KRAS (‘RAS’), as well as BRAF are found frequently in several types of cancer.

MEK inhibitors, such as type 1 MEK inhibitors, inhibit the mitogen activated protein kinase enzymes, members of the MAPK signaling pathway and have some potential for the treatment of certain cancers, particularly BRAF-mutant melanoma and K-RAS/BRAF mutant colorectal cancer. Unfortunately, it is not uncommon for cancer cells to develop resistance to MEK inhibitor therapies. Recently, preliminary success has been reported in overcoming MEK resistance by co-administering a particular ATP-competitive ERK inhibitor, together with a non-ATP-competitive (i.e., type 2) MEK inhibitor (PD6325901) to a K-RAS mutant breast cancer cell line (Hatzivassiliou et al., 2012).

Extracellular-signal-regulated kinases (ERKs) are protein kinases that are involved in cell cycle regulation, including the regulation of meiosis, mitosis, and postmitotic functions in differentiated cells. Disruption of the ERK pathway is common in cancers. However, to date, little progress has been made developing effective ERK inhibitors for the treatment of cancer.

As the understanding of the molecular basis of cancer grows, there is an increased emphasis on developing drugs that specifically target particular nodes in pathways that lead to cancer. In view of the deficiencies noted above, there is, inter alia, a need for effective molecularly targeted cancer treatments. The present invention is directed to meeting these and other needs.

### SUMMARY OF THE INVENTION

One embodiment of the present invention is a method of treating or ameliorating the effects of a cancer in a subject in need thereof. The method comprises administering to the subject an effective amount of (i) a first anti-cancer agent, which is BVD-523 or a pharmaceutically acceptable salt thereof and (ii) a second anti-cancer agent, which is a type 1 MEK inhibitor or a pharmaceutically acceptable salt thereof, to treat or ameliorate the effects of the cancer.

Another embodiment of the present invention is a method of treating or ameliorating the effects of a cancer in a subject in need thereof. The method comprises administering to the subject an effective amount of (i) a first anti-cancer agent, which is BVD-523 or a pharmaceutically acceptable salt thereof and (ii) a second anti-cancer agent, which is RO092210 (Roche) or a pharmaceutically acceptable salt thereof, to treat or ameliorate the effects of the cancer.

A further embodiment of the present invention is a method of effecting cancer cell death. The method comprises contacting the cancer cell with an effective amount of (i) a first anti-cancer agent, which is BVD-523 or a pharmaceutically acceptable salt thereof and (ii) a second anti-cancer agent, which is a type 1 MEK inhibitor or a pharmaceutically acceptable salt thereof.

An additional embodiment of the present invention is a kit for treating or ameliorating the effects of a cancer in a subject in need thereof. The kit comprises an effective amount of (i) a first anti-cancer agent, which is BVD-523 or a pharmaceutically acceptable salt thereof and (ii) a second anti-cancer agent, which is a type 1 MEK inhibitor or a pharmaceutically acceptable salt thereof, packaged together with instructions for their use.

Another embodiment of the present invention is a pharmaceutical composition for treating or ameliorating the effects of a cancer in a subject in need thereof. The pharmaceutical composition comprises a pharmaceutically acceptable diluent or carrier and an effective amount of (i) a first anti-cancer agent, which is BVD-523 or a pharmaceutically acceptable salt thereof and (ii) a second anti-cancer agent, which is a type 1 MEK inhibitor or a pharmaceutically acceptable salt thereof, wherein administration of the first and second anti-cancer agents provides a synergistic effect compared to administration of either anti-cancer agent alone.

### BRIEF DESCRIPTION OF THE DRAWINGS

FIG. 1 shows that both direct ERK substrate phosphorylation and known effector pathways are modulated following acute and prolonged treatment with BVD-523 in vitro. Western blots were performed using a variety of antibodies to detect changes in whole-cell lysates of cancer lines exposed to BVD-523. In the A375 BRAF mutant cell line (a human melanoma cell line) and in the HCT116 KRAS mutant cell line (a human colorectal carcinoma cell line), phosphorylation of ERK-dependent residues (T359/S363) in RSK 1 and 2 proteins was reduced after 4 hours of treatment with BVD-523 at micromolar concentrations. Following 24 hours of treatment, direct substrate inhibition was maintained in BRAF mutant cell lines, and the MAPK feedback phosphatase DUSP6 was greatly reduced, suggesting durable and nearly complete MAPK pathway inhibition. Lastly, consistent with cytostatic effects of BVD-523 across multiple cell line backgrounds, the MAPK effector and G1/S-cell-cycle determinant gene cyclin-D1 was greatly reduced after 24 hours of treatment. In the A375 cell line, while the apoptosis effector and ERK substrate Bim-EL was increased following prolonged treatment, increased apoptosis was not observed, consistent with a lack of PARP cleavage, as well as other observations (not shown) that additional factors influence the capacity for BVD-523 to induce cell death.

FIG. 2 shows the results of single agent proliferation assays in parental A375 and A375 NRAS (Q61K/+) cells. Proliferation results are shown for treatment with BVD-523 (FIG. 2A), SCH772984 (FIG. 2B), Trametinib (FIG. 2C), MEK-162 (FIG. 2D), GDC-0623 (FIG. 2E), GDC-0973 (FIG. 2F), and Paclitaxel (FIG. 2G).

FIG. 3 shows the results of single agent proliferation assays in parental HCT116 and A375 KRAS KO (−/+) cells. Proliferation results are shown for treatment with BVD-523 (FIG. 3A), SCH772984 (FIG. 3B), Trametinib (FIG. 3C), MEK-162 (FIG. 3D), GDC-0623 (FIG. 3E), GDC-0973 (FIG. 3F), and Paclitaxel (FIG. 3G).

FIG. 4 shows the results of single agent proliferation assays in parental RKO and RKO BRAF V600E KO (+/−/−) cells. Proliferation results are shown for treatment with BVD-523 (FIG. 4A), SCH772984 (FIG. 4B), Trametinib (FIG. 4C), MEK-162 (FIG. 4D), GDC-0623 (FIG. 4E), GDC-0973 (FIG. 4F), and Paclitaxel (FIG. 4G).

FIG. 5 shows the results of the combination of BVD-523 and Trametinib in parental A375 and A375 NRAS (Q61K/+) cells. FIG. 5A shows a dose matrix showing inhibition (%) for the combination in parental A375 cells. FIG. 5B shows Loewe excess for the combination in 5A and FIG. 5C shows Bliss excess for the combination in 5A. FIG. 5D shows a dose matrix showing inhibition (%) for the combination in A375 NRAS (Q61K/+) cells. FIG. 5E shows Loewe excess for the combination in 5D and FIG. 5F shows Bliss excess for the combination in 5D. FIG. 5G-FIG. 5H show the results of single agent proliferation assays for the combination in 5A. FIG. 51-FIG. 5J show the results of single agent proliferation assays for the combination in 5D.

FIG. 6 shows the results of the combination of SCH772984 and Trametinib in parental A375 and A375 NRAS (Q61K/+) cells. FIG. 6A shows a dose matrix showing inhibition (%) for the combination in parental A375 cells. FIG. 6B shows Loewe excess for the combination in 6A and FIG. 6C shows Bliss excess for the combination in 6A. FIG. 6D shows a dose matrix showing inhibition (%) for the combination in A375 NRAS (Q61K/+) cells. FIG. 6E shows Loewe excess for the combination in 6D and FIG. 6F shows Bliss excess for the combination in 6D. FIG. 6G-FIG. 6H show the results of single agent proliferation assays for the combination in 6A. FIG. 6I-FIG. 6J show the results of single agent proliferation assays for the combination in 6D.

FIG. 7 shows the results of the combination of BVD-523 and MEK-162 in parental A375 and A375 NRAS (Q61K/+) cells. FIG. 7A shows a dose matrix showing inhibition (%) for the combination in parental A375 cells. FIG. 7B shows Loewe excess for the combination in 7A and FIG. 7C shows Bliss excess for the combination in 7A. FIG. 7D shows a dose matrix showing inhibition (%) for the combination in A375 NRAS (Q61K/+) cells. FIG. 7E shows Loewe excess for the combination in 7D and FIG. 7F shows Bliss excess for the combination in 7D. FIG. 7G-FIG. 7H show the results of single agent proliferation assays for the combination in 7A. FIG. 7I-FIG. 7J show the results of single agent proliferation assays for the combination in 7D.

FIG. 8 shows the results of the combination of SCH772984 and MEK-162 in parental A375 and A375 NRAS (Q61 K/+) cells. FIG. 8A shows a dose matrix showing inhibition (%) for the combination in parental A375 cells. FIG. 8B shows Loewe excess for the combination in 8A and FIG. 8C shows Bliss excess for the combination in 8A. FIG. 8D shows a dose matrix showing inhibition (%) for the combination in A375 NRAS (Q61 K/+) cells. FIG. 8E shows Loewe excess for the combination in 8D and FIG. 8F shows Bliss excess for the combination in 8D. FIG. 8G-FIG. 8H show the results of single agent proliferation assays for the combination in 8A. FIG. 8I-FIG. 8J show the results of single agent proliferation assays for the combination in 8D.

FIG. 9 shows the results of the combination of BVD-523 and GDC-0623 in parental A375 and A375 NRAS (Q61 K/+) cells. FIG. 9A shows a dose matrix showing inhibition (%) for the combination in parental A375 cells. FIG. 9B shows Loewe excess for the combination in 9A and FIG. 9C shows Bliss excess for the combination in 9A. FIG. 9D shows a dose matrix showing inhibition (%) for the combination in A375 NRAS (Q61K/+) cells. FIG. 9E shows Loewe excess for the combination in 9D and FIG. 9F shows Bliss excess for the combination in 9D. FIG. 9G-FIG. 9H show the results of single agent proliferation assays for the combination in 9A. FIG. 9I-FIG. 9J show the results of single agent proliferation assays for the combination in 9D.

FIG. 10 shows the results of the combination of SCH772984 and GDC-0623 in parental A375 and A375 NRAS (Q61K/+) cells. FIG. 10A shows a dose matrix showing inhibition (%) for the combination in parental A375 cells. FIG. 10B shows Loewe excess for the combination in 10A and FIG. 10C shows Bliss excess for the combination in 10A. FIG. 10D shows a dose matrix showing inhibition (%) for the combination in A375 NRAS (Q61K/+) cells. FIG. 10E shows Loewe excess for the combination in 10D and FIG. 10F shows Bliss excess for the combination in 10D. FIG. 10G-FIG. 10H show the results of single agent proliferation assays for the combination in 10A. FIG. 10I-FIG. 10J show the results of single agent proliferation assays for the combination in 10D.

FIG. 11 shows the results of the combination of BVD-523 and Trametinib in parental HCT116 and HCT116 KRAS KO (+/−) cells. FIG. 11A shows a dose matrix showing inhibition (%) for the combination in parental HCT116 cells. FIG. 11B shows Loewe excess for the combination in 11A and FIG. 11C shows Bliss excess for the combination in 11A. FIG. 11D shows a dose matrix showing inhibition (%) for the combination in HCT116 KRAS KO (+/−) cells. FIG. 11E shows Loewe excess for the combination in 11D and FIG. 11F shows Bliss excess for the combination in 11D. FIG. 11G-FIG. 11H show the results of single agent proliferation assays for the combination in 11A. FIG. 11I-FIG. 11J show the results of single agent proliferation assays for the combination in 11D.

FIG. 12 shows the results of the combination of SCH772984 and Trametinib in parental HCT116 and HCT116 KRAS KO (+/−) cells. FIG. 12A shows a dose matrix showing inhibition (%) for the combination in parental HCT116 cells. FIG. 12B shows Loewe excess for the combination in 12A and FIG. 12C shows Bliss excess for the combination in 12A. FIG. 12D shows a dose matrix showing inhibition (%) for the combination in HCT116 KRAS KO (+/−) cells. FIG. 12E shows Loewe excess for the combination in 12D and FIG. 12F shows Bliss excess for the combination in 12D. FIG. 12G-FIG. 12H show the results of single agent proliferation assays for the combination in 12A. FIG. 12I-FIG. 12J show the results of single agent proliferation assays for the combination in 12D.

FIG. 13 shows the results of the combination of BVD-523 and MEK-162 in parental HCT116 and HCT116 KRAS KO (+/−) cells. FIG. 13A shows a dose matrix showing inhibition (%) for the combination in parental HCT116 cells. FIG. 13B shows Loewe excess for the combination in 13A and FIG. 13C shows Bliss excess for the combination in 13A. FIG. 13D shows a dose matrix showing inhibition (%) for the combination in HCT116 KRAS KO (+/−) cells. FIG. 13E shows Loewe excess for the combination in 13D and FIG. 13F shows Bliss excess for the combination in 13D. FIG. 13G-FIG. 13H show the results of single agent proliferation assays for the combination in 13A. FIG. 13I-FIG. 13J show the results of single agent proliferation assays for the combination in 13D.

FIG. 14 shows the results of the combination of SCH772984 and MEK-162 in parental HCT116 and HCT116 KRAS KO (+/−) cells. FIG. 14A shows a dose matrix showing inhibition (%) for the combination in parental HCT116 cells. FIG. 14B shows Loewe excess for the combination in 14A and FIG. 14C shows Bliss excess for the combination in 14A. FIG. 14D shows a dose matrix showing inhibition (%) for the combination in HCT116 KRAS KO (+/−) cells. FIG. 14E shows Loewe excess for the combination in 14D and FIG. 14F shows Bliss excess for the combination in 14D. FIG. 14G-FIG. 14H show the results of single agent proliferation assays for the combination in 14A. FIG. 14I-FIG. 14J show the results of single agent proliferation assays for the combination in 14D.

FIG. 15 shows the results of the combination of BVD-523 and Trametinib in parental RKO and RKO BRAF V600E KO (+/−) cells. FIG. 15A shows a dose matrix showing inhibition (%) for the combination in parental RKO cells. FIG. 15B shows Loewe excess for the combination in 15A and FIG. 15C shows Bliss excess for the combination in 15A. FIG. 15D shows a dose matrix showing inhibition (%) for the combination in RKO BRAF V600E KO (+/−/−) cells. FIG. 15E shows Loewe excess for the combination in 15D and FIG. 15F shows Bliss excess for the combination in 15D. FIG. 15G-FIG. 15H show the results of single agent proliferation assays for the combination in 15A. FIG. 15I-FIG. 15J show the results of single agent proliferation assays for the combination in 15D.

FIG. 16 shows the results of the combination of SCH772984 and Trametinib in parental RKO and RKO BRAF V600E KO (+/−/−) cells. FIG. 16A shows a dose matrix showing inhibition (%) for the combination in parental RKO cells. FIG. 16B shows Loewe excess for the combination in 16A and FIG. 16C shows Bliss excess for the combination in 16A. FIG. 16D shows a dose matrix showing inhibition (%) for the combination in RKO BRAF V600E KO (+/−/−) cells. FIG. 16E shows Loewe excess for the combination in 16D and FIG. 16F shows Bliss excess for the combination in 16D. FIG. 16G-FIG. 16H show the results of single agent proliferation assays for the combination in 16A. FIG. 16I-FIG. 16J show the results of single agent proliferation assays for the combination in 16D.

FIG. 17 shows the results of the combination of BVD-523 and MEK-162 in parental RKO and RKO BRAF V600E KO (+/−/−) cells. FIG. 17A shows a dose matrix showing inhibition (%) for the combination in parental RKO cells. FIG. 17B shows Loewe excess for the combination in 17A and FIG. 17C shows Bliss excess for the combination in 17A. FIG. 17D shows a dose matrix showing inhibition (%) for the combination in RKO BRAF V600E KO (+/−/−) cells. FIG. 17E shows Loewe excess for the combination in 17D and FIG. 17F shows Bliss excess for the combination in 17D. FIG. 17G-FIG. 17H show the results of single agent proliferation assays for the combination in 17A. FIG. 17I-FIG. 17J show the results of single agent proliferation assays for the combination in 17D.

FIG. 18 shows the results of the combination of SCH772984 and MEK-162 in parental RKO and RKO BRAF V600E KO (+/−/−) cells. FIG. 18A shows a dose matrix showing inhibition (%) for the combination in parental RKO cells. FIG. 18B shows Loewe excess for the combination in 18A and FIG. 18C shows Bliss excess for the combination in 18A. FIG. 18D shows a dose matrix showing inhibition (%) for the combination in RKO BRAF V600E KO (+/−/−) cells. FIG. 18E shows Loewe excess for the combination in 18D and FIG. 18F shows Bliss excess for the combination in 18D. FIG. 18G-FIG. 18H show the results of single agent proliferation assays for the combination in 18A. FIG. 18I-FIG. 18J show the results of single agent proliferation assays for the combination in 18D.

FIG. 19 shows the results of the combination of BVD-523 and Trametinib in G-361 cells. FIG. 19A shows a dose matrix showing inhibition (%) for the combination. FIG. 19B shows Loewe excess for the combination in 19A and FIG. 19C shows Bliss excess for the combination in 19A. FIG. 19D-FIG. 19E show the results of single agent proliferation assays for the combination in 19A.

FIG. 20 shows the results of the combination of SCH772984 and Trametinib in G-361 cells. FIG. 20A shows a dose matrix showing inhibition (%) for the combination. FIG. 20B shows Loewe excess for the combination in 20A and FIG. 20C shows Bliss excess for the combination in 20A. FIG. 20D-FIG. 20E show the results of single agent proliferation assays for the combination in 20A.

FIG. 21 shows the results of the combination of BVD-523 and MEK-162 in G-361 cells. FIG. 21A shows a dose matrix showing inhibition (%) for the combination. FIG. 21B shows Loewe excess for the combination in 21A and FIG. 21C shows Bliss excess for the combination in 21A. FIG. 21D-FIG. 21E show the results of single agent proliferation assays for the combination in 21A.

FIG. 22 shows the results of the combination of SCH772984 and MEK-162 in G-361 cells. FIG. 22A shows a dose matrix showing inhibition (%) for the combination. FIG. 22B shows Loewe excess for the combination in 22A and FIG. 22C shows Bliss excess for the combination in 22A. FIG. 22D-FIG. 22E show the results of single agent proliferation assays for the combination in 22A.

FIG. 23 shows the results of the combination of BVD-523 and GDC-0623 in G-361 cells. FIG. 23A shows a dose matrix showing inhibition (%) for the combination. FIG. 23B shows Loewe excess for the combination in 23A and FIG. 23C shows Bliss excess for the combination in 23A. FIG. 23D-FIG. 23E show the results of single agent proliferation assays for the combination in 23A.

FIG. 24 shows the results of the combination of SCH772984 and GDC-0623 in G-361 cells. FIG. 24A shows a dose matrix showing inhibition (%) for the combination. FIG. 24B shows Loewe excess for the combination in 24A and FIG. 24C shows Bliss excess for the combination in 24A. FIG. 24D-FIG. 24E show the results of single agent proliferation assays for the combination in 24A.

FIG. 25 shows the results of the combination of BVD-523 and Trametinib in A549 cells. FIG. 25A shows a dose matrix showing inhibition (%) for the combination. FIG. 25B-FIG. 25C show the results of single agent proliferation assays for the combination in 25A. FIG. 25D shows Loewe excess for the combination in 25A and FIG. 25E shows Bliss excess for the combination in 25A.

FIG. 26 shows the results of the combination of BVD-523 and Trametinib in H2122 cells. FIG. 26A shows a dose matrix showing inhibition (%) for the combination. FIG. 26B-FIG. 26C show the results of single agent proliferation assays for the combination in 26A. FIG. 26D shows Loewe excess for the combination in 26A and FIG. 26E shows Bliss excess for the combination in 26A.

FIG. 27 shows the results of the combination of BVD-523 and Trametinib in H1437 cells. FIG. 27A shows a dose matrix showing inhibition (%) for the combination. FIG. 27B-FIG. 27C show the results of single agent proliferation assays for the combination in 27A. FIG. 27D shows Loewe excess for the combination in 27A and FIG. 27E shows Bliss excess for the combination in 27A.

FIG. 28 shows the results of the combination of BVD-523 and Trametinib in H226 cells. FIG. 28A shows a dose matrix showing inhibition (%) for the combination. FIG. 28B-FIG. 28C show the results of single agent proliferation assays for the combination in 28A. FIG. 28D shows Loewe excess for the combination in 28A and FIG. 28E shows Bliss excess for the combination in 28A.

FIG. 29 shows the results of the combination of SCH772984 and Trametinib in A549 cells. FIG. 29A shows a dose matrix showing inhibition (%) for the combination. FIG. 29B-FIG. 29C show the results of single agent proliferation assays for the combination in 29A. FIG. 29D shows Loewe excess for the combination in 29A and FIG. 29E shows Bliss excess for the combination in 29A.

FIG. 30 shows the results of the combination of SCH772984 and Trametinib in H2122 cells. FIG. 30A shows a dose matrix showing inhibition (%) for the combination. FIG. 30B-FIG. 30C show the results of single agent proliferation assays for the combination in 30A. FIG. 30D shows Loewe excess for the combination in 30A and FIG. 30E shows Bliss excess for the combination in 30A.

FIG. 31 shows the results of the combination of SCH772984 and Trametinib in H1437 cells. FIG. 31A shows a dose matrix showing inhibition (%) for the combination. FIG. 31B-FIG. 31C show the results of single agent proliferation assays for the combination in 31A. FIG. 31D shows Loewe excess for the combination in 31A and FIG. 31E shows Bliss excess for the combination in 31A.

FIG. 32 shows the results of the combination of SCH772984 and Trametinib in H226 cells. FIG. 32A shows a dose matrix showing inhibition (%) for the combination. FIG. 32B-FIG. 32C show the results of single agent proliferation assays for the combination in 32A. FIG. 32D shows Loewe excess for the combination in 32A and FIG. 32E shows Bliss excess for the combination in 32A.

FIG. 33 shows the results of the combination of BVD-523 and GDC-0623 in H2122 cells. FIG. 33A shows a dose matrix showing inhibition (%) for the combination. FIG. 33B-FIG. 33C show the results of single agent proliferation assays for the combination in 33A. FIG. 33D shows Loewe excess for the combination in 33A and FIG. 33E shows Bliss excess for the combination in 33A.

FIG. 34 shows the results of the combination of BVD-523 and GDC-0623 in H1437 cells. FIG. 34A shows a dose matrix showing inhibition (%) for the combination. FIG. 34B-FIG. 34C show the results of single agent proliferation assays for the combination in 34A. FIG. 34D shows Loewe excess for the combination in 34A and FIG. 34E shows Bliss excess for the combination in 34A.

FIG. 35 shows the results of the combination of BVD-523 and GDC-0623 in H226 cells. FIG. 35A shows a dose matrix showing inhibition (%) for the combination. FIG. 35B-FIG. 35C show the results of single agent proliferation assays for the combination in 35A. FIG. 35D shows Loewe excess for the combination in 35A and FIG. 35E shows Bliss excess for the combination in 35A.

FIG. 36 shows the results of the combination of SCH772984 and GDC-0623 in A549 cells. FIG. 36A shows a dose matrix showing inhibition (%) for the combination. FIG. 36B-FIG. 36C show the results of single agent proliferation assays for the combination in 36A. FIG. 36D shows Loewe excess for the combination in 36A and FIG. 36E shows Bliss excess for the combination in 36A.

FIG. 37 shows the results of the combination of SCH772984 and GDC-0623 in H2122 cells. FIG. 37A shows a dose matrix showing inhibition (%) for the combination. FIG. 37B-FIG. 37C show the results of single agent proliferation assays for the combination in 37A. FIG. 37D shows Loewe excess for the combination in 37A and FIG. 37E shows Bliss excess for the combination in 37A.

FIG. 38 shows the results of the combination of SCH772984 and GDC-0623 in H1437 cells. FIG. 38A shows a dose matrix showing inhibition (%) for the combination. FIG. 38B-FIG. 38C show the results of single agent proliferation assays for the combination in 38A. FIG. 38D shows Loewe excess for the combination in 38A and FIG. 38E shows Bliss excess for the combination in 38A.

FIG. 39 shows the results of the combination of SCH772984 and GDC-0623 in H226 cells. FIG. 39A shows a dose matrix showing inhibition (%) for the combination. FIG. 39B-FIG. 39C show the results of single agent proliferation assays for the combination in 39A. FIG. 39D shows Loewe excess for the combination in 39A and FIG. 39E shows Bliss excess for the combination in 39A.

FIG. 40 shows the results of the combination of BVD-523 and SCH772984. FIG. 40A shows a dose matrix showing inhibition (%) for the combination in A375 cells. FIG. 40B-FIG. 40C show the results of single agent proliferation assays for the combination in 40A. FIG. 40D shows Loewe excess for the combination in 40A and FIG. 40E shows Bliss excess for the combination in 40A.

### DETAILED DESCRIPTION OF THE INVENTION

One embodiment of the present invention is a method of treating or ameliorating the effects of a cancer in a subject in need thereof. The method comprises administering to the subject an effective amount of (i) a first anti-cancer agent, which is BVD-523 or a pharmaceutically acceptable salt thereof and (ii) a second anti-cancer agent, which is a type 1 MEK inhibitor or a pharmaceutically acceptable salt thereof, to treat or ameliorate the effects of the cancer.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present invention may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population may fail to respond or respond inadequately to treatment.

As used herein, the terms “ameliorate”, “ameliorating” and grammatical variations thereof mean to decrease the severity of the symptoms of a disease in a subject.

As used herein, a “subject” is a mammal, preferably, a human. In addition to humans, categories of mammals within the scope of the present invention include, for example, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc.

In the present invention, cancers include both solid and hemotologic cancers. Non-limiting examples of solid cancers include adrenocortical carcinoma, anal cancer, bladder cancer, bone cancer (such as osteosarcoma), brain cancer, breast cancer, carcinoid cancer, carcinoma, cervical cancer, colon cancer, endometrial cancer, esophageal cancer, extrahepatic bile duct cancer, Ewing family of cancers, extracranial germ cell cancer, eye cancer, gallbladder cancer, gastric cancer, germ cell tumor, gestational trophoblastic tumor, head and neck cancer, hypopharyngeal cancer, islet cell carcinoma, kidney cancer, large intestine cancer, laryngeal cancer, leukemia, lip and oral cavity cancer, liver cancer, lung cancer, lymphoma, malignant mesothelioma, Merkel cell carcinoma, mycosis fungoides, myelodysplastic syndrome, myeloproliferative disorders, nasopharyngeal cancer, neuroblastoma, oral cancer, oropharyngeal cancer, osteosarcoma, ovarian epithelial cancer, ovarian germ cell cancer, pancreatic cancer, paranasal sinus and nasal cavity cancer, parathyroid cancer, penile cancer, pituitary cancer, plasma cell neoplasm, prostate cancer, rhabdomyosarcoma, rectal cancer, renal cell cancer, transitional cell cancer of the renal pelvis and ureter, salivary gland cancer, Sezary syndrome, skin cancers (such as cutaneous t-cell lymphoma, Kaposi's sarcoma, mast cell tumor,and melanoma), small intestine cancer, soft tissue sarcoma, stomach cancer, testicular cancer, thymoma, thyroid cancer, urethral cancer, uterine cancer, vaginal cancer, vulvar cancer, and Wilms' tumor.

Examples of hematologic cancers include, but are not limited to, leukemias, such as adult/childhood acute lymphoblastic leukemia, adult/childhood acute myeloid leukemia, chronic lymphocytic leukemia, chronic myelogenous leukemia, and hairy cell leukemia, lymphomas, such as AIDS-related lymphoma, cutaneous T-cell lymphoma, adult/childhood Hodgkin lymphoma, mycosis fungoides, adult/childhood non-Hodgkin lymphoma, primary central nervous system lymphoma, Sézary syndrome, cutaneous T-cell lymphoma, and Waldenstrom macroglobulinemia, as well as other proliferative disorders such as chronic myeloproliferative disorders, Langerhans cell histiocytosis, multiple myeloma/plasma cell neoplasm, myelodysplastic syndromes, and myelodysplastic/myeloproliferative neoplasms.

A preferred set of cancers include a cancer of the large intestine, breast cancer, pancreatic cancer, skin cancer, endometrial cancer, neuroblastoma, leukemia, lymphoma, liver cancer, lung cancer, testicular cancer, and thyroid cancer. More preferably, the cancer is melanoma.

In the present invention, BVD-523 corresponds to a compound according to formula (I):

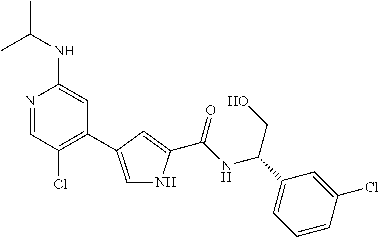 [SMILES: CC(C)Nc1cc(-c2c[nH]c(C(=O)N[C@H](CO)c3cccc(Cl)c3)c2)c(Cl)cn1]

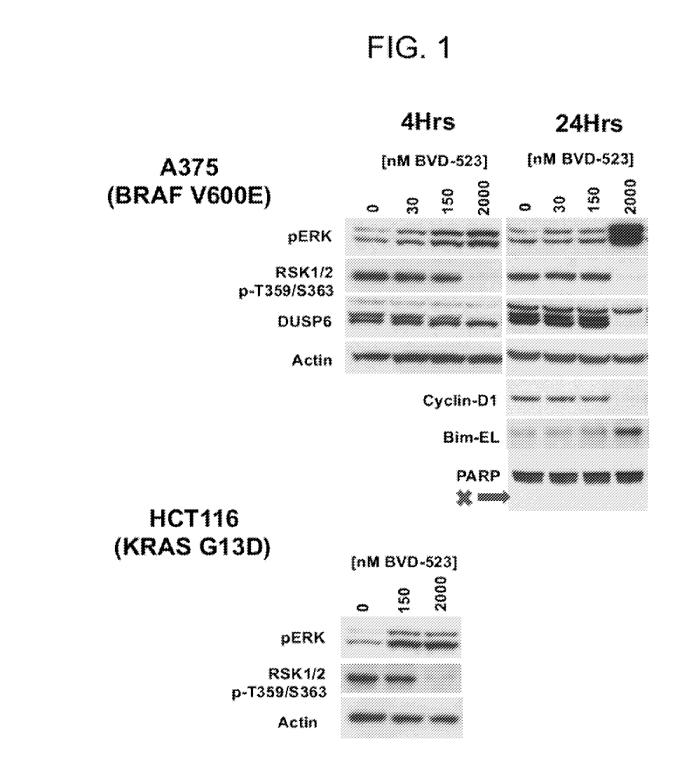

and pharmaceutically acceptable salts thereof. BVD-523 may be synthesized according to the methods disclosed, e.g., in U.S. Pat. No. 7,354,939. Enantiomers and racemic mixtures of both enantiomers of BVD-523 are also contemplated within the scope of the present invention. BVD-523's mechanism of action is believed to be, inter alia, unique and distinct from certain other ERK1/2 inhibitors, such as SCH772984 and the pyrimidinal structure used by Hatzivassiliou et al. (2012). For example, SCH772984 inhibits autophosphorylation of ERK (Morris et al., 2013), but BVD-523 allows for the autophosphorylation of ERK while still inhibiting ERK. (See, e.g., FIG. 1).

As used herein, a “MEK inhibitor” means those substances that (i) directly interact with MEK, e.g., by binding to MEK and (ii) decrease the expression or the activity of MEK. Therefore, inhibitors that act upstream of MEK, such as RAS inhibitors and RAF inhibitors, are not MEK inhibitors according to the present invention. MEK inhibitors may be classified into two types depending on whether the inhibitor competes with ATP. As used herein, “Type 1” MEK inhibitors mean those inhibitors that compete with ATP for binding to MEK. “Type 2” MEK inhibitors means those that do not compete with ATP for binding to MEK. Non-limiting examples of type 1 MEK inhibitors according to the present invention include bentamapimod (Merck KGaA), L783277 (Merck), RO092210 (Roche), pharmaceutically acceptable salts thereof, and combinations thereof. Preferably, the type 1 MEK inhibitor is RO092210 (Roche) or a pharmaceutically acceptable salt thereof.

In one aspect of this embodiment, the subject with cancer has a somatic RAS or BRAF mutation, preferably a K-RAS mutation.

As used herein, “somatic mutation” means a change occurring in any cell that is not destined to become a germ cell. The mutation may be a substitution, deletion, insertion, or a fusion. Preferably, the RAS mutation is a mutation in H-RAS, N-RAS, or K-RAS. The following Tables 1, 2 and 3 show the SEQ ID Nos. of representative nucleic acid and amino acid sequences of wild type H-RAS, K-RAS, and N-RAS from various animals, respectively. These sequences may be used in methods for identifying subjects with a mutant RAS genotype.

TABLE 1
H-RAS sequences

| SEQ ID | polypeptide or nucleic | | Other |
|---|---|---|---|
| No. | acid sequence | Organism | Information |
| 1 | nucleic acid | human | isoform 1 |
| 2 | polypeptide | human | isoform 1 |
| 3 | nucleic acid | human | isoform 2 |
| 4 | polypeptide | human | isoform 2 |
| 5 | nucleic acid | human | isoform 3 |
| 6 | polypeptide | human | isoform 3 |
| 7 | nucleic acid | rat (Rattus | variant 1 |
| | | norvegicus) | |
| 8 | polypeptide | rat (Rattus | variant 1 |
| | | norvegicus) | |
| 9 | nucleic acid | rat (Rattus | variant 2 |
| | | norvegicus) | |
| 10 | polypeptide | rat (Rattus | variant 2 |
| | | norvegicus) | |
| 11 | nucleic acid | mouse, Mus | |
| | | musculus | |
| 12 | polypeptide | mouse, Mus | |
| | | musculus | |
| 13 | nucleic acid | guinea pig, Cavia | variant 1 |
| | | porcellus | |
| 14 | polypeptide | guinea pig, Cavia | variant 1 |
| | | porcellus | |
| 15 | nucleic acid | guinea pig, Cavia | variant 2 |
| | | porcellus | |
| 16 | polypeptide | guinea pig, Cavia | variant 2 |
| | | porcellus | |
| 17 | nucleic acid | guinea pig, Cavia | variant 3 |
| | | porcellus | |
| 18 | polypeptide | guinea pig, Cavia | variant 3 |
| | | porcellus | |
| 19 | nucleic acid | guinea pig, Cavia | variant 4 |
| | | porcellus | |
| 20 | polypeptide | guinea pig, Cavia | variant 4 |
| | | porcellus | |
| 21 | nucleic acid | dog, Canis lupus | variant 1 |
| | | familiaris | |
| 22 | polypeptide | dog, Canis lupus | variant 1 |
| | | familiaris | |
| 23 | nucleic acid | dog, Canis lupus | variant 2 |
| | | familiaris | |
| 24 | polypeptide | dog, Canis lupus | variant 2 |
| | | familiaris | |
| 25 | nucleic acid | cat, Felis catus | variant 1 |
| 26 | polypeptide | cat, Felis catus | variant 1 |
| 27 | nucleic acid | cat, Felis catus | variant 2 |
| 28 | polypeptide | cat, Felis catus | variant 2 |
| 29 | nucleic acid | cow, Bos taurus | variant 1 |
| 30 | polypeptide | cow, Bos taurus | variant 1 |
| 31 | nucleic acid | cow, Bos taurus | variant 2 |
| 32 | polypeptide | cow, Bos taurus | variant 2 |
| 33 | nucleic acid | cow, Bos taurus | variant X1 |
| 34 | polypeptide | cow, Bos taurus | variant X1 |
| 35 | nucleic acid | chicken, Gallus | |
| | | gallus | |
| 36 | polypeptide | chicken, Gallus | |
| | | gallus | |

TABLE 2
K-RAS sequences

| SEQ ID | polypeptide or nucleic | | Other |
|---|---|---|---|
| No. | acid sequence | Organism | Information |
| 37 | nucleic acid | human | isoform a |
| 38 | polypeptide | human | isoform a |
| 39 | nucleic acid | human | isoform b |
| 40 | polypeptide | human | isoform b |
| 41 | nucleic acid | rat (Rattus | |
| | | norvegicus) | |
| 42 | polypeptide | rat (Rattus | |
| | | norvegicus) | |
| 43 | nucleic acid | mouse, Mus | |
| | | musculus | |
| 44 | polypeptide | mouse, Mus | |
| | | musculus | |
| 45 | nucleic acid | rabbit, Oryctolagus | |
| | | cuniculus | |
| 46 | polypeptide | rabbit, Oryctolagus | |
| | | cuniculus | |
| 47 | nucleic acid | guinea pig, Cavia | variant 1 |
| | | porcellus | |
| 48 | polypeptide | guinea pig, Cavia | variant 1 |
| | | porcellus | |
| 49 | nucleic acid | guinea pig, Cavia | variant 2 |
| | | porcellus | |
| 50 | polypeptide | guinea pig, Cavia | variant 2 |
| | | porcellus | |
| 51 | nucleic acid | dog, Canis lupus | variant 1 |
| | | familiaris | |
| 52 | polypeptide | dog, Canis lupus | variant 1 |
| | | familiaris | |
| 53 | nucleic acid | dog, Canis lupus | variant 2 |
| | | familiaris | |
| 54 | polypeptide | dog, Canis lupus | variant 2 |
| | | familiaris | |
| 55 | nucleic acid | cat, Felis catus | variant 1 |
| 56 | polypeptide | cat, Felis catus | variant 1 |
| 57 | nucleic acid | cat, Felis catus | variant 2 |
| 58 | polypeptide | cat, Felis catus | variant 2 |
| 59 | nucleic acid | cow, Bos taurus | |
| 60 | polypeptide | cow, Bos taurus | |
| 61 | nucleic acid | cow, Bos taurus | variant X2 |
| 62 | polypeptide | cow, Bos taurus | variant X2 |
| 63 | nucleic acid | cow, Bos taurus | variant X3 |
| 64 | polypeptide | cow, Bos taurus | variant X3 |
| 65 | nucleic acid | chicken, Gallus | |
| | | gallus | |
| 66 | polypeptide | chicken, Gallus | |
| | | gallus | |

TABLE 3
N-RAS sequences

| SEQ ID | polypeptide or nucleic | | Other |
|---|---|---|---|
| No. | acid sequence | Organism | Information |
| 67 | nucleic acid | human | |
| 68 | polypeptide | human | |
| 69 | nucleic acid | rat (Rattus | |
| | | norvegicus) | |
| 70 | polypeptide | rat (Rattus | |
| | | norvegicus) | |
| 71 | nucleic acid | mouse, Mus | |
| | | musculus | |
| 72 | polypeptide | mouse, Mus | |
| | | musculus | |
| 73 | nucleic acid | guinea pig, Cavia | |
| | | porcellus | |
| 74 | polypeptide | guinea pig, Cavia | |
| | | porcellus | |
| 75 | nucleic acid | guinea pig, Cavia | variant X1 |
| | | porcellus | |
| 76 | polypeptide | guinea pig, Cavia | variant X1 |
| | | porcellus | |
| 77 | nucleic acid | dog, Canis lupus | |
| | | familiaris | |
| 78 | polypeptide | dog, Canis lupus | |
| | | familiaris | |
| 79 | nucleic acid | cat, Felis catus | |
| 80 | polypeptide | cat, Felis catus | |
| 81 | nucleic acid | cow, Bos taurus | |
| 82 | polypeptide | cow, Bos taurus | |
| 83 | nucleic acid | chicken, Gallus | |
| | | gallus | |
| 84 | polypeptide | chicken, Gallus | |
| | | gallus | |

The following Table 4 shows the SEQ ID Nos. of representative nucleic acid and amino acid sequences of wild type BRAF from various animals. These sequences may be used in methods for identifying subjects with a mutant BRAF genotype.

TABLE 4
BRAF sequences

| SEQ ID | Nucleic acid or | | Other |
|---|---|---|---|
| NO | polypeptide | Organism | information |
| 85 | nucleic acid | human | |
| 86 | polypeptide | human | |
| 87 | nucleic acid | rat (Rattus norvegicus) | |
| 88 | polypeptide | rat (Rattus norvegicus) | |
| 89 | nucleic acid | mouse, Mus musculus | |
| 90 | polypeptide | mouse, Mus musculus | |
| 91 | nucleic acid | rabbit, Oryctolagus | |
| | | cuniculus | |
| 92 | polypeptide | rabbit, Oryctolagus | |
| | | cuniculus | |
| 93 | nucleic acid | guinea pig, Cavia | |
| | | porcellus | |
| 94 | polypeptide | guinea pig, Cavia | |
| | | porcellus | |
| 95 | nucleic acid | dog, Canis lupus | variant x1 |
| | | familiaris | |
| 96 | polypeptide | dog, Canis lupus | variant x1 |
| | | familiaris | |
| 97 | nucleic acid | dog, Canis lupus | variant x2 |
| | | familiaris | |
| 98 | polypeptide | dog, Canis lupus | variant x2 |
| | | familiaris | |
| 99 | nucleic acid | cat, Felis catus | |
| 100 | polypeptide | cat, Felis catus | |
| 101 | nucleic acid | cow, Bos taurus | variant X1 |
| 102 | polypeptide | cow, Bos taurus | variant X1 |
| 103 | nucleic acid | cow, Bos taurus | variant X2 |
| 104 | polypeptide | cow, Bos taurus | variant X2 |
| 105 | nucleic acid | cow, Bos taurus | variant X3 |
| 106 | polypeptide | cow, Bos taurus | variant X3 |
| 107 | nucleic acid | cow, Bos taurus | variant X4 |
| 108 | polypeptide | cow, Bos taurus | variant X4 |
| 109 | nucleic acid | cow, Bos taurus | variant X5 |
| 110 | polypeptide | cow, Bos taurus | variant X5 |
| 111 | nucleic acid | cow, Bos taurus | variant X6 |
| 112 | polypeptide | cow, Bos taurus | variant X6 |
| 113 | nucleic acid | cow, Bos taurus | variant X7 |
| 114 | polypeptide | cow, Bos taurus | variant X7 |
| 115 | nucleic acid | cow, Bos taurus | variant X8 |
| 116 | polypeptide | cow, Bos taurus | variant X8 |
| 117 | nucleic acid | cow, Bos taurus | variant X9 |
| 118 | polypeptide | cow, Bos taurus | variant X9 |
| 119 | nucleic acid | cow, Bos taurus | variant X10 |
| 120 | polypeptide | cow, Bos taurus | variant X10 |
| 121 | nucleic acid | cow, Bos taurus | variant X11 |
| 122 | polypeptide | cow, Bos taurus | variant X11 |
| 123 | nucleic acid | cow, Bos taurus | variant 2 |
| 124 | polypeptide | cow, Bos taurus | variant 2 |
| 125 | nucleic acid | horse, Equus caballus | |
| 126 | polypeptide | horse, Equus caballus | |
| 127 | nucleic acid | chicken, Gallus gallus | |
| 128 | polypeptide | chicken, Gallus gallus | |

Methods for identifying mutations in nucleic acids, such as the above identified RAS and BRAF genes, are known in the art. Nucleic acids may be obtained from biological samples. In the present invention, biological samples include, but are not limited to, blood, plasma, urine, skin, saliva, and biopsies. Biological samples are obtained from a subject by routine procedures and methods which are known in the art.

Non-limiting examples of methods for identifying mutations include PCR, sequencing, hybrid capture, in-solution capture, molecular inversion probes, fluorescent in situ hybridization (FISH) assay, and combinations thereof.

Various sequencing methods are known in the art. These include, but are not limited to, Sanger sequencing (also referred to as dideoxy sequencing) and various sequencing-by-synthesis (SBS) methods as disclosed in, e.g., Metzker 2005, sequencing by hybridization, by ligation (for example, WO 2005021786), by degradation (for example, U.S. Pat. Nos. 5,622,824 and 6,140,053) and nanopore sequencing (which is commercially available from Oxford Nanopore Technologies, UK). In deep sequencing techniques, a given nucleotide in the sequence is read more than once during the sequencing process. Deep sequencing techniques are disclosed in e.g., U.S. Patent Publication No. 20120264632 and International Patent Publication No. WO 2012125848.

PCR-based methods for detecting mutations are known in the art and employ PCR amplification, where each target sequence in the sample has a corresponding pair of unique, sequence-specific primers. For example, the polymerase chain reaction-restriction fragment length polymorphism (PCR-RFLP) method allows for rapid detection of mutations after the genomic sequences are amplified by PCR. The mutation is discriminated by digestion with specific restriction endonucleases and is identified by electrophoresis. See, e.g., Ota et al., 2007. Mutations may also be detected using real time PCR. See, e.g., International Application publication No. WO 2012046981.

Hybrid capture methods are known in the art and are disclosed in e.g., U.S. Patent Publication No. 20130203632 and U.S. Pat. Nos. 8,389,219 and 8,288,520. These methods are based on the selective hybridization of the target genomic regions to user-designed oligonucleotides. The hybridization can be to oligonucleotides immobilized on high or low density microarrays (on-array capture), or solution-phase hybridization to oligonucleotides modified with a ligand (e.g. biotin) which can subsequently be immobilized to a solid surface, such as a bead (in-solution capture).

Molecular Inversion Probe (MIP) techniques are known in the art and are disclosed in e.g., Absalan et al., 2008. This method uses MIP molecules, which are special “padlock” probes (Nilsson et al., 1994) for genotyping. A MIP molecule is a linear oligonucleotide that contains specific regions, universal sequences, restriction sites and a Tag (index) sequence (16-22 bp). A MIP hybridizes directly around the genetic marker/SNP of interest. The MIP method may also use a number of “padlock” probe sets that hybridize to genomic DNA in parallel (Hardenbol et al., 2003). In case of a perfect match, genomic homology regions are ligated by undergoing an inversion in configuration (as suggested by the name of the technique) and creating a circular molecule. After the first restriction, all molecules are amplified with universal primers. Amplicons are restricted again to ensure short fragments for hybridization on a microarray. Generated short fragments are labeled and, through a Tag sequence, hybridized to a cTag (complementary strand for index) on an array. After the formation of Tag-cTag duplex, a signal is detected.

In an additional aspect of this embodiment, the method further comprises administering to the subject at least one additional therapeutic agent selected from the group consisting of an antibody or fragment thereof, a cytotoxic agent, a drug, a toxin, a radionuclide, an immunomodulator, a photoactive therapeutic agent, a radiosensitizing agent, a hormone, an anti-angiogenesis agent, and combinations thereof.

As used herein, an “antibody” encompasses naturally occurring immunoglobulins as well as non-naturally occurring immunoglobulins, including, for example, single chain antibodies, chimeric antibodies (e.g., humanized murine antibodies), and heteroconjugate antibodies (e.g., bispecific antibodies). Fragments of antibodies include those that bind antigen, (e.g., Fab′, F(ab′)₂, Fab, Fv, and rIgG). See also, e.g., Pierce Catalog and Handbook, 1994-1995 (Pierce Chemical Co., Rockford, Ill.); Kuby, J., Immunology, 3rd Ed., W.H. Freeman & Co., New York (1998). The term antibody also includes bivalent or bispecific molecules, diabodies, triabodies, and tetrabodies. The term “antibody” further includes both polyclonal and monoclonal antibodies.

Examples of therapeutic antibodies that may be used in the present invention include rituximab (Rituxan), Cetuximab (Erbitux), bevacizumab (Avastin), and Ibritumomab (Zevalin).

Cytotoxic agents according to the present invention include DNA damaging agents, antimetabolites, anti-microtubule agents, antibiotic agents, etc. DNA damaging agents include alkylating agents, platinum-based agents, intercalating agents, and inhibitors of DNA replication. Non-limiting examples of DNA alkylating agents include cyclophosphamide, mechlorethamine, uramustine, melphalan, chlorambucil, ifosfamide, carmustine, lomustine, streptozocin, busulfan, temozolomide, pharmaceutically acceptable salts thereof, prodrugs, and combinations thereof. Non-limiting examples of platinum-based agents include cisplatin, carboplatin, oxaliplatin, nedaplatin, satraplatin, triplatin tetranitrate, pharmaceutically acceptable salts thereof, prodrugs, and combinations thereof. Non-limiting examples of intercalating agents include doxorubicin, daunorubicin, idarubicin, mitoxantrone, pharmaceutically acceptable salts thereof, prodrugs, and combinations thereof. Non-limiting examples of inhibitors of DNA replication include irinotecan, topotecan, amsacrine, etoposide, etoposide phosphate, teniposide, pharmaceutically acceptable salts thereof, prodrugs, and combinations thereof. Antimetabolites include folate antagonists such as methotrexate and premetrexed, purine antagonists such as 6-mercaptopurine, dacarbazine, and fludarabine, and pyrimidine antagonists such as 5-fluorouracil, arabinosylcytosine, capecitabine, gemcitabine, decitabine, pharmaceutically acceptable salts thereof, prodrugs, and combinations thereof. Anti-microtubule agents include without limitation vinca alkaloids, paclitaxel (Taxol®), docetaxel (Taxotere®), and ixabepilone (Ixempra®). Antibiotic agents include without limitation actinomycin, anthracyclines, valrubicin, epirubicin, bleomycin, plicamycin, mitomycin, pharmaceutically acceptable salts thereof, prodrugs, and combinations thereof.

Cytotoxic agents according to the present invention also include an inhibitor of the PI3K/Akt pathway. Non-limiting examples of an inhibitor of the PI3K/Akt pathway include A-674563 (CAS #552325-73-2), AGL 2263, AMG-319 (Amgen, Thousand Oaks, Calif.), AS-041164 (5-benzo[1,3]dioxol-5-ylmethylene-thiazolidine-2,4-dione), AS-604850 (5-(2,2-Difluoro-benzo[1,3]dioxol-5-ylmethylene)-thiazolidine-2,4-dione), AS-605240 (5-quinoxilin-6-methylene-1,3-thiazolidine-2,4-dione), AT7867 (CAS #857531-00-1), benzimidazole series, Genentech (Roche Holdings Inc., South San Francisco, Calif.), BML-257 (CAS #32387-96-5), CAL-120 (Gilead Sciences, Foster City, Calif.), CAL-129 (Gilead Sciences), CAL-130 (Gilead Sciences), CAL-253 (Gilead Sciences), CAL-263 (Gilead Sciences), CAS #612847-09-3, CAS #681281-88-9, CAS #75747-14-7, CAS #925681-41-0, CAS #98510-80-6, CCT128930 (CAS #885499-61-6), CH5132799 (CAS #1007207-67-1), CHR-4432 (Chroma Therapeutics, Ltd., Abingdon, UK), FPA 124 (CAS #902779-59-3), GS-1101 (CAL-101) (Gilead Sciences), GSK 690693 (CAS #937174-76-0), H-89 (CAS #127243-85-0), Honokiol, IC87114 (Gilead Science), IPI-145 (Intellikine Inc.), KAR-4139 (Karus Therapeutics, Chilworth, UK), KAR-4141 (Karus Therapeutics), KIN-1 (Karus Therapeutics), KT 5720 (CAS #108068-98-0), Miltefosine, MK-2206 dihydrochloride (CAS #1032350-13-2), ML-9 (CAS #105637-50-1), Naltrindole Hydrochloride, OXY-111A (NormOxys Inc., Brighton, Mass.), perifosine, PHT-427 (CAS #1191951-57-1), PI3 kinase delta inhibitor, Merck KGaA (Merck & Co., Whitehouse Station, N.J.), PI3 kinase delta inhibitors, Genentech (Roche Holdings Inc.), PI3 kinase delta inhibitors, Incozen (Incozen Therapeutics, Pvt. Ltd., Hyderabad, India), PI3 kinase delta inhibitors-2, Incozen (Incozen Therapeutics), PI3 kinase inhibitor, Roche-4 (Roche Holdings Inc.), PI3 kinase inhibitors, Roche (Roche Holdings Inc.), PI3 kinase inhibitors, Roche-5 (Roche Holdings Inc.), PI3-alpha/delta inhibitors, Pathway Therapeutics (Pathway Therapeutics Ltd., South San Francisco, Calif.), PI3-delta inhibitors, Cellzome (Cellzome AG, Heidelberg, Germany), PI3-delta inhibitors, Intellikine (Intellikine Inc., La Jolla, Calif.), PI3-delta inhibitors, Pathway Therapeutics-1 (Pathway Therapeutics Ltd.), PI3-delta inhibitors, Pathway Therapeutics-2 (Pathway Therapeutics Ltd.), PI3-delta/gamma inhibitors, Cellzome (Cellzome AG), PI3-delta/gamma inhibitors, Cellzome (Cellzome AG), PI3-delta/gamma inhibitors, Intellikine (Intellikine Inc.), PI3-delta/gamma inhibitors, Intellikine (Intellikine Inc.), PI3-delta/gamma inhibitors, Pathway Therapeutics (Pathway Therapeutics Ltd.), PI3-delta/gamma inhibitors, Pathway Therapeutics (Pathway Therapeutics Ltd.), PI3-gamma inhibitor Evotec (Evotec), PI3-gamma inhibitor, Cellzome (Cellzome AG), PI3-gamma inhibitors, Pathway Therapeutics (Pathway Therapeutics Ltd.), PI3K delta/gamma inhibitors, Intellikine-1 (Intellikine Inc.), PI3K delta/gamma inhibitors, Intellikine-1 (Intellikine Inc.), pictilisib (Roche Holdings Inc.), PIK-90 (CAS #677338-12-4), SC-103980 (Pfizer, New York, N.Y.), SF-1126 (Semafore Pharmaceuticals, Indianapolis, Ind.), SH-5, SH-6, Tetrahydro Curcumin, TG100-115 (Targegen Inc., San Diego, Calif.), Triciribine, X-339 (Xcovery, West Palm Beach, Fla.), XL-499 (Evotech, Hamburg, Germany), pharmaceutically acceptable salts thereof, and combinations thereof.

In the present invention, the term “toxin” means an antigenic poison or venom of plant or animal origin. An example is diphtheria toxin or portions thereof.

In the present invention, the term “radionuclide” means a radioactive substance administered to the patient, e.g., intravenously or orally, after which it penetrates via the patient's normal metabolism into the target organ or tissue, where it delivers local radiation for a short time. Examples of radionuclides include, but are not limited to, I-125, At-211, Lu-177, Cu-67, I-131, Sm-153, Re-186, P-32, Re-188, In-114m, and Y-90.

In the present invention, the term “immunomodulator” means a substance that alters the immune response by augmenting or reducing the ability of the immune system to produce antibodies or sensitized cells that recognize and react with the antigen that initiated their production. Immunomodulators may be recombinant, synthetic, or natural preparations and include cytokines, corticosteroids, cytotoxic agents, thymosin, and immunoglobulins. Some immunomodulators are naturally present in the body, and certain of these are available in pharmacologic preparations. Examples of immunomodulators include, but are not limited to, granulocyte colony-stimulating factor (G-CSF), interferons, imiquimod and cellular membrane fractions from bacteria, IL-2, IL-7, IL-12, CCL3, CCL26, CXCL7, and synthetic cytosine phosphate-guanosine (CpG).

In the present invention, “photoactive therapeutic agent” means compounds and compositions that become active upon exposure to light. Certain examples of photoactive therapeutic agents are disclosed, e.g., in U.S. Patent Application Serial No. 2011/0152230 A1, “Photoactive Metal Nitrosyls For Blood Pressure Regulation And Cancer Therapy.”

In the present invention, “radiosensitizing agent” means a compound that makes tumor cells more sensitive to radiation therapy. Examples of radiosensitizing agents include misonidazole, metronidazole, tirapazamine, and trans sodium crocetinate.

In the present invention, the term “hormone” means a substance released by cells in one part of a body that affects cells in another part of the body. Examples of hormones include, but are not limited to, prostaglandins, leukotrienes, prostacyclin, thromboxane, amylin, antimullerian hormone, adiponectin, adrenocorticotropic hormone, angiotensinogen, angiotensin, vasopressin, atriopeptin, brain natriuretic peptide, calcitonin, cholecystokinin, corticotropin-releasing hormone, encephalin, endothelin, erythropoietin, follicle-stimulating hormone, galanin, gastrin, ghrelin, glucagon, gonadotropin-releasing hormone, growth hormone-releasing hormone, human chorionic gonadotropin, human placental lactogen, growth hormone, inhibin, insulin, somatomedin, leptin, liptropin, luteinizing hormone, melanocyte stimulating hormone, motilin, orexin, oxytocin, pancreatic polypeptide, parathyroid hormone, prolactin, prolactin releasing hormone, relaxin, renin, secretin, somatostain, thrombopoietin, thyroid-stimulating hormone, testosterone, dehydroepiandrosterone, androstenedione, dihydrotestosterone, aldosterone, estradiol, estrone, estriol, cortisol, progesterone, calcitriol, and calcidiol.

Some compounds interfere with the activity of certain hormones or stop the production of certain hormones. These hormone-interfering compounds include, but are not limited to, tamoxifen (Nolvadex®), anastrozole (Arimidex®), letrozole (Femara®), and fulvestrant (Faslodex®). Such compounds are also within the meaning of hormone in the present invention.

As used herein, an “anti-angiogenesis” agent means a substance that reduces or inhibits the growth of new blood vessels, such as, e.g., an inhibitor of vascular endothelial growth factor (VEGF) and an inhibitor of endothelial cell migration. Anti-angiogenesis agents include without limitation 2-methoxyestradiol, angiostatin, bevacizumab, cartilage-derived angiogenesis inhibitory factor, endostatin, IFN-α, IL-12, itraconazole, linomide, platelet factor-4, prolactin, SU5416, suramin, tasquinimod, tecogalan, tetrathiomolybdate, thalidomide, thrombospondin, thrombospondin, TNP-470, ziv-aflibercept, pharmaceutically acceptable salts thereof, prodrugs, and combinations thereof.

In an additional aspect of this embodiment, administration of the first and second anti-cancer agents provides a synergistic effect compared to administration of either anti-cancer agent alone. As used herein, “synergistic” means more than additive. Synergistic effects may be measured by various assays known in the art, including but not limited to those disclosed herein, such as the excess over bliss assay.

Another embodiment of the present invention is a method of treating or ameliorating the effects of a cancer in a subject in need thereof. The method comprises administering to the subject an effective amount of (i) a first anti-cancer agent, which is BVD-523 or a pharmaceutically acceptable salt thereof and (ii) a second anti-cancer agent, which is RO092210 (Roche) or a pharmaceutically acceptable salt thereof, to treat or ameliorate the effects of the cancer.

Suitable and preferred subjects are as disclosed herein. In this embodiment, the methods may be used to treat the cancers disclosed above, including those cancers with the mutational backgrounds identified above. Methods of identifying such mutations are also as set forth above.

In another aspect of this embodiment, the BVD-523 or a pharmaceutically acceptable salt thereof is administered in the form of a pharmaceutical composition further comprising a pharmaceutically acceptable carrier or diluent.

In an additional aspect of this embodiment, the RO092210 (Roche) or a pharmaceutically acceptable salt thereof is administered in the form of a pharmaceutical composition further comprising a pharmaceutically acceptable carrier or diluent.

In another aspect of this embodiment, the method further comprises administering to the subject at least one additional therapeutic agent, preferably an inhibitor of the PI3K/Akt pathway, as disclosed herein.

In another aspect of this embodiment, administration of the first and second anti-cancer agents provides a synergistic effect compared to administration of either anti-cancer agent alone.

An additional embodiment of the present invention is a method of effecting cancer cell death. The method comprises contacting the cancer cell with an effective amount of (i) a first anti-cancer agent, which is BVD-523 or a pharmaceutically acceptable salt thereof and (ii) a second anti-cancer agent, which is a type 1 MEK inhibitor or a pharmaceutically acceptable salt thereof.

Suitable and preferred type 1 MEK inhibitors are as disclosed herein. In this embodiment, effecting cancer cell death may be accomplished in cancer cells having various mutational backgrounds and/or that are characterized as disclosed above. Methods of identifying such mutations are also as set forth above.

The methods of this embodiment, which may be carried out in vitro or in vivo, may be used to effect cancer cell death, by e.g., killing cancer cells, in cells of the types of cancer disclosed herein.

In one aspect of this embodiment, the cancer cell is a mammalian cancer cell. Preferably, the mammalian cancer cell is obtained from a mammal selected from the group consisting of humans, primates, farm animals, and domestic animals. More preferably, the mammalian cancer cell is a human cancer cell.

In another aspect of this embodiment, the method further comprises administering at least one additional therapeutic agent, preferably an inhibitor of the PI3K/Akt pathway, as disclosed herein.

In a further aspect of this embodiment, contacting the cancer cell with the first and second anti-cancer agents provides a synergistic effect compared to contacting the cancer cell with either anti-cancer agent alone. In this embodiment, “contacting” means bringing BVD-523, the type 1 MEK inhibitors, and optionally one or more additional therapeutic agents into close proximity to the cancer cells. This may be accomplished using conventional techniques of drug delivery to mammals or in the in vitro situation by, e.g., providing BVD-523, the type 1 MEK inhibitors, and optionally other therapeutic agents to a culture media in which the cancer cells are located.

Another embodiment of the present invention is a kit for treating or ameliorating the effects of a cancer in a subject in need thereof. The kit comprises an effective amount of (i) a first anti-cancer agent, which is BVD-523 or a pharmaceutically acceptable salt thereof and (ii) a second anti-cancer agent, which is a type 1 MEK inhibitor or a pharmaceutically acceptable salt thereof, packaged together with instructions for their use.

The kits may also include suitable storage containers, e.g., ampules, vials, tubes, etc., for each anti-cancer agent of the present invention (which may e.g., may be in the form of pharmaceutical compositions) and other reagents, e.g., buffers, balanced salt solutions, etc., for use in administering the anti-cancer agents to subjects. The anti-cancer agents of the invention and other reagents may be present in the kits in any convenient form, such as, e.g., in a solution or in a powder form. The kits may further include a packaging container, optionally having one or more partitions for housing the pharmaceutical composition and other optional reagents.

For use in the kits of the invention, suitable and preferred type 1 MEK inhibitors and subjects are as set forth above. In this embodiment, the kit may be used to treat the cancers disclosed above, including those cancers with the mutational backgrounds identified herein. Methods of identifying such mutations are as set forth above.

In one aspect of this embodiment, the kit further comprises at least one additional therapeutic agent, preferably an inhibitor of the PI3K/Akt pathway, as disclosed herein.

In a further aspect of this embodiment, administration of the first and second anti-cancer agents provides a synergistic effect compared to administration of either anti-cancer agent alone.

An additional embodiment of the present is a pharmaceutical composition for treating or ameliorating the effects of a cancer in a subject in need thereof. The pharmaceutical composition comprises a pharmaceutically acceptable diluent or carrier and an effective amount of (i) a first anti-cancer agent, which is BVD-523 or a pharmaceutically acceptable salt thereof and (ii) a second anti-cancer agent, which is a type 1 MEK inhibitor or a pharmaceutically acceptable salt thereof, wherein administration of the first and second anti-cancer agents provides a synergistic effect compared to administration of either anti-cancer agent alone.

Suitable and preferred subjects and type 1 MEK inhibitors are as disclosed herein. The pharmaceutical compositions of the invention may be used to treat the cancers disclosed above, including those cancers with the mutational backgrounds identified herein. Methods of identifying such mutations are also as set forth above.

In another aspect of this embodiment, the pharmaceutical composition further comprises at least one additional therapeutic agent, preferably an inhibitor of the PI3K/Akt pathway, as disclosed herein.

The pharmaceutical compositions according to the present invention may be in a unit dosage form comprising both anti-cancer agents. In another aspect of this embodiment, the first anti-cancer agent is in a first unit dosage form and the second anti-cancer agent is in a second unit dosage form, separate from the first.

The first and second anti-cancer agents may be co-administered to the subject, either simultaneously or at different times, as deemed most appropriate by a physician. If the first and second anti-cancer agents are administered at different times, for example, by serial administration, the first anti-cancer agent may be administered to the subject before the second anti-cancer agent. Alternatively, the second anti-cancer agent may be administered to the subject before the first anti-cancer agent.

In the present invention, an “effective amount” or a “therapeutically effective amount” of an anti-cancer agent of the invention including pharmaceutical compositions containing same that are disclosed herein is an amount of such agent or composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of an agent or composition according to the invention will be that amount of the agent or composition, which is the lowest dose effective to produce the desired effect. The effective dose of an agent or composition of the present invention may be administered as two, three, four, five, six or more sub-doses, administered separately at appropriate intervals throughout the day.

A suitable, non-limiting example of a dosage of BVD-523, a type 1 MEK inhibitor, or another anti-cancer agent disclosed herein is from about 1 mg/kg to about 2400 mg/kg per day, such as from about 1 mg/kg to about 1200 mg/kg per day, 75 mg/kg per day to about 300 mg/kg per day, including from about 1 mg/kg to about 100 mg/kg per day. Other representative dosages of such agents include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, and 2300 mg/kg per day. The effective dose of BVD-523, a type 1 MEK inhibitor, or another anti-cancer agent may be administered as two, three, four, five, six or more sub-doses, administered separately at appropriate intervals throughout the day.

The BVD-523, type 1 MEK inhibitors, or other anti-cancer agents or pharmaceutical compositions containing same of the present invention may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, BVD-523, type 1 MEK inhibitors, or other anti-cancer agents or pharmaceutical compositions containing same of the present invention may be administered in conjunction with other treatments. The BVD-523, type 1 MEK inhibitors, or other anti-cancer agents or the pharmaceutical compositions containing same of the present invention may be encapsulated or otherwise protected against gastric or other secretions, if desired.

The pharmaceutical compositions of the invention comprise one or more active ingredients, e.g. anti-cancer agents, in admixture with one or more pharmaceutically-acceptable diluents or carriers and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.).

Pharmaceutically acceptable diluents or carriers are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable diluent or carrier used in a pharmaceutical composition of the invention must be “acceptable” in the sense of being compatible with the other ingredients of the formulation and not injurious to the subject. Diluents or carriers suitable for a selected dosage form and intended route of administration are well known in the art, and acceptable diluents or carriers for a chosen dosage form and method of administration can be determined using ordinary skill in the art.

The pharmaceutical compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

The pharmaceutical compositions of the present invention suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable diluents or carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

The pharmaceutical compositions of the present invention for rectal or vaginal administration may be presented as a suppository, which may be prepared by mixing one or more active ingredient(s) with one or more suitable nonirritating diluents or carriers which are solid at room temperature, but liquid at body temperature and, therefore, will melt in the rectum or vaginal cavity and release the active compound. The pharmaceutical compositions of the present invention which are suitable for vaginal administration also include pessaries, tampons, creams, gels, pastes, foams or spray formulations containing such pharmaceutically-acceptable diluents or carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable diluent or carrier. The ointments, pastes, creams and gels may contain excipients. Powders and sprays may contain excipients and propellants.

The pharmaceutical compositions of the present invention suitable for parenteral administrations may comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These pharmaceutical compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid diluent or carrier, for example water for injection, immediately prior to use. Extemporaneous injection solutions and suspensions may be prepared from sterile powders, granules and tablets of the type described above.

The present invention provides combinations shown to enhance the effects of ERK inhibitors. Herein, applicants have also shown that the combination of different ERK inhibitors is likewise synergistic. Therefore, it is contemplated that the effects of the combinations described herein can be further improved by the use of one or more additional ERK inhibitors. Accordingly, some embodiments of the present invention include one or more additional ERK inhibitors.

The following examples are provided to further illustrate the methods of the present invention. These examples are illustrative only and are not intended to limit the scope of the invention in any way.

### EXAMPLES

### Example 1

### BVD-523 Altered Markers of MAPK Kinase Activity and Effector Function

For Western blot studies, HCT116 cells (5×10⁶) were seeded into 10 cm dishes in McCoy's 5A plus 10% FBS. A375 cells (2.5×10⁶) were seeded into 10 cm dishes in DMEM plus 10% FBS. Cells were allowed to adhere overnight prior to addition of the indicated amount of test compound (BVD-523) or vehicle control. Cells were treated for either 4 or 24 hours before isolation of whole-cell protein lysates, as specified below. Cells were harvested by trypsinisation, pelleted and snap frozen. Lysates were prepared with RIPA (Radio-Immunoprecipitation Assay) buffer, clarified by centrifugation and quantitated by bicinchoninic acid assay (BCA) assay. 20-50 μg of protein was resolved by SDS-PAGE electrophoresis, blotted onto PVDF membrane and probed using the antibodies detailed in Table 5 (for the 4-hour treatment) and Table 6 (for the 24-hour treatment) below.

TABLE 5
Antibody Details

| | | | | | Incubation/ | |
|---|---|---|---|---|---|---|
| | Size | | | | Block | |
| Antigen | (kDa) | Supplier | Cat No | Dilution | Conditions | Secondary |
| pRSK1/2 | 90 | Cell | 9335 | 1:1000 | o/n 4° C. 5% | anti-rabbit |
| pS380 | | Signaling | | | BSA | |
| pRSK1/2 | 90 | Cell | 11989 | 1:2000 | o/n 4° C. 5% | anti-rabbit |
| pS380 | | Signaling | | | BSA | |
| pRSK- | 90 | Millipore | 04-419 | 1:40000 | o/n 4° C. 5% | anti-rabbit |
| T359/S363 | | | | | BSA | |
| Total RSK | 90 | Cell | 9333 | 1:1000 | o/n 4° C. 5% | anti-rabbit |
| | | Signaling | | | BSA | |
| pErk 1/2 | 42/44 | Cell | 9106S | 1:500 | o/n 4° C. 5% | anti-mouse |
| | | Signaling | | | milk | |
| Total ERK | 42/44 | Cell | 9102 | 1:2000 | o/n 4° C. 5% | anti-rabbit |
| | | Signaling | | | milk | |
| pMEK1/2 | 45 | Cell | 9154 | 1:1000 | o/n 4° C. 5% | anti-rabbit |
| | | Signaling | | | BSA | |
| Total MEK | 45 | Cell | 9126 | 1:1000 | o/n 4° C. 5% | anti-rabbit |
| | | Signaling | | | BSA | |
| pS6- | 32 | Cell | 2211S | 1:3000 | o/n 4° C. 5% | anti-rabbit |
| pS235 | | Signaling | | | milk | |
| Total S6 | 32 | Cell | 2217 | 1:2000 | o/n 4° C. 5% | anti-rabbit |
| | | Signaling | | | milk | |
| DUSP6 | 48 | Cell | 3058S | 1:1000 | o/n 4° C. 5% | anti-rabbit |
| | | Signaling | | | BSA | |
| Total | 73 | BD Bio- | 610152 | 1:2000 | o/n 4° C. 5% | anti-mouse |
| CRAF | | sciences | | | milk | |
| pCRAF- | 73 | Cell | 9427 | 1:1000 | o/n 4° C. 5% | anti-rabbit |
| Ser338 | | Signaling | | | BSA | |
| pRB | 105 | Cell | 9307 | 1:2000 | o/n 4° C. 5% | anti-rabbit |
| (Ser780) | | Signaling | | | BSA | |
| β-Actin | 42 | Sigma | A5441 | 1:500,000 | o/n 4° C. 5% | anti-mouse |
| | | | | | milk | |

TABLE 6
Antibody details

| | | | | | Incubation/ | |
|---|---|---|---|---|---|---|
| | Size | | | | Block | |
| Antigen | (kDa) | Supplier | Cat No | Dilution | Conditions | Secondary |
| pRB | 105 | Cell | 9307 | 1:2000 | o/n 4° C. 5% | anti-rabbit |
| (Ser780) | | Signaling | | | BSA | |
| CCND1 | 34 | Abcam | ab6152 | 1:500 | o/n 4° C. 5% | anti-mouse |
| | | | | | milk | |
| Bim-EL | 23 | Millipore | AB17003 | 1:1000 | o/n 4° C. 5% | anti-rabbit |
| | | | | | BSA | |
| Bim-EL | 23 | Cell | 2933 | 1:1000 | o/n 4° C. 5% | anti-rabbit |
| | | Signaling | | | BSA | |
| BCL-xL | 30 | Cell | 2762 | 1:2000 | o/n 4° C. 5% | anti-rabbit |
| | | Signaling | | | BSA | |
| PARP | 116/89 | Cell | 9542 | 1:1000 | o/n 4° C. 5% | anti-rabbit |
| | | Signaling | | | milk | |
| Cleaved | 17, 19 | Cell | 9664X | 1:1000 | o/n 4° C. 5% | anti-rabbit |
| Caspase 3 | | Signaling | | | milk | |
| DUSP6 | 48 | Cell | 3058S | 1:1000 | o/n 4° C. 5% | anti-rabbit |
| | | Signaling | | | BSA | |
| pRSK1/2 | 90 | Cell | 9335 | 1:1000 | o/n 4° C. 5% | anti-rabbit |
| pS380 | | Signaling | | | BSA | |
| pRSK1/2 | 90 | Cell | 11989 | 1:2000 | o/n 4° C. 5% | anti-rabbit |
| pS380 | | Signaling | | | BSA | |
| pRSK- | 90 | Millipore | 04-419 | 1:40000 | o/n 4° C. 5% | anti-rabbit |
| T359/S363 | | | | | BSA | |
| Total RSK | 90 | Cell | 9333 | 1:1000 | o/n 4° C. 5% | anti-rabbit |
| | | Signaling | | | BSA | |
| pErk 1/2 | 42/44 | Cell | 9106S | 1:500 | o/n 4° C. 5% | anti-mouse |
| | | Signaling | | | milk | |
| Total ERK | 42/44 | Cell | 9102 | 1:2000 | o/n 4° C. 5% | anti-rabbit |
| | | Signaling | | | milk | |
| B-Actin | 42 | Sigma | A5441 | 1:500,000 | o/n 4° C. 5% | anti-mouse |
| | | | | | milk | |

FIG. 1 shows Western blot analyses of cells treated with BVD-523 at various concentrations for the following: 1) MAPK signaling components in A375 cells after 4 hours; 2) cell cycle and apoptosis signaling in A375 24 hours treatment with various amounts of BVD-523; and 3) MAPK signaling in HCT-116 cells treated for 4 hours. The results show that acute and prolonged treatment with BVD-523 in RAF and RAS mutant cancer cells in-vitro affects both substrate phosphorylation and effector targets of ERK kinases. The concentrations of BVD-523 required to induce these changes is typically in the low micromolar range.

Changes in several specific activity markers are noteworthy. First, the abundance of slowly migrating isoforms of ERK kinase increase following BVD-523 treatment; modest changes can be observed acutely, and increase following prolonged treatment. While this could indicate an increase in enzymatically active, phosphorylated forms of ERK, it remains noteworthy that multiple proteins subject to both direct and indirect regulation by ERK remain “off” following BVD-523 treatment. First, RSK1/2 proteins exhibit reduced phosphorylation at residues that are strictly dependent on ERK for protein modification (T359/S363). Second, BVD-523 treatment induces complex changes in the MAPK feedback phosphatase, DUSP6: slowly migrating protein isoforms are reduced following acute treatment, while total protein levels are greatly reduced following prolonged BVD-523 treatment. Both of these findings are consistent with reduced activity of ERK kinases, which control DUSP6 function through both post-translational and transcriptional mechanisms. Overall, despite increases in cellular forms of ERK that are typically thought to be active, it appears likely that cellular ERK enzyme activity is fully inhibited following either acute or prolonged treatment with BVD-523.

Consistent with these observations, effector genes that require MAPK pathway signaling are altered following treatment with BVD-523. The G1/S cell-cycle apparatus is regulated at both post-translational and transcriptional levels by MAPK signaling, and cyclin-D1 protein levels are greatly reduced following prolonged BVD-523 treatment. Similarly, gene expression and protein abundance of apoptosis effectors often require intact MAPK signaling, and total levels of Bim-EL increase following prolonged BVD-523 treatment. As noted above, however, PARP protein cleavage and increased apoptosis were not noted in the A375 cell background; this suggests that additional factors may influence whether changes in BVD-523/ERK-dependent effector signaling are translated into definitive events such as cell death and cell cycle arrest.

Consistent with the cellular activity of BVD-523, marker analysis suggests that ERK inhibition alters a variety of molecular signaling events in cancer cells, making them susceptible to both decreased cell proliferation and survival.

In sum, FIG. 1 shows that BVD-523 inhibits the MAPK signaling pathway and may be more favorable compared to RAF or MEK inhibition in this setting.

Finally, properties of BVD-523 may make this a preferred agent for use as an ERK inhibitor, compared to other agents with a similar activity. It is known that kinase inhibitor drugs display unique and specific interactions with their enzyme targets, and that drug efficacy is strongly influenced by both the mode of direct inhibition, as well as susceptibility to adaptive changes that occur following treatment. For example, inhibitors of ABL, KIT, EGFR and ALK kinases are effective only when their cognate target is found in active or inactive configurations. Likewise, certain of these inhibitors are uniquely sensitive to either secondary genetic mutation, or post-translational adaptive changes, of the protein target. Finally, RAF inhibitors show differential potency to RAF kinases present in certain protein complexes and/or subcellular localizations. In summary, as ERK kinases are similarly known to exist in diverse, variable, and complex biochemical states, it appears likely that BVD-523 may interact with and inhibit these targets in a fashion that is distinct and highly preferable to other agents.

### Example 2

### BVD-523/MEK Inhibitor Combinations are Effective to Inhibit the Growth of Cancer Cell Lines In Vitro

Cancer cell lines are maintained in cell culture under standard media and serum conditions.

For all combination studies, HCT116 cells (KRas mutation human colorectal carcinoma cells) are seeded into triplicate 96-well plates at a cell density of 1500 cells/well in McCoy's 5A Medium plus 10% fetal bovine serum (FBS). A375 cells (BRAF V600E human malignant melanoma) are seeded at a density of 3000 cells/well in Dulbecco's Modified Eagle Medium (DMEM) plus 10% FBS. Cells are allowed to adhere overnight prior to addition of test compound or vehicle control.

For RO092210 studies, the following combinations are tested using a 10×8 dose matrix: RO092210 (ranging from 10-1000 nM) with BVD-0523 (ranging from 0 to 10 μM), RO092210 (ranging from 10-1000 nM) with dabrafenib (ranging from 0 to 1 μM), and RO092210 (ranging from 10-1000 nM) with trametinib (ranging from 0 to 0.010 μM). The final concentration of DMSO is 0.2%. The compounds are incubated with the cells for 96 hours.

For L783277 (another type 1 MEK inhibitor) studies, the following combinations are tested using a 10×8 dose matrix: L783277 (ranging from 0.5 nM-100 nM) with BVD-0523 (0 to 10 μM), L783277 (ranging from 0.5 nM-100 nM) with dabrafenib (ranging from 0 to 1 μM), and L783277 (ranging from 0.5 nM-100 nM) with trametinib (ranging from 0 to 0.1 μM). The final concentration of DMSO is 0.2%. The compounds are incubated with the cells for 96 hours.

For bentamapimod (another type 1 MEK inhibitor) studies, the following combinations are tested using a 10×8 dose matrix: bentamapimod (ranging from 10 nM-1000 nM) with BVD-0523 (0 to 10 μM), bentamapimod (ranging from 10 nM-1000 nM) with dabrafenib (ranging from 0 to 1 μM), and bentamapimod (ranging from 10 nM-1000 nM) with trametinib (ranging from 0 to 0.1 μM). The final concentration of DMSO is 0.2%. The compounds are incubated with the cells for 96 hours.

Next, Alamar Blue 10% (v/v) is added and incubated with the cells for 4 hours prior to reading on a fluorescent plate reader. After reading Alamar Blue, the medium/Alamar Blue mix is flicked off, 100 μl of CellTiter-Glo/PBS (1:1) is added, and the plates are processed as per the manufacturer's instructions (Promega, Madison, Wis.). Media only background values are subtracted before the data is analyzed.

### Caspase-Glo 3/7 Assays

In brief, HCT116 cells are seeded in triplicate in white 96-well plates at a cell density of 5000 cells/well in McCoy's 5A plus 10% FBS. A375 cells are seeded at a density of 5000 cells/well in DMEM plus 10% FBS. Cells are allowed to adhere overnight prior to addition of test compound or vehicle control. The final concentration of DMSO is 0.2%, and 800 nM staurosporine is included as a positive control. 24 and 48 hour assay incubation periods are used. Then, Caspase-Glo® 3/7 50% (v/v) is added, plates are mixed for 5 minutes on an orbital shaker and incubated for 1 hour at room temperature prior to reading on a luminescent plate reader. Media only background values are subtracted before the data is analysed.

### Data Analysis

The combination data may be presented as dose-response curves generated in GraphPad Prism (plotted using % viability relative to DMSO only treated controls).

Predicted fractional inhibition values for combined inhibition are calculated using the equation C_{bliss}=A+B−(A×B) where A and B are the fractional inhibitions obtained by drug A alone or drug B alone at specific concentrations. C_{bliss} is the fractional inhibition that would be expected if the combination of the two drugs is exactly additive. C_{bliss} values are subtracted from the experimentally observed fractional inhibition values to give an ‘excess over Bliss’ value. Excess over Bliss values greater than 0 indicate synergy, whereas values less than 0 indicate antagonism. Excess over Bliss values may be plotted as heat maps±SD.

It is expected that the combinations of RO092210, L783277, or bentamapimod with BVD-523 will be effective in inhibiting the growth of A375 and HCT116 cells. Dose response curves will be obtained. It is expected that the IC₅₀ of BVD-523 in these cell lines will be approximately 150 nM. It is also expected that the IC₅₀ of RO092210, L783277, and bentamapimod in these cell lines will be approximately 59 nM (Williams et al., 1998), 4 nM (Zhao et al., 1992), and 150 nM (Halazy et al., 2006) (Ferrandi et al., 2011) (Bhagwat et al., 2007), respectively.

### Example 3

### BVD-523/MEK Inhibitor Combinations are Effective to Inhibit the Growth of Cancer Cell Lines In Vivo

### Mice

Female athymic nude mice (Crl:NU(Ncr)-Foxn/^{nu}, Charles River) are nine weeks old with a body weight (BW) range of about 15 to about 30 grams on Day 1 of the study. The animals are fed ad libitum water (reverse osmosis, 1 ppm Cl), and NIH 31 Modified and Irradiated Lab Diet® consisting of 18.0% crude protein, 5.0% crude fat, and 5.0% crude fiber. The mice are housed on irradiated Enrich-o'cobs™ Laboratory Animal Bedding in static microisolators on a 12-hour light cycle at 20-22° C. (68-72° F.) and 40-60% humidity. The recommendations of the Guide for Care and Use of Laboratory Animals with respect to restraint, husbandry, surgical procedures, feed and fluid regulation, and veterinary care are complied with.

### In Vivo Implantation and Tumor Growth

HCT116 human colon carcinoma cells are cultured in RPMI-1640 medium supplemented with 10% fetal bovine serum, 2 mM glutamine, 100 units/mL penicillin G sodium, 100 μg/mL streptomycin sulfate, and 25 μg/mL gentamicin. The tumor cells are grown in tissue culture flasks in a humidified incubator at 37° C., in an atmosphere of 5% CO₂ and 95% air.

The HCT116 cells used for implantation are harvested during exponential growth and resuspended in 50% Matrigel (BD Biosciences): 50% phosphate buffered saline at a concentration of 2.5×10⁷ cells/mL. On the day of tumor implant, each test mouse is injected subcutaneously in the right flank with 5×10⁶ cells (0.2 mL cell suspension), and tumor growth is monitored as the average size approaches the target range of 100 to 150 mm³. Tumors are measured in two dimensions using calipers, and volume is calculated using the formula:

Tumor Volume (mm³)=(w²×l)/2

where w=width and l=length, in mm, of the tumor. Tumor weight may be estimated with the assumption that 1 mg is equivalent to 1 mm³ of tumor volume.

Ten days after tumor implantation, designated as Day 1 of the study, the animals are sorted into ten groups, each described below.

### Treatment

On Day 1 of the study, mice are sorted into groups each consisting of fifteen mice and one group consisting of ten mice, and dosing is initiated. All doses are given by oral gavage (p.o.) except paclitaxel, which is given intravenously (i.v.). For each agent, the dosing volume of 10 mL/kg (0.2 mL per 20 grams of BW) is scaled to the BW of the individual animal. The RO092210/L783277/bentamapimod doses are to be given once daily (qd) until study end (qd to end), whereas the vehicle and BVD-523 doses are to be given twice daily (bid) until study end (bid to end). For bid dosing, dosing is initiated in the afternoon of Day 1, so that one dose is given on the first day (“first day 1 dose”).

### Controls

One group receives 1% CMC vehicle p.o. bid to end, and serves as the control group for calculation of % TGD. Another group receives paclitaxel i.v. at 30 mg/kg once every other day (qod) for five doses (qod×5), and serves as the positive control for the model.

### Monotherapy Treatments

Two groups receive RO0092210 at 30 and 100 mg/kg. Two groups receive 50 and 100 mg/kg BVD-523 p.o. bid to end.

### Combination Treatments

Each one of two groups receives a combination of 50 mg/kg BVD-523 with one of two different concentrations of RO092210 (30 or 100 mg/kg). Two other groups receive 100 mg/kg BVD-523 with one of two different concentrations of RO092210 (30 or 100 mg/kg).

### Endpoint and Tumor Growth Delay (TGD) Analysis

Tumors are measured using calipers twice per week, and each animal is euthanized when its tumor reaches the pre-determined tumor volume endpoint of 2000 mm³ or on the final day, whichever comes first. Animals that exit the study for tumor volume endpoint are documented as euthanized for tumor progression (TP), with the date of euthanasia. The time to endpoint (TTE) for analysis is calculated for each mouse by the following equation:

TTE=[log₁₀(endpoint volume)−b]/m

where TTE is expressed in days, endpoint volume is expressed in mm³, b is the intercept, and m is the slope of the line obtained by linear regression of a log-transformed tumor growth data set. The data set consists of the first observation that exceeds the endpoint volume used in analysis and the three consecutive observations that immediately precede the attainment of this endpoint volume. The calculated TTE is usually less than the TP date, the day on which the animal is euthanized for tumor size. Animals with tumors that do not reach the endpoint volume are assigned a TTE value equal to the last day of the study. Any animal classified as having died from NTR (non-treatment-related) causes due to accident (NTRa) or due to unknown etiology (NTRu) are excluded from TTE calculations (and all further analyses). Animals classified as TR (treatment-related) deaths or NTRm (non-treatment-related death due to metastasis) are assigned a TTE value equal to the day of death.

Treatment outcome is evaluated from TGD, defined as the increase in the median TTE in a treatment group compared to the control group:

TGD=T−C,

expressed in days, or as a percentage of the median TTE of the control group:

% TGD=[(T−C)/C]×100

where:
T=median TTE for a treatment group, and
C=median TTE for the designated control group.

### Criteria for Regression Responses

Treatment efficacy may be determined from the incidence and magnitude of regression responses observed during the study. Treatment may cause partial regression (PR) or complete regression (CR) of the tumor in an animal. In a PR response, the tumor volume is 50% or less of its Day 1 volume for three consecutive measurements during the course of the study, and equal to or greater than 13.5 mm³ for one or more of these three measurements. In a CR response, the tumor volume is less than 13.5 mm³ for three consecutive measurements during the course of the study. An animal with a CR response at the termination of the study is additionally classified as a tumor-free survivor (TFS). Animals are monitored for regression responses.

### Toxicity

Animals are weighed daily on Days 1-5, then twice per week until completion of the study. The mice are observed frequently for overt signs of any adverse, TR side effects, and clinical signs are recorded when observed. Individual BW loss is monitored as per protocol, and any animal whose weight exceeds the limits for acceptable BW loss is euthanized. Group mean BW loss also is monitored as per protocol. Dosing is to be suspended in any group that exceeds the limits for acceptable mean BW loss. If mean BW recovers, then dosing is to be resumed in that group, but at a lower dosage or less frequent dosing schedule. Acceptable toxicity for the maximum tolerated dose (MTD) is defined as a group mean BW loss of less than 20% during the study and not more than 10% TR deaths. A death is classified as TR if attributable to treatment side effects as evidenced by clinical signs and/or necropsy, or may also be classified as TR if due to unknown causes during the dosing period or within 14 days of the last dose. A death is classified as NTR if there is no evidence that death is related to treatment side effects. NTR deaths may be further characterized based on cause of death. A death is classified as NTRa if it results from an accident or human error. A death is classified as NTRm if necropsy indicates that it may result from tumor dissemination by invasion and/or metastasis. A death is classified as NTRu if the cause of death is unknown and there is no available evidence of death related to treatment side effects, metastasis, accident or human error, although death due to treatment side effects cannot be excluded.

### Statistical and Graphical Analyses

Prism (GraphPad) for Windows 3.03 is used for graphical presentations and statistical analyses.

The logrank test, which evaluates overall survival experience, is used to analyze the significance of the differences between the TTE values of two groups. Logrank analysis includes the data for all animals in a group except those assessed as NTR deaths. Two-tailed statistical analyses are conducted at significance level P=0.05. The statistical tests are not adjusted for multiple comparisons. Prism summarizes test results as not significant (ns) at P>0.05, significant (symbolized by “*”) at 0.01<P<0.05, very significant (“**”) at 0.001<P≦0.01, and extremely significant (“***”) at P≦0.001. Groups with regimens above the MTD are not evaluated statistically.

A scatter plot is constructed to show TTE values for individual mice, by group. Group mean tumor volumes are plotted as a function of time. When an animal exits the study due to tumor size, the final tumor volume recorded for the animal is included with the data used to calculate the mean volume at subsequent time points. Error bars (when present) indicate one standard error of the mean (SEM). Tumor growth plots exclude the data for NTR deaths, and are truncated after 50% of the assessable animals in a group exit the study or after the second TR death in a group, whichever comes first. Kaplan-Meier plots show the percentage of animals in each group remaining in the study versus time. The Kaplan-Meier plot and logrank test share the same TTE data sets. Percent mean BW changes from Day 1 are calculated for each group for each day of BW measurement, and are plotted as a function of time. BW plots exclude the data for NTR deaths, and are truncated after 50% of the assessable animals in a group exit the study.

### Results

It is expected that the combination of RO092210 with BVD-523 will be effective against HCT116 cell-derived tumors and that the results are statistically significant. It is also expected that the side effects associated with BVD-523/type 1 MEK inhibitor treatment will be minimal.

### Example 4

### Cell Culture Studies of MEK and ERK Inhibitors

### Single Agent Proliferation Assay

Cells were seeded in 96-well plates at the densities and media conditions indicated in Table 7 and allowed to adhere overnight prior to addition of compound or vehicle control. Compounds were prepared from DMSO stocks to give the desired final concentrations The final DMSO concentration was constant at 0.1%. Test compounds were incubated with the cells for 72 h at 37° C., 5% CO2 in a humidified atmosphere. CellTiter-Glo® reagent (Promega, Madison, Wis.) was added according to manufacturer's instructions and luminescence detected using the BMG FLUOstar plate reader (BMG Labtech, Ortenberg, Germany). The average media only background value was deducted and the data analysed using a 4-parameter logistic equation in GraphPad Prism (GraphPad Software, La Jolla, Calif.).

### Combination Proliferation Assay

Cells were seeded in triplicate 96-well plates at the densities and media conditions indicated in Table 7 and allowed to adhere overnight prior to addition of compound or vehicle control. Compounds were prepared from DMSO stocks to give the desired final concentrations The final DMSO concentration was constant at 0.2%. Combinations were tested using a 10×8 dose matrix or a 10×6 dose matrix. Test compounds were incubated with the cells for 72 h at 37° C., 5% CO2 in a humidified atmosphere. CellTiter-Glo® reagent (Promega, Madison, Wis.) was added according to manufacturer's instructions and luminescence detected using the BMG FLUOstar plate reader (BMG Labtech, Ortenberg, Germany). The average media only background value was deducted and the data analysed.

Combination interactions across the dose matrix were determined by the Loewe Additivity and Bliss independence models using Chalice™ Combination Analysis Software (Horizon Discovery Group, Cambridge, Mass.) as outlined in the user manual (available at chalice. horizondiscovery.com/chalice-portal/documentation/analyzer/home.jsp). Synergy is determined by comparing the experimentally observed level of inhibition at each combination point with the value expected for additivity, which is derived from the single-agent responses along the edges of the matrix. Potential synergistic interactions were identified by displaying the calculated excess inhibition over that predicted as being additive across the dose matrix as a heat map, and by reporting a quantitative ‘Synergy Score’ based on the Loewe model. The single agent data derived from the combination assay plates were presented as dose-response curves generated in GraphPad Prism (GraphPad Software, La Jolla, Calif.) (plotted using percentage viability relative to DMSO only treated controls).

TABLE 7
Cell Line Seeding Density and Growth Media

| | Seeding Density | |
|---|---|---|
| Cell Line | (cells/well) | Media |
| HCT116 Parental | 1000 | McCoy's 5A + 10% FBS |
| HCT116 KRAS KO (+/−) | 2000 | McCoy's 5A + 10% FBS |
| RKO Parental | 2000 | McCoy's 5A + 10% FBS |
| RKO BRAF KO (+/−/−) | 2000 | McCoy's 5A + 10% FBS |
| A375 Parental | 2000 | DMEM + 10% FBS |
| A375 NRAS (Q61K/+/+) | 2000 | DMEM + 10% FBS |
| G-361 | 5000 | McCoy's 5A + 10% FBS |
| A549 | 750 | RPMI 1640 + 10% FBS |
| H2212 | 4000 | RPMI 1640 + 10% FBS |
| H1437 | 1500 | RPMI 1640 + 10% FBS |
| H226 | 750 | RPMI 1640 + 10% FBS |

### Results

The aim of this study was to assess the effects on cell viability of combining ERK inhibitors with MEK inhibitors in a panel of isogenic and non-isogenic cancer cell lines (Table 8).

TABLE 8
Description of Cell Lines Studied

| Cell Line | Cancer Type | Description |
|---|---|---|
| HCT116 | CRC | Heterozygous parental cells containing |
| Parental | | one mutant KRAS allele (G13D) and one |
| | | wild type allele |
| HCT116 | CRC | Knock out of mutant KRAS allele in |
| KRAS KO | | heterozygous parental cells |
| (+/−) | | |
| RKO Parental | CRC | Triploid parental cells containing two |
| | | mutant BRAF alleles (V600E) and one wild |
| | | type allele |
| RKO BRAF | CRC | Knock out of both mutant BRAF alleles |
| KO (+/−/−) | | (V600E) in triploid parental cells |
| A375 Parental | Melanoma | Hypotriploid parental line carrying BRAF |
| | | (V600E) mutation |
| A375 NRAS | Melanoma | Heterozygous knock-in of NRAS |
| (Q61K/+/+) | | activating mutation (Q61K) |
| G-361 | Melanoma | BRAF (V600E) mutant |
| A549 | NSCLC | BRAF mutant |
| H2212 | NSCLC | BRAF mutant |
| H1437 | NSCLC | KRAS wild type |
| H226 | NSCLC | KRAS wild type |

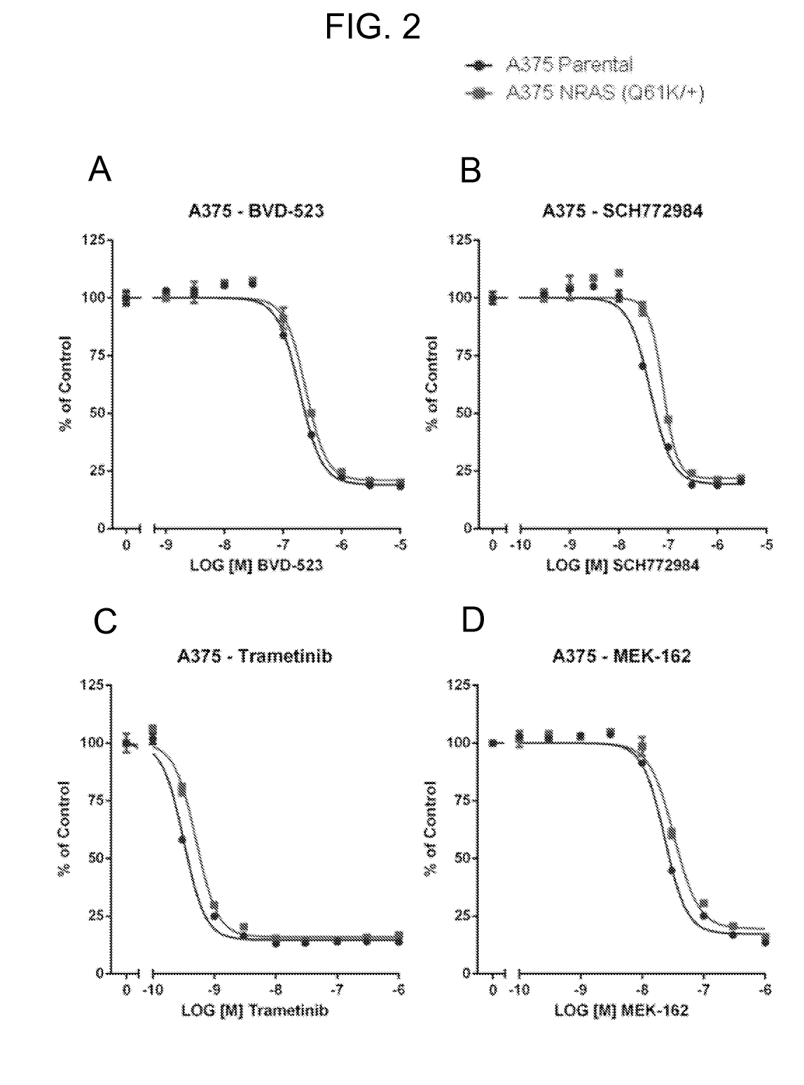

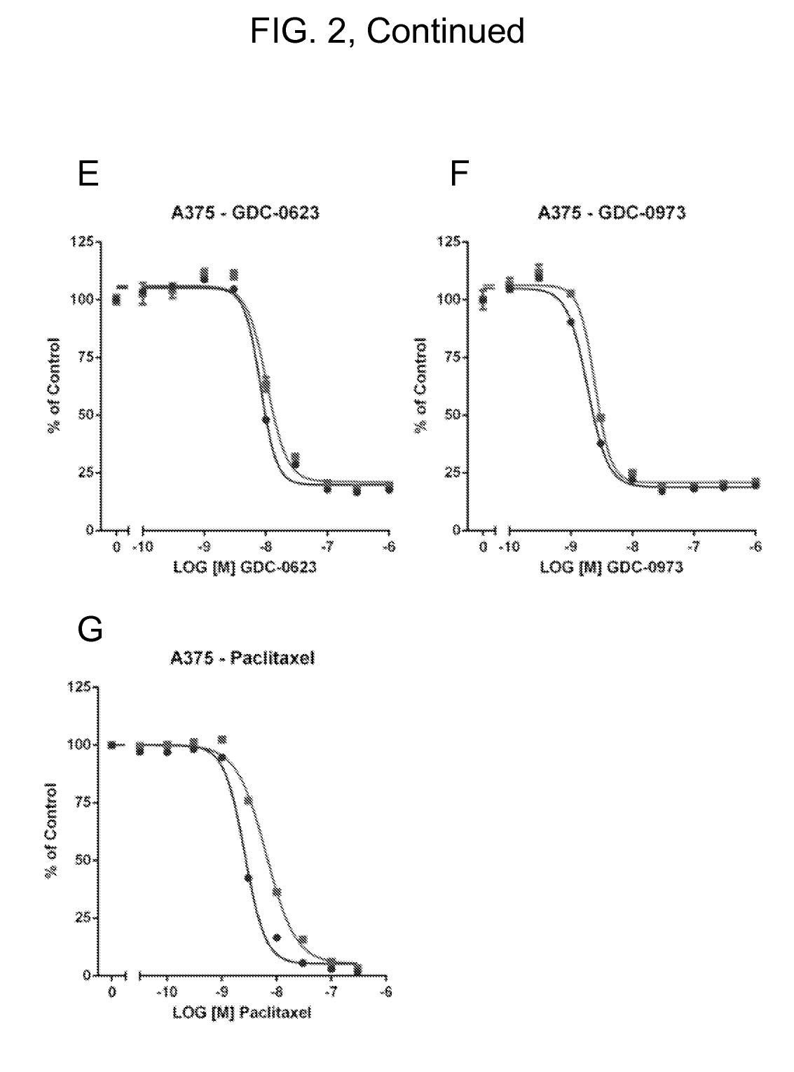

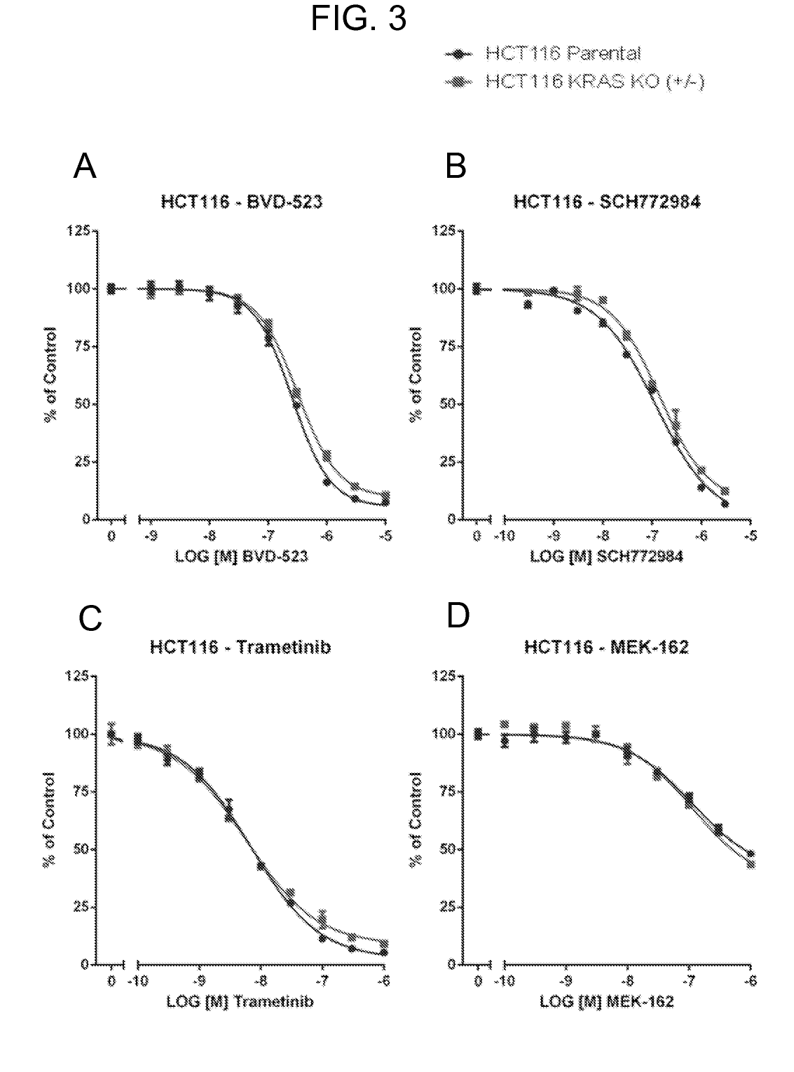

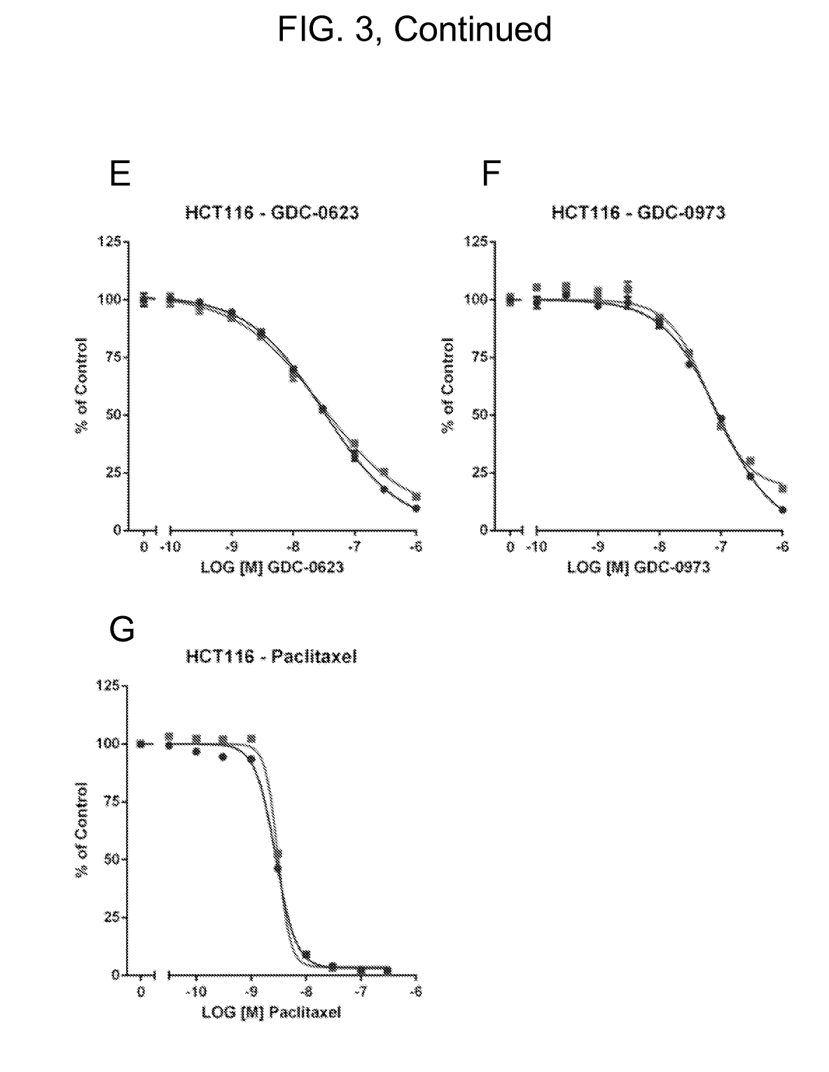

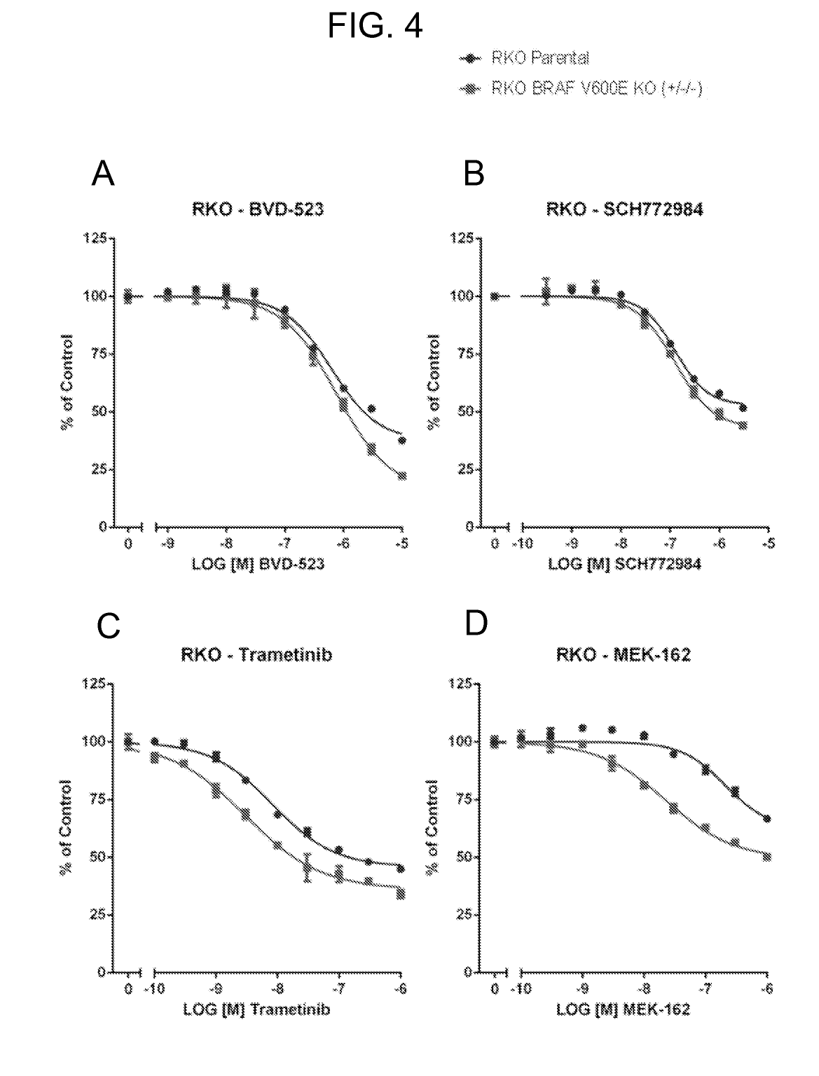

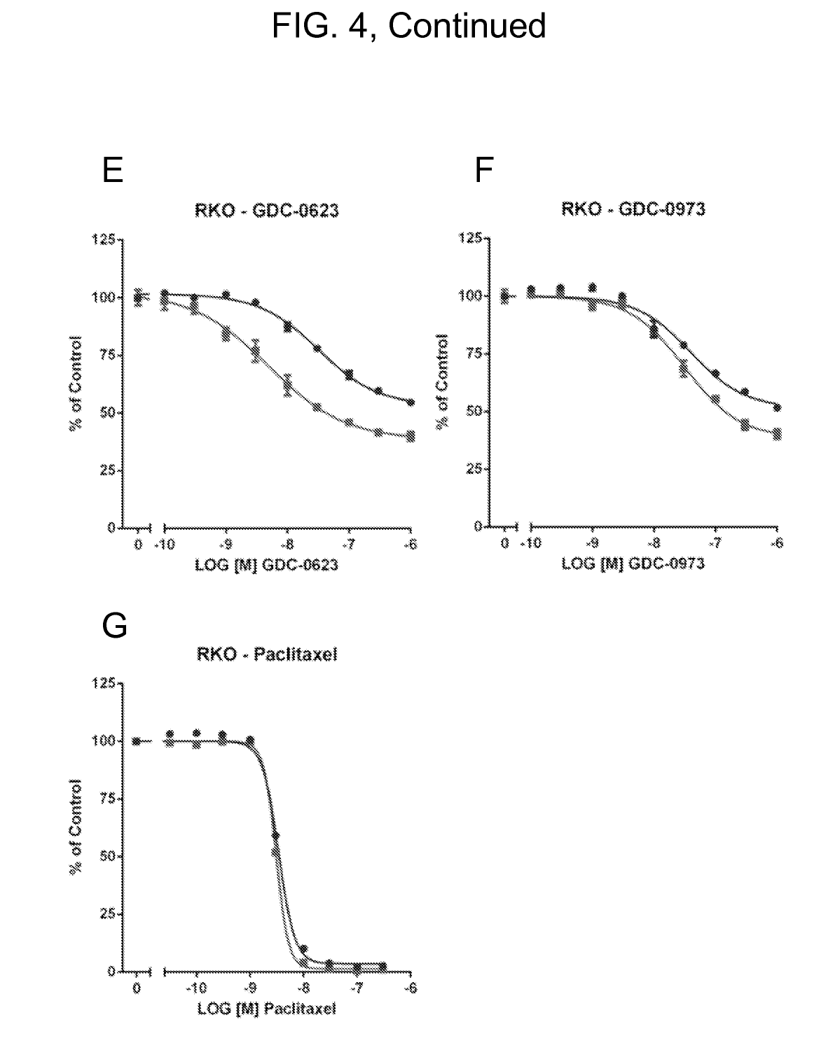

An initial round of single agent assays was performed in the A375 (FIG. 2), HCT116 (FIG. 3) and RKO-isogenic (FIG. 4) cell line pairs. IC₅₀ values are shown in Table 9. These revealed no differentials in response to ERK or MEK inhibition between the two cell lines within the A375 and HCT116 isogenic pair. This suggests that under the assay conditions tested 1) the knocked-in mutant NRAS allele does not drive resistance to MEK or ERK inhibition in A375 cells and 2) sensitivity of HCT116 to MEK/ERK inhibition is not coupled to the mutant KRAS allele.

TABLE 9
Single Agent IC₅₀ Values

| | A375 | | HCT116 | | RKO | |
|---|---|---|---|---|---|---|
| | Par- | NRAS | Par- | KRAS KO | Par- | BRAF KO |
| Compound | ental | (Q61K/+) | ental | (+/−) | ental | (+/−/−) |
| BVD-523 | 0.193 | 0.243 | 0.256 | 0.316 | 0.621 | 0.762 |
| SCH772984 | 0.043 | 0.079 | 0.116 | 0.141 | 0.126 | 0.125 |
| Trametinib | 0.0003 | 0.0005 | 0.007 | 0.006 | 0.008 | 0.003 |
| MEK-162 | 0.023 | 0.033 | 0.114 | 0.113 | 0.210 | 0.023 |
| GDC-0623 | 0.008 | 0.010 | 0.031 | 0.029 | 0.032 | 0.005 |
| GDC-0973 | 0.002 | 0.003 | 0.090 | 0.061 | 0.040 | 0.031 |
| Paclitaxel | 0.003 | 0.006 | 0.003 | 0.003 | 0.003 | 0.003 |

TABLE 10
Bliss Volumes

| | | | | | HCT116 | | RKO BRAF | | | | |
|---|---|---|---|---|---|---|---|---|---|---|---|
| | | | | | KRAS KO | HCT116 | V600E KO | RKO | A375 NRAS | A375 | |
| | A549 | H1437 | H2122 | H226 | (+/−) | Parental | (+/−/−) | Parental | (Q61K/+) | Parental | G-361 |
| BVD-523 × GDC-0623 | nt | 0.29 | 0.633 | −0.505 | nt | nt | nt | nt | 0.014 | −0.963 | 4.02 |
| BVD-523 × MEK-162 | nt | nt | nt | nt | −0.221 | 1.09 | −0.781 | −0.748 | −0.117 | −0.488 | 1.29 |
| BVD-523 × Trametinib | −1.06 | −0.324 | 0.361 | 0.364 | 0.811 | 0.606 | −1.88 | −2.15 | 0.188 | −1.83 | 0.774 |
| SCH772984 × GDC-0623 | −0.0669 | 0.525 | 0.244 | −0.792 | nt | nt | nt | nt | 0.442 | −0.444 | 4.29 |
| SCH772984 × MEK-162 | nt | nt | nt | nt | 1.25 | 1.4 | −2.47 | 0.378 | −0.697 | −0.261 | 1.53 |
| SCH772984 × Trametinib | −0.436 | −1.44 | −0.0333 | −3.15 | 1.94 | 2.09 | −4.01 | −1.59 | 0.0516 | −0.256 | 2.42 |

TABLE 11
Loewe Volumes

| | | | | | HCT116 | | RKO BRAF | | | | |
|---|---|---|---|---|---|---|---|---|---|---|---|
| | | | | | KRAS KO | HCT116 | V600E KO | RKO | A375 NRAS | A375 | |
| | A549 | H1437 | H2122 | H226 | (+/−) | Parental | (+/−/−) | Parental | (Q61K/+) | Parental | G-361 |
| BVD-523 × GDC-0623 | nt | 0.899 | 1.1 | 0.731 | nt | nt | nt | nt | −0.0852 | −0.217 | 4.39 |
| BVD-523 × MEK-162 | nt | nt | nt | nt | 1.3 | 1.93 | 3.08 | 0.596 | 1.18 | 0.821 | 1.94 |
| BVD-523 × Trametinib | 1.69 | 2.35 | 1.61 | 2.77 | 3.1 | 2.05 | 2.99 | 1.43 | 2.2 | 0.294 | 1.65 |
| SCH772984 × GDC-0623 | 0.846 | 1.52 | 1.1 | 1.22 | nt | nt | nt | nt | 0.0892 | 0.256 | 4.74 |
| SCH772984 × MEK-162 | nt | nt | nt | nt | 3.27 | 3.08 | 2.56 | 1.96 | 0.685 | 1.34 | 1.95 |
| SCH772984 × Trametinib | 2.4 | 2.4 | 2 | 2.1 | 4.94 | 4.23 | 2.52 | 2.71 | 2.1 | 1.95 | 2.72 |

TABLE 12
Synergy Scores

| | | | | | HCT116 | | RKO BRAF | | | | |
|---|---|---|---|---|---|---|---|---|---|---|---|
| | | | | | KRAS KO | HCT116 | V600E KO | RKO | A375 NRAS | A375 | |
| | A549 | H1437 | H2122 | H226 | (+/−) | Parental | (+/−/−) | Parental | (Q61K/+) | Parental | G-361 |
| BVD-523 × GDC-0623 | nt | 0.562 | 0.483 | 0.578 | nt | nt | nt | nt | 0.465 | 0.498 | 2.5 |
| BVD-523 × MEK-162 | nt | nt | nt | nt | 1.68 | 2.28 | 2.53 | 0.777 | 1.43 | 1.49 | 1.88 |
| BVD-523 × Trametinib | 1.59 | 1.51 | 0.748 | 1.35 | 3.23 | 2.46 | 2.82 | 1.06 | 1.28 | 0.731 | 1.23 |
| SCH772984 × GDC-0623 | 0.897 | 0.695 | 0.546 | 0.679 | nt | nt | nt | nt | 0.695 | 0.673 | 2.74 |
| SCH772984 × MEK-162 | nt | nt | nt | nt | 3.2 | 3.4 | 2.06 | 1.26 | 1.22 | 1.54 | 2.08 |
| SCH772984 × Trametinib | 2 | 1.39 | 0.927 | 1.23 | 4.92 | 4.32 | 1.97 | 1.81 | 1.29 | 1.19 | 1.53 |

Surprisingly, deletion of the mutant BRAF (V600E) alleles in RKO cells increased the sensitivity to several of the MEK inhibitors, but did not markedly alter the response to ERK inhibition (FIG. 4). This is consistent with the general observation that upstream modulations of the MAPK pathway that alter sensitivity to MEK inhibitors do not markedly affect sensitivity to ERK inhibition.

Combination interactions between two compounds were assessed across a matrix of concentrations using the Loewe Additivity and Bliss Independence Models with Chalice™ Bioinformatics Software (Horizon Discovery Group, Cambridge, Mass.). Chalice™ enables potential synergistic interactions to be identified by displaying the calculated excess inhibition over that predicted as being additive across the dose matrix as a heat map, and by reporting a quantitative ‘Synergy Score’ based on the Loewe model.

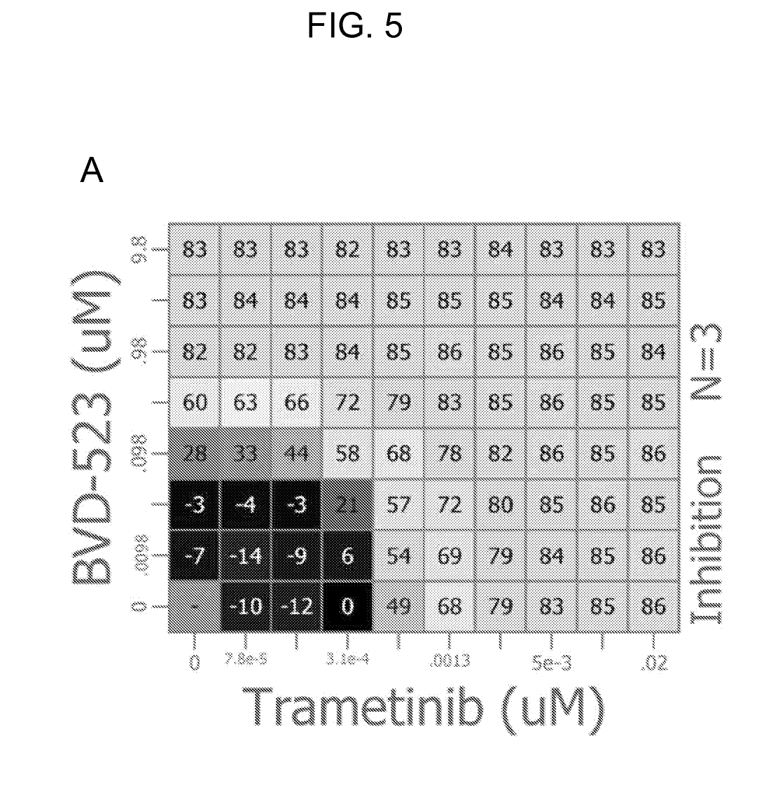

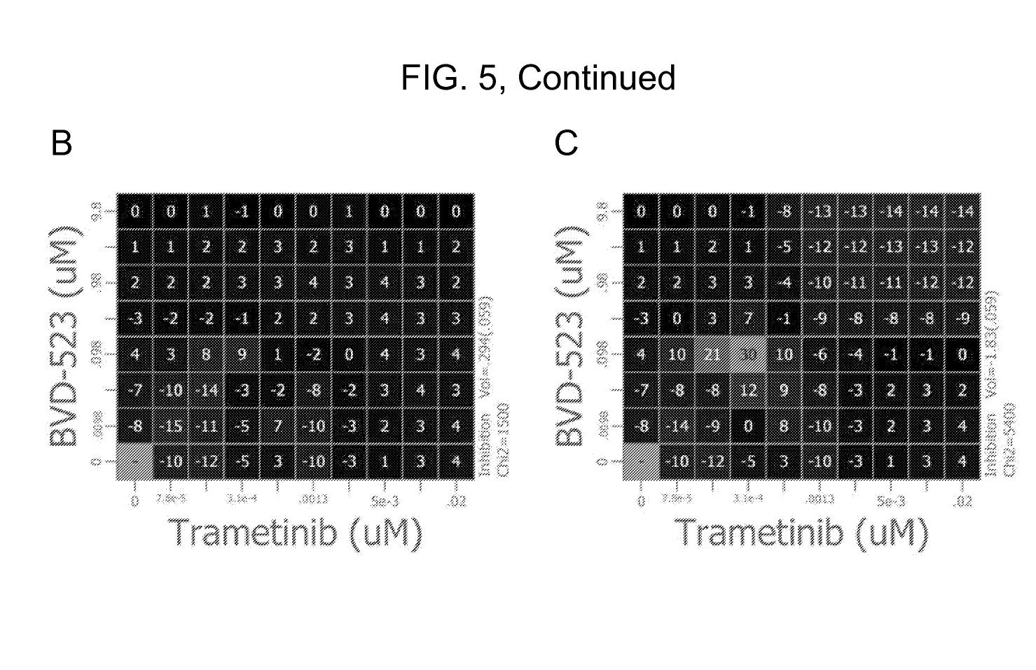

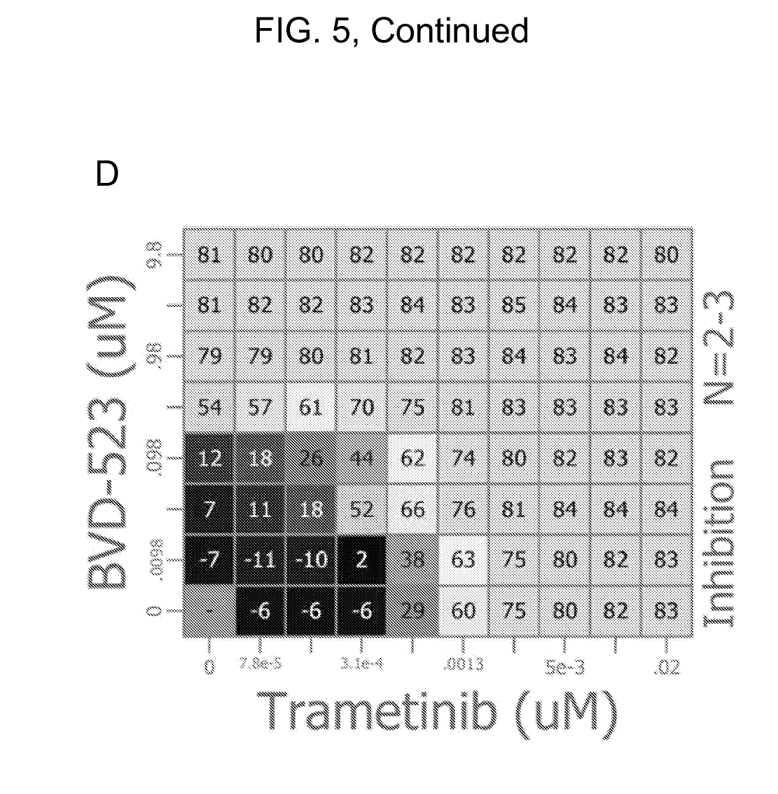

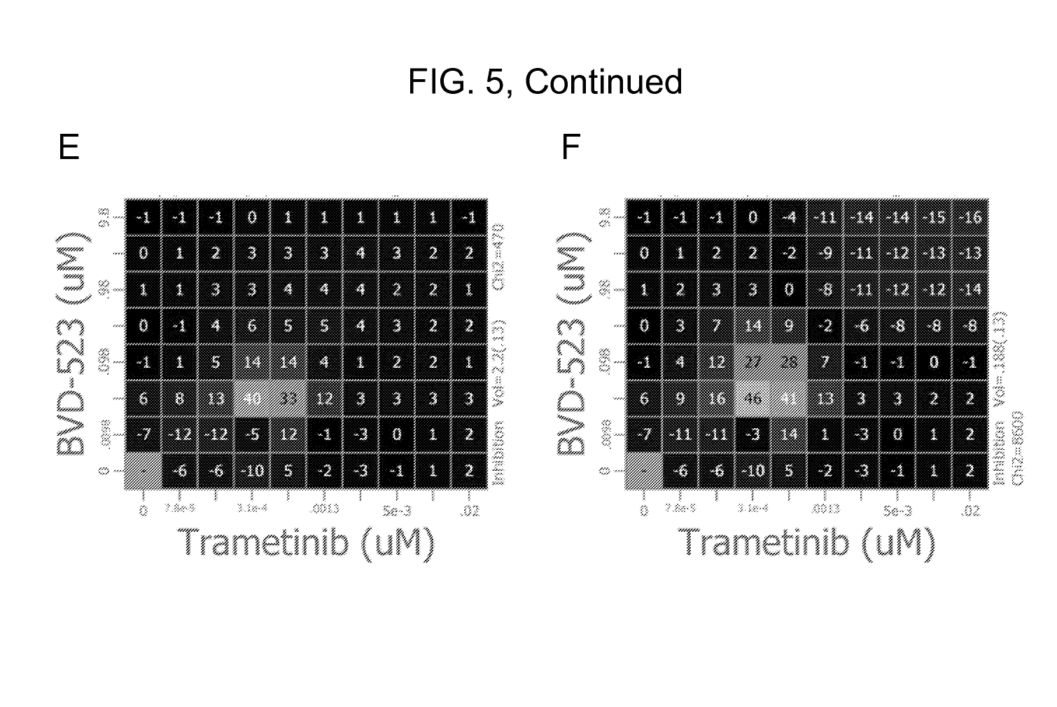

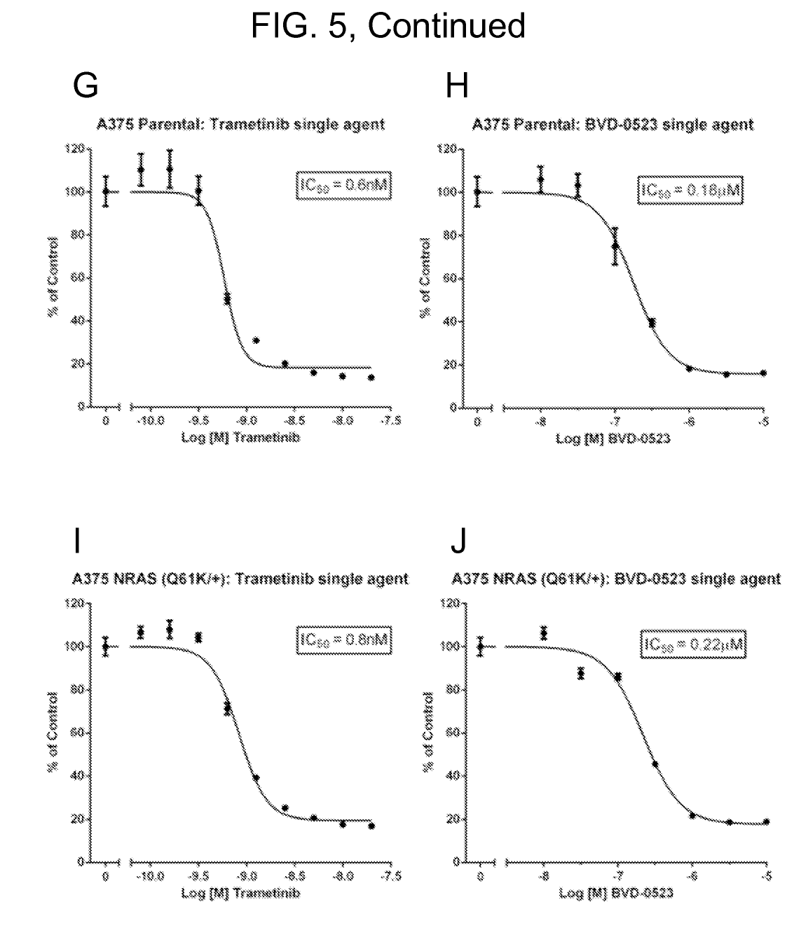

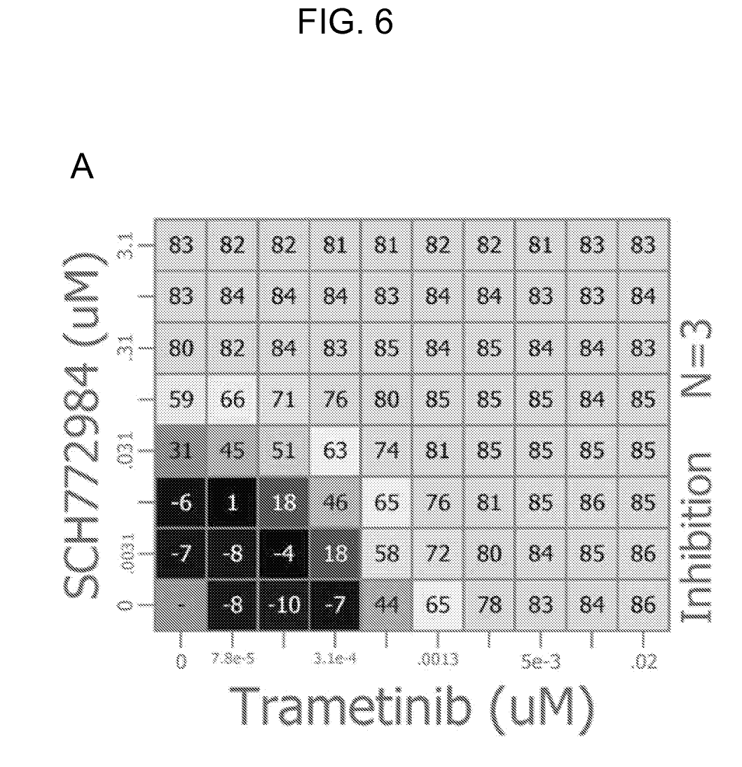

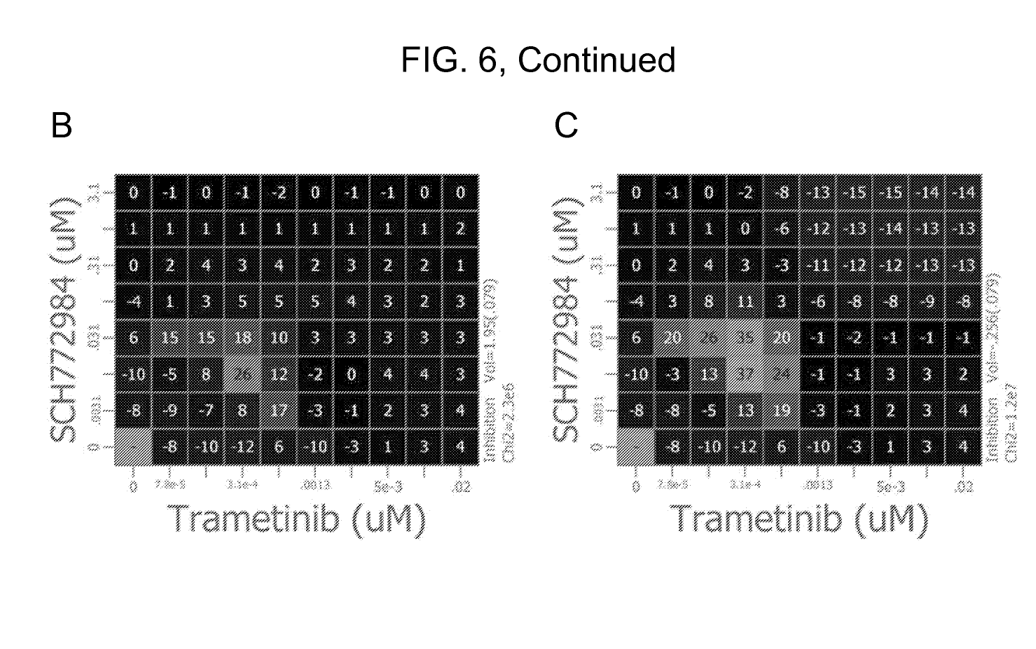

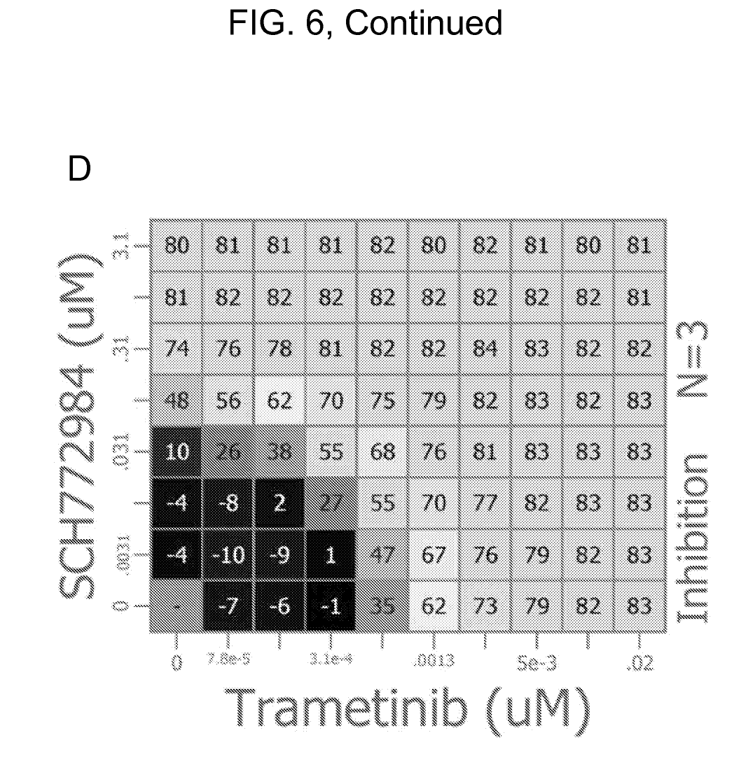

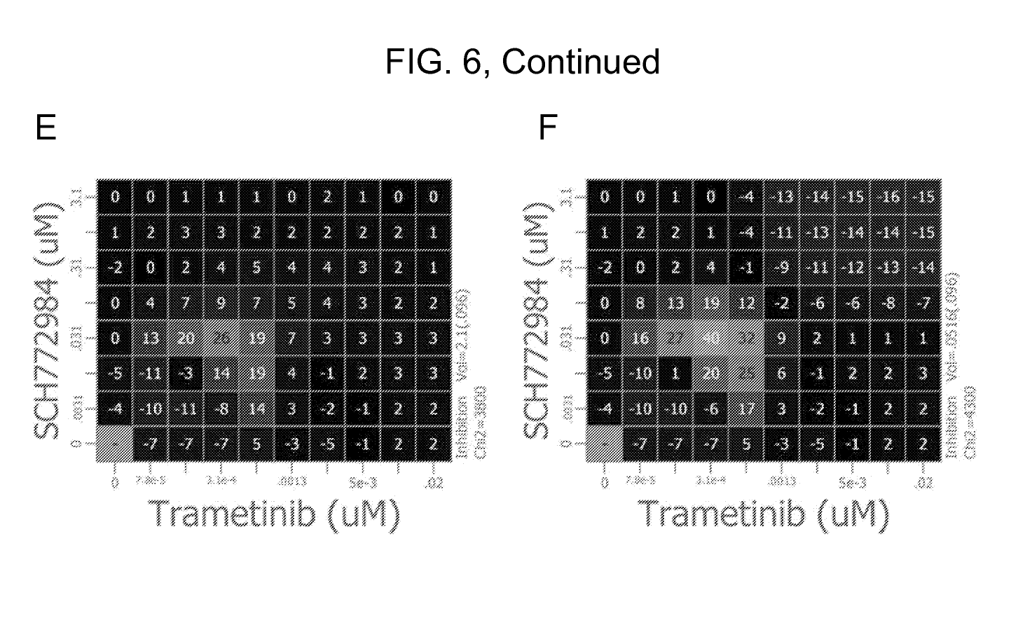

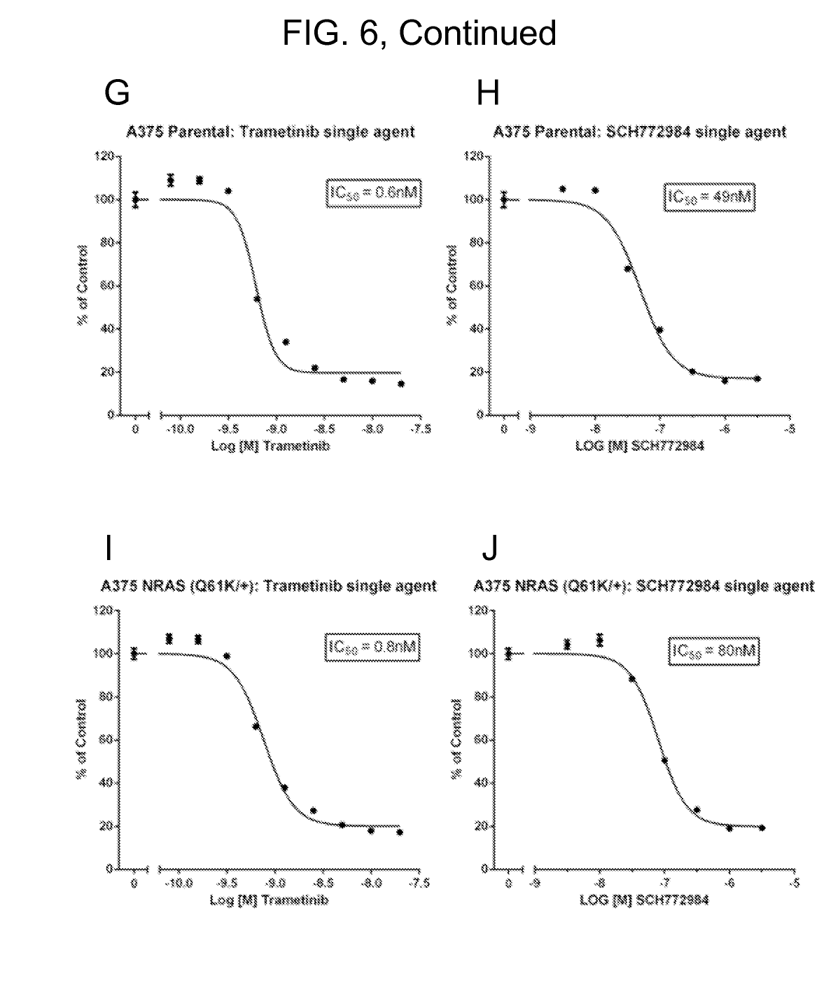

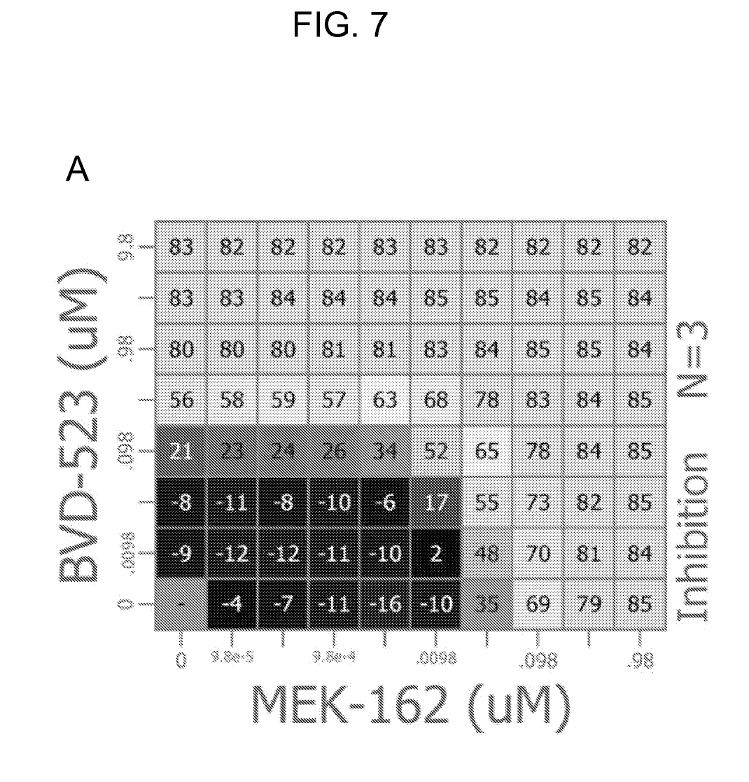

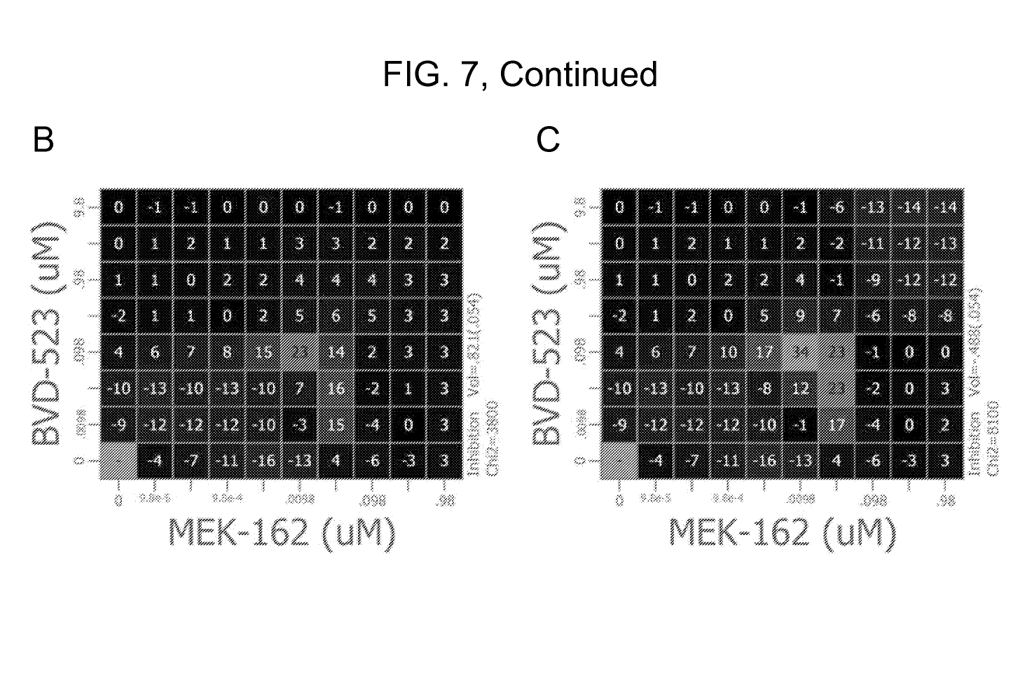

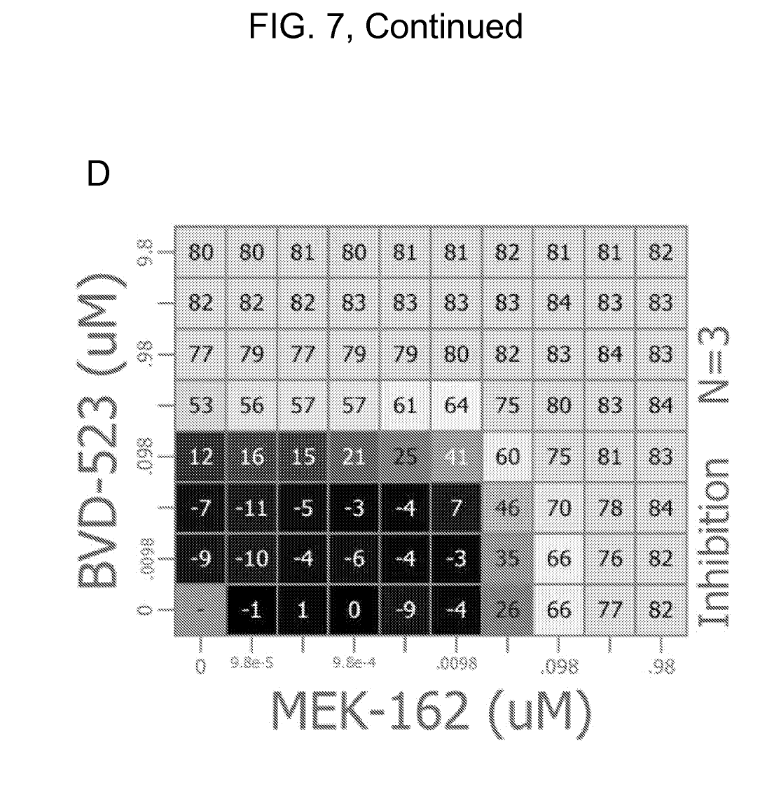

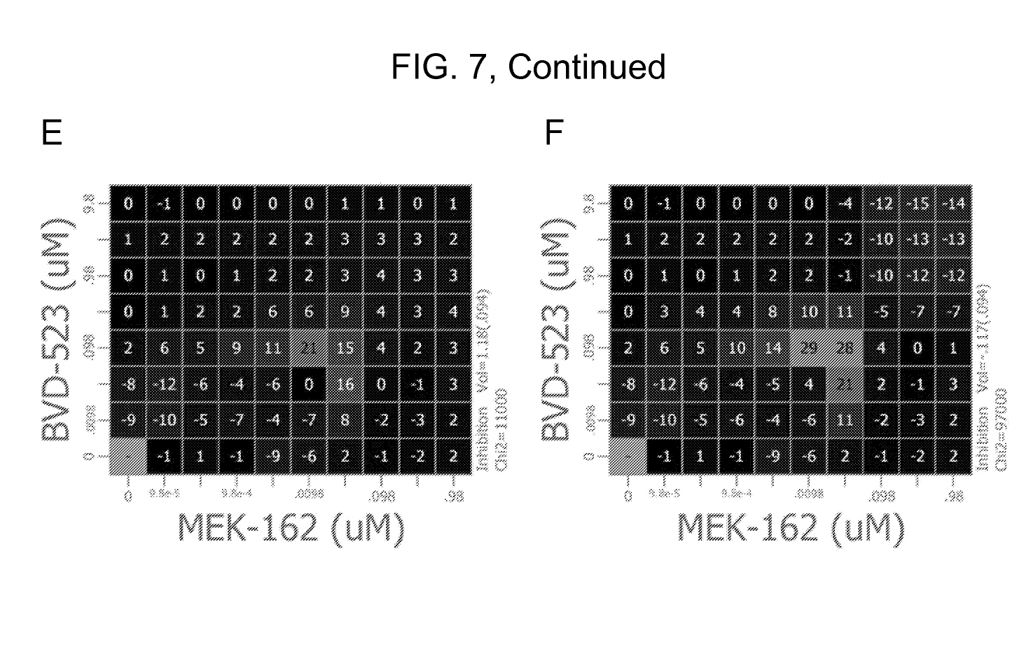

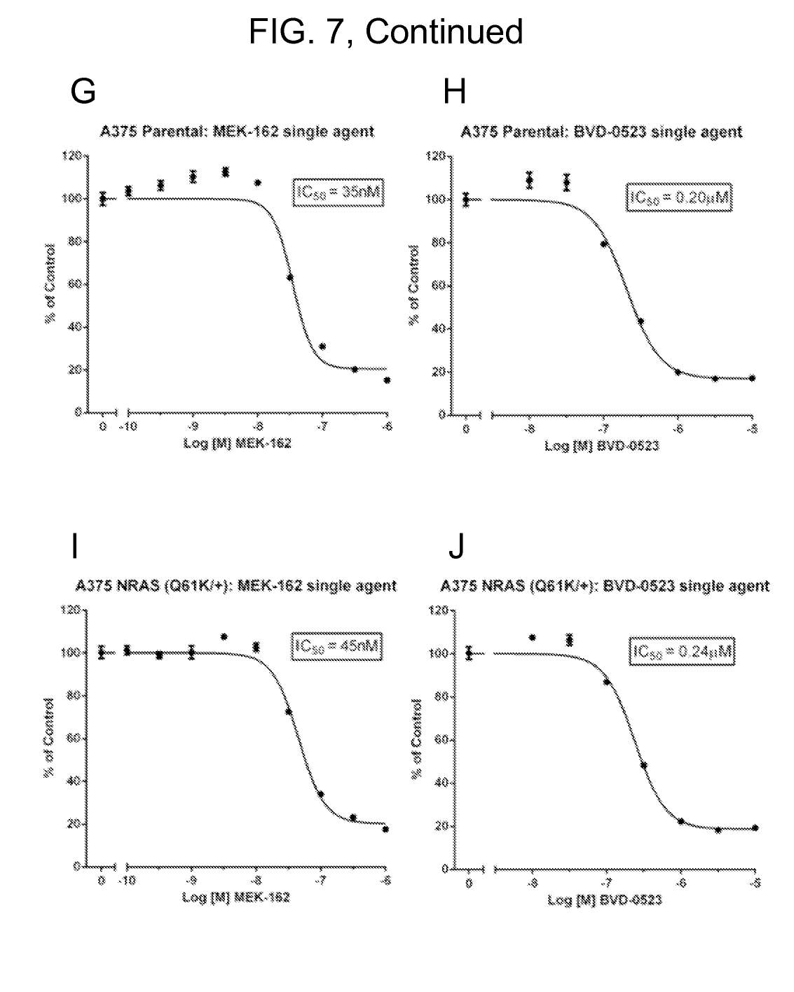

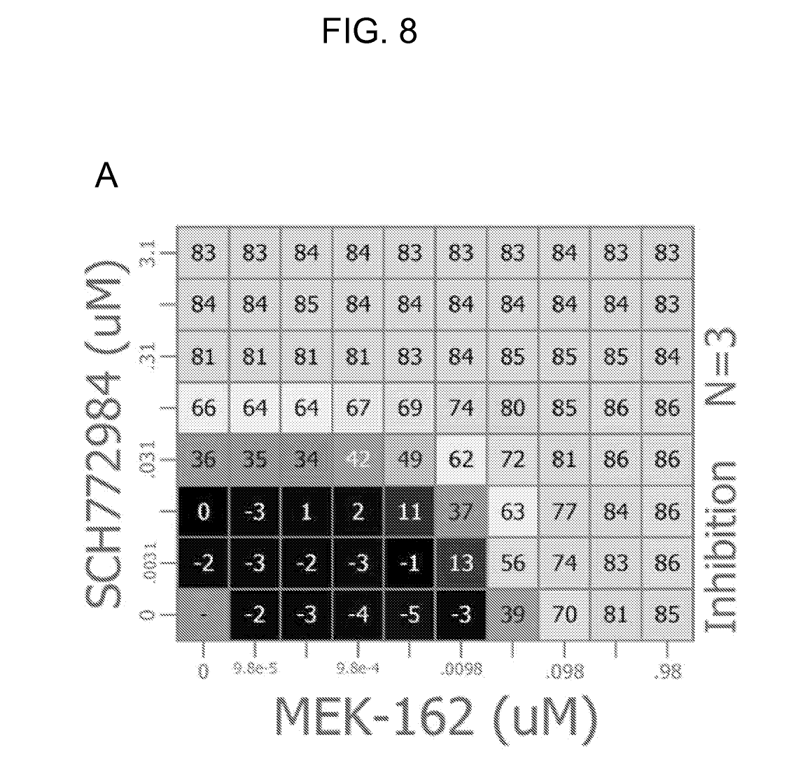

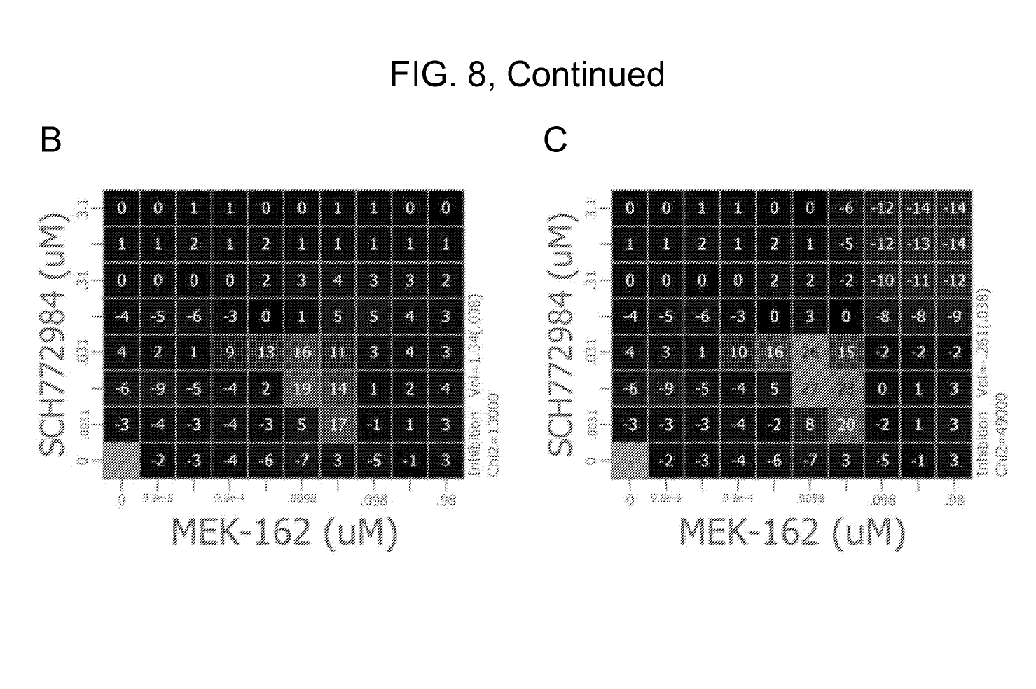

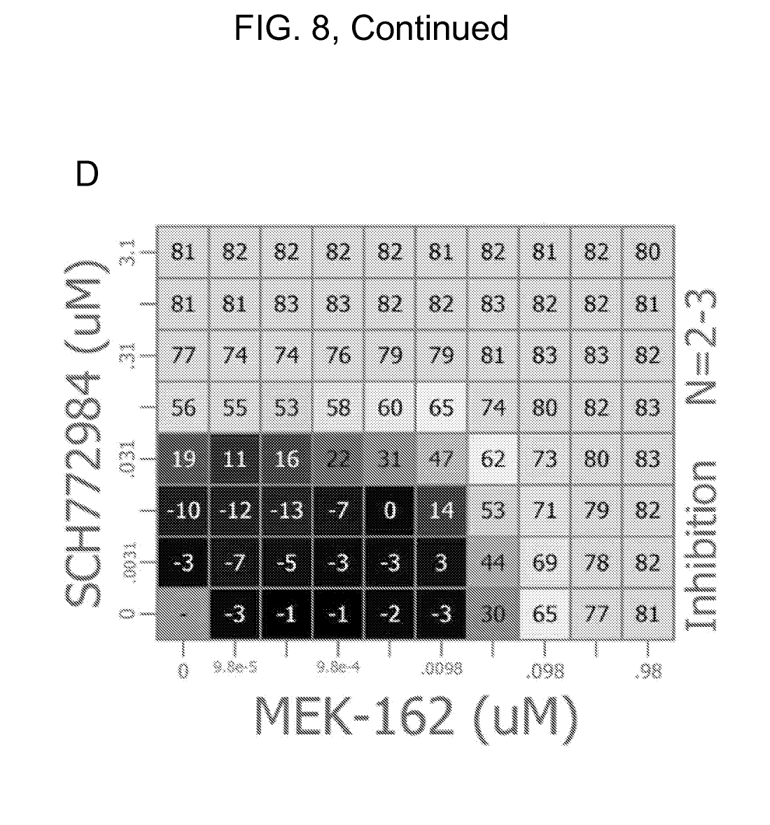

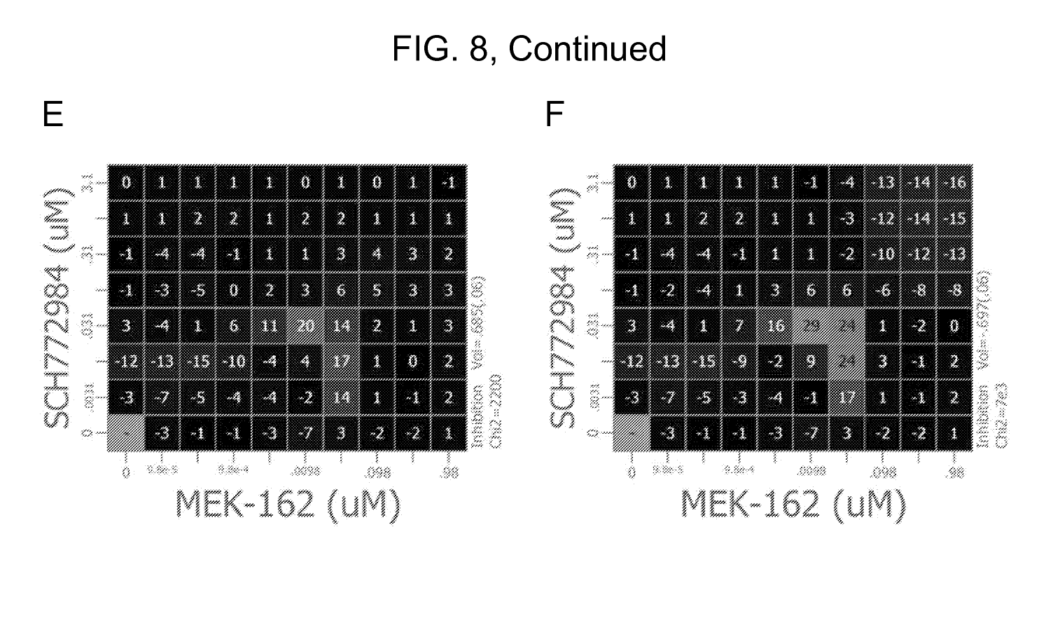

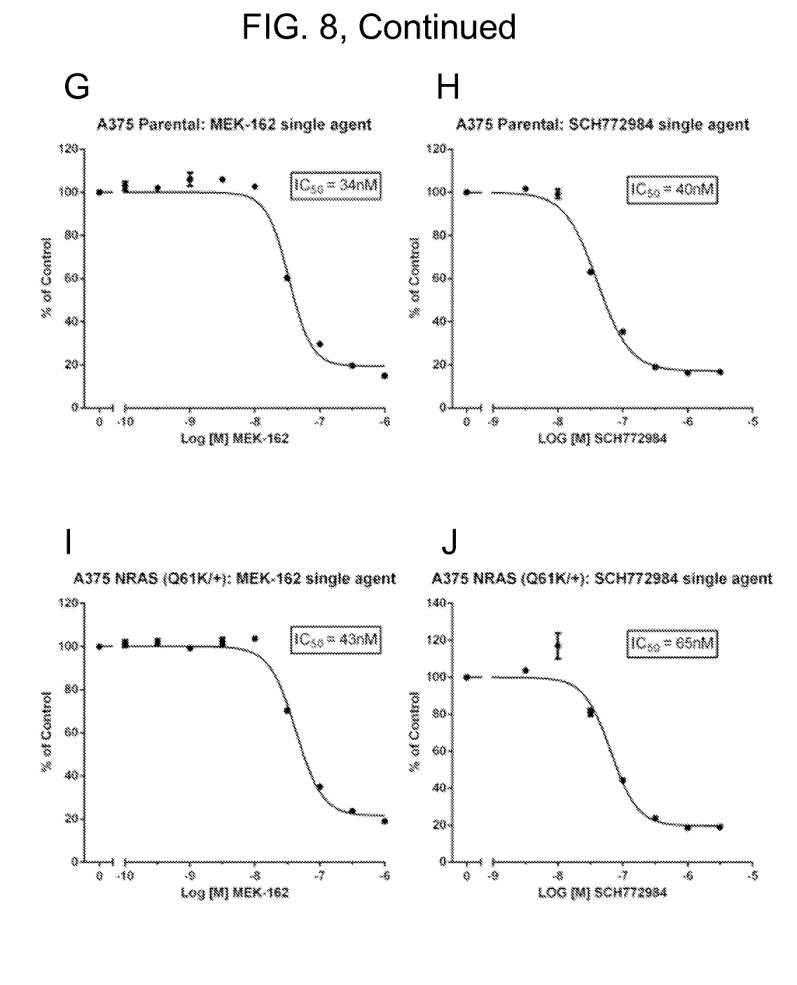

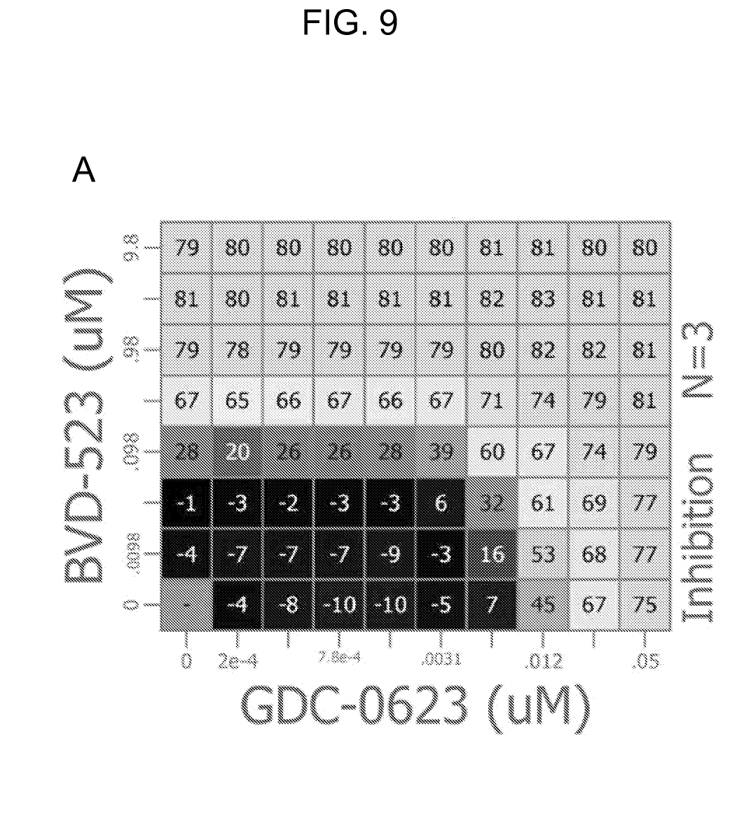

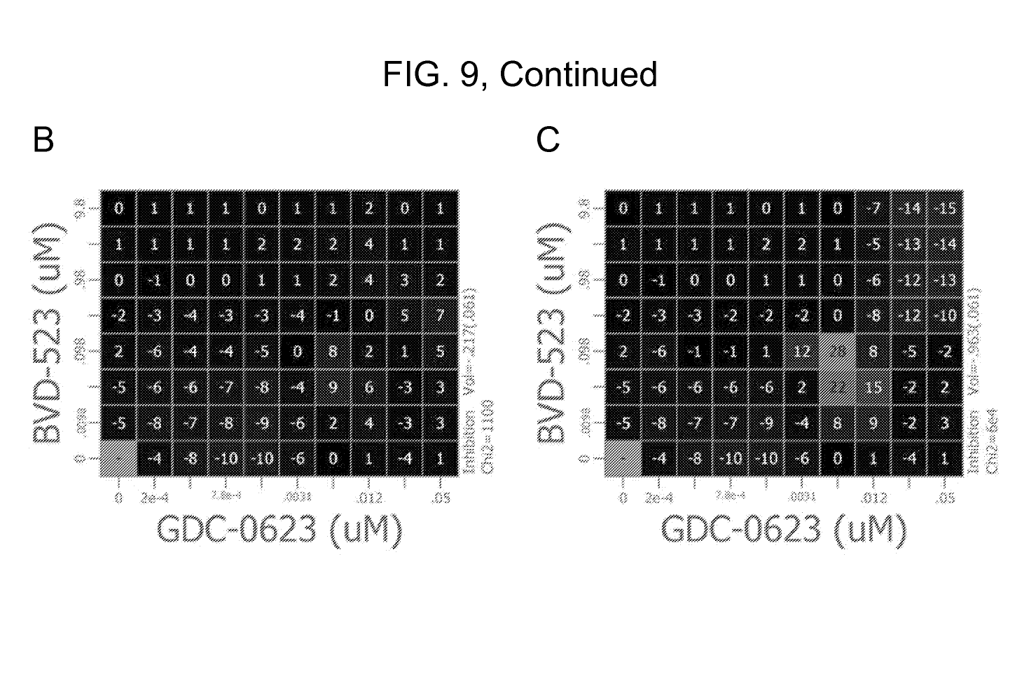

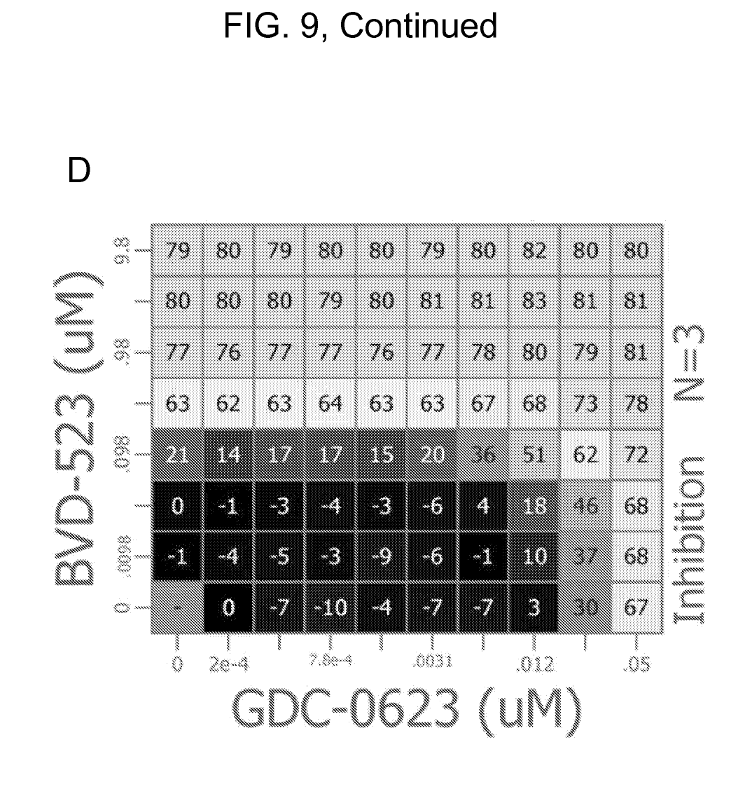

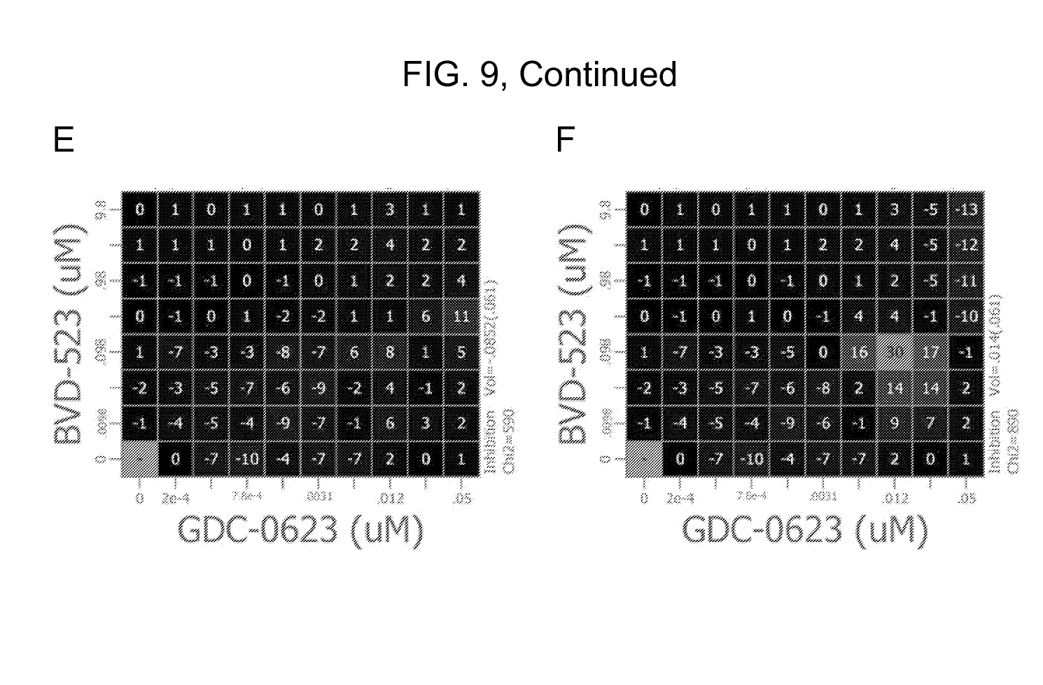

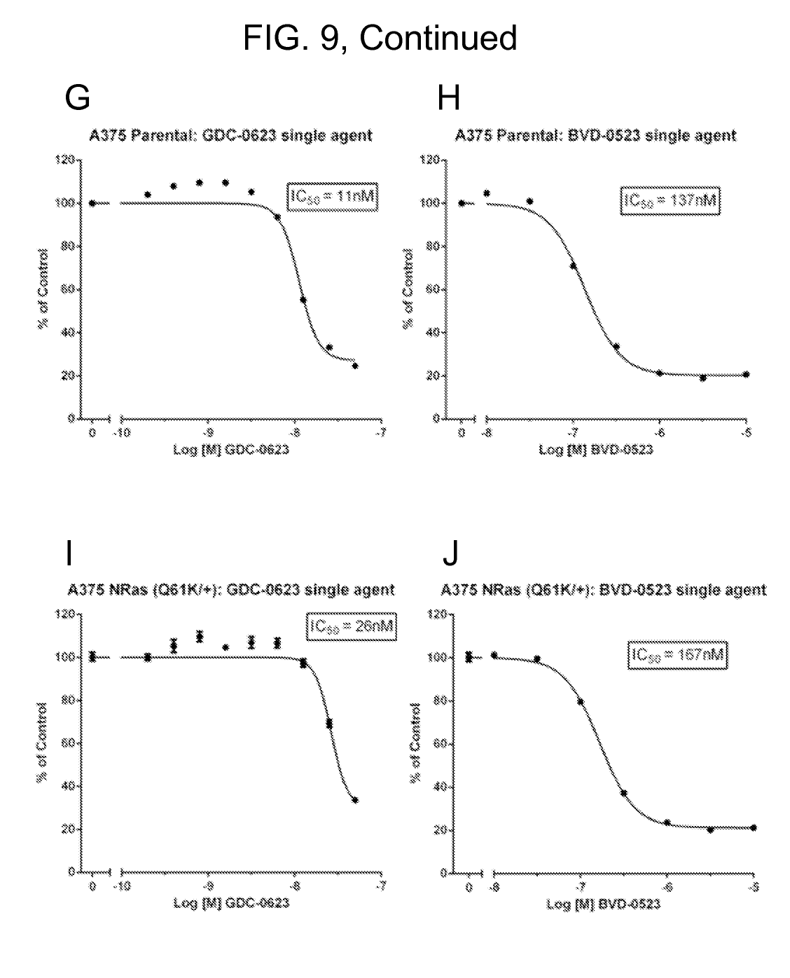

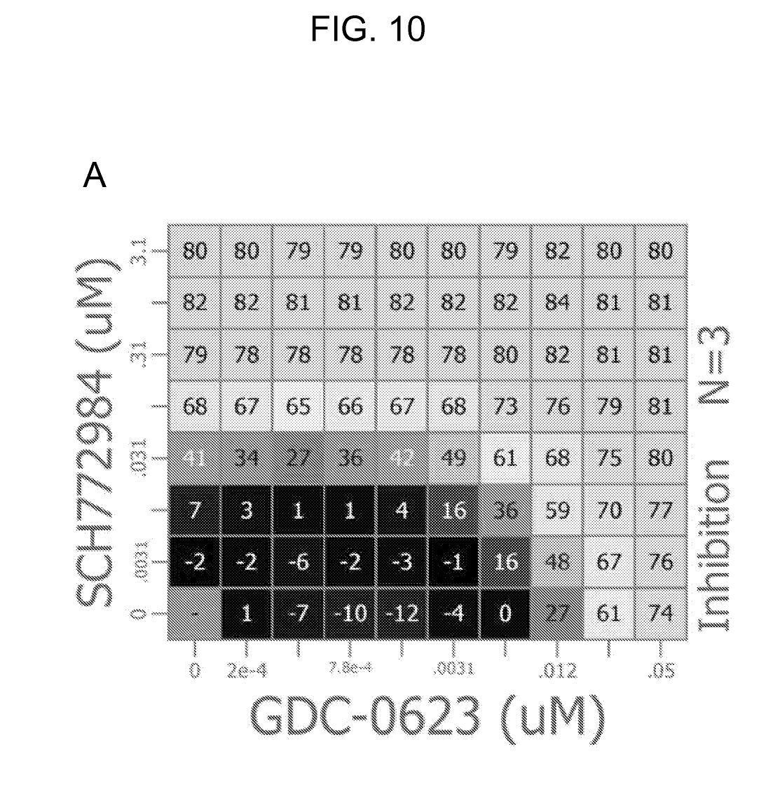

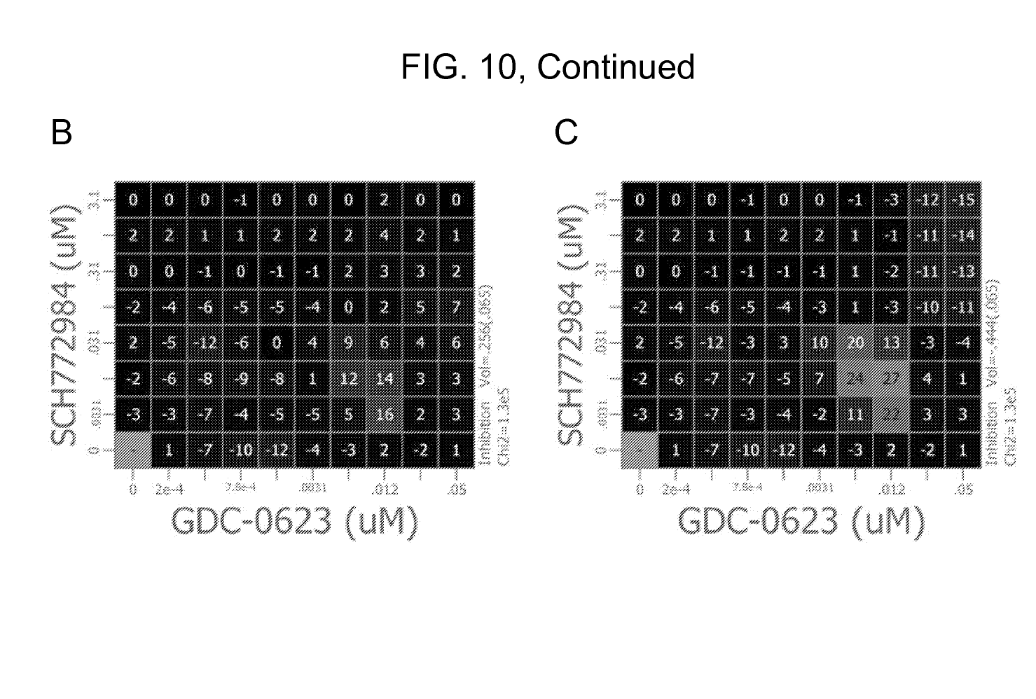

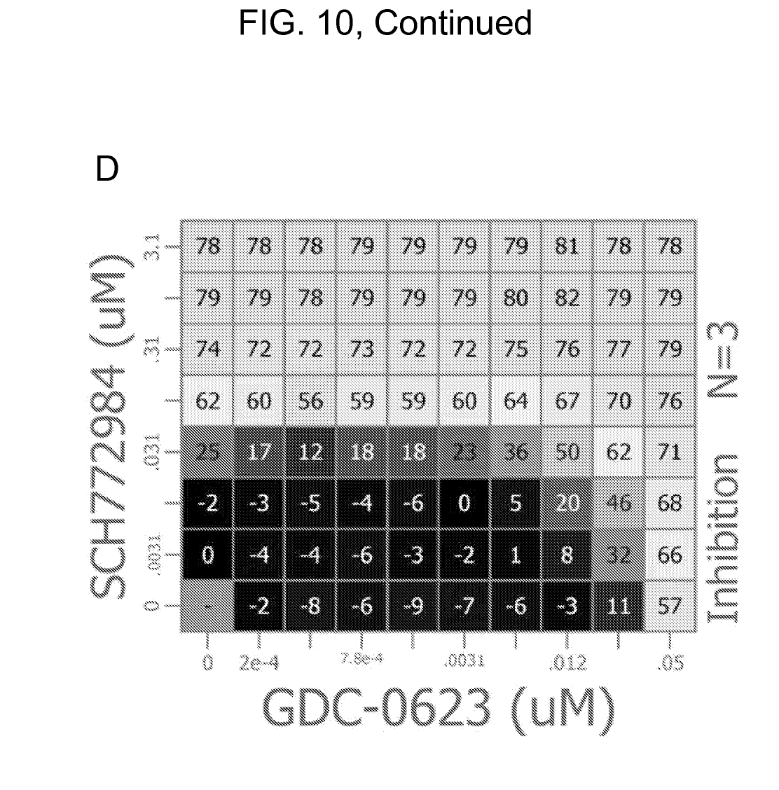

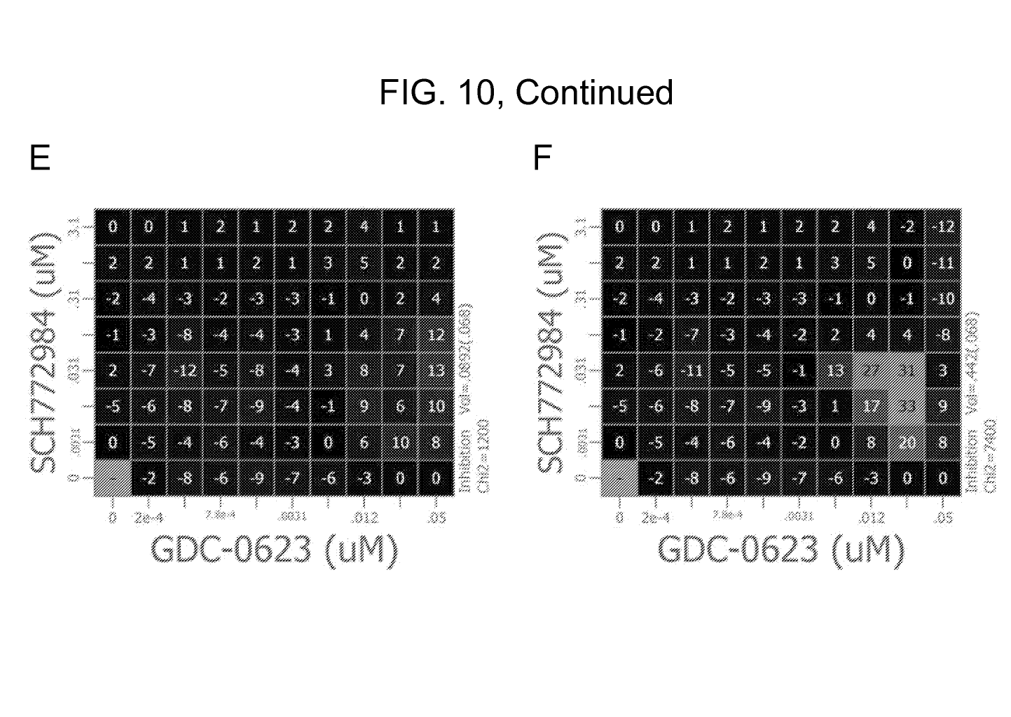

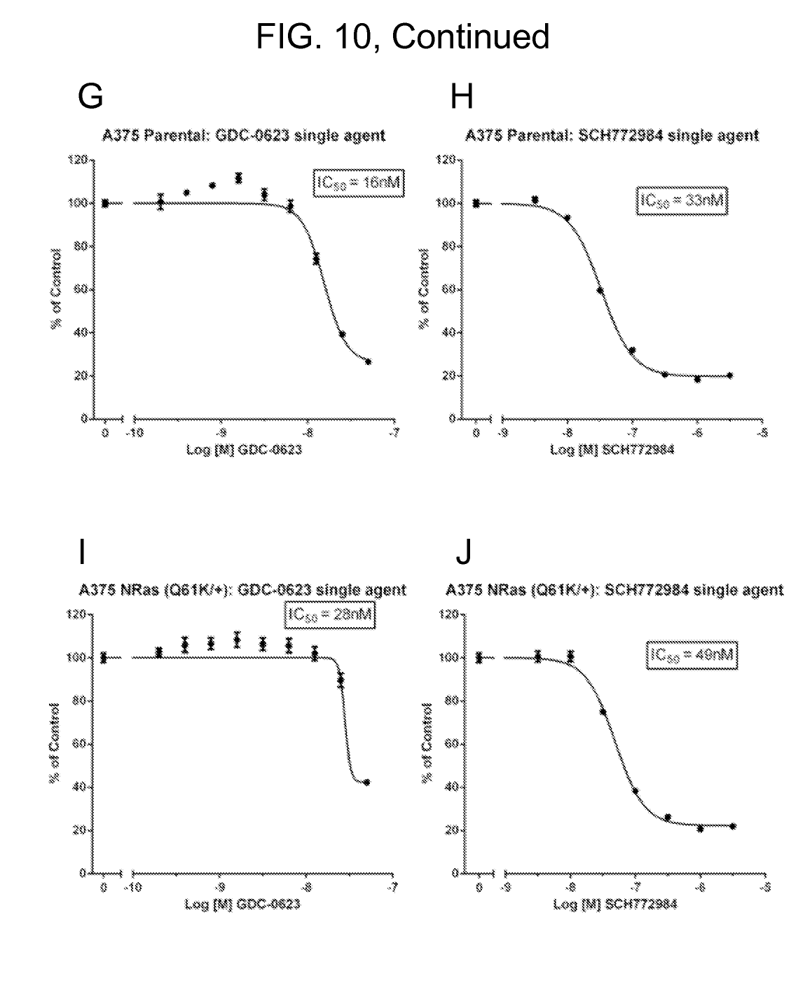

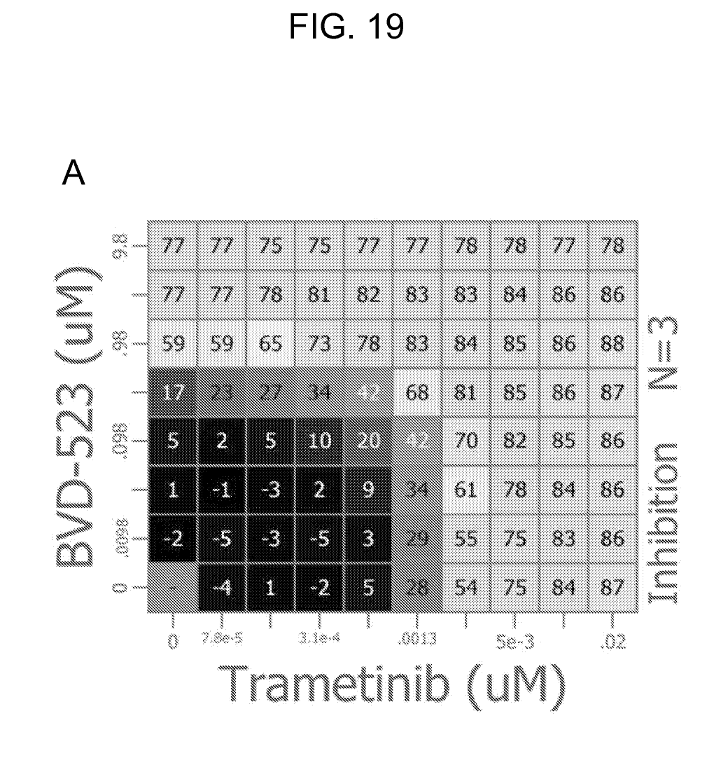

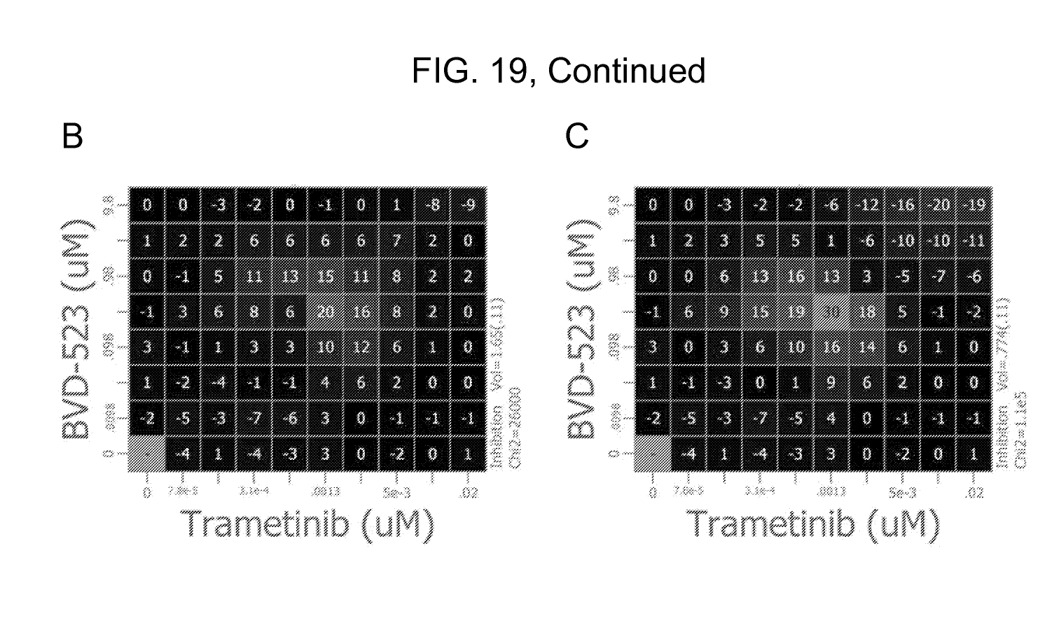

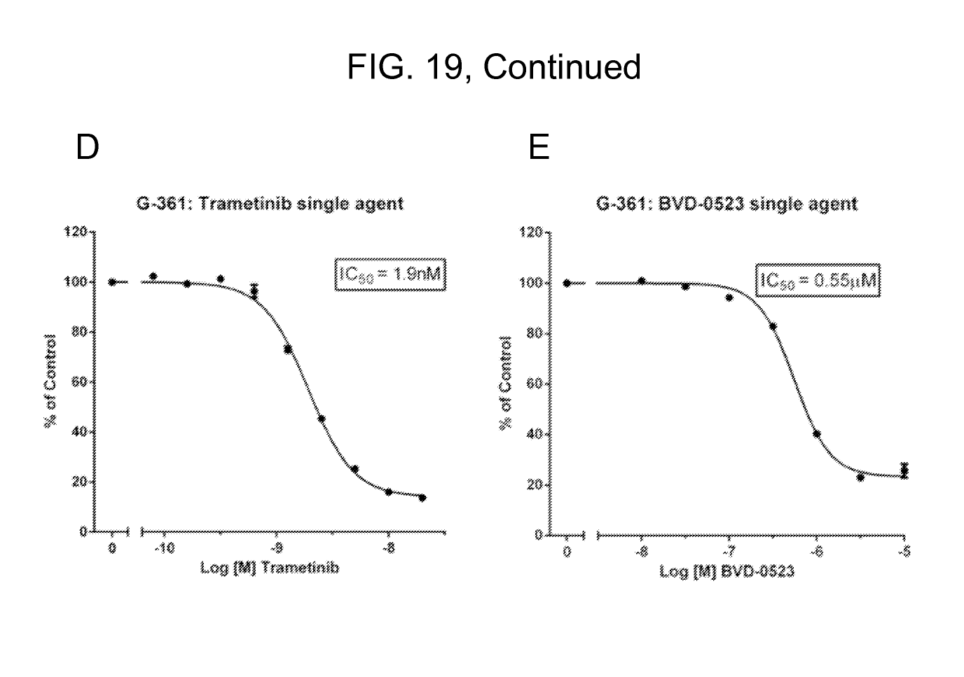

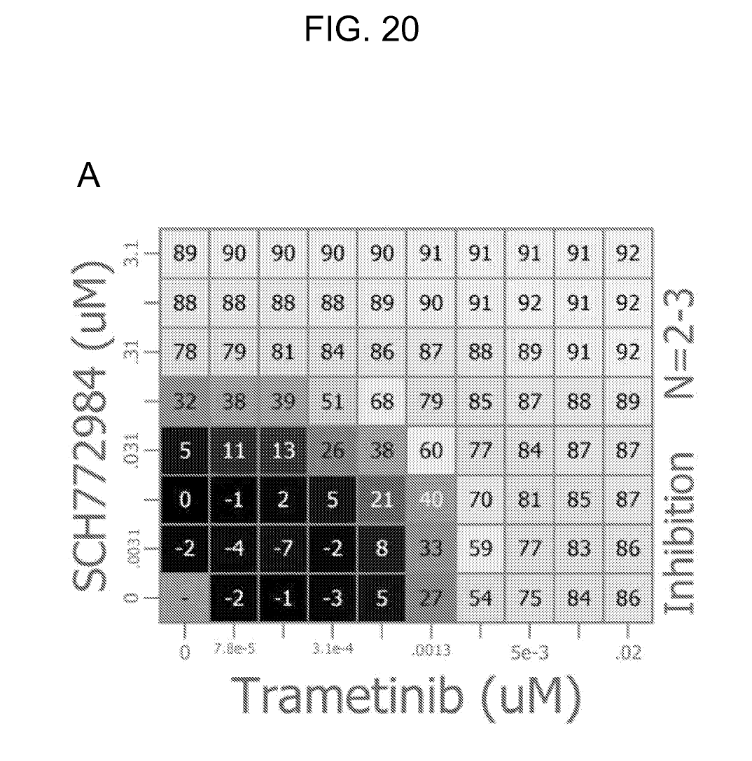

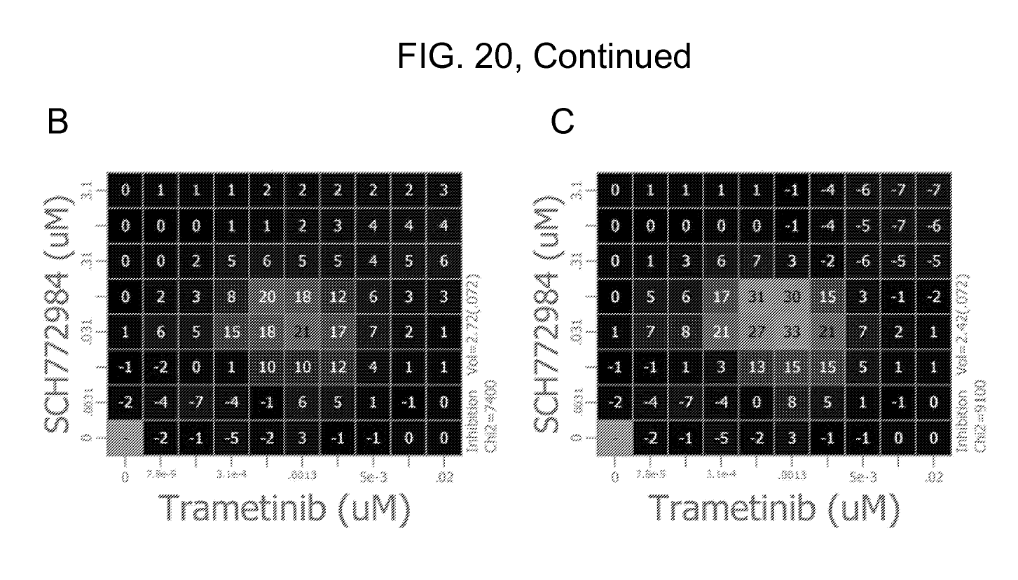

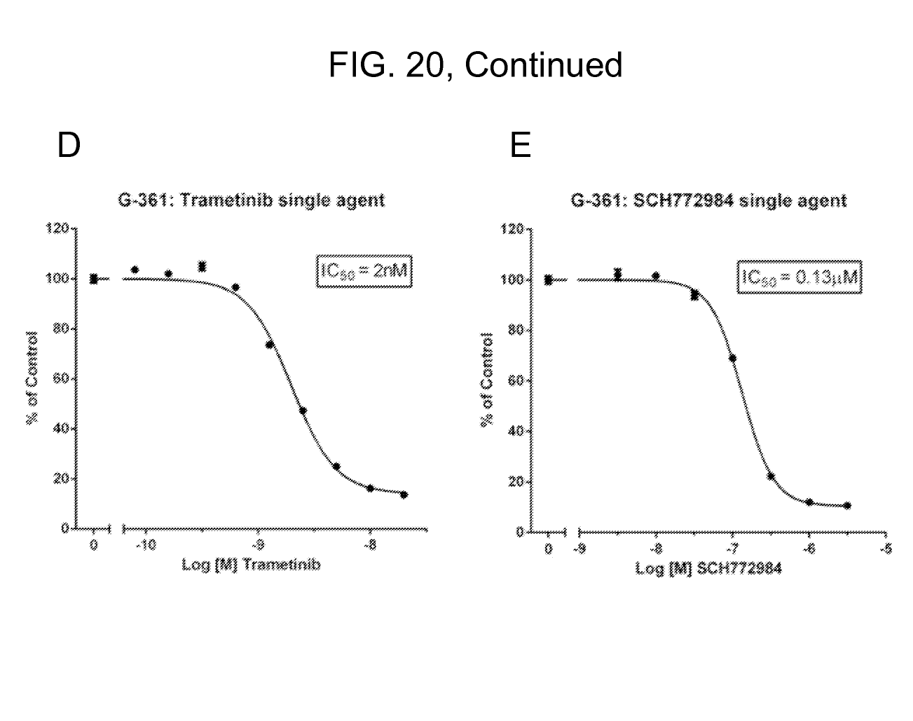

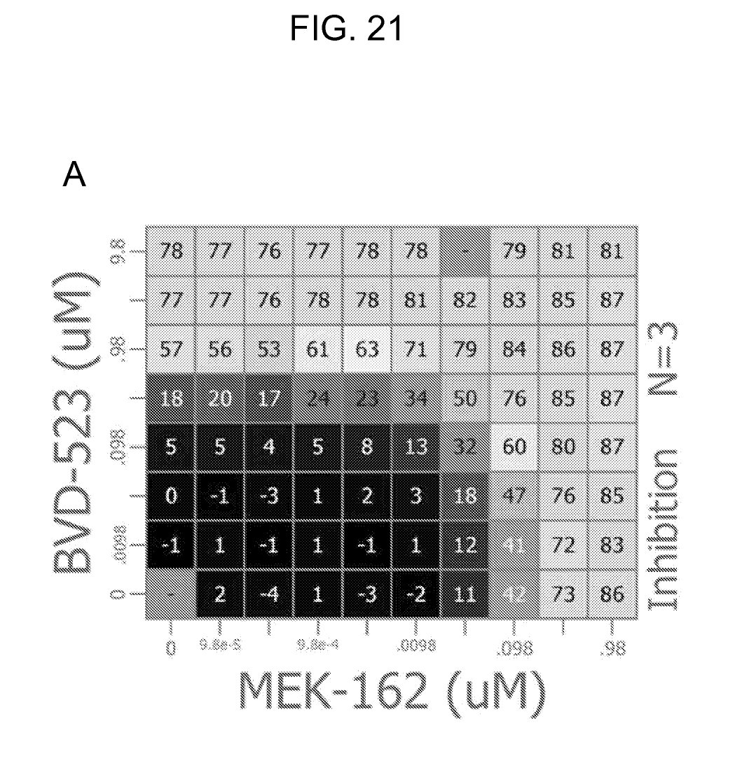

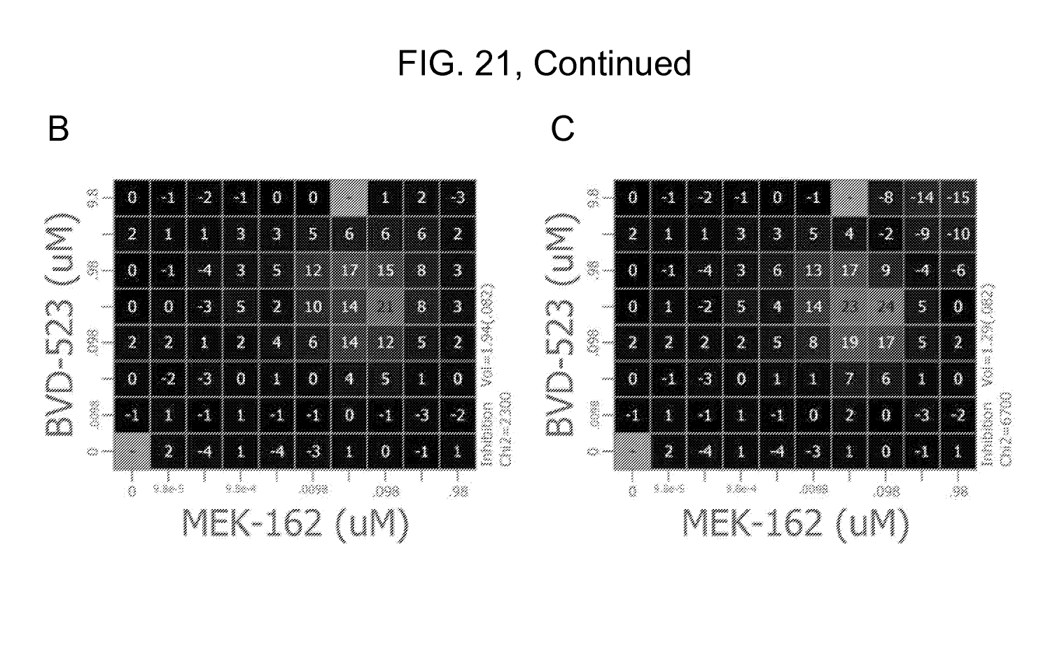

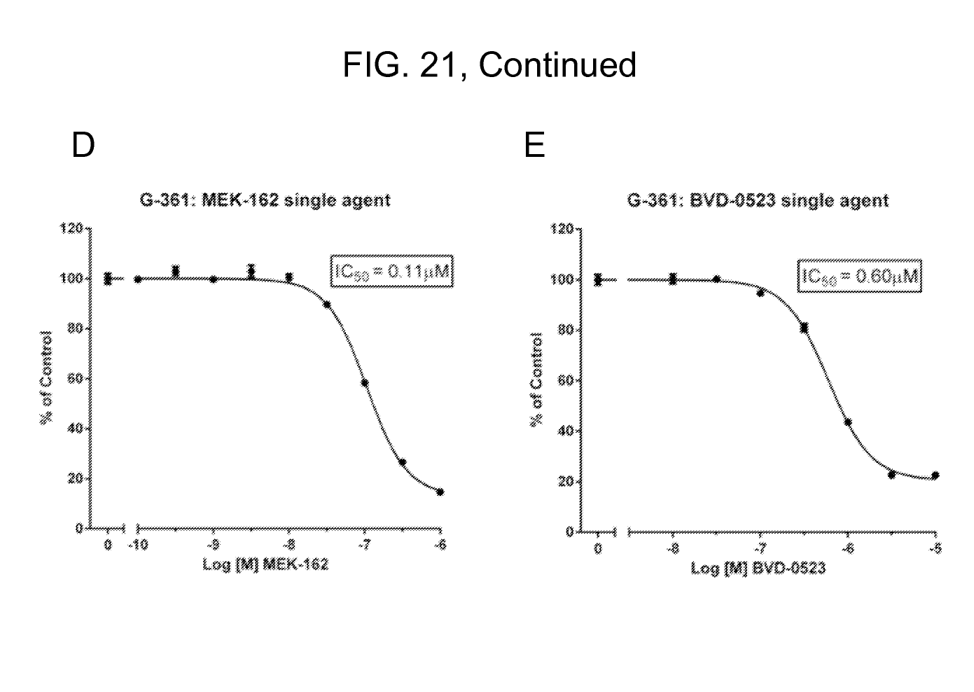

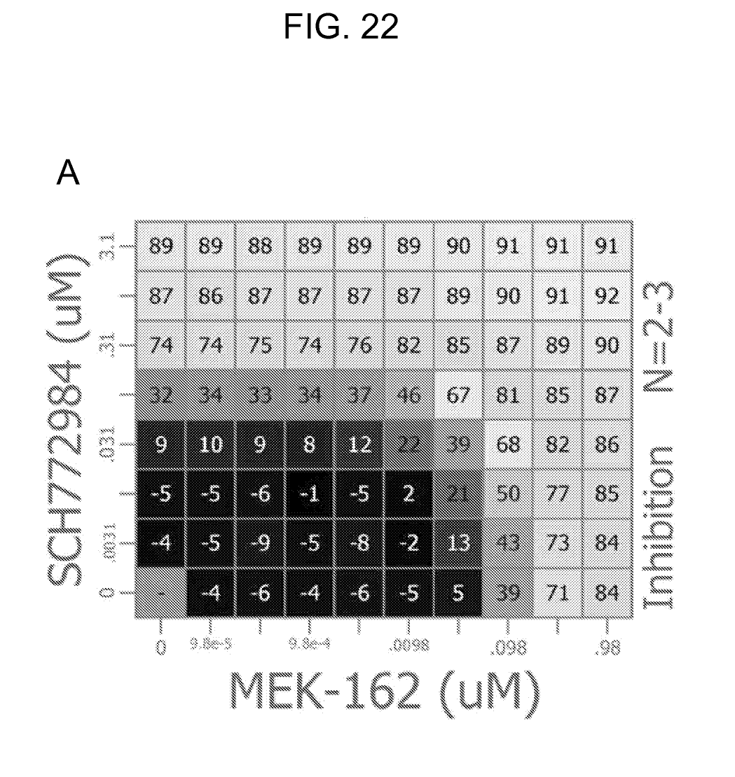

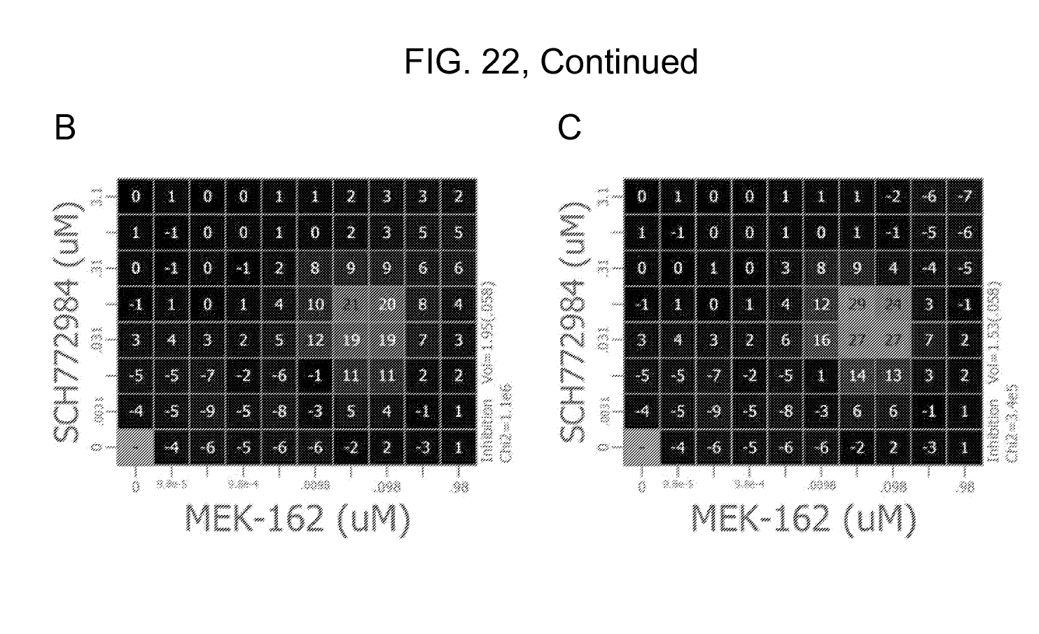

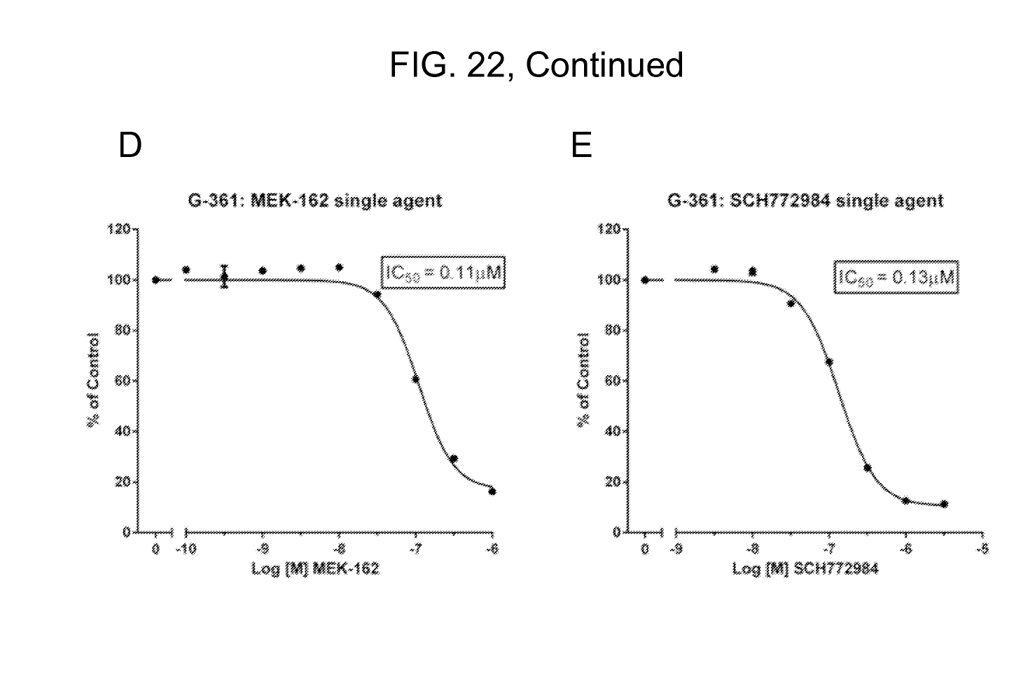

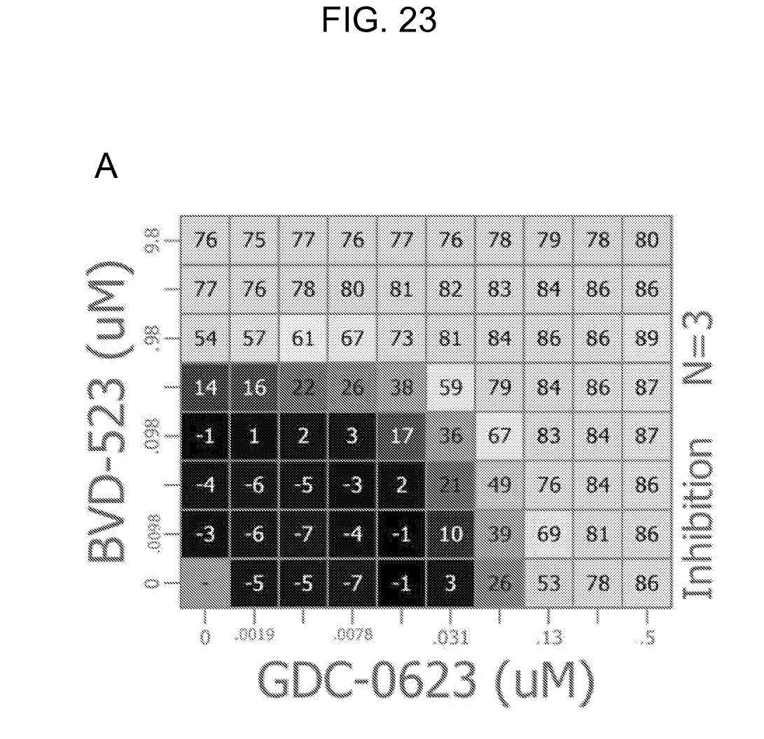

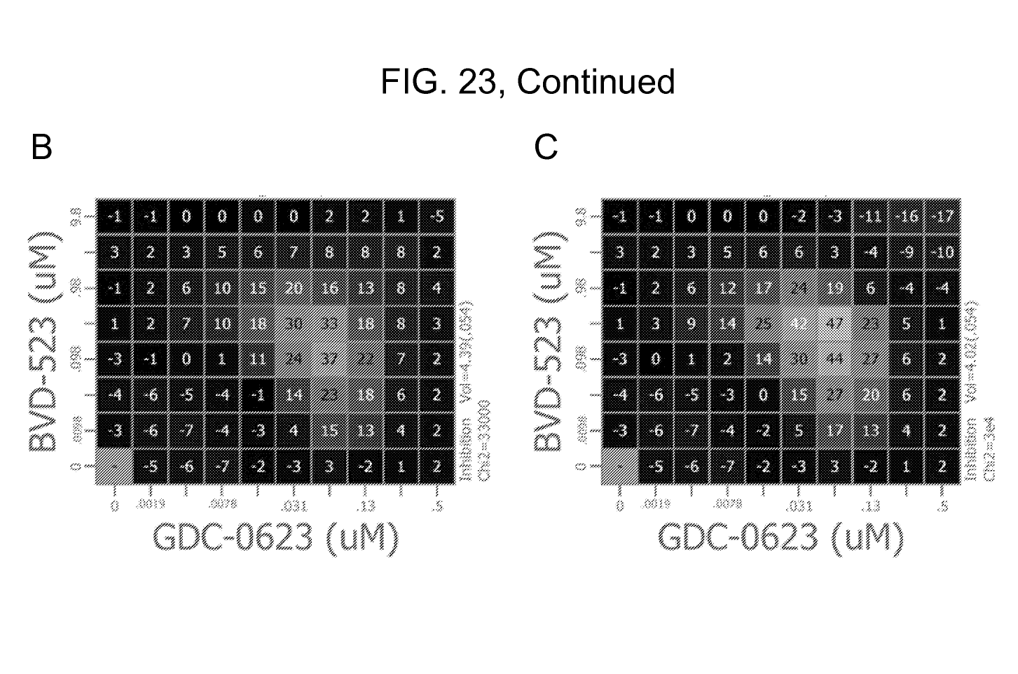

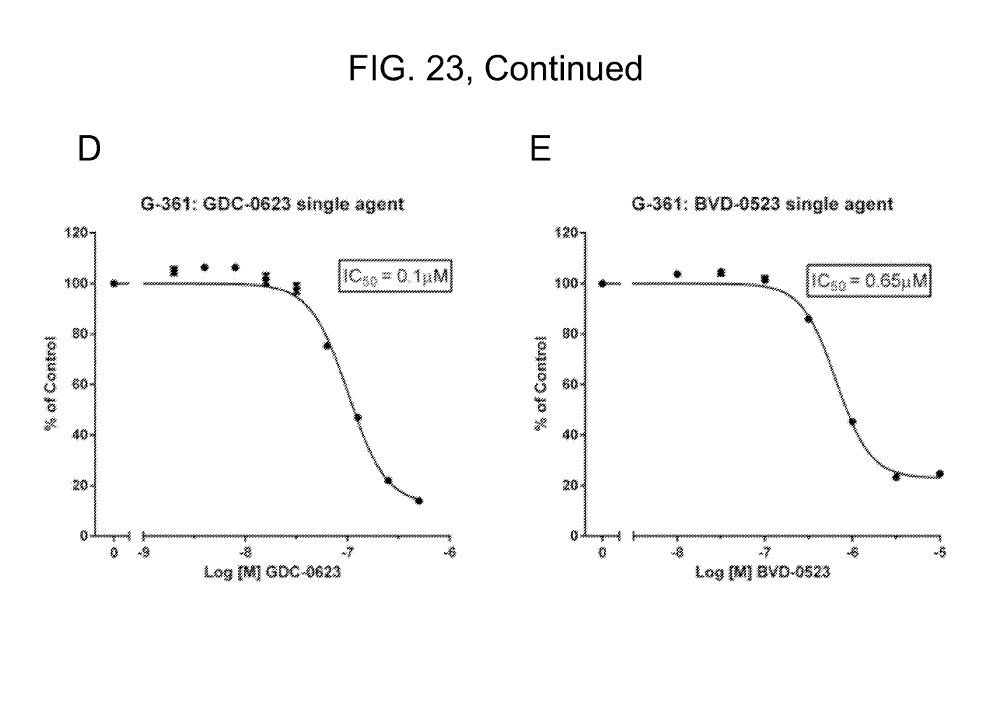

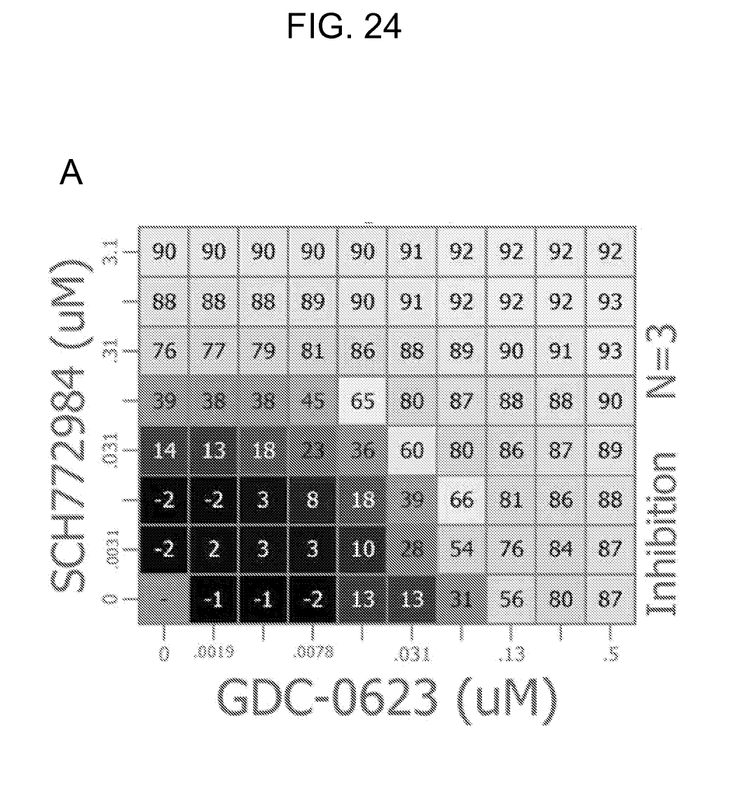

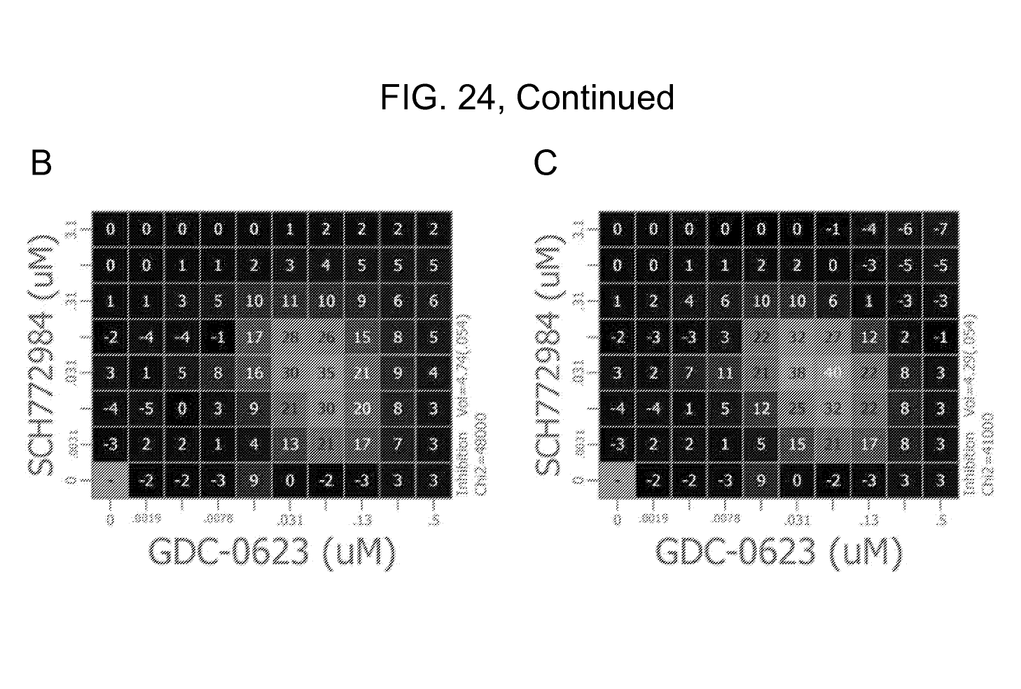

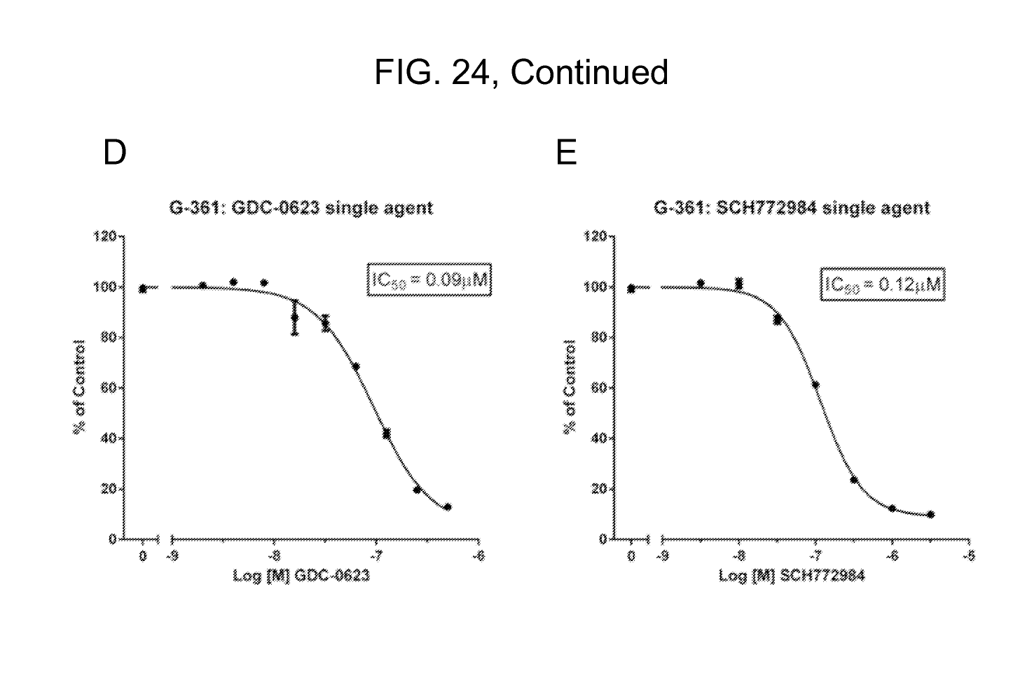

Visualization of the Bliss ‘excess inhibition’ heat maps for the A375 parental and NRAS mutant (Q61K) cell lines revealed a small window of synergy between BVD-523 and all three MEK inhibitors tested (FIG. 5, FIG. 7, FIG. 9). These observations were confirmed in a second BRAF mutant cell line G-361 (FIG. 19, FIG. 21, FIG. 23) and using a second benchmark ERK inhibitor SCH772984 (FIG. 6, FIG. 8, FIG. 10 and FIG. 20, FIG. 22, FIG. 24, respectively). Although not as strong, these windows of synergy were also mostly detected when the data was analyzed using the Loewe model.

In summary, these results suggest that interactions between BVD-523 and MEK inhibitors may potentially be synergistic in melanoma cell lines mutated for BRAF.

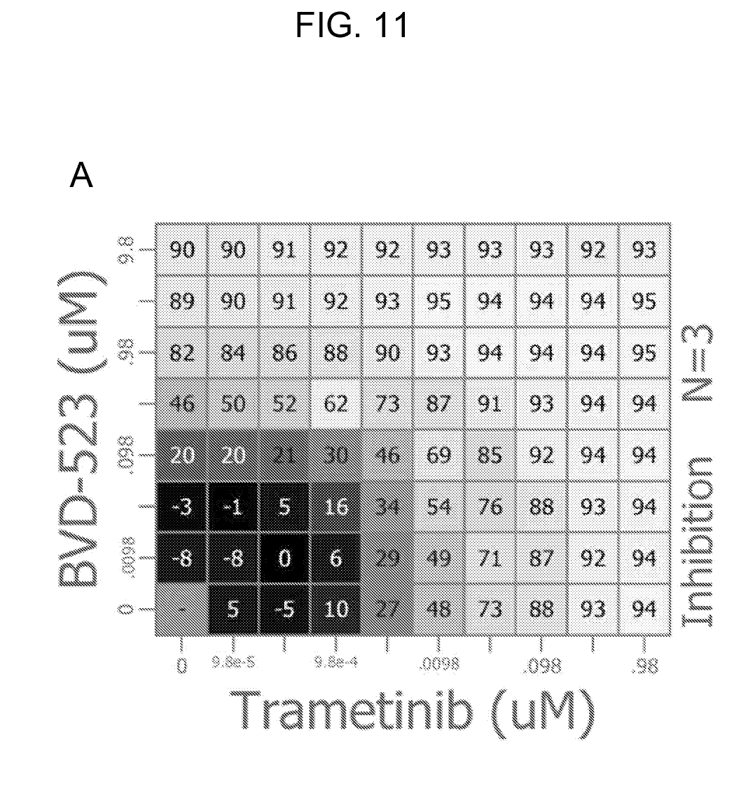

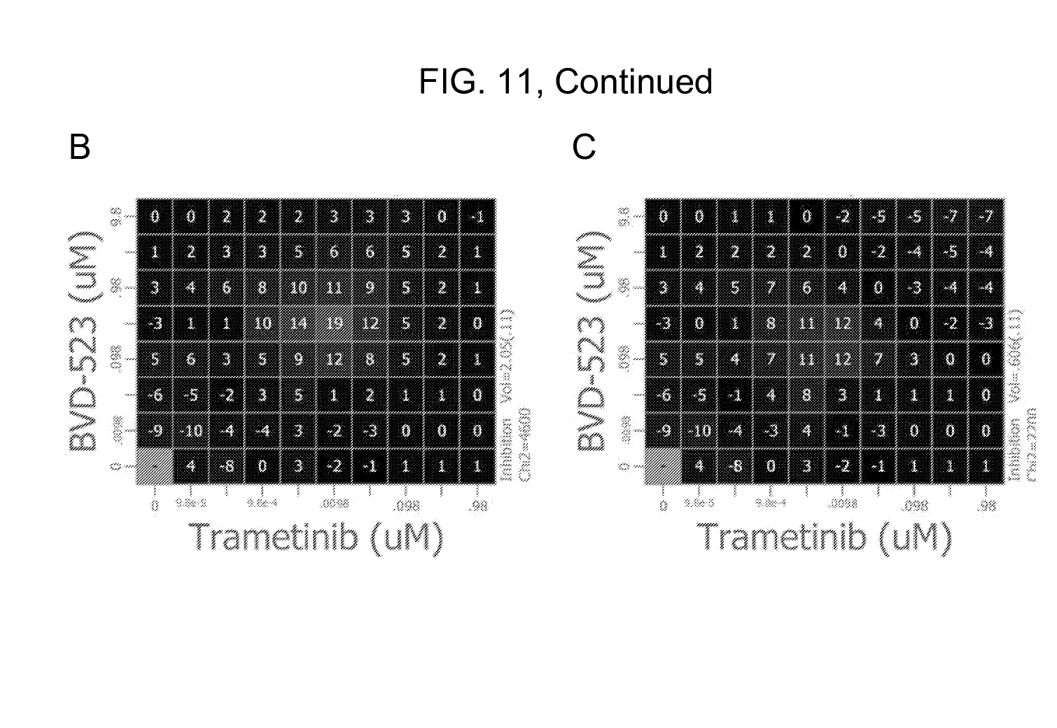

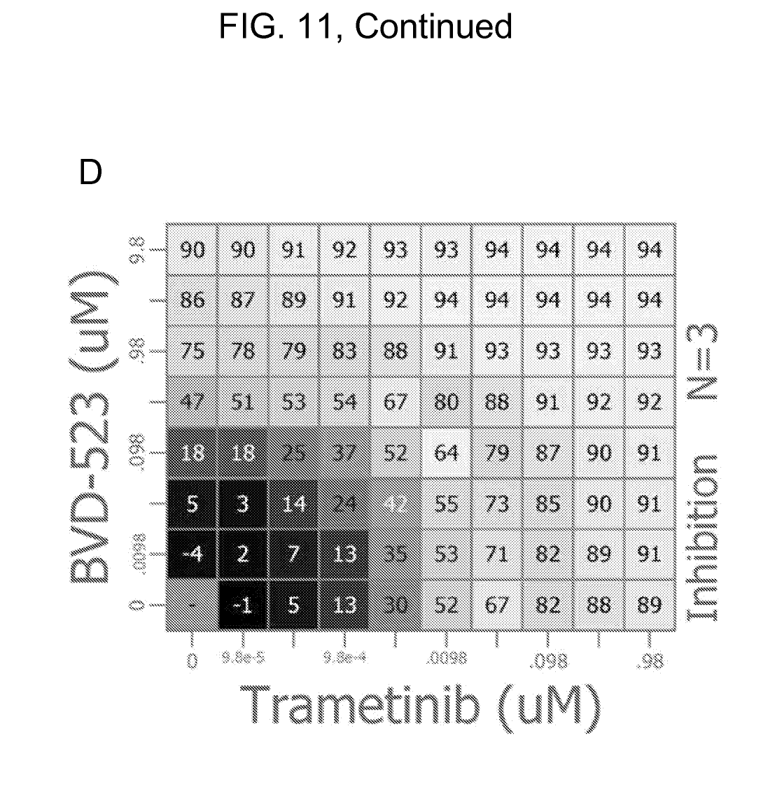

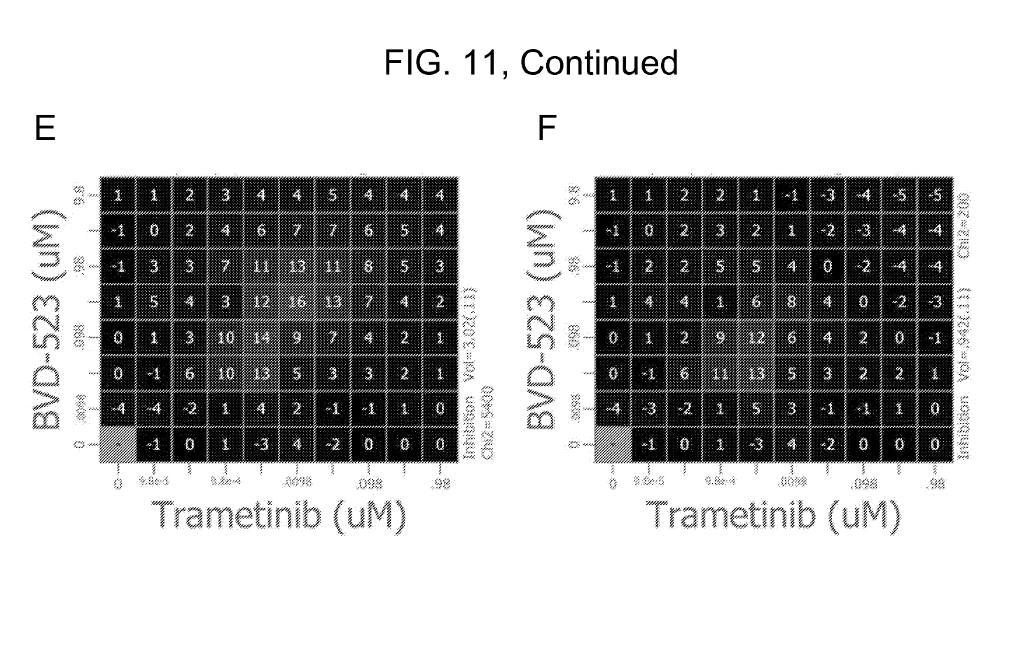

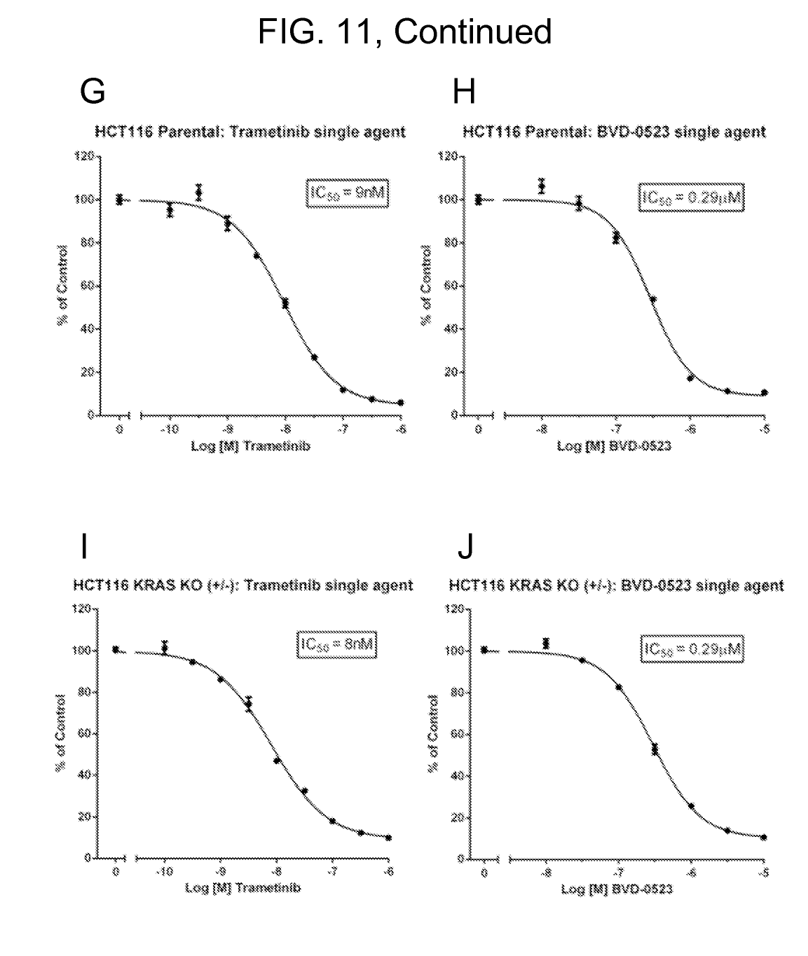

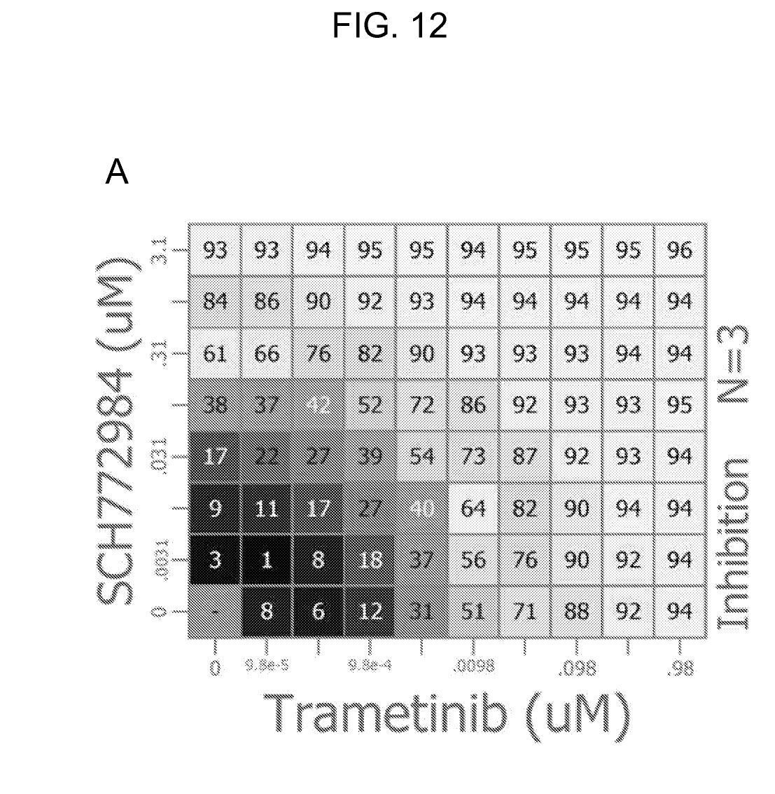

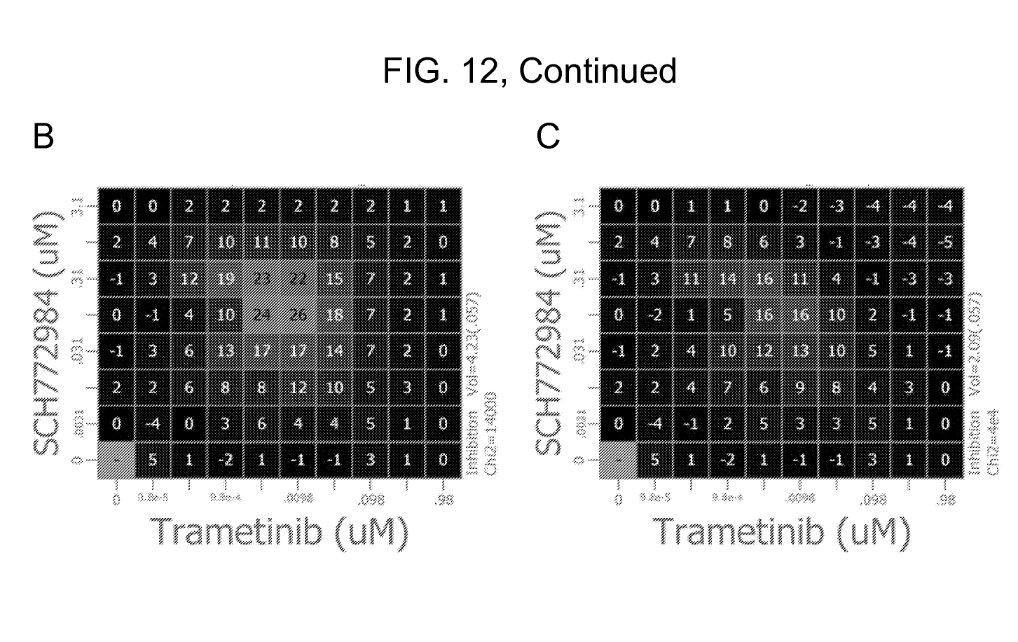

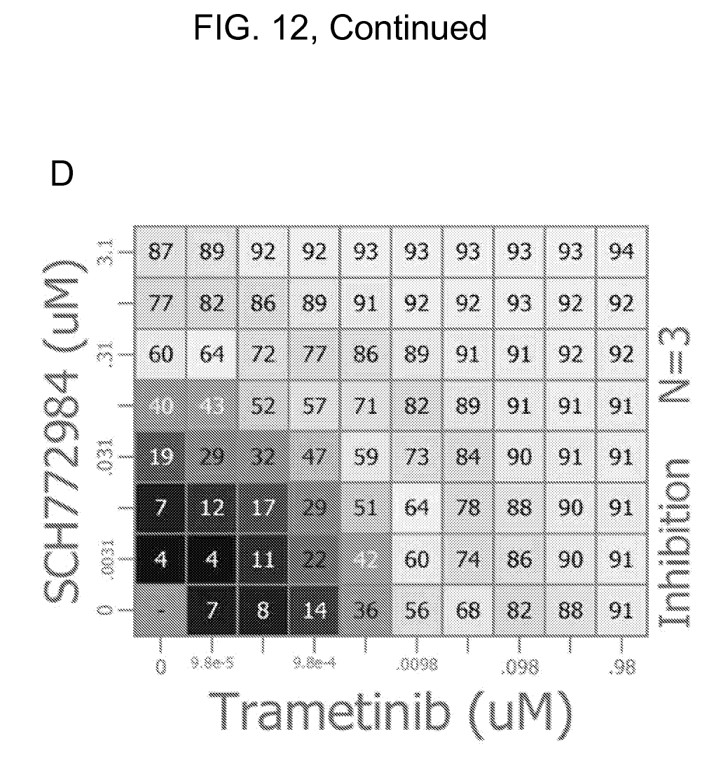

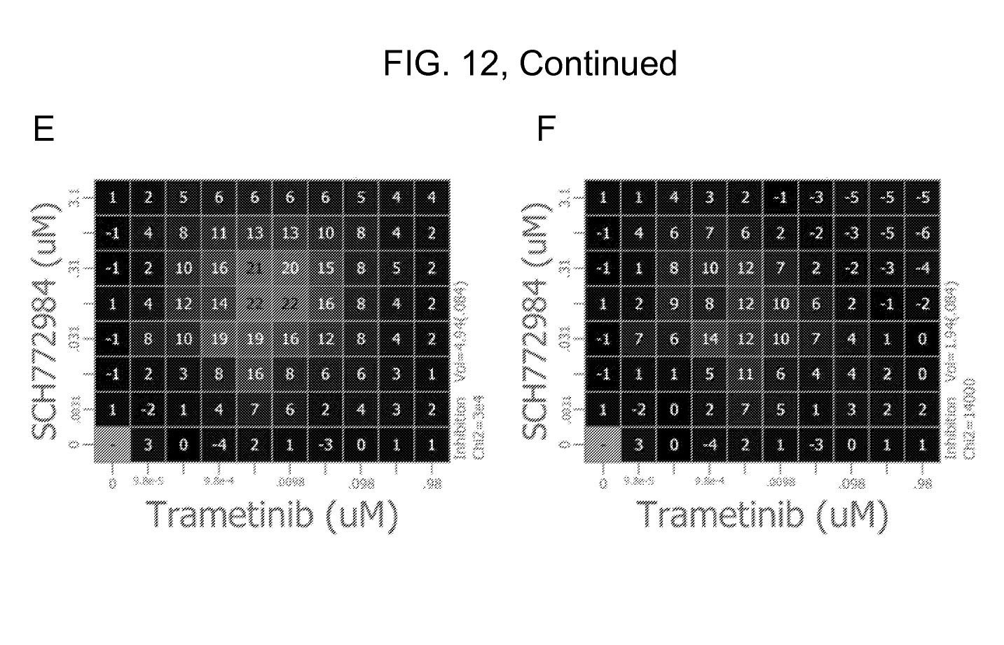

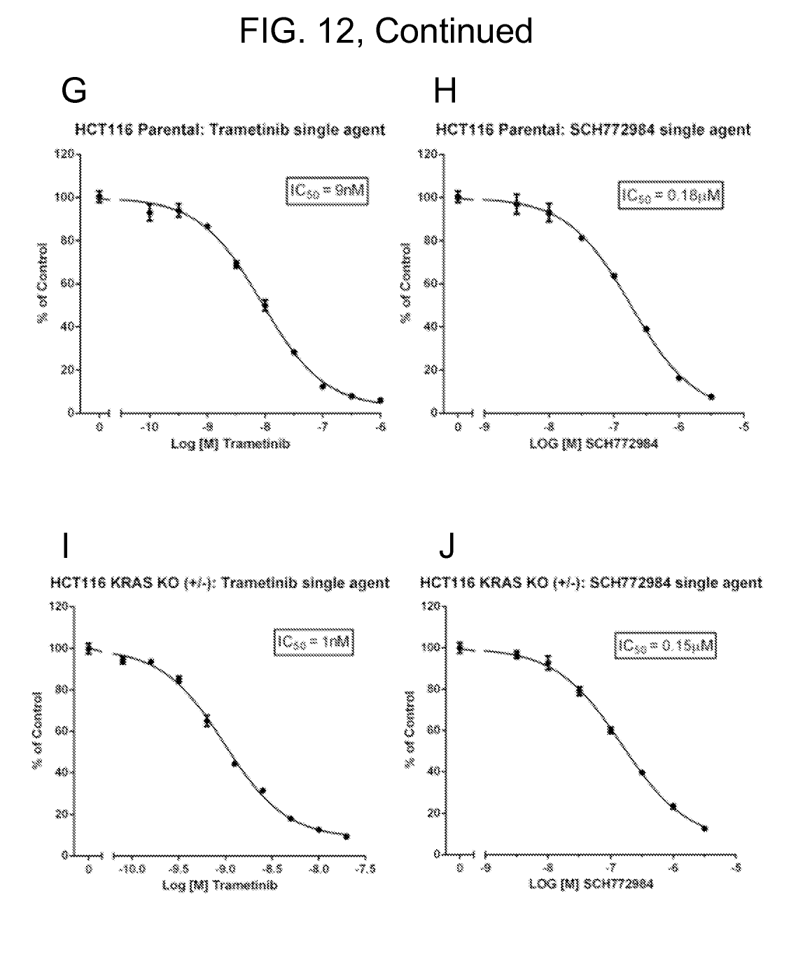

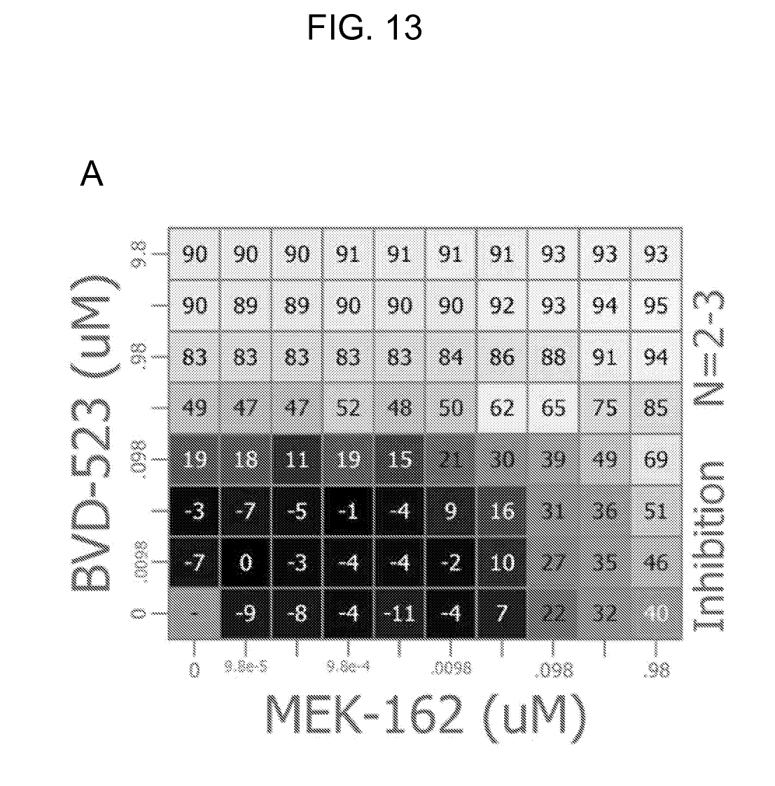

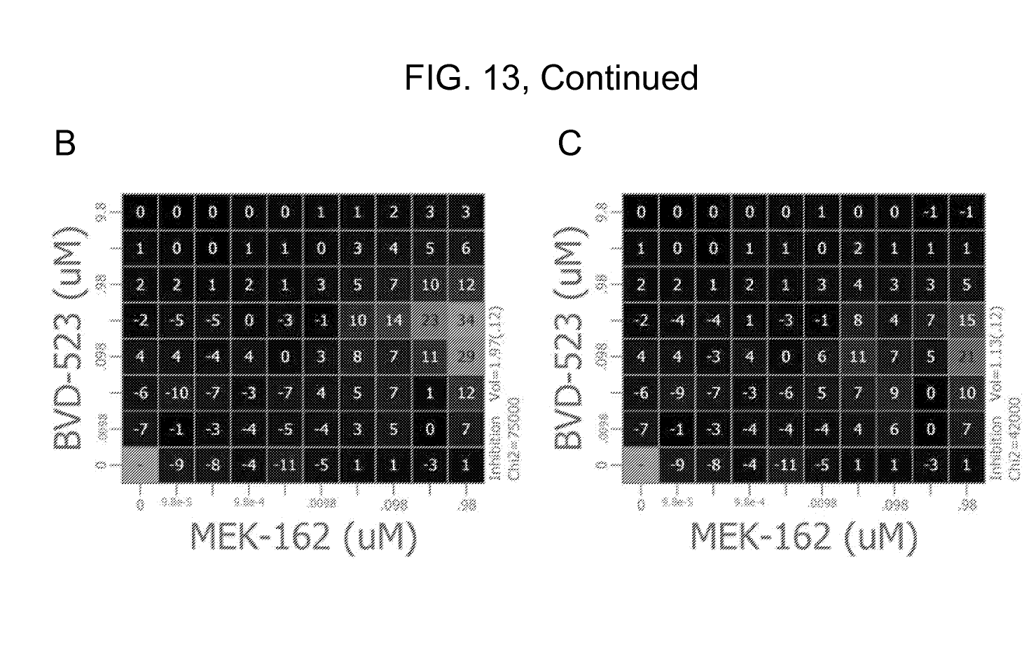

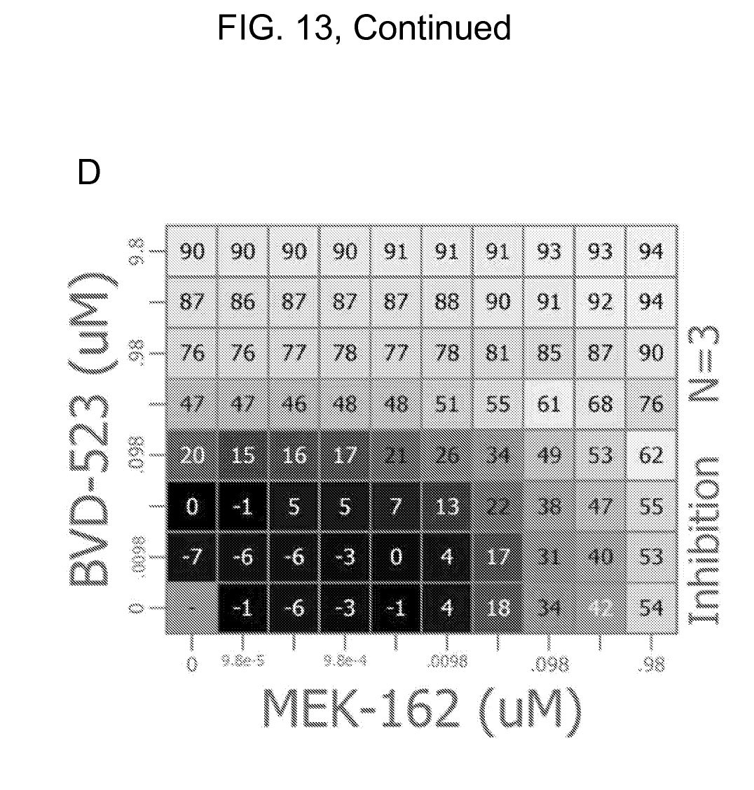

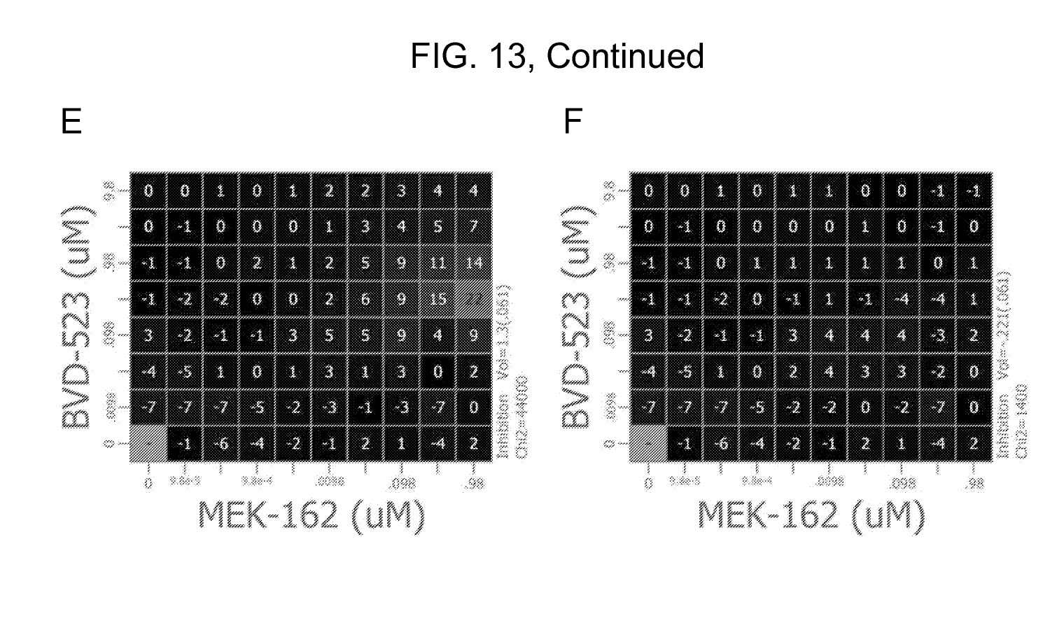

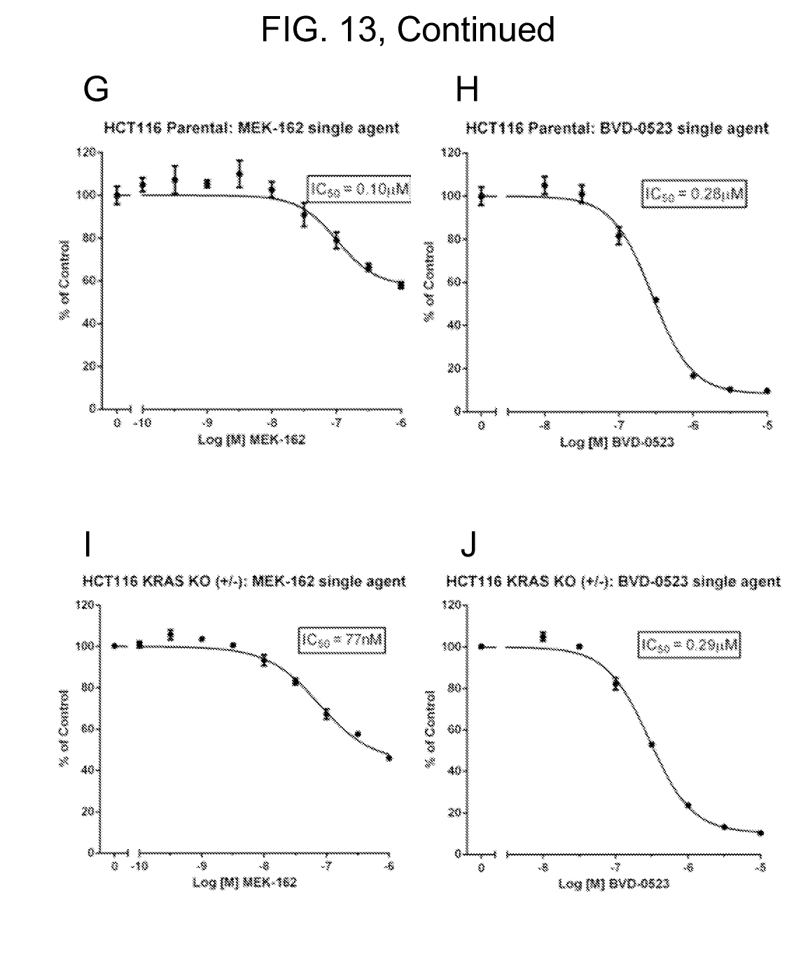

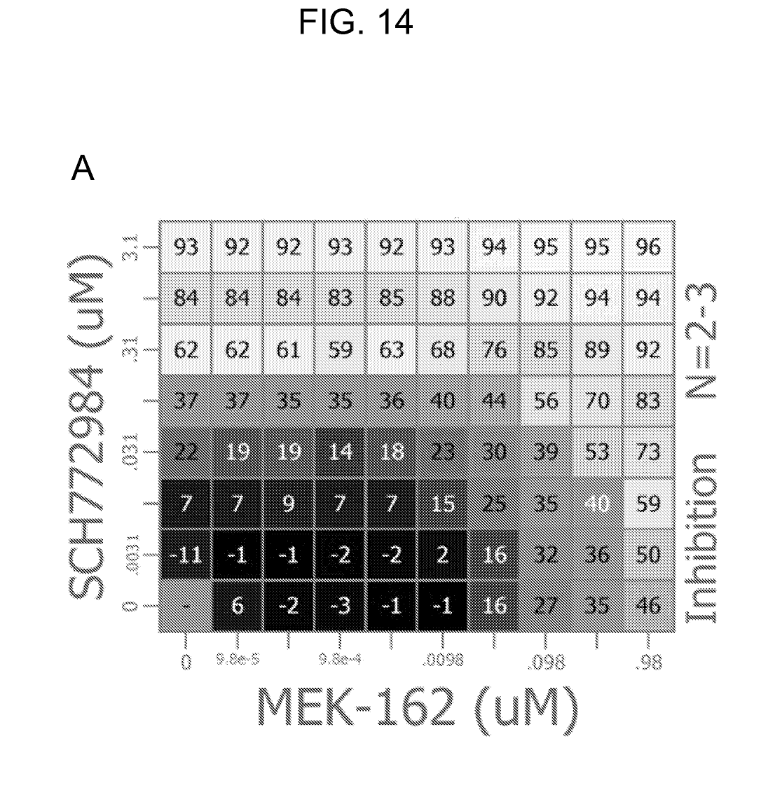

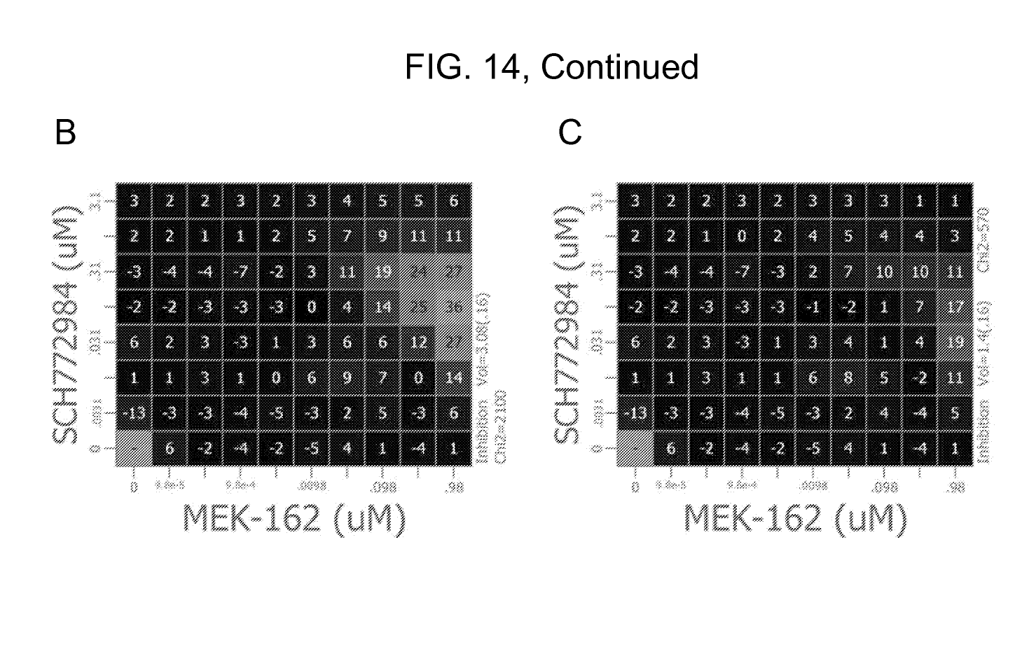

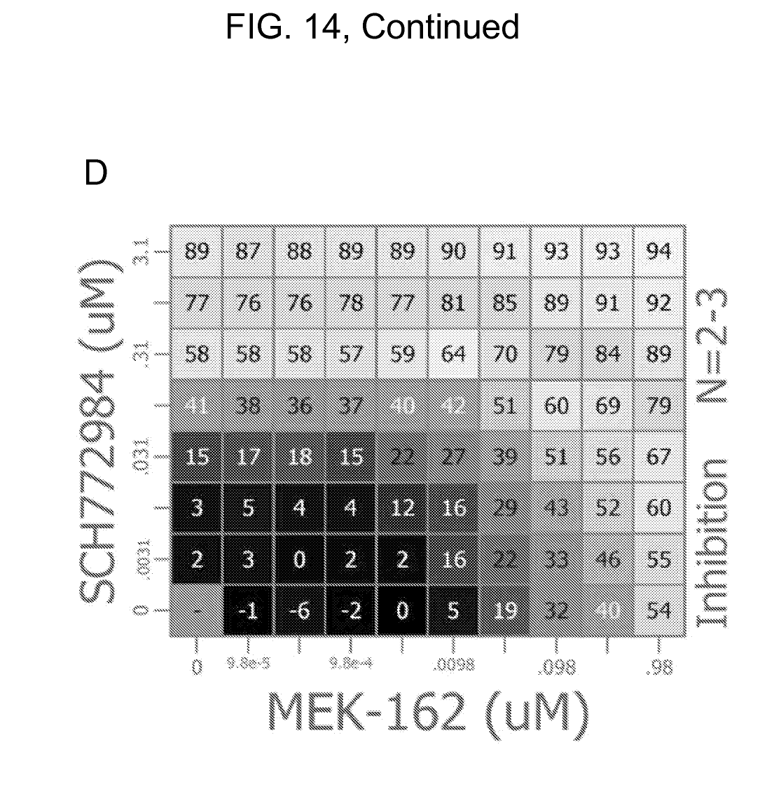

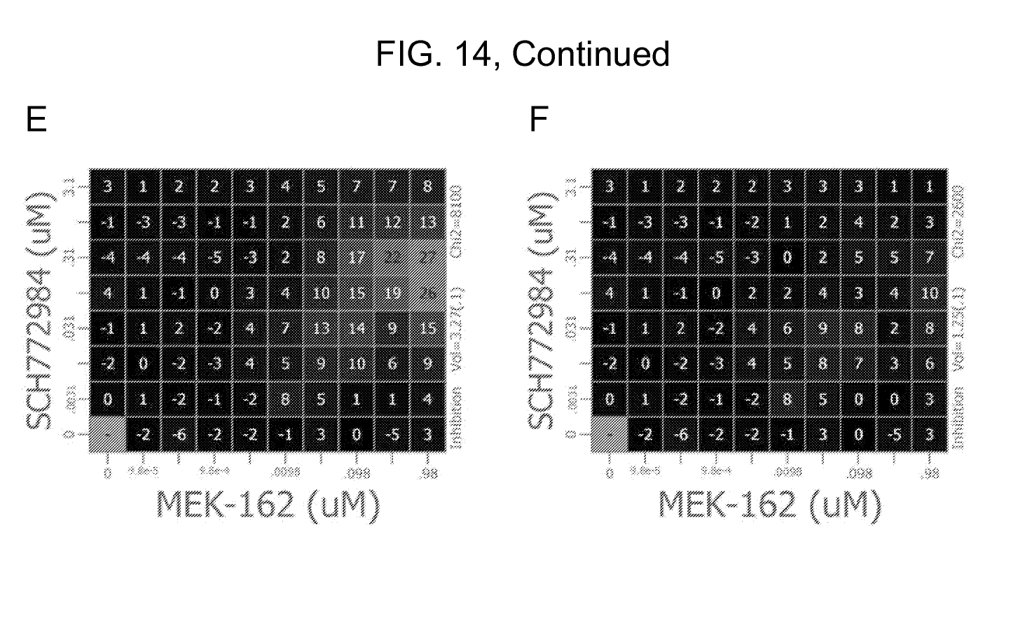

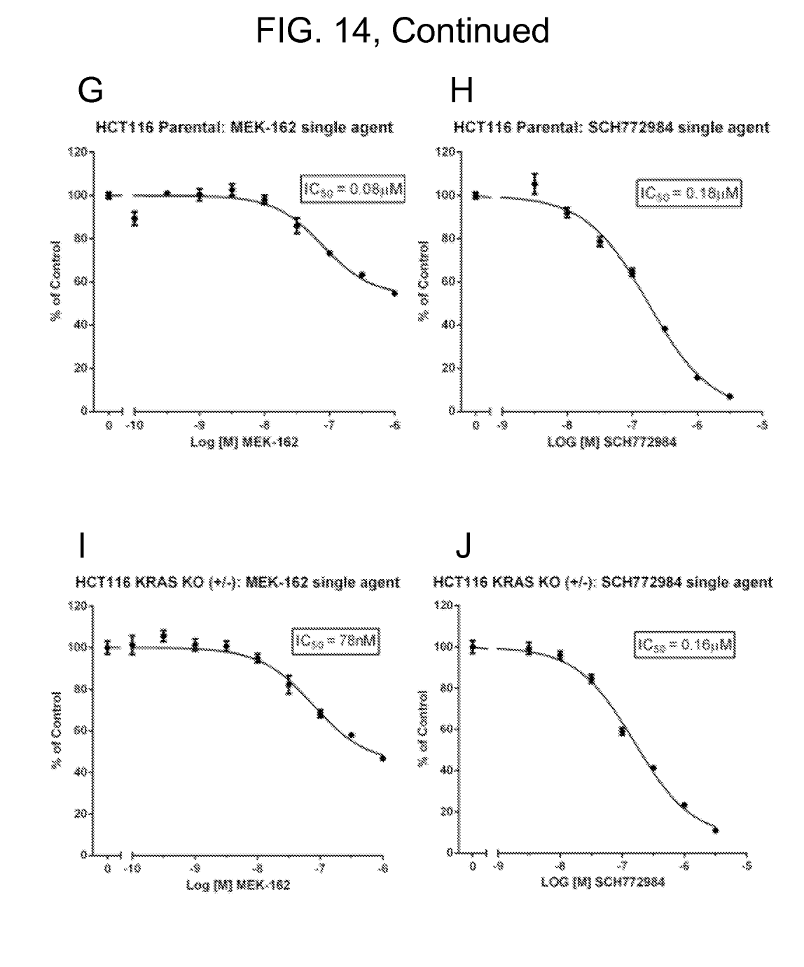

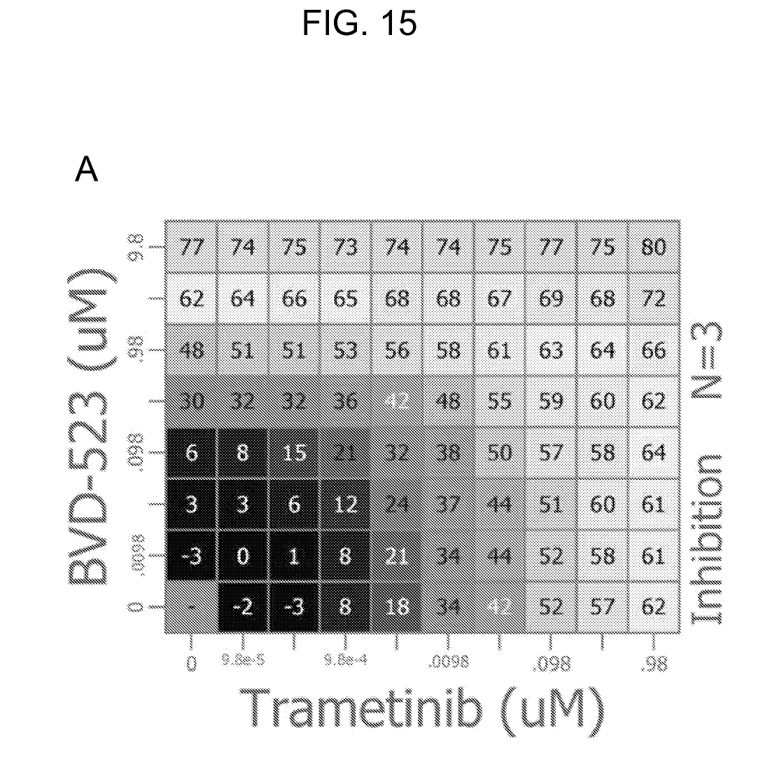

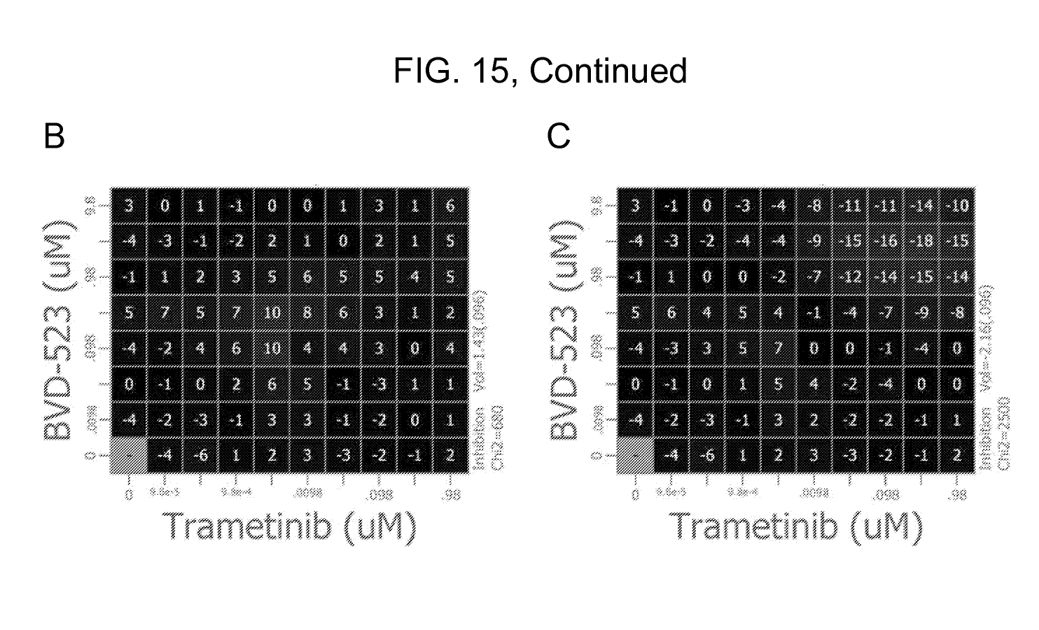

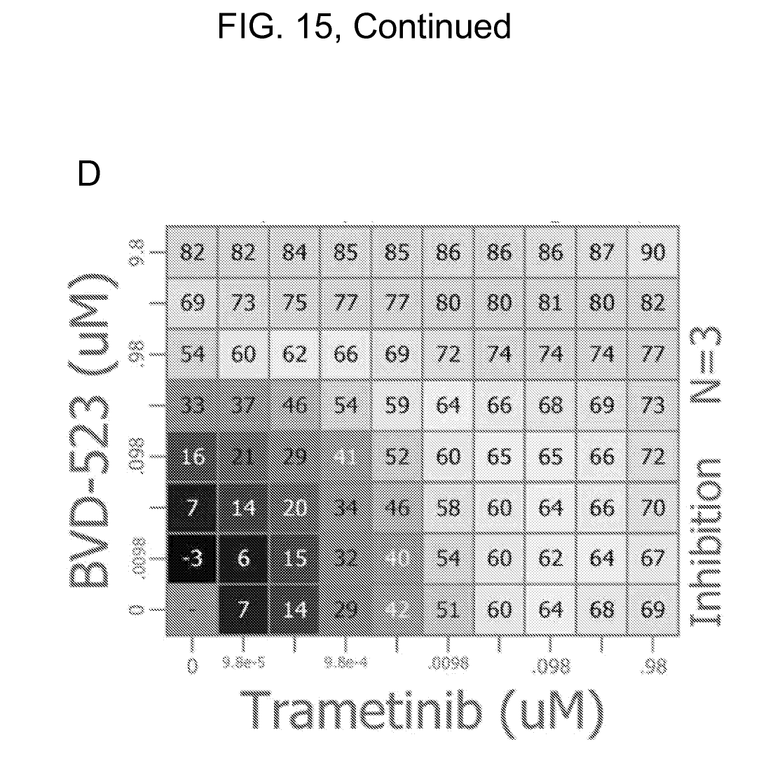

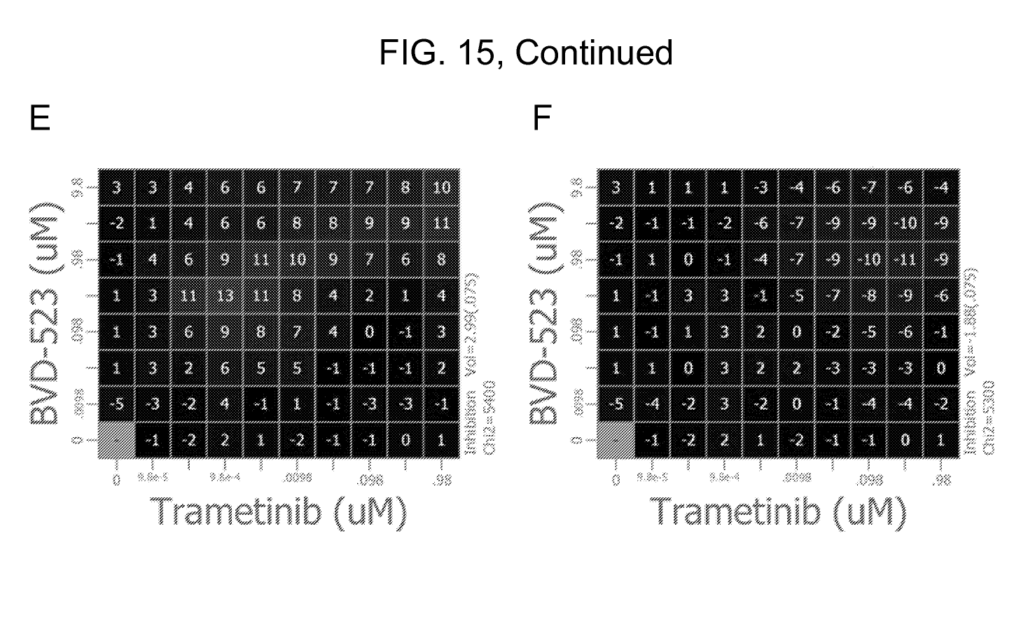

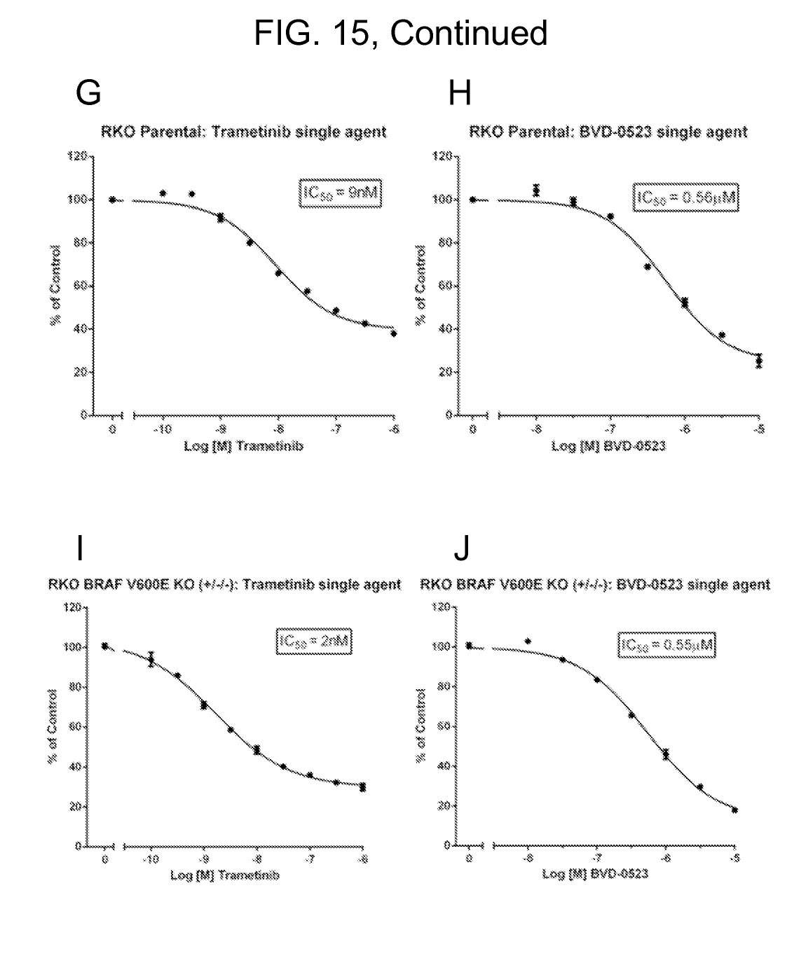

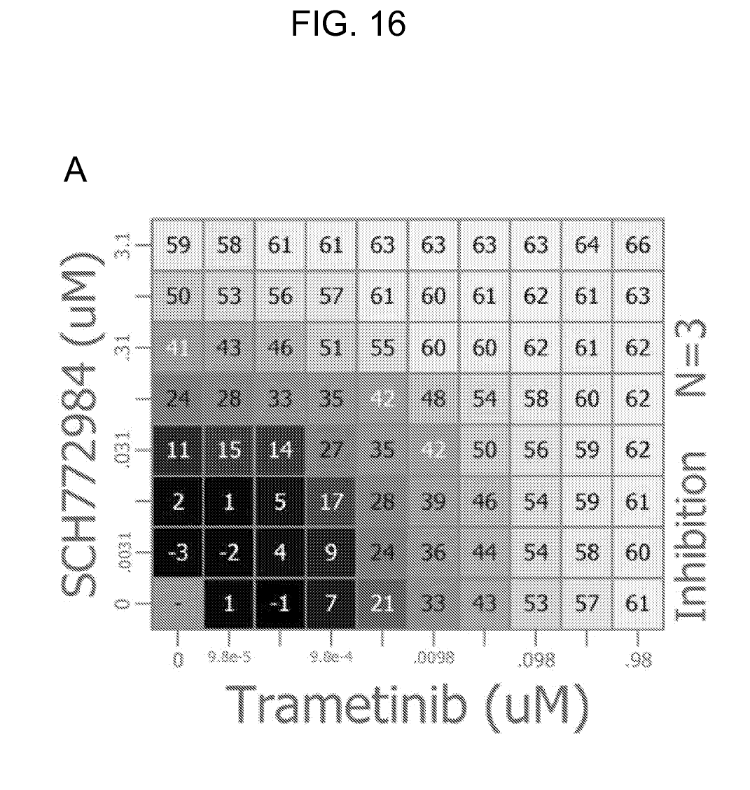

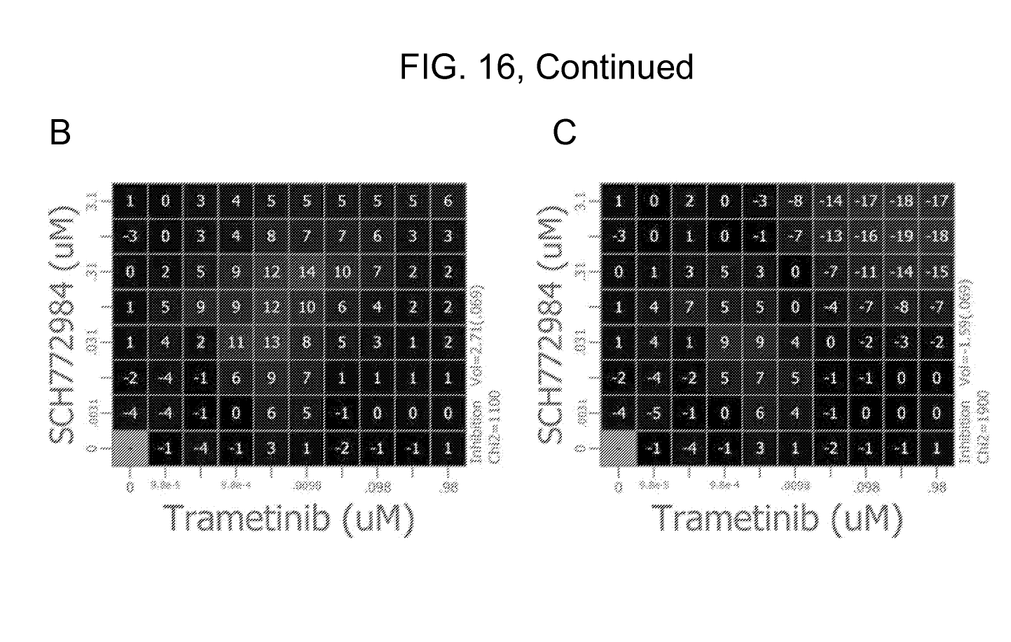

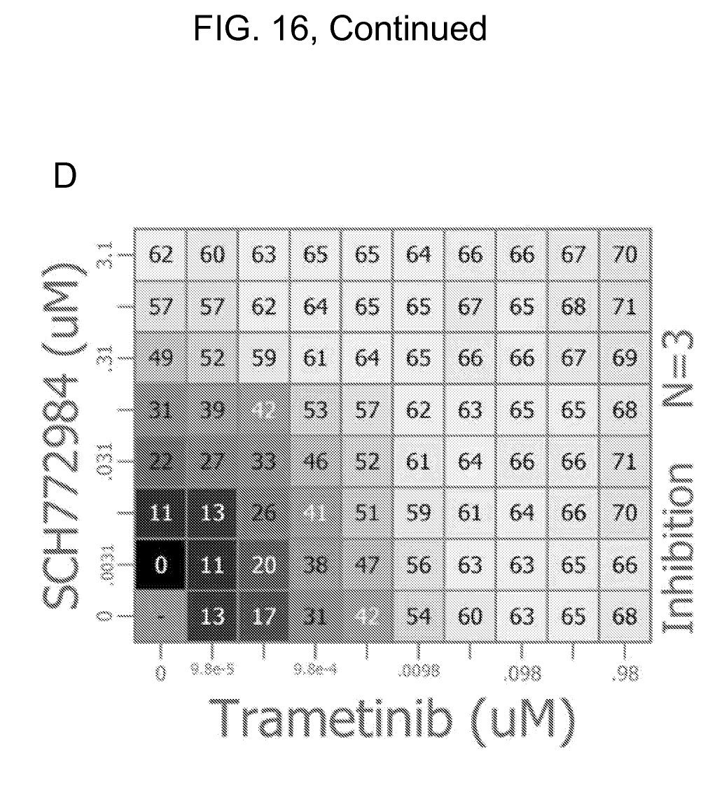

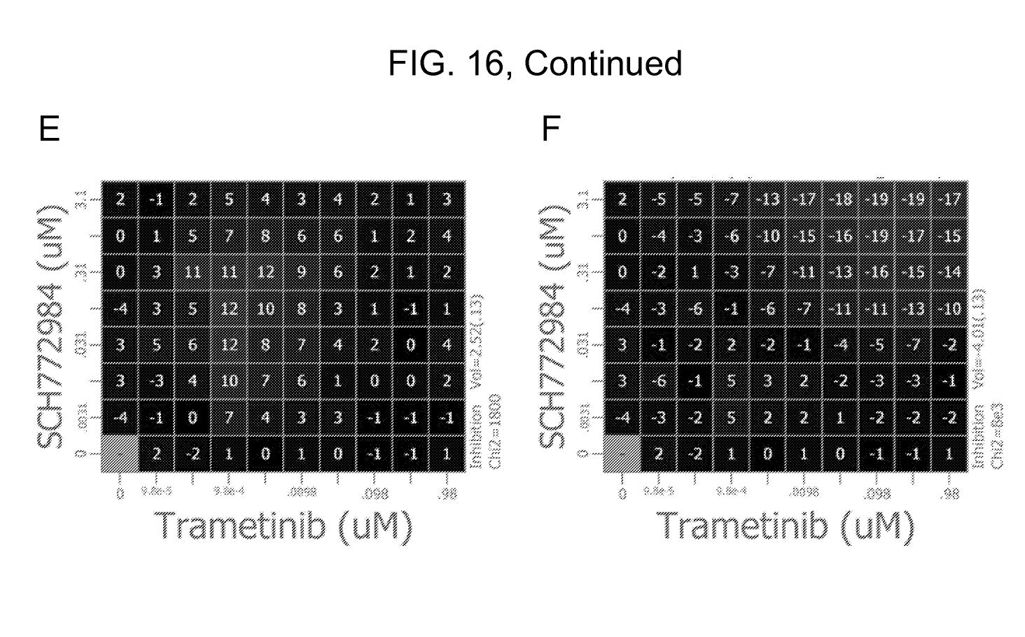

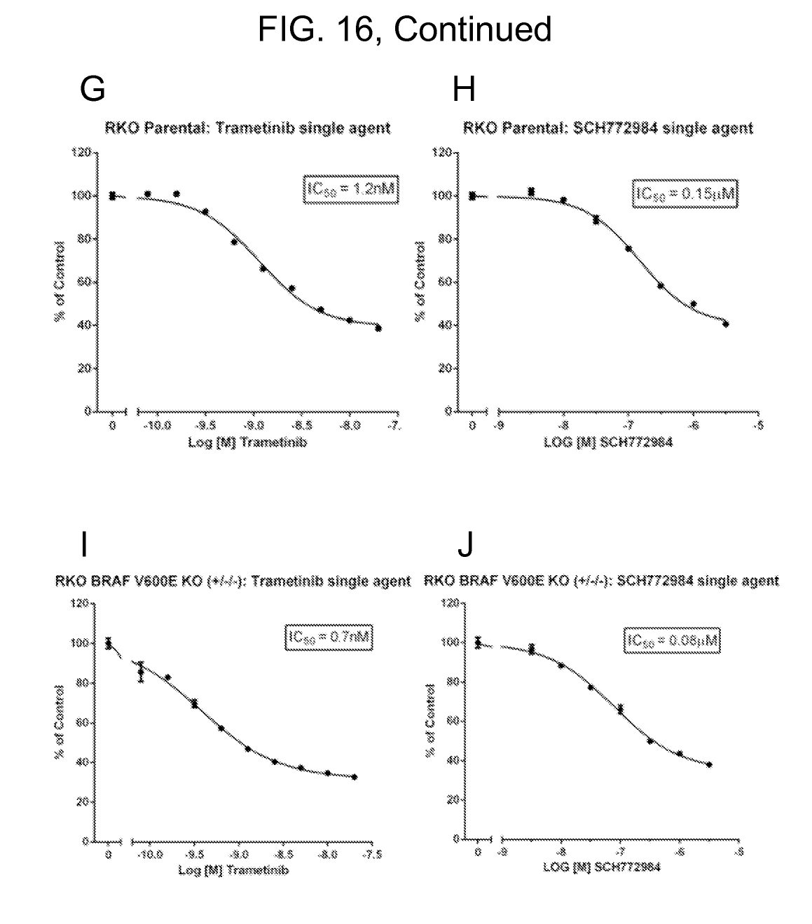

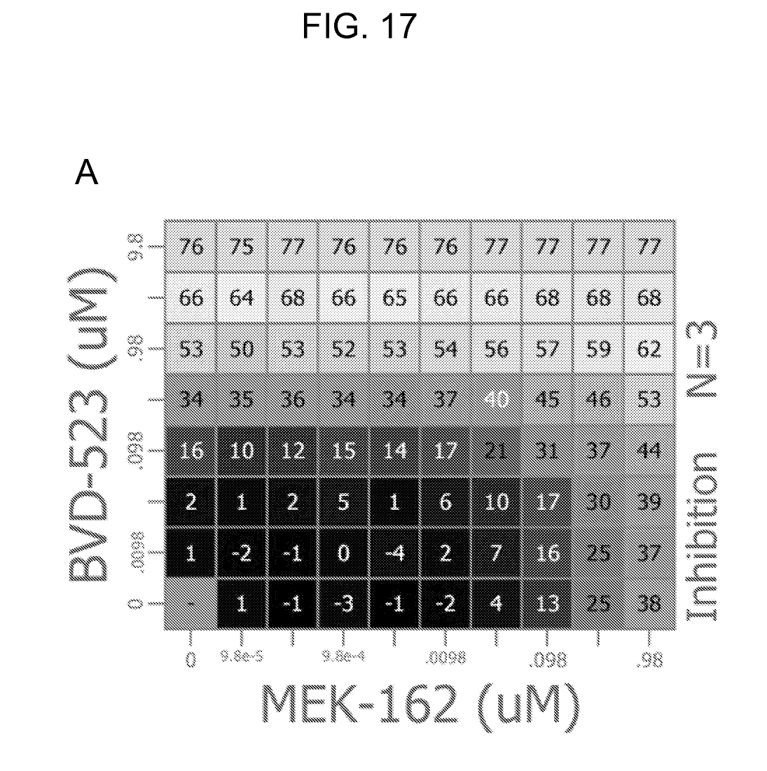

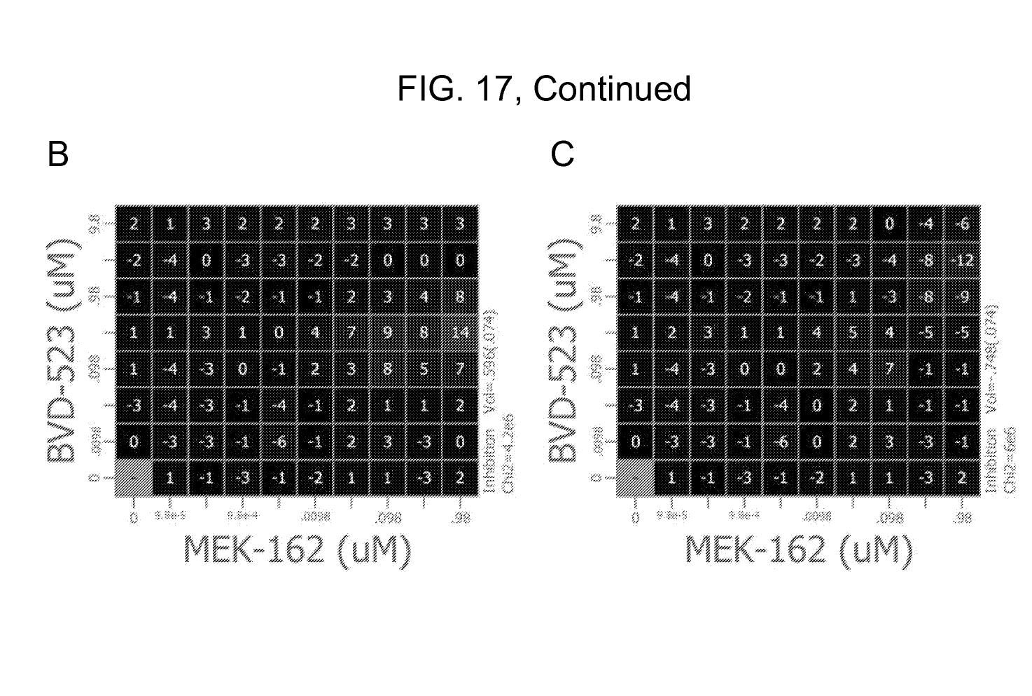

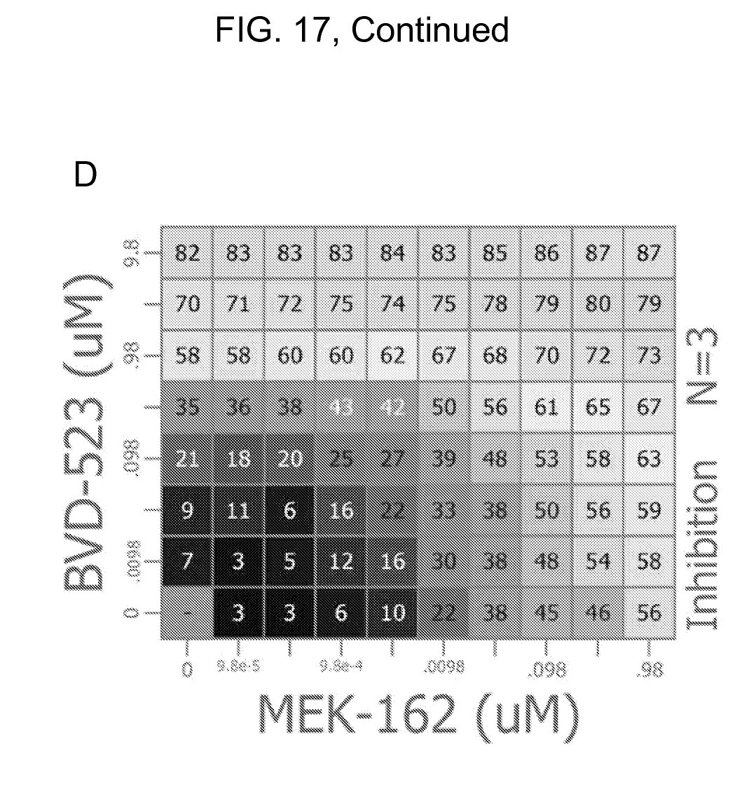

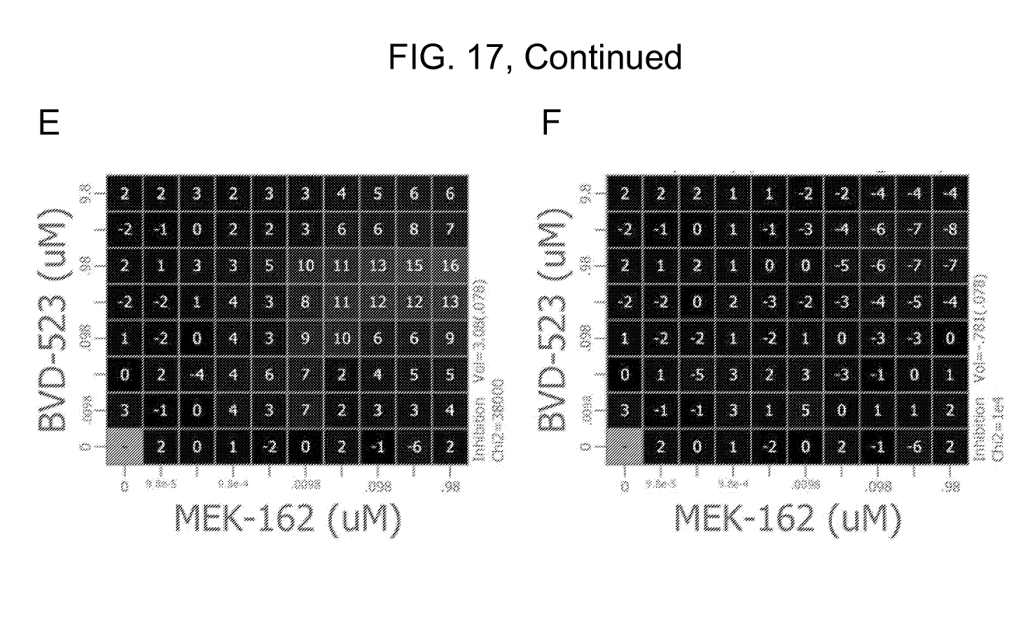

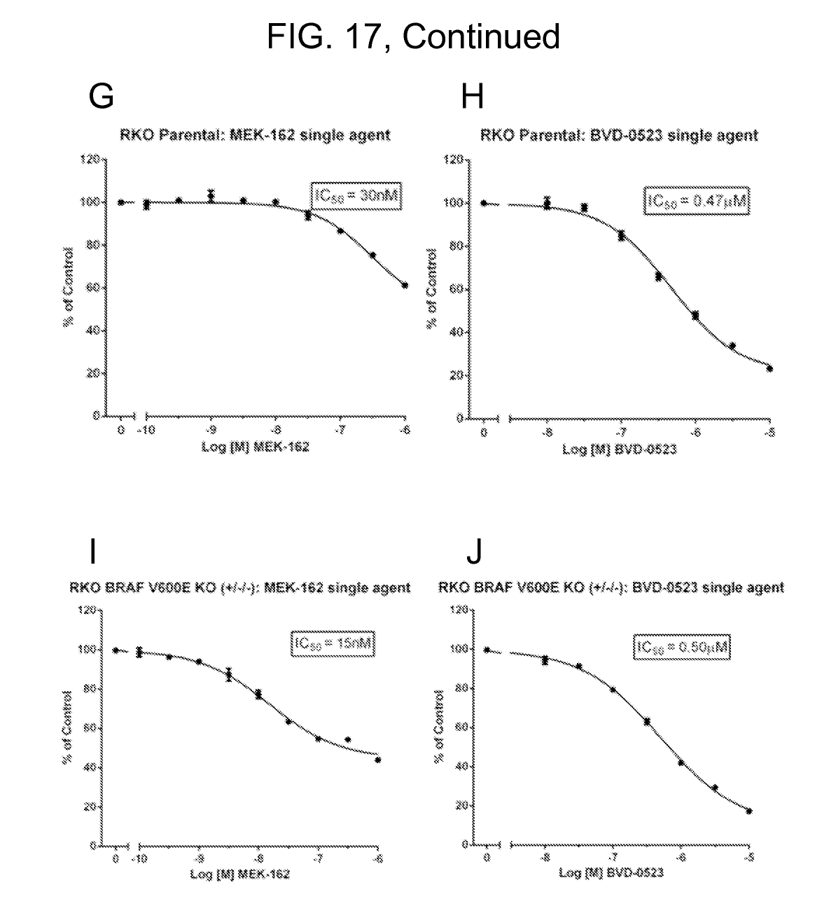

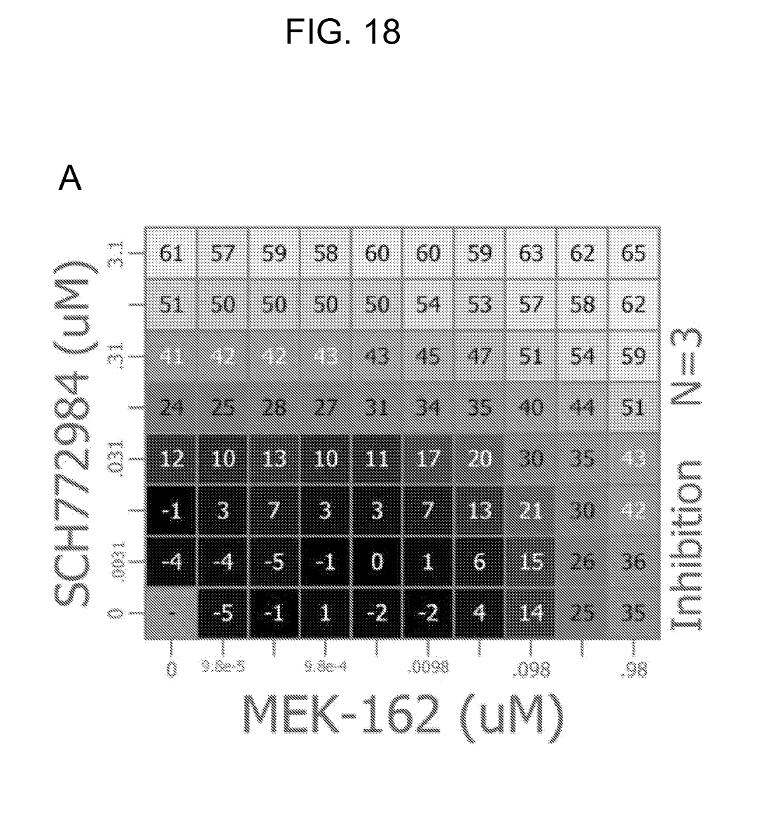

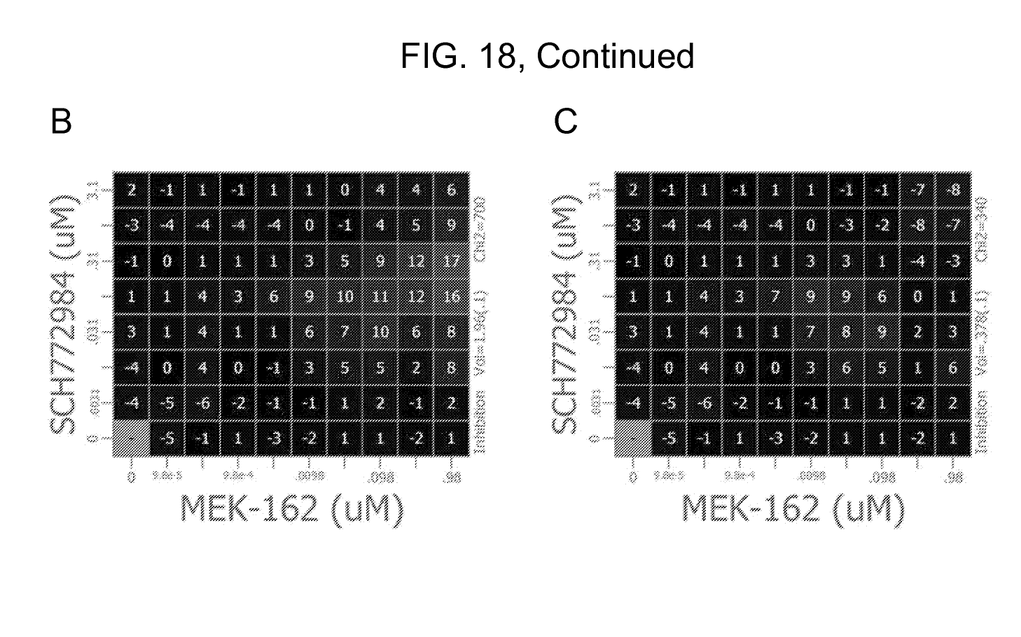

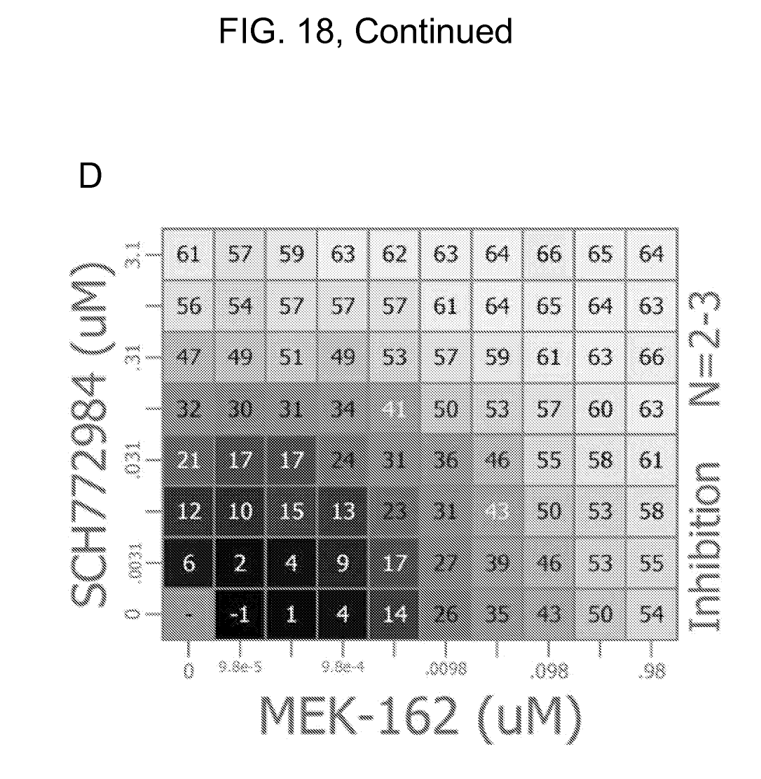

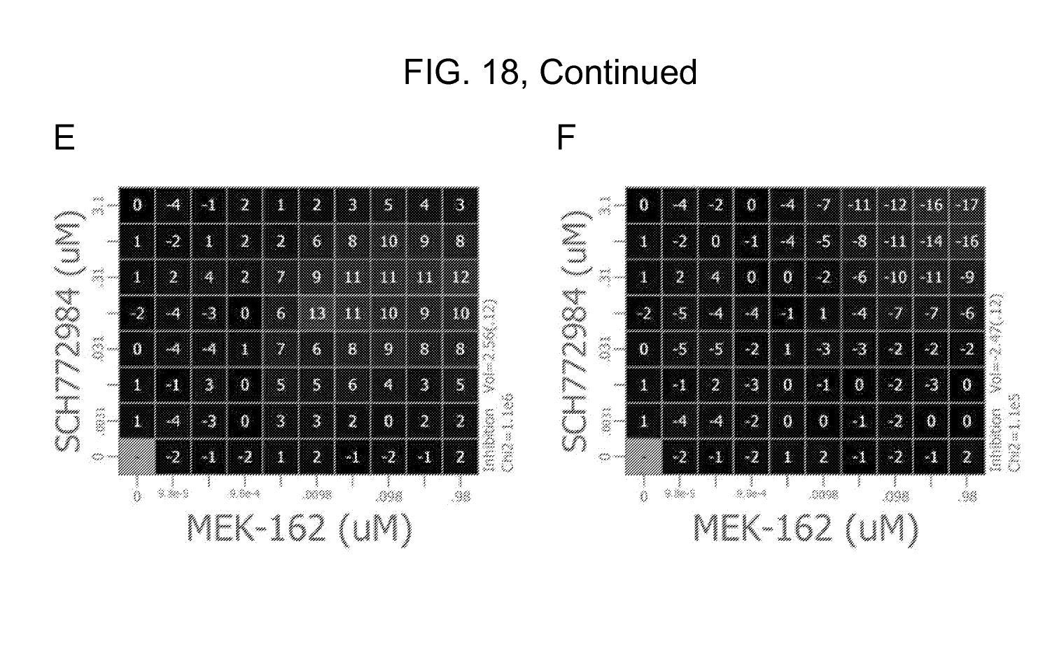

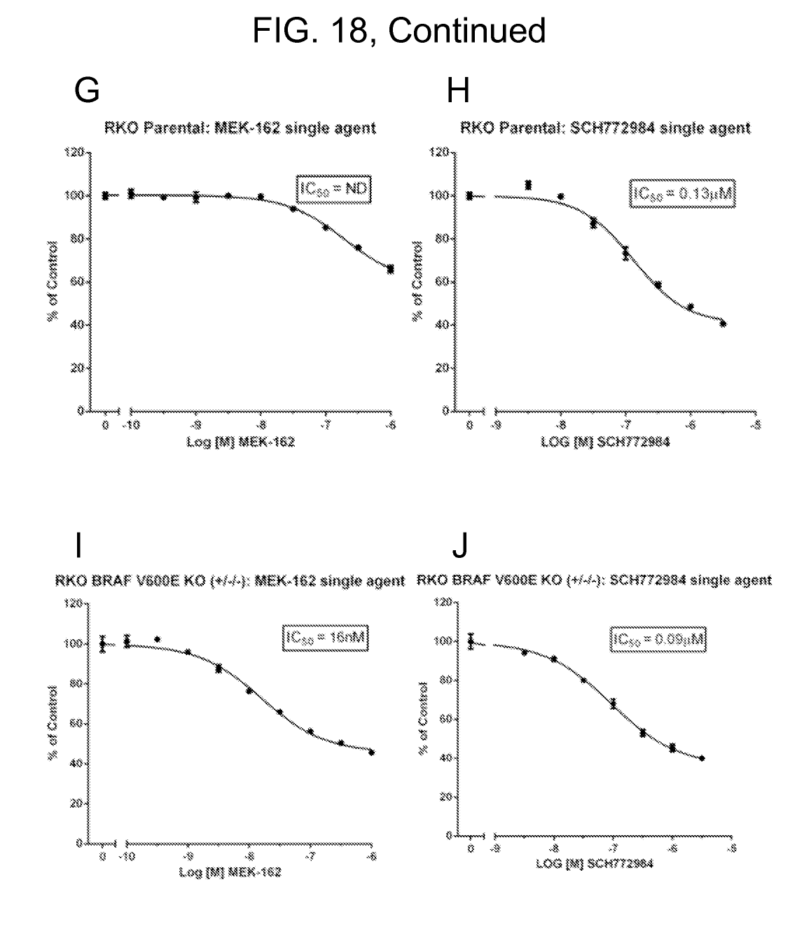

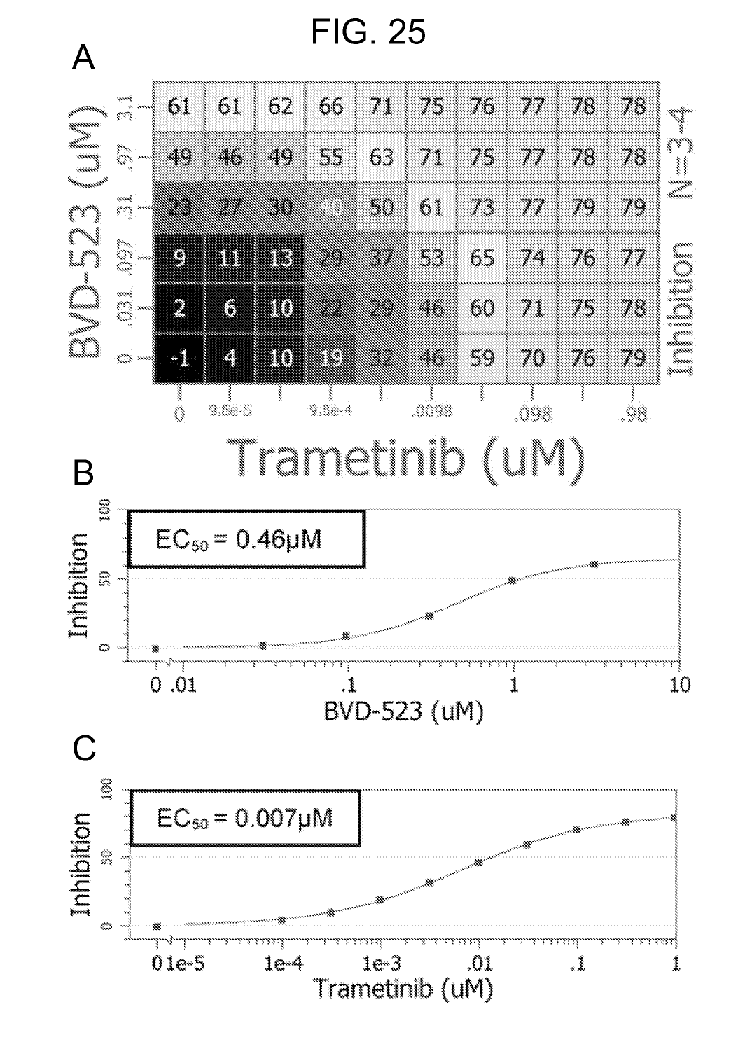

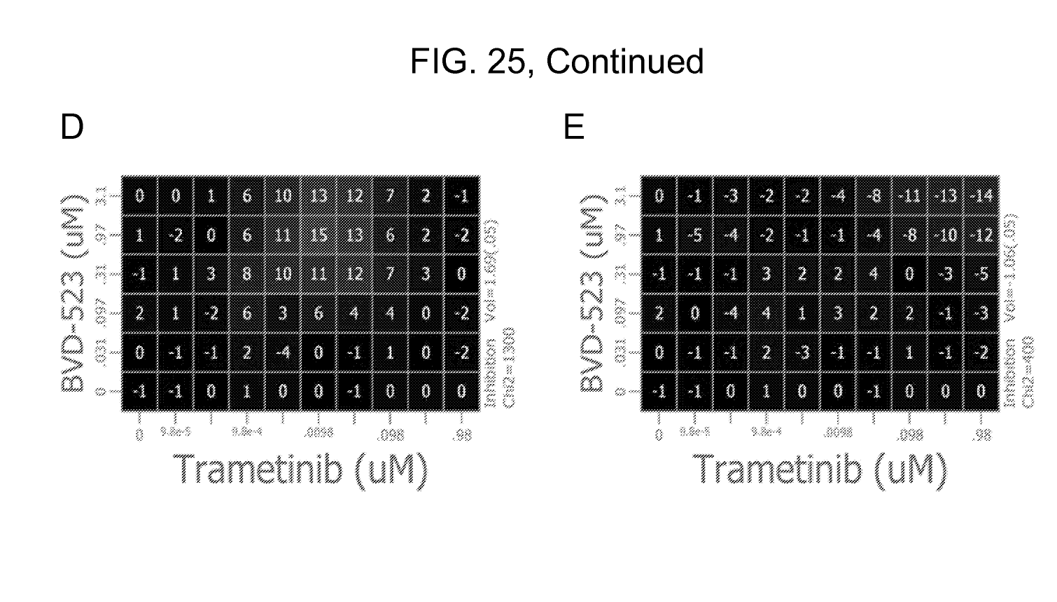

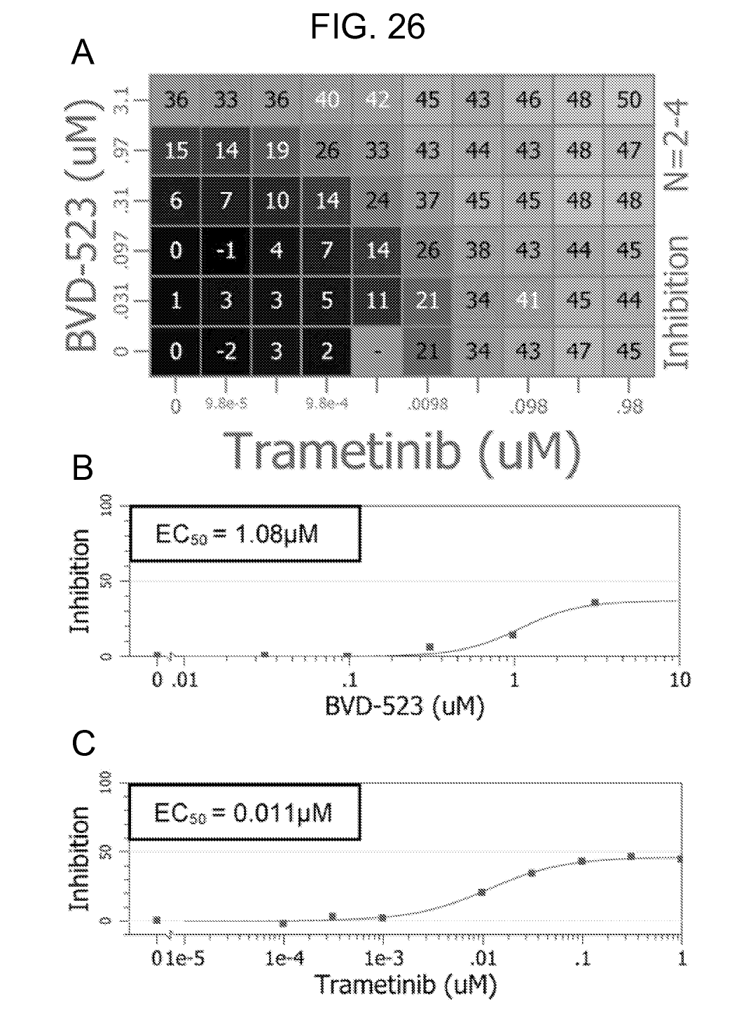

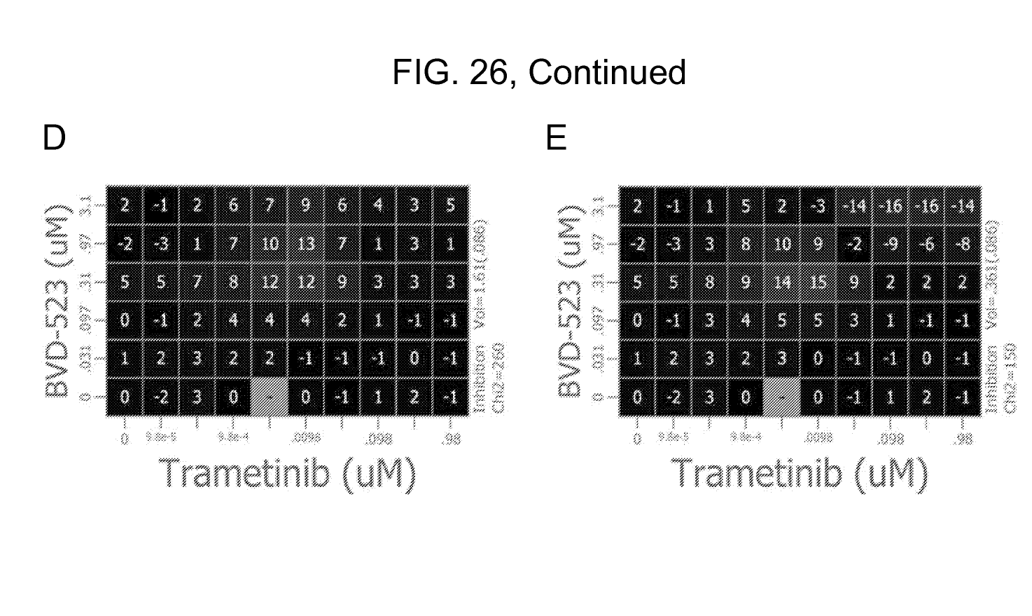

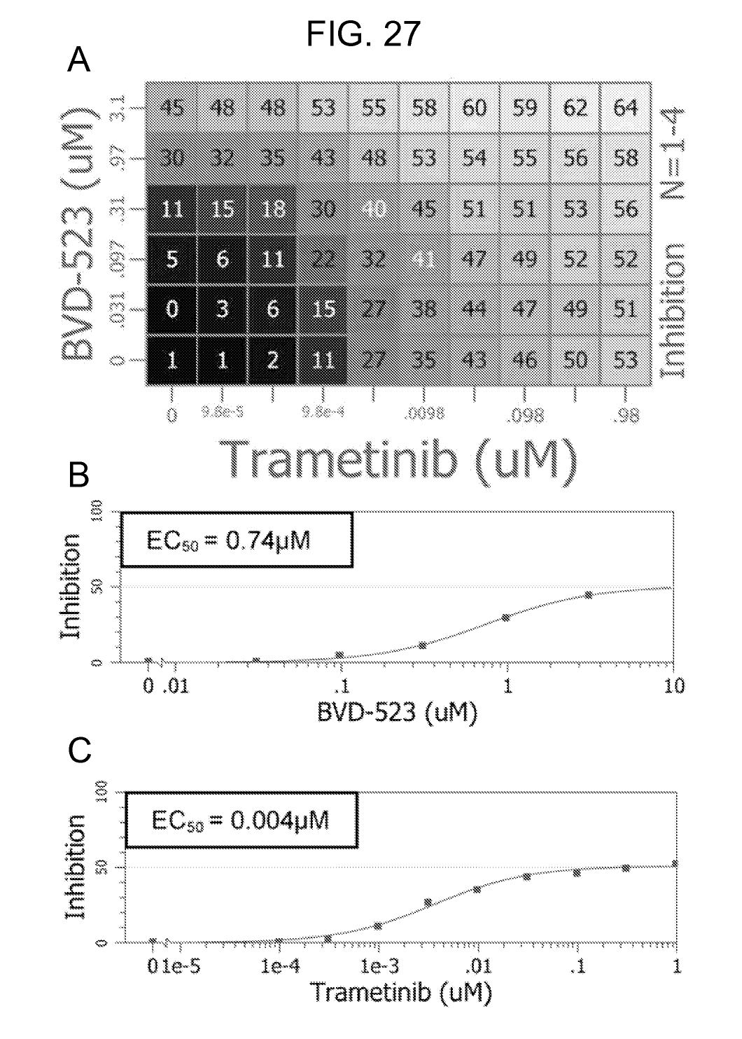

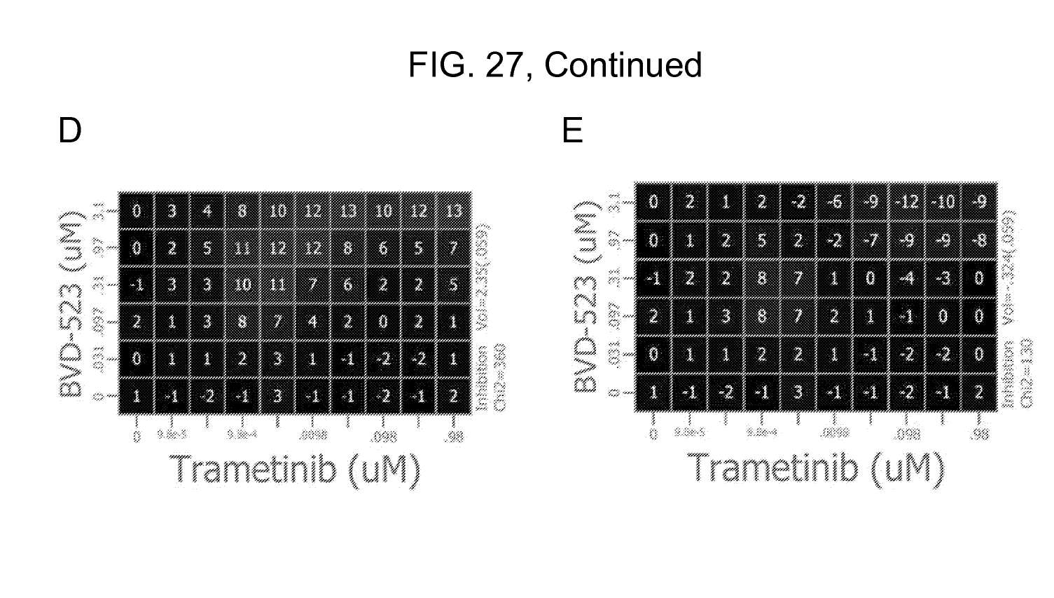

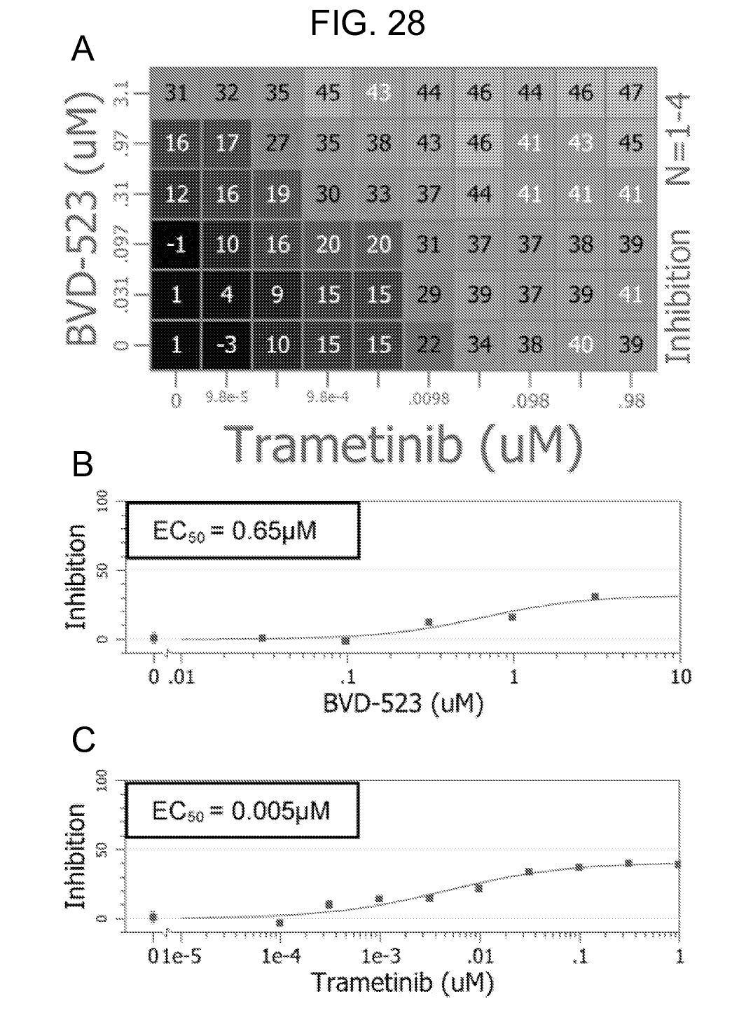

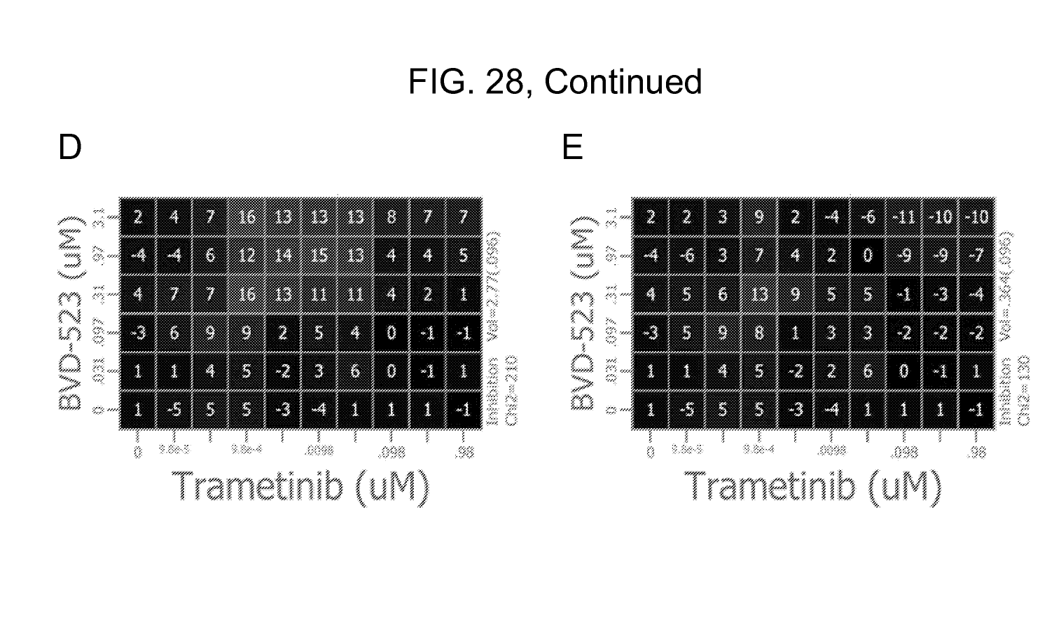

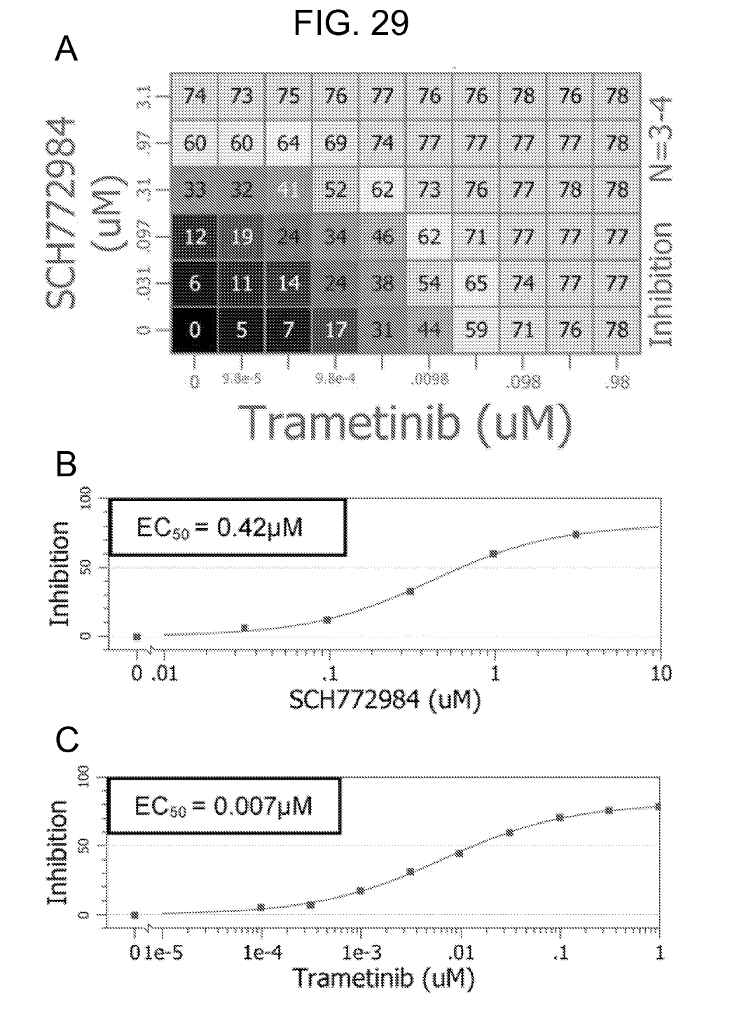

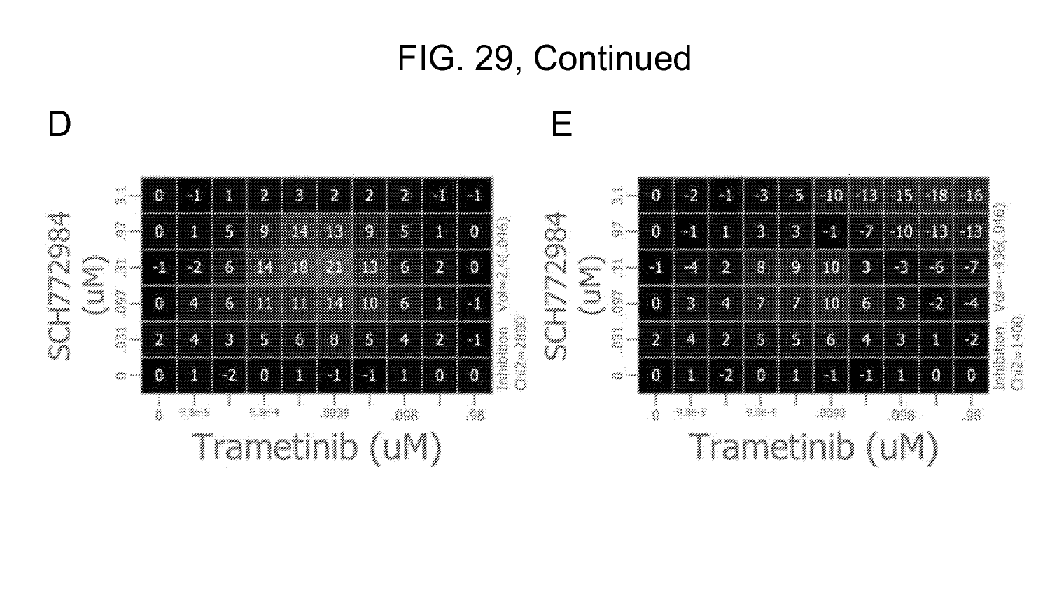

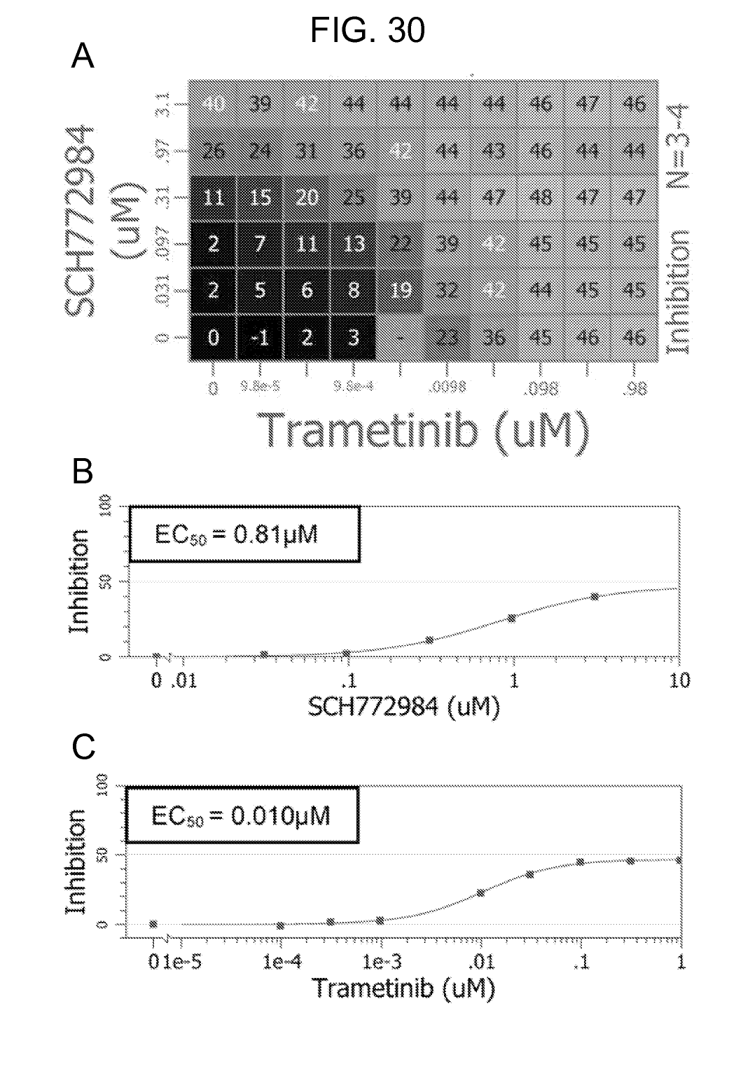

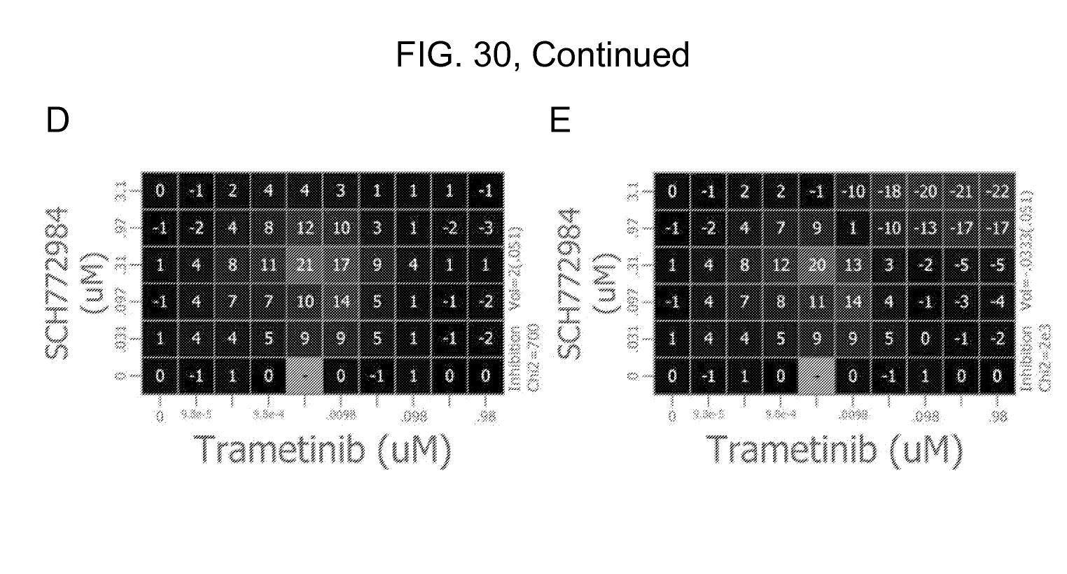

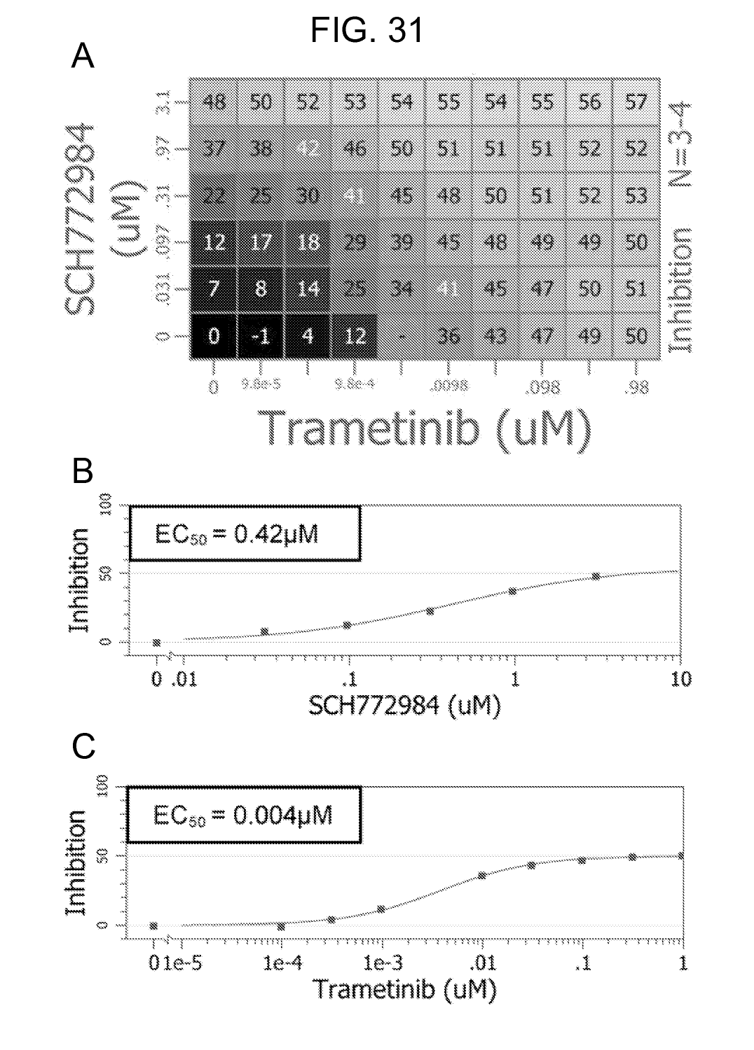

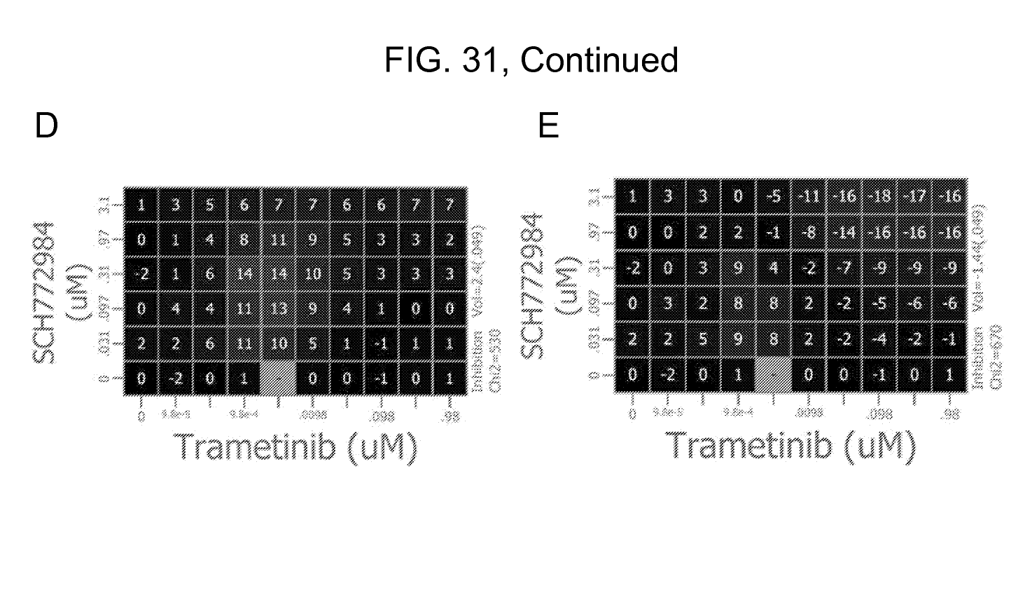

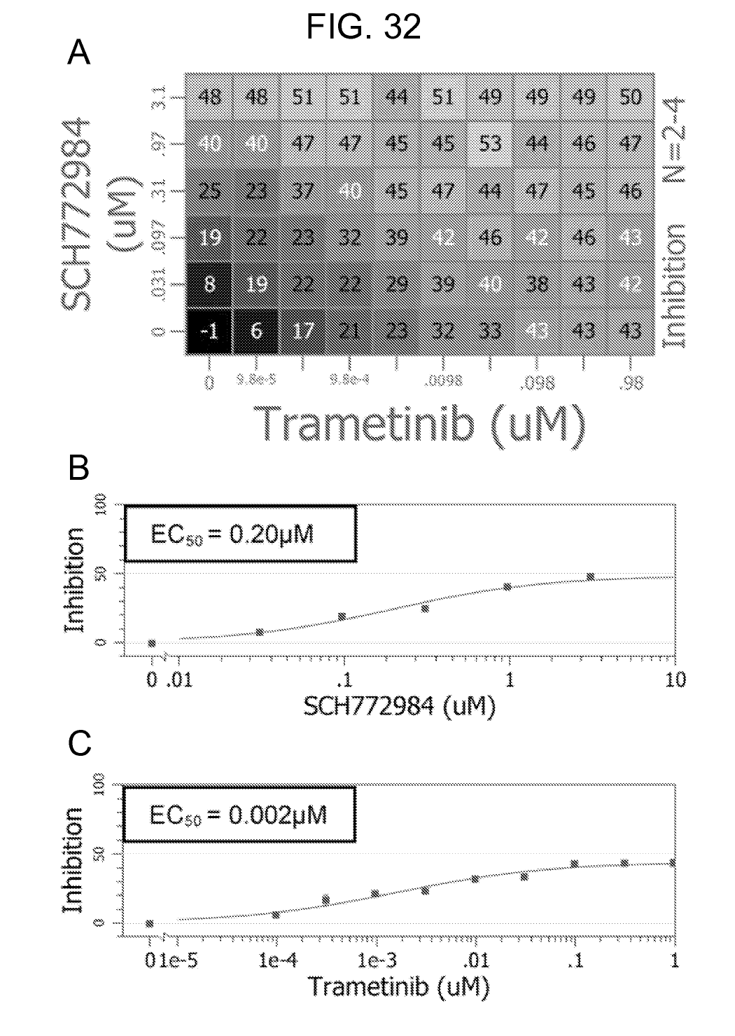

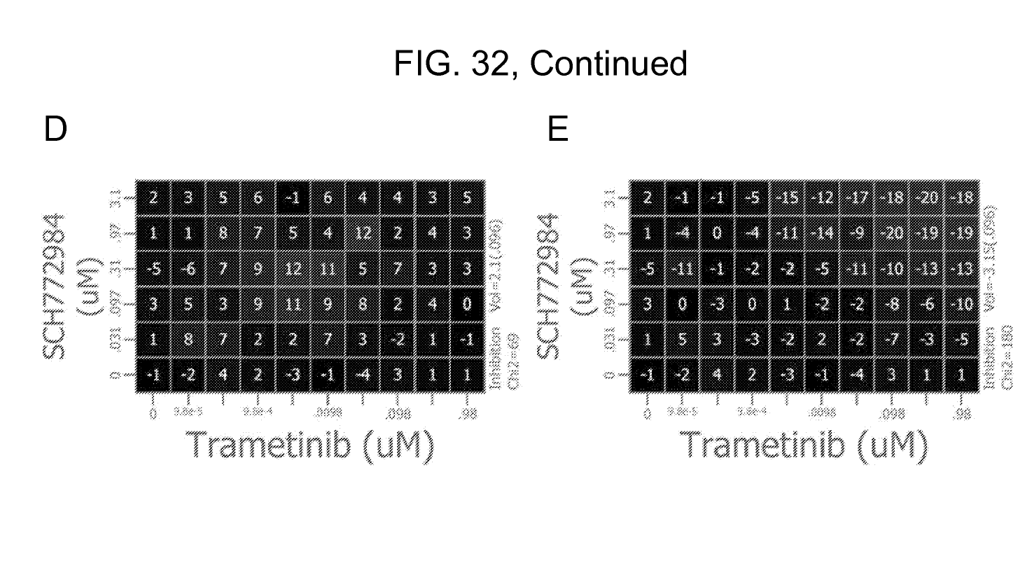

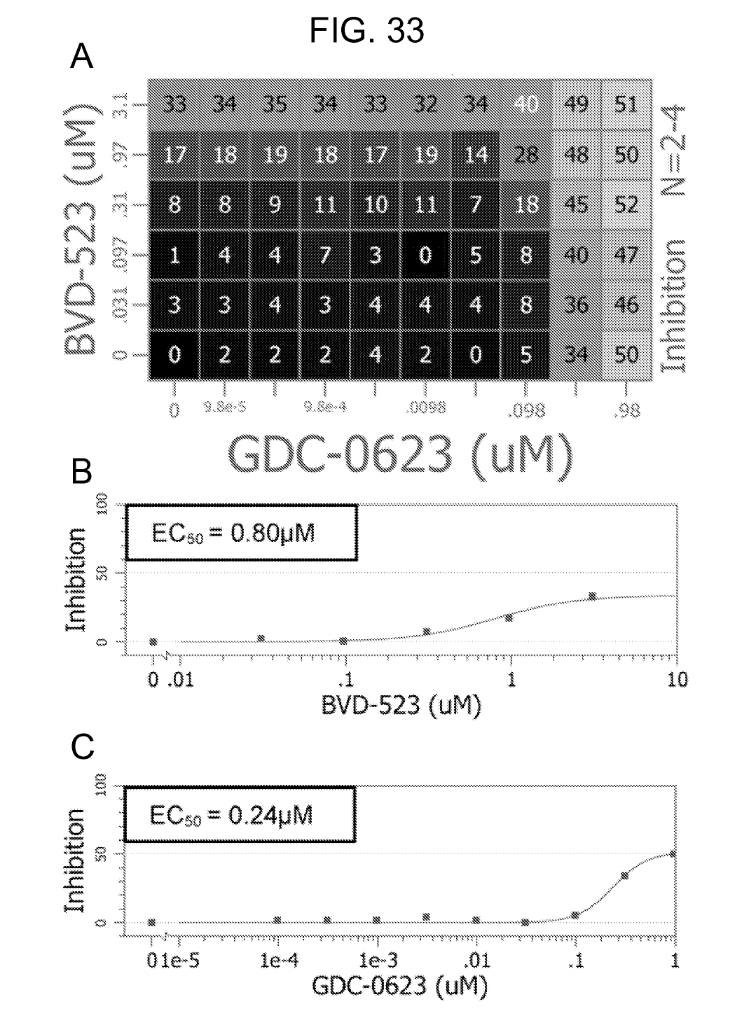

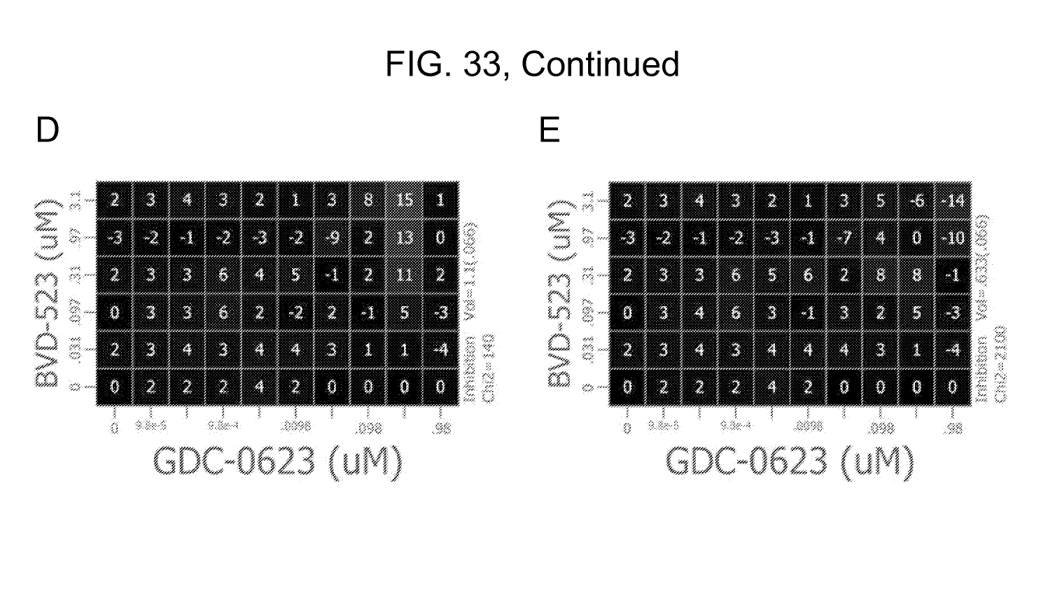

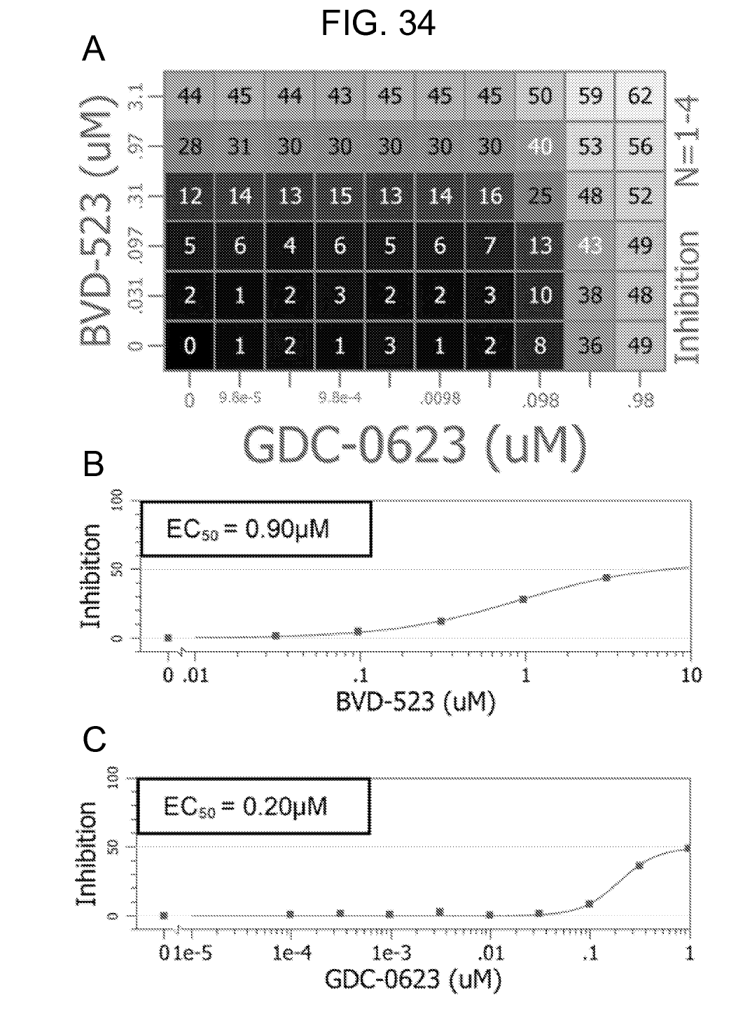

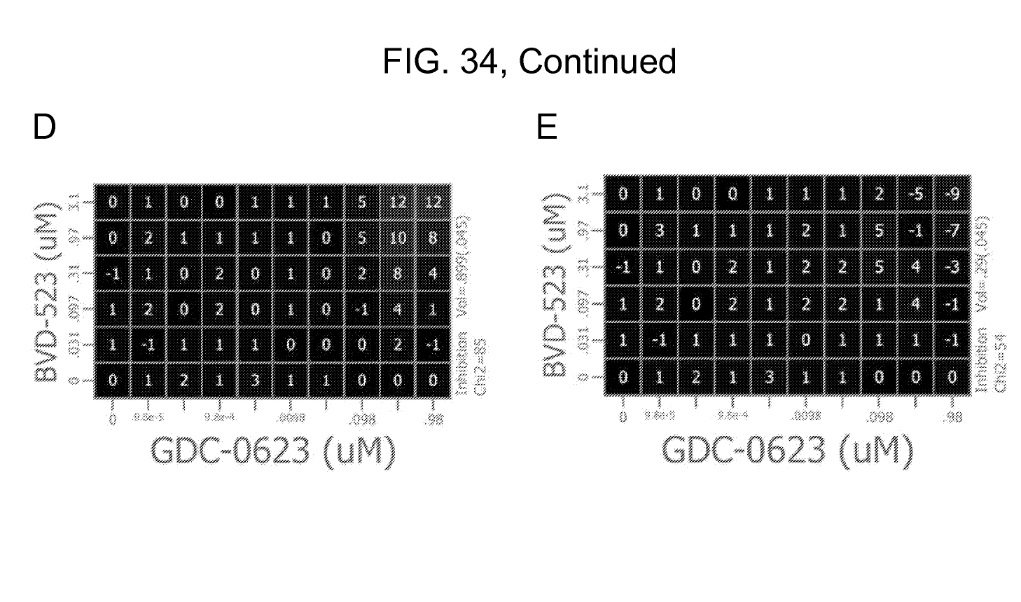

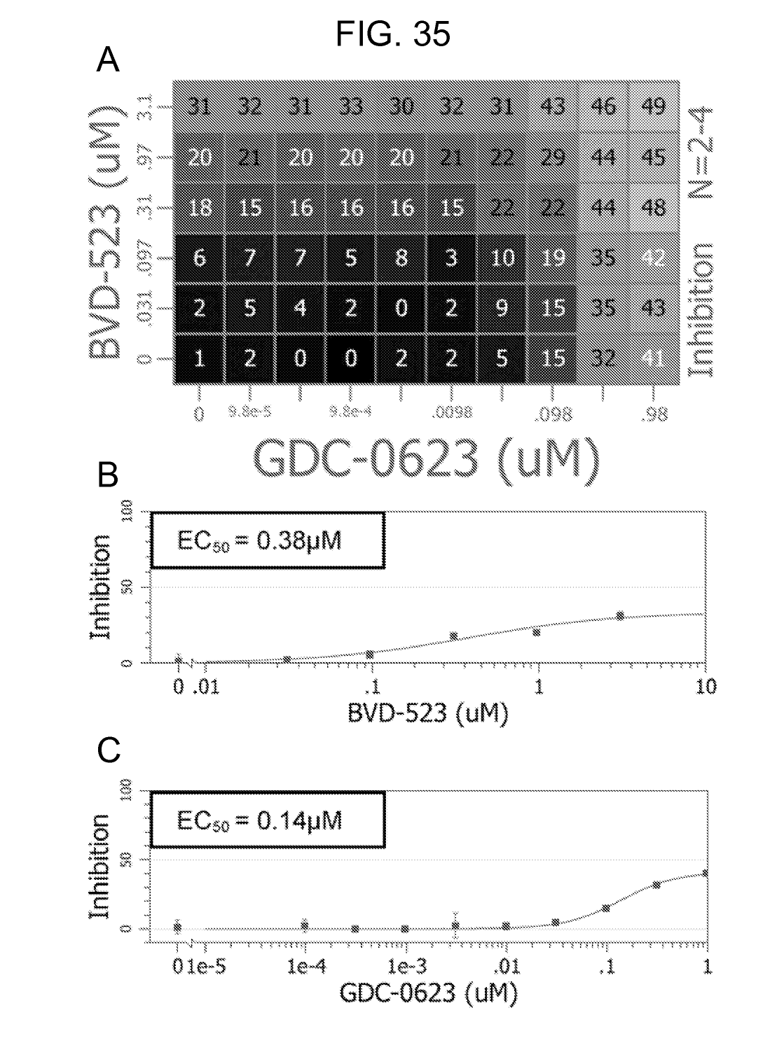

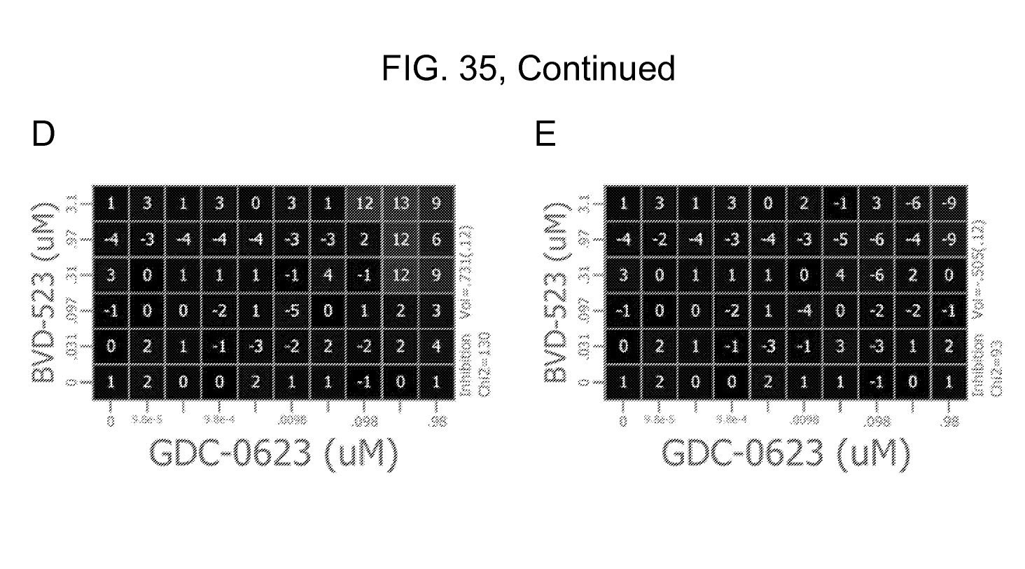

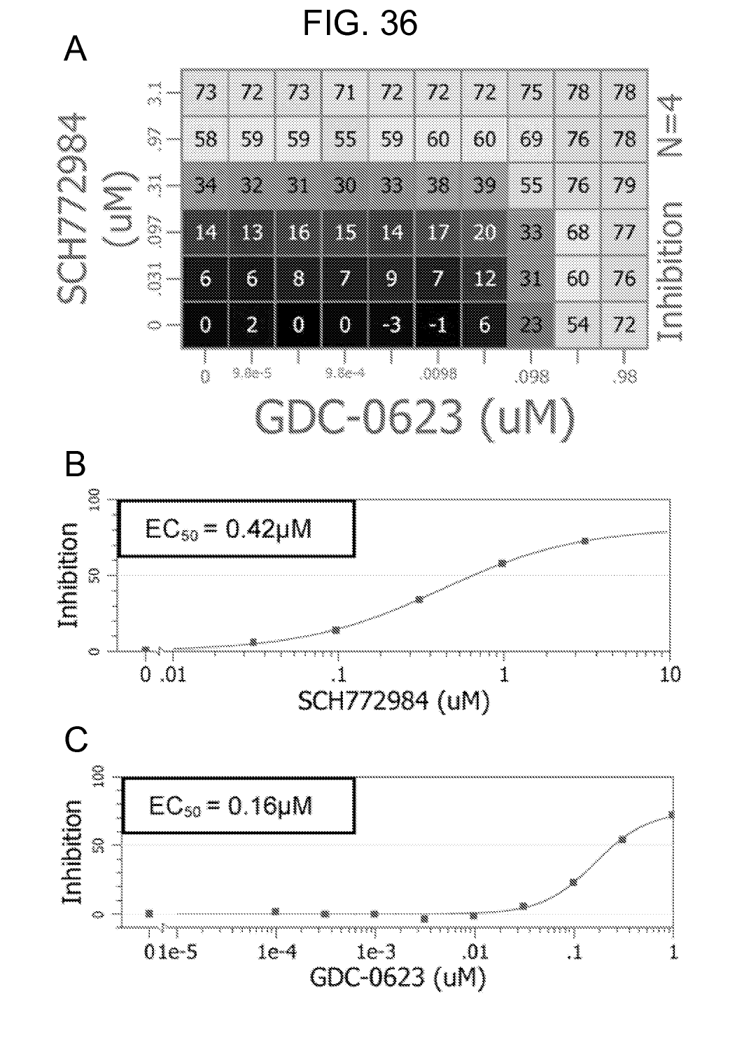

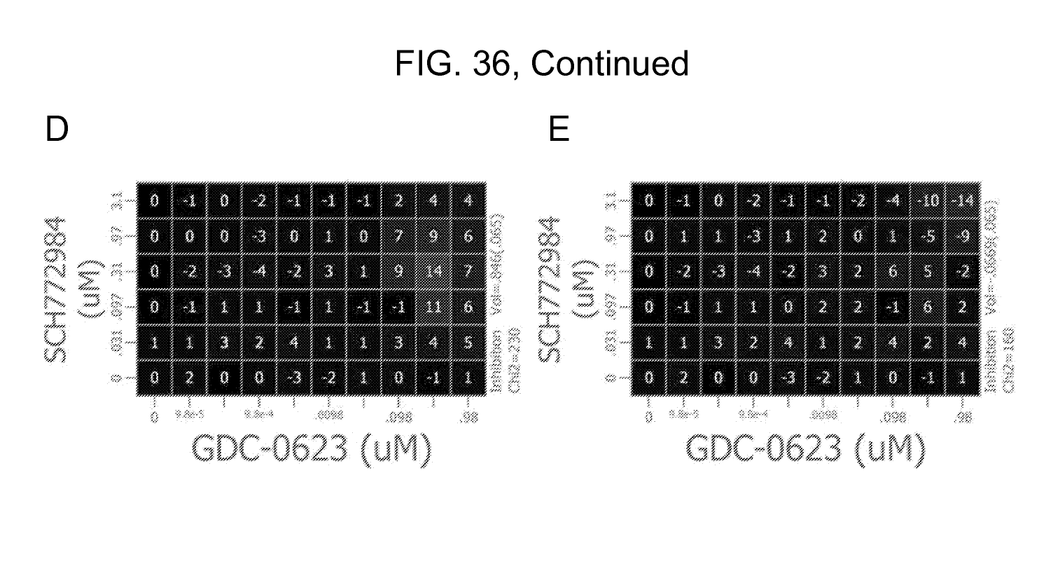

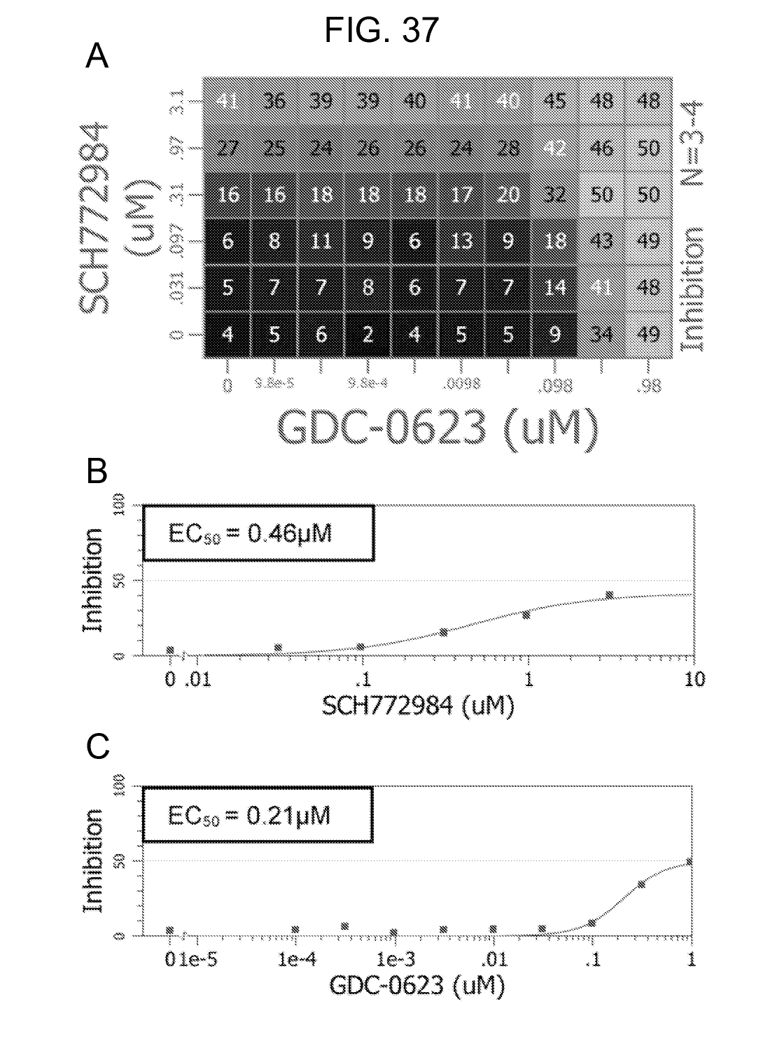

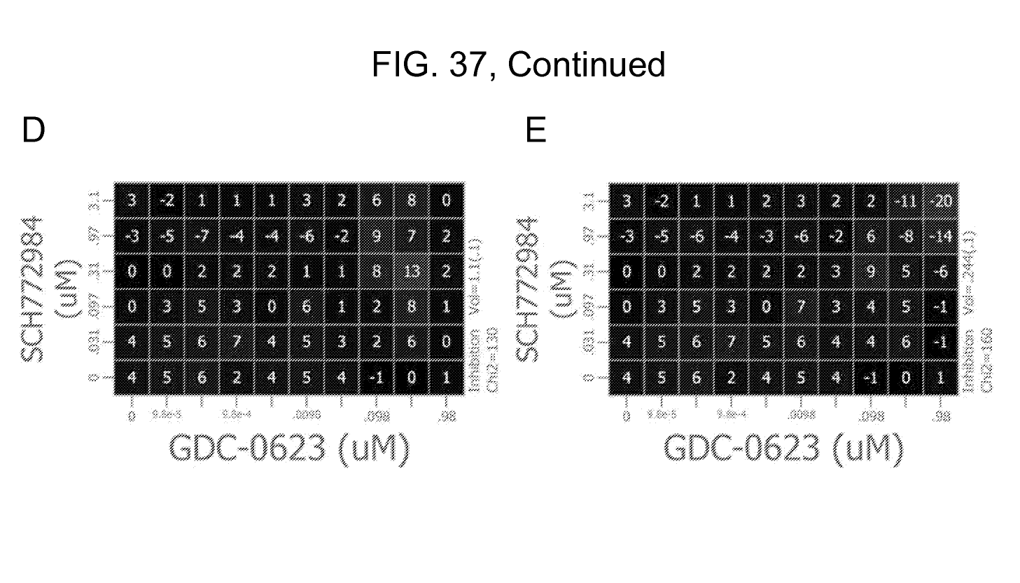

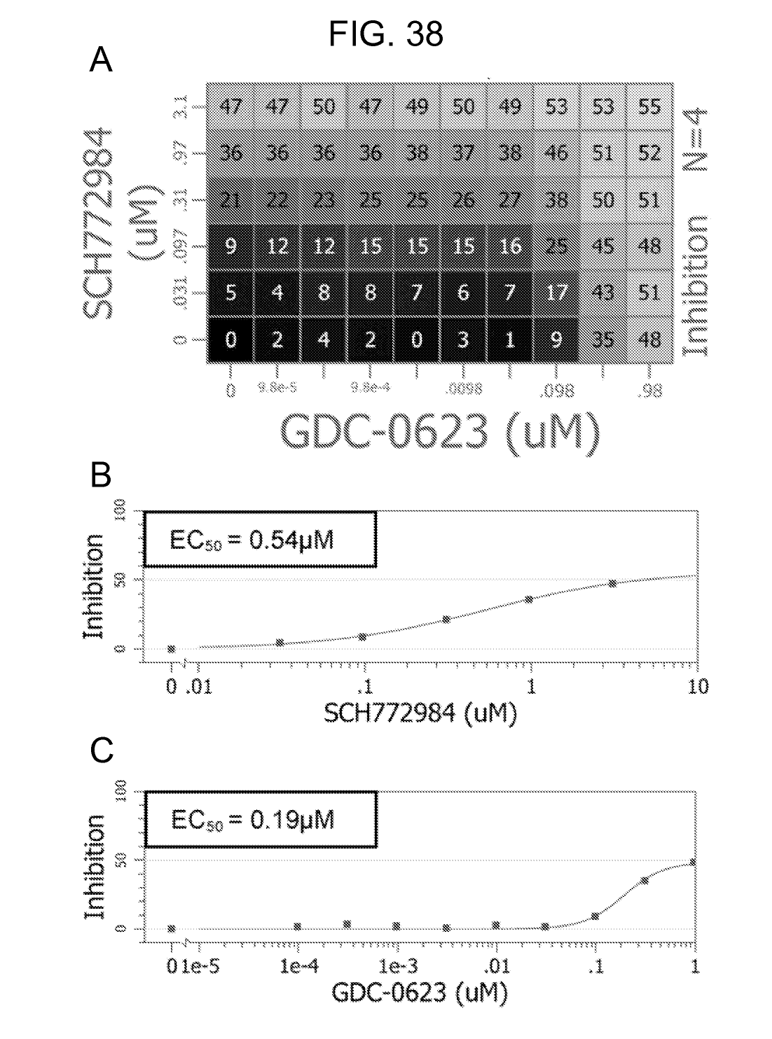

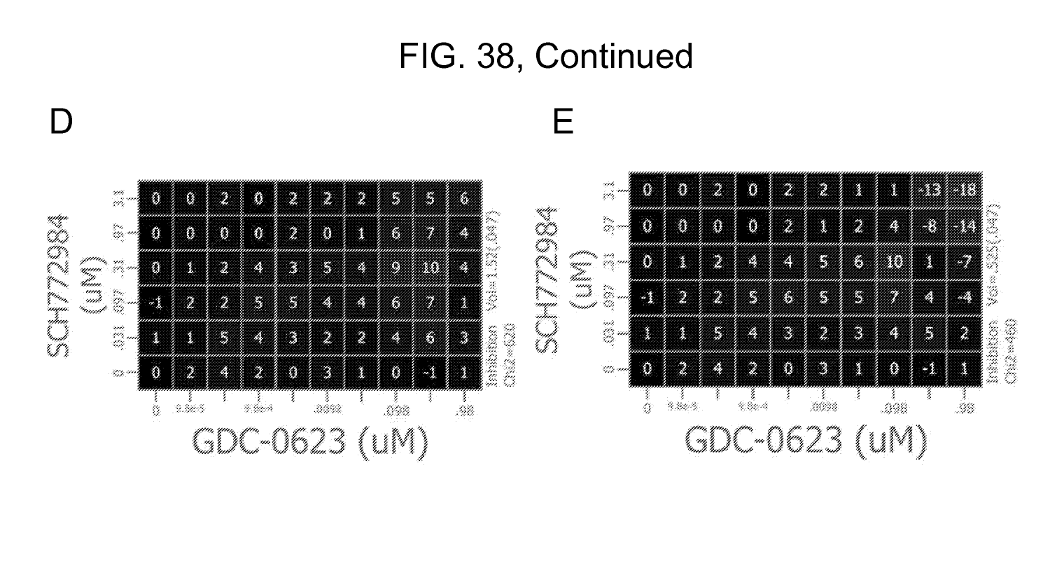

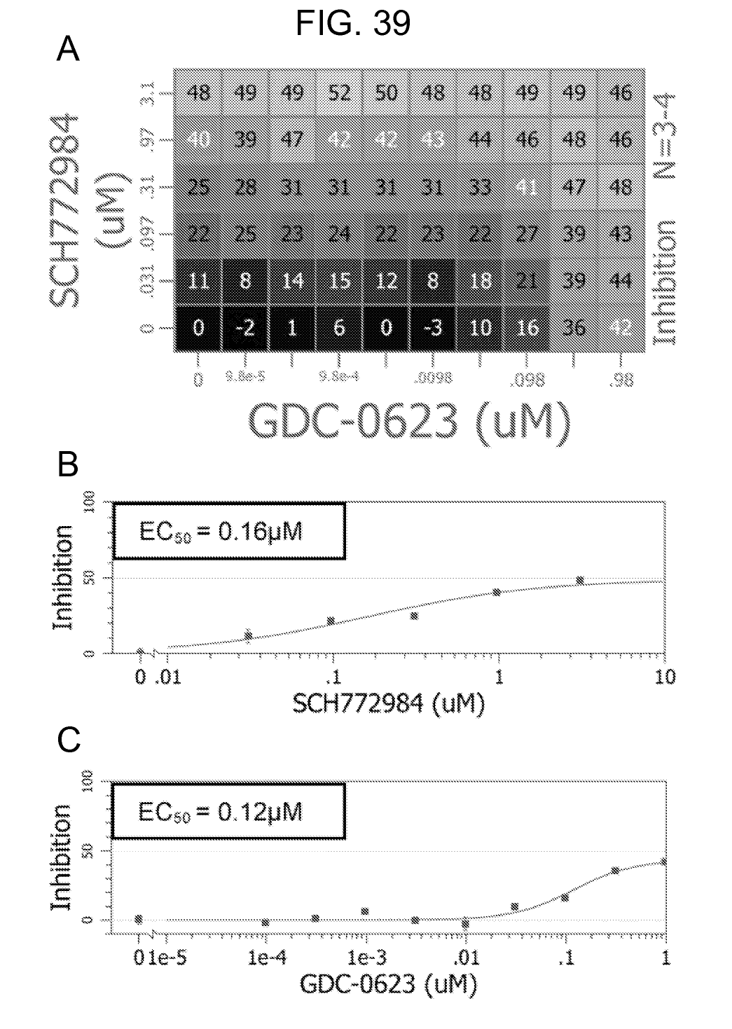

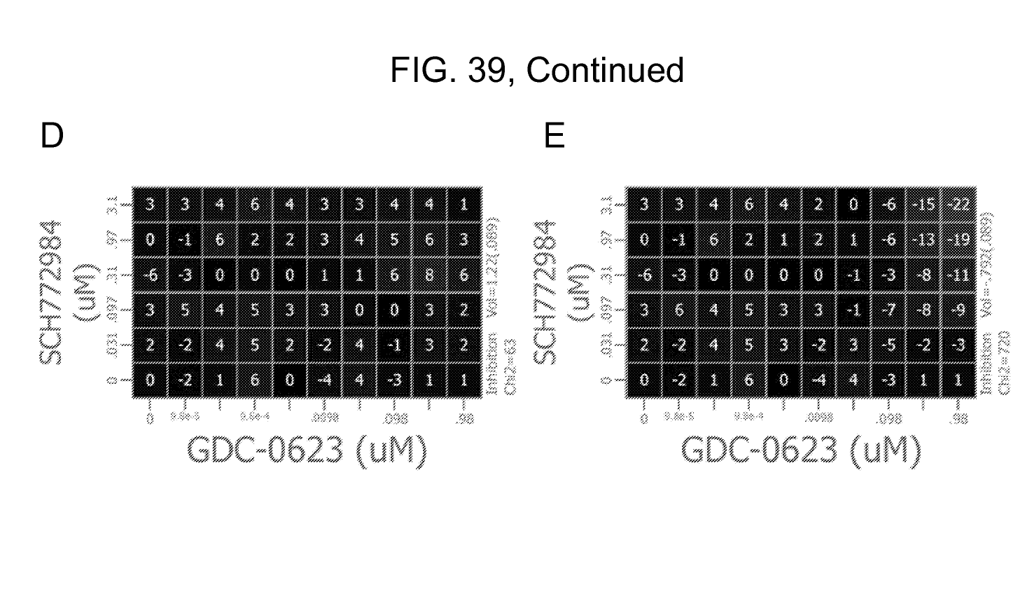

In contrast, when assessed using the Bliss model, interactions between BVD-523 or SCH772984 and MEK inhibitors in HCT116 (FIG. 11-FIG. 14) and the lung lines (FIG. 25-FIG. 39) appeared to be mostly additive. In the RKO cells (FIG. 15-FIG. 18) there were pockets of mild antagonism at higher concentrations. Excess scores were generally more positive, but still mainly additive, when the BVD-523 combinations were analyzed using the Loewe model. Similar results were also obtained for the SCH772984 combinations in these cell lines using the Bliss model, however, the Loewe model suggested the possible presence of regions of synergy in HCT116 and some of the lung lines that were not apparent from the Bliss model.

Synergistic interactions were scored in two ways. Excess activity over that predicted if a combination was additive can be calculated using a simple volume score, which calculates the volume between the measured and the predicted response surface. This volume score shows whether the overall response to a combination is synergistic (positive values), antagonistic (negative values) or additive (values˜0). Table 10 shows Bliss volumes and Table 11 shows Loewe volumes; nt=not tested. Additionally, a ‘Synergy Score’, a positive-gated inhibition-weighted volume over Loewe additivity, is calculated and results are shown in Table 12; nt=not tested. This provides an additional prioritization favoring combinations whose synergy occurs at high effect levels, ignoring antagonistic portions of the response surface.

### Example 5

### Combination Interactions Between ERK Inhibitors

RAF mutant melanoma cell line A375 cells were cultured in DMEM with 10% FBS and seeded into triplicate 96-well plates at an initial density of 2000 cells per well. Combination interactions between ERK inhibitors BVD-523 and SCH772984 were analized after 72 hours as described above in Example 4. Viability was determined using CellTiter-Glo® reagent (Promega, Madison, Wis.) according to manufacturer's instructions and luminescence was detected using the BMG FLUOstar plate reader (BMG Labtech, Ortenberg, Germany).

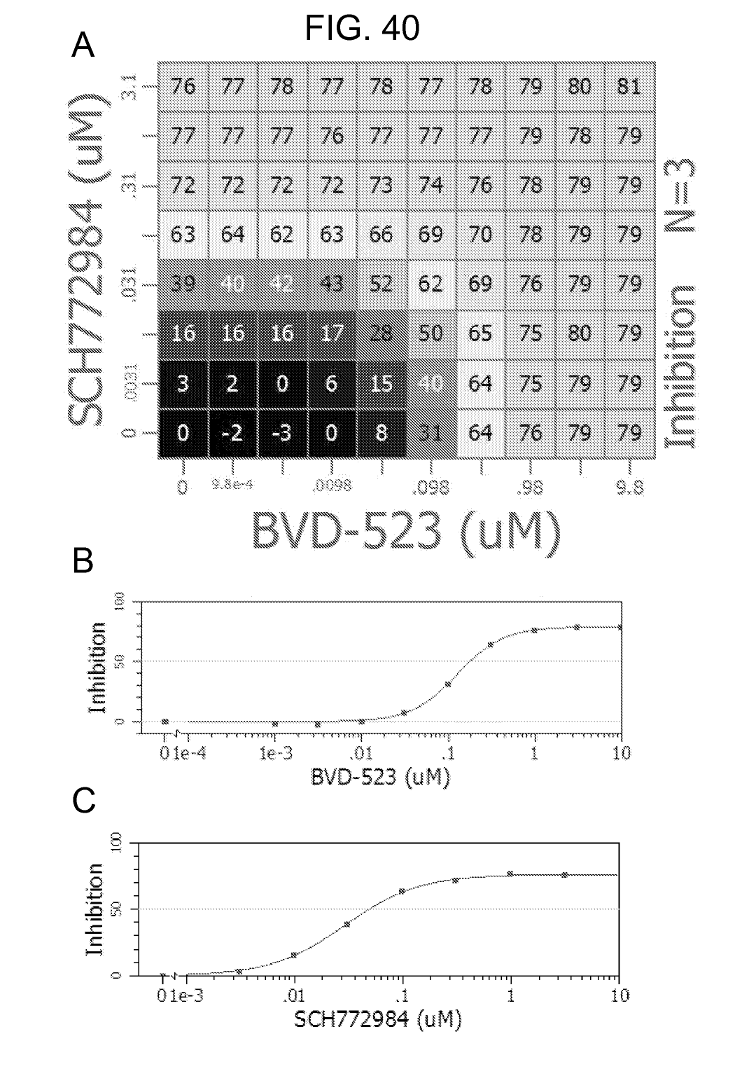

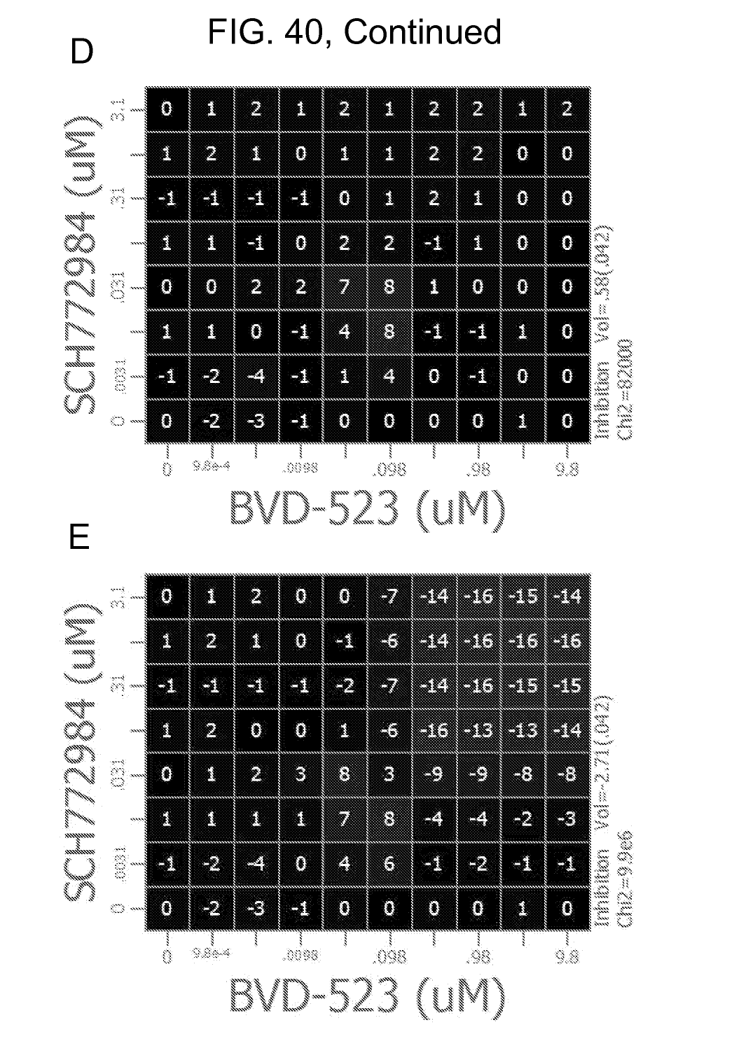

Visualization of the Loewe and Bliss ‘excess inhibition’ heat maps suggested that the combination of BVD-523 and SCH772984 was mainly additive with windows of potential synergy in mid-range doses (FIG. 40).

In summary, these results suggest that interactions between BVD-523 and SCH772984 are at least additive, and in some cases synergistic.

### Documents

AVRUCH, J. et al., Ras activation of the Raf kinase: tyrosine kinase recruitment of the MAP kinase cascade. Recent Prog. Horm. Res., 2001, pp. 127-155.
BHAGWAT, S. S. et al. (2007). Chapter 17: MAP kinase inhibitors in inflammation and autoimmune disorders. Ann Rep Med Chem 42: 265-278. BROSE et al., BRAF and RAS mutations in human lung cancer and melanoma. Cancer Res., 2002, 62, 6997-7000.
DAVIES et al., Mutations of the BRAF gene in human cancer. Nature, 2002, 417, 949-954.
FERRANDI, C. et al. (2011). Characterization of immune cell subsets during the active phase of multiple sclerosis reveals disease and c-Jun N-terminal kinase pathway biomarkers. Mult Scler 17: 143-56.
FRANSEN et al., Mutation analysis of the BRAF, ARAF and RAF-1 genes in human colorectal adenocarcinomas. Carcinogenesis, 2004, 25, 527-533.
GARNETT, M. J. et al. Wildtype and mutant B-RAF activate C-RAF through distinct mechanisms involving heterodimerization. Mol. Cell, 2005, 20, 963-969.
HALAZY, S. (2006). Designing heterocyclic selective kinase inhibitors: from concept to new drug candidates. ARKIVOC vii: 496-508.
HATZIVASSILIOU, G. et al. “RAF inhibitors prime wild-type RAF to activate the MAPK pathway and enhance growth.” Nature 464.7287 (2010): 431-435.
HOCKER et al., Ultraviolet radiation and melanoma: A systematic review and analysis of reported sequence variants. Hum. Mutat., 2007, 28, 578-588.
LI et al., Recent advances in the research and development of B-Raf Inhibitors. Current Medicinal Chemistry, 2010, 17:1618-1634.
LONG G. V., Menzies A M, Nagrial A M et al. Prognostic and Clinicopathologic Associations of Oncogenic BRAF in Metastatic Melanoma. J Clin Oncol., 2011.
RUSHWORTH, L. K. et al., Regulation and role of Raf-1/B-Raf heterodimerization. Mol. Cell Biol., 2006, 26, 2262-2272.
SETH et al., Concomitant mutations and splice variants in KRAS and BRAF demonstrate complex perturbation of the Ras/Raf signalling pathway in advanced colorectal cancer, Gut 2009;58:1234-1241.
WAN et al., Mechanism of activation of the RAF-ERK signaling pathway by oncogenic mutations of B-RAF. Cell, 2004, 116, 855-867.
WEBER, C. K. et al., Active Ras induces heterodimerization of cRaf and BRaf. Cancer Res., 2001, 61, 3595-3598.
WELLBROCK C, Karasarides M, Marais R. The RAF proteins take centre stage. Nat Rev Mol Cell Biol. 2004; 5:875-85.
WELLBROCK, C. et al., The RAF proteins take centre stage. Nat. Rev. Mol. Cell Biol., 2004, 5, 875-885.
WILLIAMS, D. H., et al. (1998). RO 09-2210 exhibits potent anti-proliferative effects on activated T cells by selectively blocking MKK activity. Biochemistry 37(26): 9579-85.
XU et al., High prevalence of BRAF gene mutation in papillary thyroid carcinomas and thyroid tumor cell lines. Cancer Res., 2003, 63, 4561-4567.
ZHAO, A. et al. (1999). Resorcylic acid lactones: naturally occurring potent and selective inhibitors of MEK. The Journal of Antibiotics 52(12): 1086-1094.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method of treating or ameliorating the effects of a cancer in a subject in need thereof comprising administering to the subject an effective amount of (i) a first anti-cancer agent, which is BVD-523 or a pharmaceutically acceptable salt thereof and (ii) a second anti-cancer agent, which is a type 1 MEK inhibitor or a pharmaceutically acceptable salt thereof, to treat or ameliorate the effects of the cancer.

2. The method according to claim 1, wherein the subject is a mammal.

3. The method according to claim 2, wherein the mammal is selected from the group consisting of humans, primates, farm animals, and domestic animals.

4. The method according to claim 2, wherein the mammal is a human.

5. The method according to claim 1, wherein the type 1 MEK inhibitor is selected from the group consisting of bentamapimod (Merck KGaA), L783277 (Merck), RO092210 (Roche), pharmaceutically acceptable salts thereof, and combinations thereof.

6. The method according to claim 1, wherein the type 1 MEK inhibitor is RO092210 (Roche) or a pharmaceutically acceptable salt thereof.

7. The method according to claim 1, wherein the subject with cancer has a somatic RAS or BRAF mutation.

8. The method according to claim 1, wherein the cancer is selected from the group consisting of a cancer of the large intestine, breast cancer, pancreatic cancer, skin cancer, endometrial cancer, neuroblastoma, leukemia, lymphoma, liver cancer, lung cancer, testicular cancer, and thyroid cancer.

9. The method according to claim 1, wherein the cancer is melanoma.

10. The method according to claim 1 further comprising administering to the subject at least one additional therapeutic agent selected from the group consisting of an antibody or fragment thereof, a cytotoxic agent, a toxin, a radionuclide, an immunomodulator, a photoactive therapeutic agent, a radiosensitizing agent, a hormone, an anti-angiogenesis agent, and combinations thereof.

11. The method according to claim 10, wherein the additional therapeutic agent is an inhibitor of the PI3K/Akt pathway.

12. The method according to claim 11, wherein the inhibitor of the PI3K/Akt pathway is selected from the group consisting of A-674563 (CAS #552325-73-2), AGL 2263, AMG-319 (Amgen, Thousand Oaks, Calif.), AS-041164 (5-benzo[1,3]dioxol-5-ylmethylene-thiazolidine-2,4-dione), AS-604850 (5-(2,2-Difluoro-benzo[1,3]dioxol-5-ylmethyleneythiazolidine-2,4-dione), AS-605240 (5-quinoxilin-6-methylene-1,3-thiazolidine-2,4-dione), AT7867 (CAS #857531-00-1), benzimidazole series, Genentech (Roche Holdings Inc., South San Francisco, Calif.), BML-257 (CAS #32387-96-5), CAL-120 (Gilead Sciences, Foster City, Calif.), CAL-129 (Gilead Sciences), CAL-130 (Gilead Sciences), CAL-253 (Gilead Sciences), CAL-263 (Gilead Sciences), CAS #612847-09-3, CAS #681281-88-9, CAS #75747-14-7, CAS #925681-41-0, CAS #98510-80-6, CCT128930 (CAS #885499-61-6), CH5132799 (CAS #1007207-67-1), CHR-4432 (Chroma Therapeutics, Ltd., Abingdon, UK), FPA 124 (CAS #902779-59-3), GS-1101 (CAL-101) (Gilead Sciences), GSK 690693 (CAS #937174-76-0), H-89 (CAS #127243-85-0), Honokiol, IC87114 (Gilead Science), IPI-145 (Intellikine Inc.), KAR-4139 (Karus Therapeutics, Chilworth, UK), KAR-4141 (Karus Therapeutics), KIN-1 (Karus Therapeutics), KT 5720 (CAS #108068-98-0), Miltefosine, MK-2206 dihydrochloride (CAS #1032350-13-2), ML-9 (CAS #105637-50-1), Naltrindole Hydrochloride, OXY-111A (NormOxys Inc., Brighton, Mass.), perifosine, PHT-427 (CAS #1191951-57-1), PI3 kinase delta inhibitor, Merck KGaA (Merck & Co., Whitehouse Station, N.J.), PI3 kinase delta inhibitors, Genentech (Roche Holdings Inc.), PI3 kinase delta inhibitors, Incozen (Incozen Therapeutics, Pvt. Ltd., Hyderabad, India), PI3 kinase delta inhibitors-2, Incozen (Incozen Therapeutics), PI3 kinase inhibitor, Roche-4 (Roche Holdings Inc.), PI3 kinase inhibitors, Roche (Roche Holdings Inc.), PI3 kinase inhibitors, Roche-5 (Roche Holdings Inc.), PI3-alpha/delta inhibitors, Pathway Therapeutics (Pathway Therapeutics Ltd., South San Francisco, Calif.), PI3-delta inhibitors, Cellzome (Cellzome AG, Heidelberg, Germany), PI3-delta inhibitors, Intellikine (Intellikine Inc., La Jolla, Calif.), PI3-delta inhibitors, Pathway Therapeutics-1 (Pathway Therapeutics Ltd.), PI3-delta inhibitors, Pathway Therapeutics-2 (Pathway Therapeutics Ltd.), PI3-delta/gamma inhibitors, Cellzome (Cellzome AG), PI3-delta/gamma inhibitors, Cellzome (Cellzome AG), PI3-delta/gamma inhibitors, Intellikine (Intellikine Inc.), PI3-delta/gamma inhibitors, Intellikine (Intellikine Inc.), PI3-delta/gamma inhibitors, Pathway Therapeutics (Pathway Therapeutics Ltd.), PI3-delta/gamma inhibitors, Pathway Therapeutics (Pathway Therapeutics Ltd.), PI3-gamma inhibitor Evotec (Evotec), PI3-gamma inhibitor, Cellzome (Cellzome AG), PI3-gamma inhibitors, Pathway Therapeutics (Pathway Therapeutics Ltd.), PI3K delta/gamma inhibitors, Intellikine-1 (Intellikine Inc.), PI3K delta/gamma inhibitors, Intellikine-1 (Intellikine Inc.), pictilisib (Roche Holdings Inc.), PIK-90 (CAS #677338-12-4), SC-103980 (Pfizer, New York, N.Y.), SF-1126 (Semafore Pharmaceuticals, Indianapolis, Ind.), SH-5, SH-6, Tetrahydro Curcumin, TG100-115 (Targegen Inc., San Diego, Calif.), Triciribine, X-339 (Xcovery, West Palm Beach, Fla.), XL-499 (Evotech, Hamburg, Germany), pharmaceutically acceptable salts thereof, and combinations thereof.

13. The method according to claim 1, wherein administration of the first and second anti-cancer agents provides a synergistic effect compared to administration of either anti-cancer agent alone.

14. A method of treating or ameliorating the effects of a cancer in a subject in need thereof comprising administering to the subject an effective amount of (i) a first anti-cancer agent, which is BVD-523 or a pharmaceutically acceptable salt thereof and (ii) a second anti-cancer agent, which is RO092210 (Roche) or a pharmaceutically acceptable salt thereof, to treat or ameliorate the effects of the cancer.

15. The method according to claim 14, wherein the subject is a mammal.

16. The method according to claim 15, wherein the mammal is selected from the group consisting of humans, primates, farm animals, and domestic animals.

17. The method according to claim 15, wherein the mammal is a human.

18. The method according to claim 14, wherein the BVD-523 or a pharmaceutically acceptable salt thereof is administered in the form of a pharmaceutical composition further comprising a pharmaceutically acceptable carrier or diluent.

19. The method according to claim 14, wherein the RO092210 (Roche) or a pharmaceutically acceptable salt thereof is administered in the form of a pharmaceutical composition further comprising a pharmaceutically acceptable carrier or diluent.

20. The method according to claim 14, wherein the subject with cancer has a somatic RAS mutation or BRAF mutation.

21. The method according to claim 14, wherein the cancer is selected from the group consisting of a cancer of the large intestine, breast cancer, pancreatic cancer, skin cancer, endometrial cancer, neuroblastoma, leukemia, lymphoma, liver cancer, lung cancer, testicular cancer, and thyroid cancer.

22. The method according to claim 14, wherein the cancer is melanoma.

23. The method according to claim 14 further comprising administering to the subject at least one additional therapeutic agent selected from the group consisting of an antibody or fragment thereof, a cytotoxic agent, a toxin, a radionuclide, an immunomodulator, a photoactive therapeutic agent, a radiosensitizing agent, a hormone, an anti-angiogenesis agent, and combinations thereof.

24. The method according to claim 23, wherein the additional therapeutic agent is an inhibitor of the PI3K/Akt pathway.

25. The method according to claim 24, wherein the inhibitor of the PI3K/Akt pathway is selected from the group consisting of A-674563 (CAS #552325-73-2), AGL 2263, AMG-319 (Amgen, Thousand Oaks, Calfi.), AS-041164 (5-benzo[1,3]dioxol-5-ylmethylene-thiazolidine-2,4-dione), AS-604850 (5-(2,2-Difluoro-benzo[1,3]dioxol-5-ylmethyleneythiazolidine-2,4-dione), AS-605240 (5-quinoxilin-6-methylene-1,3-thiazolidine-2,4-dione), AT7867 (CAS #857531-00-1), benzimidazole series, Genentech (Roche Holdings Inc., South San Francisco, Calif.), BML-257 (CAS #32387-96-5), CAL-120 (Gilead Sciences, Foster City, Calif.), CAL-129 (Gilead Sciences), CAL-130 (Gilead Sciences), CAL-253 (Gilead Sciences), CAL-263 (Gilead Sciences), CAS #612847-09-3, CAS #681281-88-9, CAS #75747-14-7, CAS #925681-41-0, CAS #98510-80-6, CCT128930 (CAS #885499-61-6), CH5132799 (CAS #1007207-67-1), CHR-4432 (Chroma Therapeutics, Ltd., Abingdon, UK), FPA 124 (CAS #902779-59-3), GS-1101 (CAL-101) (Gilead Sciences), GSK 690693 (CAS #937174-76-0), H-89 (CAS #127243-85-0), Honokiol, IC87114 (Gilead Science), IPI-145 (Intellikine Inc.), KAR-4139 (Karus Therapeutics, Chilworth, UK), KAR-4141 (Karus Therapeutics), KIN-1 (Karus Therapeutics), KT 5720 (CAS #108068-98-0), Miltefosine, MK-2206 dihydrochloride (CAS #1032350-13-2), ML-9 (CAS #105637-50-1), Naltrindole Hydrochloride, OXY-111A (NormOxys Inc., Brighton, Mass.), perifosine, PHT-427 (CAS #1191951-57-1), PI3 kinase delta inhibitor, Merck KGaA (Merck & Co., Whitehouse Station, N.J.), PI3 kinase delta inhibitors, Genentech (Roche Holdings Inc.), PI3 kinase delta inhibitors, Incozen (Incozen Therapeutics, Pvt. Ltd., Hyderabad, India), PI3 kinase delta inhibitors-2, Incozen (Incozen Therapeutics), PI3 kinase inhibitor, Roche-4 (Roche Holdings Inc.), PI3 kinase inhibitors, Roche (Roche Holdings Inc.), PI3 kinase inhibitors, Roche-5 (Roche Holdings Inc.), PI3-alpha/delta inhibitors, Pathway Therapeutics (Pathway Therapeutics Ltd., South San Francisco, Calif.), PI3-delta inhibitors, Cellzome (Cellzome AG, Heidelberg, Germany), PI3-delta inhibitors, Intellikine (Intellikine Inc., La Jolla, Calif.), PI3-delta inhibitors, Pathway Therapeutics-1 (Pathway Therapeutics Ltd.), PI3-delta inhibitors, Pathway Therapeutics-2 (Pathway Therapeutics Ltd.), PI3-delta/gamma inhibitors, Cellzome (Cellzome AG), PI3-delta/gamma inhibitors, Cellzome (Cellzome AG), PI3-delta/gamma inhibitors, Intellikine (Intellikine Inc.), PI3-delta/gamma inhibitors, Intellikine (Intellikine Inc.), PI3-delta/gamma inhibitors, Pathway Therapeutics (Pathway Therapeutics Ltd.), PI3-delta/gamma inhibitors, Pathway Therapeutics (Pathway Therapeutics Ltd.), PI3-gamma inhibitor Evotec (Evotec), PI3-gamma inhibitor, Cellzome (Cellzome AG), PI3-gamma inhibitors, Pathway Therapeutics (Pathway Therapeutics Ltd.), PI3K delta/gamma inhibitors, Intellikine-1 (Intellikine Inc.), PI3K delta/gamma inhibitors, Intellikine-1 (Intellikine Inc.), pictilisib (Roche Holdings Inc.), PIK-90 (CAS #677338-12-4), SC-103980 (Pfizer, New York, N.Y.), SF-1126 (Semafore Pharmaceuticals, Indianapolis, Ind.), SH-5, SH-6, Tetrahydro Curcumin, TG100-115 (Targegen Inc., San Diego, Calif.), Triciribine, X-339 (Xcovery, West Palm Beach, Fla.), XL-499 (Evotech, Hamburg, Germany), pharmaceutically acceptable salts thereof, and combinations thereof.

26. The method according to claim 14, wherein administration of the first and second anti-cancer agents provides a synergistic effect compared to administration of either anti-cancer agent alone.

27. A method of effecting cancer cell death comprising contacting the cancer cell with an effective amount of (i) a first anti-cancer agent, which is BVD-523 or a pharmaceutically acceptable salt thereof and (ii) a second anti-cancer agent, which is a type 1 MEK inhibitor or a pharmaceutically acceptable salt thereof.

28. The method according to clam 27, wherein the cancer cell is a mammalian cancer cell.

29. The method according to claim 28, wherein the mammalian cancer cell is obtained from a mammal selected from the group consisting of humans, primates, farm animals, and domestic animals.

30. The method according to claim 28, wherein the mammalian cancer cell is a human cancer cell.

31. The method according to claim 27, wherein the type 1 MEK inhibitor is selected from the group consisting of bentamapimod (Merck KGaA), L783277 (Merck), RO092210 (Roche), pharmaceutically acceptable salts thereof, and combinations thereof.

32. The method according to claim 27, wherein the type 1 MEK inhibitor is RO092210 (Roche) or a pharmaceutically acceptable salt thereof.

33. The method according to claim 27, wherein the subject with cancer has a somatic RAS mutation or BRAF mutation.

34. The method according to claim 27, wherein the cancer is selected from the group consisting of a cancer of the large intestine, breast cancer, pancreatic cancer, skin cancer, endometrial cancer, neuroblastoma, leukemia, lymphoma, liver cancer, lung cancer, testicular cancer, and thyroid cancer.

35. The method according to claim 27, wherein the cancer is melanoma.

36. The method according to claim 27 further comprising administering to the subject at least one additional therapeutic agent selected from the group consisting of an antibody or fragment thereof, a cytotoxic agent, a toxin, a radionuclide, an immunomodulator, a photoactive therapeutic agent, a radiosensitizing agent, a hormone, an anti-angiogenesis agent, and combinations thereof.

37. The method according to claim 36, wherein the additional therapeutic agent is an inhibitor of the PI3K/Akt pathway.

38. The method according to claim 37, wherein the inhibitor of the PI3K/Akt pathway is selected from the group consisting of A-674563 (CAS #552325-73-2), AGL 2263, AMG-319 (Amgen, Thousand Oaks, Calif.), AS-041164 (5-benzo[1,3]dioxol-5-ylmethylene-thiazolidine-2,4-dione), AS-604850 (542,2-Difluoro-benzo[1,3]dioxol-5-ylmethyleneythiazolidine-2,4-dione), AS-605240 (5-quinoxilin-6-methylene-1,3-thiazolidine-2,4-dione), AT7867 (CAS #857531-00-1), benzimidazole series, Genentech (Roche Holdings Inc., South San Francisco, Calif.), BML-257 (CAS #32387-96-5), CAL-120 (Gilead Sciences, Foster City, Calif.), CAL-129 (Gilead Sciences), CAL-130 (Gilead Sciences), CAL-253 (Gilead Sciences), CAL-263 (Gilead Sciences), CAS #612847-09-3, CAS #681281-88-9, CAS #75747-14-7, CAS #925681-41-0, CAS #98510-80-6, CCT128930 (CAS #885499-61-6), CH5132799 (CAS #1007207-67-1), CHR-4432 (Chroma Therapeutics, Ltd., Abingdon, UK), FPA 124 (CAS #902779-59-3), GS-1101 (CAL-101) (Gilead Sciences), GSK 690693 (CAS #937174-76-0), H-89 (CAS #127243-85-0), Honokiol, IC87114 (Gilead Science), IPI-145 (Intellikine Inc.), KAR-4139 (Karus Therapeutics, Chilworth, UK), KAR-4141 (Karus Therapeutics), KIN-1 (Karus Therapeutics), KT 5720 (CAS #108068-98-0), Miltefosine, MK-2206 dihydrochloride (CAS #1032350-13-2), ML-9 (CAS #105637-50-1), Naltrindole Hydrochloride, OXY-111A (NormOxys Inc., Brighton, Mass.), perifosine, PHT-427 (CAS #1191951-57-1), PI3 kinase delta inhibitor, Merck KGaA (Merck & Co., Whitehouse Station, N.J.), PI3 kinase delta inhibitors, Genentech (Roche Holdings Inc.), PI3 kinase delta inhibitors, Incozen (Incozen Therapeutics, Pvt. Ltd., Hyderabad, India), PI3 kinase delta inhibitors-2, Incozen (Incozen Therapeutics), PI3 kinase inhibitor, Roche-4 (Roche Holdings Inc.), PI3 kinase inhibitors, Roche (Roche Holdings Inc.), PI3 kinase inhibitors, Roche-5 (Roche Holdings Inc.), PI3-alpha/delta inhibitors, Pathway Therapeutics (Pathway Therapeutics Ltd., South San Francisco, Calif.), PI3-delta inhibitors, Cellzome (Cellzome AG, Heidelberg, Germany), PI3-delta inhibitors, Intellikine (Intellikine Inc., La Jolla, Calif.), PI3-delta inhibitors, Pathway Therapeutics-1 (Pathway Therapeutics Ltd.), PI3-delta inhibitors, Pathway Therapeutics-2 (Pathway Therapeutics Ltd.), PI3-delta/gamma inhibitors, Cellzome (Cellzome AG), PI3-delta/gamma inhibitors, Cellzome (Cellzome AG), PI3-delta/gamma inhibitors, Intellikine (Intellikine Inc.), PI3-delta/gamma inhibitors, Intellikine (Intellikine Inc.), PI3-delta/gamma inhibitors, Pathway Therapeutics (Pathway Therapeutics Ltd.), PI3-delta/gamma inhibitors, Pathway Therapeutics (Pathway Therapeutics Ltd.), PI3-gamma inhibitor Evotec (Evotec), PI3-gamma inhibitor, Cellzome (Cellzome AG), PI3-gamma inhibitors, Pathway Therapeutics (Pathway Therapeutics Ltd.), PI3K delta/gamma inhibitors, Intellikine-1 (Intellikine Inc.), PI3K delta/gamma inhibitors, Intellikine-1 (Intellikine Inc.), pictilisib (Roche Holdings Inc.), PIK-90 (CAS #677338-12-4), SC-103980 (Pfizer, New York, N.Y.), SF-1126 (Semafore Pharmaceuticals, Indianapolis, Ind.), SH-5, SH-6, Tetrahydro Curcumin, TG100-115 (Targegen Inc., San Diego, Calif.), Triciribine, X-339 (Xcovery, West Palm Beach, Fla.), XL-499 (Evotech, Hamburg, Germany), pharmaceutically acceptable salts thereof, and combinations thereof.

39. The method according to claim 27, wherein contacting the cancer cell with the first and second anti-cancer agents provides a synergistic effect compared to contacting the cancer cell with either anti-cancer agent alone.

40. A kit for treating or ameliorating the effects of a cancer in a subject in need thereof comprising an effective amount of (i) a first anti-cancer agent, which is BVD-523 or a pharmaceutically acceptable salt thereof and (ii) a second anti-cancer agent, which is a type 1 MEK inhibitor or a pharmaceutically acceptable salt thereof, packaged together with instructions for their use.

41. The kit according to clam 40, wherein the subject is a mammal.

42. The kit according to claim 41, wherein the mammal is selected from the group consisting of humans, primates, farm animals, and domestic animals.

43. The kit according to claim 41, wherein the mammal is a human.

44. The kit according to claim 40, wherein the type 1 MEK inhibitor is selected from the group consisting of bentamapimod (Merck KGaA), L783277 (Merck), RO092210 (Roche), pharmaceutically acceptable salts thereof, and combinations thereof.

45. The kit according to claim 40, wherein the type 1 MEK inhibitor is RO092210 (Roche) or a pharmaceutically acceptable salt thereof.

46. The kit according to claim 40, wherein the subject with cancer has a somatic RAS mutation or BRAF mutation.

47. The kit according to claim 40, wherein the cancer is selected from the group consisting of a cancer of the large intestine, breast cancer, pancreatic cancer, skin cancer, endometrial cancer, neuroblastoma, leukemia, lymphoma, liver cancer, lung cancer, testicular cancer, and thyroid cancer.

48. The kit according to claim 40, wherein the cancer is melanoma.

49. The kit according to claim 40 further comprising at least one additional therapeutic agent selected from the group consisting of an antibody or fragment thereof, a cytotoxic agent, a toxin, a radionuclide, an immunomodulator, a photoactive therapeutic agent, a radiosensitizing agent, a hormone, an anti-angiogenesis agent, and combinations thereof.

50. The kit according to claim 49, wherein the additional therapeutic agent is an inhibitor of the PI3K/Akt pathway.

51. The kit according to claim 50, wherein the inhibitor of the PI3K/Akt pathway is selected from the group consisting of A-674563 (CAS #552325-73-2), AGL 2263, AMG-319 (Amgen, Thousand Oaks, Calif.), AS-041164 (5-benzo[1,3]dioxol-5-ylmethylene-thiazolidine-2,4-dione), AS-604850 (5-(2,2-Difluoro-benzo[1,3]dioxol-5-ylmethyleneythiazolidine-2,4-dione), AS-605240 (5-quinoxilin-6-methylene-1,3-thiazolidine-2,4-dione), AT7867 (CAS #857531-00-1), benzimidazole series, Genentech (Roche Holdings Inc., South San Francisco, Calif.), BML-257 (CAS #32387-96-5), CAL-120 (Gilead Sciences, Foster City, Calif.), CAL-129 (Gilead Sciences), CAL-130 (Gilead Sciences), CAL-253 (Gilead Sciences), CAL-263 (Gilead Sciences), CAS #612847-09-3, CAS #681281-88-9, CAS #75747-14-7, CAS #925681-41-0, CAS #98510-80-6, CCT128930 (CAS #885499-61-6), CH5132799 (CAS #1007207-67-1), CHR-4432 (Chroma Therapeutics, Ltd., Abingdon, UK), FPA 124 (CAS #902779-59-3), GS-1101 (CAL-101) (Gilead Sciences), GSK 690693 (CAS #937174-76-0), H-89 (CAS #127243-85-0), Honokiol, IC87114 (Gilead Science), IPI-145 (Intellikine Inc.), KAR-4139 (Karus Therapeutics, Chilworth, UK), KAR-4141 (Karus Therapeutics), KIN-1 (Karus Therapeutics), KT 5720 (CAS #108068-98-0), Miltefosine, MK-2206 dihydrochloride (CAS #1032350-13-2), ML-9 (CAS #105637-50-1), Naltrindole Hydrochloride, OXY-111A (NormOxys Inc., Brighton, Mass.), perifosine, PHT-427 (CAS #1191951-57-1), PI3 kinase delta inhibitor, Merck KGaA (Merck & Co., Whitehouse Station, N.J.), PI3 kinase delta inhibitors, Genentech (Roche Holdings Inc.), PI3 kinase delta inhibitors, Incozen (Incozen Therapeutics, Pvt. Ltd., Hyderabad, India), PI3 kinase delta inhibitors-2, Incozen (Incozen Therapeutics), PI3 kinase inhibitor, Roche-4 (Roche Holdings Inc.), PI3 kinase inhibitors, Roche (Roche Holdings Inc.), PI3 kinase inhibitors, Roche-5 (Roche Holdings Inc.), PI3-alpha/delta inhibitors, Pathway Therapeutics (Pathway Therapeutics Ltd., South San Francisco, Calif.), PI3-delta inhibitors, Cellzome (Cellzome AG, Heidelberg, Germany), PI3-delta inhibitors, Intellikine (Intellikine Inc., La Jolla, Calif.), PI3-delta inhibitors, Pathway Therapeutics-1 (Pathway Therapeutics Ltd.), PI3-delta inhibitors, Pathway Therapeutics-2 (Pathway Therapeutics Ltd.), PI3-delta/gamma inhibitors, Cellzome (Cellzome AG), PI3-delta/gamma inhibitors, Cellzome (Cellzome AG), PI3-delta/gamma inhibitors, Intellikine (Intellikine Inc.), PI3-delta/gamma inhibitors, Intellikine (Intellikine Inc.), PI3-delta/gamma inhibitors, Pathway Therapeutics (Pathway Therapeutics Ltd.), PI3-delta/gamma inhibitors, Pathway Therapeutics (Pathway Therapeutics Ltd.), PI3-gamma inhibitor Evotec (Evotec), PI3-gamma inhibitor, Cellzome (Cellzome AG), PI3-gamma inhibitors, Pathway Therapeutics (Pathway Therapeutics Ltd.), PI3K delta/gamma inhibitors, Intellikine-1 (Intellikine Inc.), PI3K delta/gamma inhibitors, Intellikine-1 (Intellikine Inc.), pictilisib (Roche Holdings Inc.), PIK-90 (CAS #677338-12-4), SC-103980 (Pfizer, New York, N.Y.), SF-1126 (Semafore Pharmaceuticals, Indianapolis, IN), SH-5, SH-6, Tetrahydro Curcumin, TG100-115 (Targegen Inc., San Diego, Calif.), Triciribine, X-339 (Xcovery, West Palm Beach, Fla.), XL-499 (Evotech, Hamburg, Germany), pharmaceutically acceptable salts thereof, and combinations thereof.

52. The kit according to claim 40, wherein administration of the first and second anti-cancer agents provides a synergistic effect compared to administration of either anti-cancer agent alone.

53. A pharmaceutical composition for treating or ameliorating the effects of a cancer in a subject in need thereof, the pharmaceutical composition comprising a pharmaceutically acceptable diluent or carrier and an effective amount of (i) a first anti-cancer agent, which is BVD-523 or a pharmaceutically acceptable salt thereof and (ii) a second anti-cancer agent, which is a type 1 MEK inhibitor or a pharmaceutically acceptable salt thereof, wherein administration of the first and second anti-cancer agents provides a synergistic effect compared to administration of either anti-cancer agent alone.

54. The pharmaceutical composition according to claim 53, wherein the subject is a mammal.

55. The pharmaceutical composition according to claim 54, wherein the mammal is selected from the group consisting of humans, primates, farm animals, and domestic animals.

56. The pharmaceutical composition according to claim 54, wherein the mammal is a human.

57. The pharmaceutical composition according to claim 53, wherein the type 1 MEK inhibitor is selected from the group consisting of bentamapimod (Merck KGaA), L783277 (Merck), RO092210 (Roche), pharmaceutically acceptable salts thereof, and combinations thereof.

58. The pharmaceutical composition according to claim 53, wherein the type 1 MEK inhibitor is RO092210 (Roche) or a pharmaceutically acceptable salt thereof.

59. The pharmaceutical composition according to claim 53, wherein the subject with cancer has a somatic RAS mutation or BRAF mutation

60. The pharmaceutical composition according to claim 53, wherein the cancer is selected from the group consisting of a cancer of the large intestine, breast cancer, pancreatic cancer, skin cancer, endometrial cancer, neuroblastoma, leukemia, lymphoma, liver cancer, lung cancer, testicular cancer, and thyroid cancer.

61. The pharmaceutical composition according to claim 53, wherein the cancer is melanoma.

62. The pharmaceutical composition according to claim 53 further comprising at least one additional therapeutic agent selected from the group consisting of an antibody or fragment thereof, a cytotoxic agent, a toxin, a radionuclide, an immunomodulator, a photoactive therapeutic agent, a radiosensitizing agent, a hormone, an anti-angiogenesis agent, and combinations thereof.

63. The pharmaceutical composition according to claim 62, wherein the additional therapeutic agent is an inhibitor of the PI3K/Akt pathway.

64. The pharmaceutical composition according to claim 63, wherein the inhibitor of the PI3K/Akt pathway is selected from the group consisting of A-674563 (CAS #552325-73-2), AGL 2263, AMG-319 (Amgen, Thousand Oaks, Calif.), AS-041164 (5-benzo[1,3]dioxol-5-ylmethylene-thiazolidine-2,4-dione), AS-604850 (5-(2,2-Difluoro-benzo[1,3]dioxol-5-ylmethylene)-thiazolidine-2,4-dione), AS-605240 (5-quinoxilin-6-methylene-1,3-thiazolidine-2,4-dione), AT7867 (CAS #857531-00-1), benzimidazole series, Genentech (Roche Holdings Inc., South San Francisco, Calif.), BML-257 (CAS #32387-96-5), CAL-120 (Gilead Sciences, Foster City, Calif.), CAL-129 (Gilead Sciences), CAL-130 (Gilead Sciences), CAL-253 (Gilead Sciences), CAL-263 (Gilead Sciences), CAS #612847-09-3, CAS #681281-88-9, CAS #75747-14-7, CAS #925681-41-0, CAS #98510-80-6, CCT128930 (CAS #885499-61-6), CH5132799 (CAS #1007207-67-1), CHR-4432 (Chroma Therapeutics, Ltd., Abingdon, UK), FPA 124 (CAS #902779-59-3), GS-1101 (CAL-101) (Gilead Sciences), GSK 690693 (CAS #937174-76-0), H-89 (CAS #127243-85-0), Honokiol, IC87114 (Gilead Science), IPI-145 (Intellikine Inc.), KAR-4139 (Karus Therapeutics, Chilworth, UK), KAR-4141 (Karus Therapeutics), KIN-1 (Karus Therapeutics), KT 5720 (CAS #108068-98-0), Miltefosine, MK-2206 dihydrochloride (CAS #1032350-13-2), ML-9 (CAS #105637-50-1), Naltrindole Hydrochloride, OXY-111A (NormOxys Inc., Brighton, Mass.), perifosine, PHT-427 (CAS #1191951-57-1), PI3 kinase delta inhibitor, Merck KGaA (Merck & Co., Whitehouse Station, N.J.), PI3 kinase delta inhibitors, Genentech (Roche Holdings Inc.), PI3 kinase delta inhibitors, Incozen (Incozen Therapeutics, Pvt. Ltd., Hyderabad, India), PI3 kinase delta inhibitors-2, Incozen (Incozen Therapeutics), PI3 kinase inhibitor, Roche-4 (Roche Holdings Inc.), PI3 kinase inhibitors, Roche (Roche Holdings Inc.), PI3 kinase inhibitors, Roche-5 (Roche Holdings Inc.), PI3-alpha/delta inhibitors, Pathway Therapeutics (Pathway Therapeutics Ltd., South San Francisco, Calif.), PI3-delta inhibitors, Cellzome (Cellzome AG, Heidelberg, Germany), PI3-delta inhibitors, Intellikine (Intellikine Inc., La Jolla, Calif.), PI3-delta inhibitors, Pathway Therapeutics-1 (Pathway Therapeutics Ltd.), PI3-delta inhibitors, Pathway Therapeutics-2 (Pathway Therapeutics Ltd.), PI3-delta/gamma inhibitors, Cellzome (Cellzome AG), PI3-delta/gamma inhibitors, Cellzome (Cellzome AG), PI3-delta/gamma inhibitors, Intellikine (Intellikine Inc.), PI3-delta/gamma inhibitors, Intellikine (Intellikine Inc.), PI3-delta/gamma inhibitors, Pathway Therapeutics (Pathway Therapeutics Ltd.), PI3-delta/gamma inhibitors, Pathway Therapeutics (Pathway Therapeutics Ltd.), PI3-gamma inhibitor Evotec (Evotec), PI3-gamma inhibitor, Cellzome (Cellzome AG), PI3-gamma inhibitors, Pathway Therapeutics (Pathway Therapeutics Ltd.), PI3K delta/gamma inhibitors, Intellikine-1 (Intellikine Inc.), PI3K delta/gamma inhibitors, Intellikine-1 (Intellikine Inc.), pictilisib (Roche Holdings Inc.), PIK-90 (CAS #677338-12-4), SC-103980 (Pfizer, New York, N.Y.), SF-1126 (Semafore Pharmaceuticals, Indianapolis, Ind.), SH-5, SH-6, Tetrahydro Curcumin, TG100-115 (Targegen Inc., San Diego, Calif.), Triciribine, X-339 (Xcovery, West Palm Beach, Fla.), XL-499 (Evotech, Hamburg, Germany), pharmaceutically acceptable salts thereof, and combinations thereof.

65. The pharmaceutical composition according to claim 53, which is in a unit dosage form comprising both anti-cancer agents.

66. The pharmaceutical composition according to claim 53 in which the first anti-cancer agent is in a first unit dosage form and the second anti-cancer agent is in a second unit dosage form, separate from the first.

67. The pharmaceutical composition according to claim 53, wherein the first and second anti-cancer agents are co-administered to the subject.

68. The pharmaceutical composition according to claim 53, wherein the first and second anti-cancer agents are administered to the subject serially.

69. The pharmaceutical composition according to claim 68, wherein the first anti-cancer agent is administered to the subject before the second anti-cancer agent.

70. The pharmaceutical composition according to claim 68, wherein the second anti-cancer agent is administered to the subject before the first anti-cancer agent.
